(19) United States
(12) Patent Application Publication
(10) Pub. No.: US 2007/0287699 A1
(43) Pub. Date: Dec. 13, 2007

(54) ANTIVIRAL AGENTS

(75) Inventors: Dange V. Kumar, Salt Lake City, UT (US); Roopa Rai, San Carlos, CA (US); Wendy Young, San Mateo, CA (US); Huiyong Hu, San Diego, CA (US); Jennifer R. Riggs, Cardiff by the Sea, CA (US); Tony Loc Ton, Fremont, CA (US); Michael J. Green, Half Moon Bay, CA (US); Barry P. Hart, Palo Alto, CA (US); Kenneth A. Brameld, San Mateo, CA (US); Jeffrey M. Dener, Millbrae, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ViroBay, Inc., Menlo Park, CA (US)

(21) Appl. No.: 11/742,461

(22) Filed: Apr. 30, 2007

Related U.S. Application Data
(60) Provisional application No. 60/796,943, filed on May 1, 2006.

(51) Int. Cl. A61K 31/4709; A61K 31/496; A61K 31/5355; A61K 31/541; A61K 31/551; A61P 31/14; C07D 215/233; C07D 243/08; C07D 279/12; C07D 401/04; C07D 413/04; C07D 455/02; C07D 471/04

(52) U.S. Cl. 514/218; 514/228.2; 514/234.5; 514/235.2; 514/253.08; 514/300; 514/306; 514/312; 540/575; 544/128; 544/238; 544/58.2; 546/123; 546/138; 546/156

(57) ABSTRACT

Compounds are provided having utility for the treatment of viral infections, particularly HCV.

### CROSS-REFERENCES TO RELATED APPLICATIONS

This application claims the benefit of U.S. Ser. No. 60/796,943 filed May 1, 2006, the content of which is incorporated herein by reference in its entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Hepatitis C virus (HCV) is a (+)-sense single-stranded RNA virus that is a major cause of non-A, non-B hepatitis worldwide. A large percentage of people infected with HCV develop chronic liver disease, the so called chronic hepatitis C which in turn makes them at high risk for developing serious liver disease such as liver cirrhosis, hepatocellular carcinoma and terminal liver disease leading to death. Currently, hepatitis C is treated with either interferon alone or in combination with Ribavirin or with pegylated forms of interferon such as PEG-Intron® and Pegasys®. These therapies, however, induce severe side effects such as retinopathy, thyroiditis, acute pancreatitis, depression. Therefore, there is a need for safe, oral drugs for the treatment of hepatitis C. The present invention fulfils this and related needs.

### BRIEF SUMMARY OF THE INVENTION

The present invention is directed to compounds that are antiviral agents. Specifically the compounds of the present invention inhibit replication of HCV and are therefore useful in treating hepatitis C. The present invention is also directed to pharmaceutical compositions comprising these compounds and processes for preparing them.

An aspect of the present invention is a compound of Formula I, II or III: 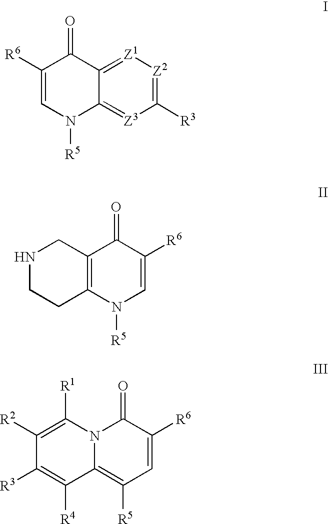
in which:

Z¹, Z² and Z³ are CR¹, N and N, respectively, or

Z¹, Z² and Z³ are N, CR², and N, respectively, or

Z¹, Z² and Z³ are CR¹, N and CR⁴, respectively, or

Z¹, Z² and Z³ are CR¹, CR² and N, respectively, or

Z¹, Z² and Z³ are CR¹, CR² and CR⁴, respectively;

R¹ is halo, —R⁸ or —OR⁵, where R⁸ is hydrogen, alkyl or halo-alkyl, or R¹ together with R² form methylenedioxy or ethylenedioxy;

R² is as defined above or is hydrogen, halo, alkyl, halo-alkyl, —OR⁹, —X²COOR⁹, —NR¹⁰C(O)NR⁹R¹⁰ or —X²C(O)NR⁹R¹⁰, where X² is a bond or alkylene, R⁹ is hydrogen, alkyl, halo-alkyl or heterocycloalkylalkyl and R¹⁰ is hydrogen or alkyl, or R² and R³ together form methylenedioxy, ethylenedioxy, 1,3-diazaprop-1-enylene, 1,3-diazaprop-2-enylene, 1-azaprop-2-enylene or 3-azaprop-1-enylene;

R³ is as defined above or is halo, —X³R¹¹, —X³OR¹², —X³C(O)R¹³, —X³C(O)OR¹², —X³OC(O)R¹³, —X³C(O)OR¹², —X³NR¹⁴R¹², —X³NR¹⁵R¹⁶, —X³OR¹⁵, —X³SR¹⁵, —X³NR¹⁴C(O)R¹³—X³C(O)NR¹²R¹⁴, —X³S(O)₀₋₂R¹², —X³S(O)₂NR¹²R¹⁴, —X³NR¹⁴C(O)NR¹²R¹⁴ or —X³NR¹⁴C(O)OR¹², wherein X³ is a bond or alkylene, R¹² is hydrogen, alkyl or —X⁴R¹¹, R¹³ is alkyl or —X⁴R¹¹, R¹⁴ is hydrogen or alkyl and R¹⁵ is hydrogen, alkyl or —X⁵R¹⁷ and R¹⁶ is —X⁵R¹⁷, wherein X⁴ is a bond or alkylene, X⁵ is lower alkylene, R¹¹ is aryl, heteroaryl, cycloalkyl or heterocycloalkyl and R¹⁷ is —NR¹⁸R¹⁹, —C(O)OR¹⁸, —C(O)NR¹⁸R¹⁸, —OR¹⁹ or —SR¹⁹, wherein R¹⁸ is hydrogen or alkyl and R¹⁹ is hydrogen, alkyl or —NR²⁰R²¹, —OR²⁰ or —SR²⁰, wherein R²⁰ and R²¹ independently are hydrogen or alkyl, or R³ and R⁴ together form methylenedioxy or ethylenedioxy, wherein within R¹¹ any aromatic or alicyclic ring may be substituted with one R²² and within R³ any aliphatic moiety may be substituted with one to three halo, any alicyclic ring may be substituted with one to three R²³ and any aromatic ring may be substituted with one to three R²⁴;

R⁴ is as defined above or is halo, —R²⁵ or —OR²⁵, where R²⁵ is hydrogen, alkyl or halo-alkyl, or R⁴ together with R⁵ form ethylene, trimethylene or heteroalkylene which may be substituted with one R²⁶;

R⁵ is as defined above or is alkyl or —X⁷R²⁷ where X⁷ is —NH—, lower alkylene or cycloprop-1,1-ylene and R²⁷ is cycloalkyl, heterocycloalkyl, heteroaryl or aryl, where within R⁵ any aliphatic moiety may be substituted with one to three halo, any alicyclic ring may be substituted with one to three R²⁸ and any aromatic ring may be substituted with one to three R²⁹;

R⁶ is selected from: 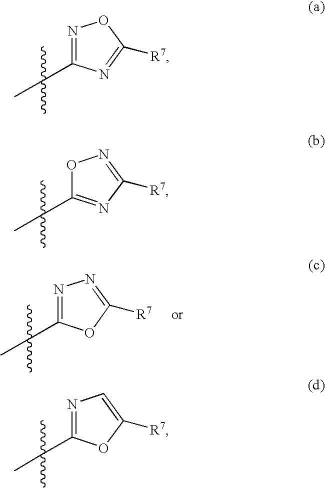
wherein R⁷ is isopropyl, tert-butyl, pentyl or —X⁸R³⁰, where X⁸ is methylene, —C(CH₃)₂—, —C(CH₂OH)₂—, cycloprop-1,1-ylene, or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring and wherein within R⁷ any aliphatic moiety may be substituted with one to three halo;

R²² is aryl, heteroaryl, cycloalkyl or heterocycloalkyl;

R²³ and R²⁸ at each occurrence independently are alkyl, halo-alkyl, hydroxy, alkoxy, acetyl or oxo;

R²⁴ and R²⁹ at each occurrence independently are alkyl, halo-alkyl, hydroxy, alkoxy or acetyl;

R²⁶ is —X⁷R²⁷ where X⁷ and R²⁷ are as defined above;

R³³ at each occurrence independently are methyl or halo; and

R³⁴ is lower alkyl, —CH₂COOR³⁵, —CH₂CONHR³⁵ or —CH₂NH₂ wherein R³⁵ is hydrogen or alkyl; or a pharmaceutically acceptable salt thereof.

An aspect of the present invention is a compound of Formula IV: 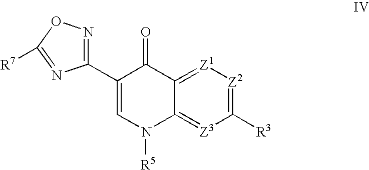
in which:

Z¹, Z² and Z³ are CR¹, N and N, respectively, or

Z¹, Z² and Z³ are N, CR² and N, respectively, or

Z¹, Z² and Z³ are CR¹, N and CR⁴, respectively, or

Z¹, Z² and Z³ are CR¹, CR² and N, respectively, or

Z¹, Z² and Z³ are CR¹, CR² and CR⁴, respectively;

R¹ is halo, —R⁸ or —OR⁸, where R⁸ is hydrogen, alkyl or halo-alkyl, or R¹ together with R² form methylenedioxy or ethylenedioxy;

R² is as defined above or is hydrogen, halo, alkyl, halo-alkyl, —OR⁹, —X²COOR⁹, —NR¹⁰C(O)NR⁹R¹⁰ or —X²C(O)NR⁹R¹⁰, where X² is a bond or alkylene, R⁹ is hydrogen, alkyl, halo-alkyl or heterocycloalkylalkyl and R¹⁰ is hydrogen or alkyl, or R² and R³ together form methylenedioxy, ethylenedioxy, 1,3-diazaprop-1-enylene, 1,3-diazaprop-2-enylene, 1-azaprop-2-enylene or 3-azaprop-1-enylene;

R³ is as defined above or is halo, —X³R¹¹, —X³OR¹², —X³C(O)R¹³, —X³C(O)OR¹², —X³OC(O)R¹³, —X³C(O)OR¹², —X³NR¹⁴R¹², —X³NR¹⁵R¹⁶, —X³OR¹⁵, —X³SR¹⁵, —X³NR¹⁴C(O)R¹³, —X³C(O)NR¹²R¹⁴, —X³S(O)₀₋₂R¹², —X³S(O)₂NR¹²R¹⁴, —X³NR¹⁴C(O)NR¹²R¹⁴ or —X³NR¹⁴C(O)OR¹², wherein X³ is a bond or alkylene, R¹² is hydrogen, alkyl or —X⁴R¹¹, R¹³ is alkyl or —X⁴R¹¹, R¹⁴ is hydrogen or alkyl and R¹⁵ is hydrogen, alkyl or —X⁵R¹⁷ and R¹⁶ is —X⁵R¹⁷, wherein X⁴ is a bond or alkylene, X⁵ is lower alkylene, R¹¹ is aryl, heteroaryl, cycloalkyl or heterocycloalkyl and R¹⁷ is —NR¹⁸R¹⁹, —C(O)OR¹⁸, —C(O)NR¹⁸R¹⁸, —OR¹⁹ or —SR¹⁹, wherein R¹⁸ is hydrogen or alkyl and R¹⁹ is hydrogen, alkyl or —NR²⁰R²¹, —OR²⁰ or —SR²⁰, wherein R²⁰ and R²¹ independently are hydrogen or alkyl, or R³ and R⁴ together form methylenedioxy or ethylenedioxy, wherein within R¹¹ any aromatic or alicyclic ring may be substituted with one R²² and within R³ any aliphatic moiety may be substituted with one to three halo, any alicyclic ring may be substituted with one to three R²³ and any aromatic ring may be substituted with one to three R²⁴;

R⁴ is as defined above or is halo, —R²⁵ or —OR²⁵, where R²⁵ is hydrogen, alkyl or halo-alkyl, or R⁴ together with R⁵ form ethylene, trimethylene or heteroalkylene which may be substituted with one R²⁶;

R⁵ is as defined above or is alkyl or —X⁷R²⁷ where X⁷ is —NH—, lower alkylene or cycloprop-1,1-ylene and R²⁷ is cycloalkyl, heterocycloalkyl, heteroaryl or aryl, where within R⁵ any aliphatic moiety may be substituted with one to three halo, any alicyclic ring may be substituted with one to three R²⁸ and any aromatic ring may be substituted with one to three R²⁹;

R⁷ is isopropyl, tert-butyl, pentyl or —X⁸R³⁰, where X⁸ is methylene, —C(CH₂OH)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring and wherein within R⁷ any aliphatic moiety may be substituted with one to three halo;

R²² is aryl, heteroaryl, cycloalkyl or heterocycloalkyl;

R²³ and R²⁸ at each occurrence independently are alkyl, halo-alkyl, hydroxy, alkoxy, acetyl or oxo;

R²⁴ and R²⁹ at each occurrence independently are alkyl, halo-alkyl, hydroxy, alkoxy or acetyl;

R²⁶ is —X⁷R²⁷ where X⁷ and R²⁷ are as defined above;

R³³ at each occurrence independently are methyl or halo; and

R³⁴ is lower alkyl, —CH₂COOR³⁵, —CH₂CONHR³⁵ or —CH₂NH₂ wherein R³⁵ is hydrogen or alkyl; or a pharmaceutically acceptable salt thereof.

In another aspect, the present invention provides a pharmaceutical composition comprising a compound of Formula I, II, III or IV or a pharmaceutically acceptable salt thereof in admixture with one or more suitable excipients.

In yet another aspect, the present invention provides a method for treating hepatitis C in an animal which method comprises administering to the animal a pharmaceutical composition comprising a therapeutically effective amount of a compound of Formula I, II, III or IV or a pharmaceutically acceptable salt thereof in admixture with one or more suitable excipients.

In still another aspect, the present invention provides processes for preparing compounds of Formula I, II, III or IV.

### BRIEF DESCRIPTION OF THE DRAWINGS

FIGS. 1-27 provide reaction schemes described in detail below.

### DETAILED DESCRIPTION OF THE INVENTION

Definitions:

Unless otherwise stated, the following terms used in the specification and claims are defined for the purposes of this Application and have the following meanings.

“Alicyclic” means a moiety characterized by arrangement of the atoms in a closed non-aromatic ring structure. Alicyclic includes saturated or partially unsaturated monocyclic rings or fused ring systems or partially unsaturated rings fused to an aromatic ring. Alicyclic includes cycloalkyl, cycloalkylene, heterocycloalkyl, heterocycloalkylene, the ring moieties formed by alkylene, e.g., ethylene or trimethylene, heteroalkylene, methylenedioxy and ethylenedioxy and any partially unsaturated ring as part of a fused aryl or heteroaryl radical, e.g., cyclohexyl, morpholinyl, cycloprop-1,1-ylene, piperid-4,4-ylene, the partially unsaturated ring as part of 1,2,3,4-tetrahydro-naphthyl or 5,6,7,8-tetrahydro-quinolyl, and the like.

“Aliphatic” means a moiety characterized by arrangement of the carbon atoms in open chains. Aliphatic includes alkyl, lower alkyl, alkylene, lower alkylene and any alkyl as part of a more complex moiety, e.g., ethyl, ethylene, pentyl, pentylene, hexyl, hexylene, hexenyl, hexenylene, hexynyl, hexynylene, 2-methylpentyl, 2-methylpentylene, the alkyl as part of arylalkyl, and the like.

“Alkyl,” unless indicated otherwise, by itself means a straight or branched, saturated or unsaturated aliphatic radical containing one to eight carbon atoms, unless otherwise indicated, e.g., alkyl includes methyl, ethyl, propyl, isopropyl, butyl, sec-butyl, isobutyl, tert-butyl, and the like, and as a part of a more complex moiety, e.g., halo-alkyl or heterocycloalkyl-alkyl, has the meaning of alkylene.

“Alkylene,” unless indicated otherwise, means a straight or branched, saturated or unsaturated aliphatic, divalent radical having the one to eight carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Methylene,” means the divalent radical having one carbon atom substituted with two hydrogens (—CH₂—)

“Alkoxy” means the radical —OR where R is alkyl, e.g., methoxy, ethoxy, and the like.

“Amino” means the radical —NH₂.

“Animal” means humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring, all atoms in the ring are sp² hybridized and the total number of pi electrons is equal to 4n+2. Aromatic includes any unsaturated ring as part of an aryl, heteroaryl, arylene or heteroarylene radical, e.g., phenyl, phenylene, pyridinyl, pyridinylene, the unsaturated ring as part of 1,2,3,4-tetrahydro-naphthalene, and the like.

“Aryl” by itself or as part of a more complex moiety, e.g., arylalkyl, means a monovalent, monocyclic or fused bicyclic hydrocarbon radical of 6 to 12 ring atoms, wherein the ring as part of a monocyclic radical ring is aromatic and wherein at least one of the fused rings as part of a bicyclic radical is aromatic. Fused bicyclic hydrocarbon radical includes bridged ring systems. Unless otherwise stated, the valency of the group may be located on any atom of any ring within the radical, valency rules permitting. More specifically the term aryl includes, but is not limited to, phenyl, naphthyl, indanyl (including, for example, indan-5-yl, or indan-2-yl, and the like) or tetrahydronaphthyl (including, for example, tetrahydronapth-1-yl, or tetrahydronapth-2-yl, and the like), and the like.

“Carbamoyl” means the radical —C(O)NH₂.

“Carboxy” means the radical —C(O)OH.

“Cycloalkyl” by itself or as part of a more complex moiety, e.g., cycloalkylalkyl, means a saturated or partially unsaturated monovalent monocyclic radical of 3 to 8 ring atoms or fused bicyclic radical of 5 to 12 ring carbon atoms. One or two ring carbon atoms may be replaced by a —C(O)—, —C(S)— or —C(═NR)— group, wherein R is hydrogen or alkyl. Fused bicyclic radical includes bridged ring systems. Unless otherwise stated, the valency may be located on any atom of any ring within the radical, valency rules permitting. More specifically the term cycloalkyl includes, but is not limited to, cyclopentyl, cyclopentenyl, cyclohexyl, 2-oxocyclohexyl, thioxocyclohexyl, and the like.

“Cycloprop-1,1-ylene” means a divalent cyclopropane radical wherein both valencies are on the same ring carbon. For example, the radical —X⁷R²⁷, wherein X⁷ is cycloprop-1,1-ylene, has the following structure: 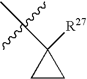
wherein R²⁷ is defined as in the Summary of the Invention.

“Disease” means any unhealthy condition of an animal or part thereof and includes an unhealthy condition that may be caused by, or incident to, medical or veterinary therapy applied to that animal, i.e., the “side effects” of such therapy.

“Ethylenedioxy” means a divalent radical —OCH₂CH₂O—.

“Halo” refers to fluoro, chloro, bromo or iodo.

“Halo-alkyl” means alkyl as defined above substituted by one or more, preferably one to seven, halo atoms. Halo-alkyl includes monohaloalkyl, dihaloalkyl, trihaloalkyl, perhaloalkyl and the like, e.g., chloromethyl, dichloromethyl, difluoromethyl, trifluoromethyl, 2,2,2-trifluoroethyl, perfluoroethyl, 2,2,2-trifluoro-1,1-dichloroethyl, and the like.

“Heteroalkylene” means a straight, saturated or unsaturated aliphatic, divalent radical having two to three atoms in which one or more of the atoms are heteroatoms independently selected from —O—, —S(O)ₙ— (n is 0, 1 or 2), —N—, —N(R)—, —Si(R′)₂—, —P(═S)(R′)— and —P(═O)(R′)—, wherein R is hydrogen or alkyl and R′ is alkyl, and any remaining ring atoms being carbon atoms. More specifically, the term heteroalkylene includes —NHCH₂—, —CH₂OCH₂—, and the like.

“Heteroaryl” by itself or as part of a more complex moiety, e.g., heteroarylalkyl, means a monocyclic, fused bicyclic, or fused tricyclic, monovalent radical of 5 to 14 ring atoms containing one or more, preferably one, two, three, or four ring heteroatoms independently selected from —O—, —S(O)ₙ— (n is 0, 1, or 2), —N—, —N(R)—, —Si(alkyl)₂-, —P(═S)(R′)— and —P(═O)(R′)—, wherein R is hydrogen or alkyl and R′ is alkyl, and the remaining ring atoms being carbon atoms, wherein the ring as part of a monocyclic radical is aromatic and wherein at least one of the fused rings as part of a bicyclic or tricyclic radical is aromatic. One or two ring carbon atoms of any nonaromatic rings as part of a bicyclic or tricyclic radical may be replaced by a —C(O)—, —C(S)—, or —C(═NR″)— group, wherein R″ is hydrogen or alkyl. Fused bicyclic radical includes bridged ring systems. Unless stated otherwise, the valency may be located on any atom of any ring of the heteroaryl radical, valency rules permitting. In particular, when the point of valency is located on a nitrogen or phosphorous atom, R or R′, respectively, is absent. More specifically, the term heteroaryl includes, but is not limited to, 1,2,4-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), pyrazinyl, pyrimidinyl, pyridazinyl, oxazolyl, isooxazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, methylenedioxyphenyl (including, for example, methylenedioxyphen-5-yl), and N-oxide or protected derivatives thereof.

“Heterocycloalkyl” by itself or as part of a more complex moiety, e.g., heterocycloalkylalkyl, means a saturated or partially unsaturated monovalent monocyclic radical of 3 to 8 ring atoms or fused bicyclic radical of 5 to 12 ring atoms in which one or more, preferably one, two, three or four of the ring atoms are heteroatoms independently selected from —O—, —S(O)ₙ— (n is 0, 1 or 2), —N—, —N(R)—, —Si(R′)₂—, —P(═S)(R′)— and —P(═O)(R′—, wherein R is hydrogen or alkyl and R′ is alkyl, the remaining ring atoms being carbon atoms. One or two ring carbon atoms may be replaced by a —C(O)—, —C(S)— or —C(═NR″)— group, wherein R″ is hydrogen or alkyl. Fused bicyclic radical includes bridged ring systems. Unless otherwise stated, the valency may be located on any atom of any ring within the radical, valency rules permitting. In particular, when the point of valency is located on a nitrogen or phosphorous atom, R or R′, respectively, is absent. More specifically the term heterocycloalkyl includes, but is not limited to, pyrrolidinyl, 2,5-dihydro-1H-pyrrolyl, piperidinyl, morpholinyl, piperazinyl, tetrahydropyranyl, 2-oxopiperidinyl, and thiomorpholinyl, and N-oxide or protected derivatives thereof.

“Hydroxy” means —OH.

“Isomers” mean compounds of Formula I, II, III and IV having identical molecular formulae but differ in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center that has two enantiomeric forms of opposite chirality is termed a “racemic mixture.” A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry,” 4th edition, March, Jerry, John Wiley & Sons, New York, 1992). It is understood that the names and illustration used in this application to describe compounds of Formula I, II, III and IV are meant to be encompassed all possible stereoisomers.

“Methylenedioxy” means a divalent radical —OCH₂O—.

“Lower alkyl” means a straight or branched, saturated or unsaturated aliphatic radical containing one to four carbon atoms, unless otherwise indicated, e.g., alkyl includes methyl, ethyl, propyl, isopropyl, butyl, sec-butyl, isobutyl, tert-butyl, and the like.

“Lower alkylene” means a straight or branched, saturated or unsaturated aliphatic, divalent radical having the one to three carbon atoms, e.g., lower alkylene includes methylene (—CH₂—), ethylene (—CH₂CH₂—), trimethylene (—CH₂CH₂CH₂—), 1-methylethylene (—CH(CH₃)CH₂)—, and the like.

“Optional,” “optionally” or “may be” means that the subsequently described event or circumstance may or may not occur, and that the description includes instances where the event or circumstance occurs and instances in which it does not. For example, the phrase “within R³ any alkylene and alkyl may be substituted with one to three halo” means that the alkylene and alkyl group may or may not be substituted with halo in order to fall within the scope of the invention.

The present invention also includes N-oxide derivatives of a compound of Formula I, II, III and IV. N-oxide derivative means a compound in which a nitrogen atom is in an oxidized state (i.e., N→O) e.g., pyridine N-oxide, and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula I, II, III or IV which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxy-ethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of Formula I, II, III or IV. Prodrug means a compound that is convertible in vivo by metabolic means (e.g. by hydrolysis) to a compound of Formula I, II, III or IV. For example, an ester of a compound of Formula I, II, III or IV containing a hydroxy group may be convertible by hydrolysis in vivo to the parent molecule. Alternatively an ester of a compound of Formula I, II, III or IV containing a carboxy group may be convertible by hydrolysis in vivo to the parent molecule. Suitable esters of compounds of Formula I, II, III or IV containing a hydroxy group, are for example acetates, citrates, lactates, tartrates, malonates, oxalates, salicylates, propionates, succinates, fumarates, maleates, methylene-bis-βb-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of Formula I, II, III or IV containing a carboxy group, are for example those described by Leinweber, F. J. Drug Metab. Res., 1987, 18, page 379. An especially useful class of esters of compounds of Formula I, II, III or IV containing a hydroxy group, may be formed from acid moieties selected from those described by Bundgaard et al., J. Med. Chem., 1989, 32, pp 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates.

“Protected derivatives” means derivatives of compounds of Formula I, II, III or IV in which a reactive site or sites are blocked with protecting groups. Protected derivatives of compounds of Formula I, II, III, or IV are useful in the preparation of compounds of Formula I, II, III or IV or in themselves may be active cathepsin S inhibitors. A comprehensive list of suitable protecting groups can be found in T. W. Greene, Protective Groups in Organic Synthesis, 3rd edition, John Wiley & Sons, Inc. 1999.

“Sulfamoyl” means —S(O)₂NH₂.

“Therapeutically effective amount” means that amount which, when administered to an animal for treating a disease, is sufficient to effect such treatment for the disease.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

“Ureido” means the radical —NHC(O)NH₂.

The radical names defined in this application can be combined with other radical names to form more complex meanings that will be commensurate with traditional rules of nomenclature. For example, “amino” when used as part of “alkylamino” or “dialkylamino” means —NR′R″ where R′ and/or R″ is alkyl, “amino” as part of “aminoalkyl” means the radical —XNH₂ where X is alkylene, “alkyoxy” as part of “alkoxycarbonylalkyl” means the radical —XC(O)OR where X is alkylene and R is alkyl, and the like.

The compounds of Formulae I, II and III and the intermediates and starting materials used in their preparation are named in accordance with IUPAC rules of nomenclature in which the characteristic groups have decreasing priority for citation as the principle group as follows: acids, esters, amides, etc. Alternatively, the compounds are named by AutoNom 4.0 (Beilstein Information Systems, Inc.) or by ChemDraw Ultra, Version 10.0. For example, a compound of Formula I having the following structure: 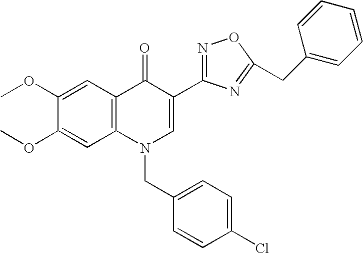
is named 3-(5-benzyl-[1,2,4]oxadiazol-3-yl)-1-(4-chloro-benzyl)-6,7-dimethoxy-1H-quinolin-4-one.

### EMBODIMENTS OF THE INVENTION

### Compounds

Within Formulae I, II, III and IV as provided in the Summary of the Invention are a number of selected aspects and embodiments of the invention.

In one selected aspect, the present invention provides compounds of Formula I. In some preferred embodiments, Z¹, Z² and Z³ are CR¹, CR² and CR⁴, respectively. In further preferred embodiments, R⁵ is —X⁷R²⁷ wherein X⁷ is lower alkylene and R²⁷ is phenyl, and wherein the phenyl is optionally substituted with one to three R²⁹. In still further preferred embodiments, R⁶ has subformula (a). In yet further preferred embodiments, R⁷ is —X⁸R³⁰, where X⁸ is —CH₂—, —C(CH₃)₂—, cycloprop-1,1-ylene, —C(CH₂OH)₂—, or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring. In still further preferred embodiments, R⁷ is —X⁸R³⁰, where X⁸ is —C(CH₃)₂—, —C(CH₂OH)₂—, or cycloprop-1,1-ylene, and R³⁰ is phenyl, optionally substituted with one to two R³³ at any position on the ring, or with one R³⁴ at the 2- or 3-position on the ring. In some particularly preferred embodiments, R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

In some related embodiments, the compounds have formula I in which Z¹, Z² and Z³ are CR¹, CR² and CR⁴, respectively; R⁵ is —X⁷R²⁷ wherein X⁷ is lower alkylene and R²⁷ is phenyl, and wherein the phenyl is optionally substituted with one to three R²⁹; R⁶ is subformula (b); and R⁷ is —X⁸R³⁰, where X⁸ is —CH₂—, —C(CH₃)₂—, cycloprop-1,1-ylene, —C(CH₂OH)₂—, or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring. Preferred compounds in this group of embodiments are those in which R⁷ is —X⁸R³⁰, where X⁸ is —C(CH₃)₂—, —C(CH₂OH)₂—, or cycloprop-1,1-ylene, and R³⁰ is phenyl, optionally substituted with one to two R³³ at any position on the ring, or with one R³⁴ at the 2- or 3-position on the ring. Still further preferred are those compounds in which R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

In some related embodiments, the compounds have formula I in which Z¹, Z² and Z³ are CR¹, CR² and CR⁴, respectively; R⁵ is —X⁷R²⁷ wherein X⁷ is lower alkylene and R²⁷ is phenyl, and wherein the phenyl is optionally substituted with one to three R²⁹; R⁶ is subformula (c); and R⁷ is —X⁸R³⁰, where X⁸ is —CH₂—, —C(CH₃)₂—, —C(CH₂OH)₂—, cycloprop-1,1-ylene or —CHR³¹—, wherein R³ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring. Preferred compounds in this group of embodiments are those in which R⁷ is —X⁸R³⁰, where X⁸ is —C(CH₃)₂—, —C(CH₂OH)₂—, or cycloprop-1,1-ylene, and R³⁰ is phenyl, optionally substituted with one to two R³³ at any position on the ring, or with one R³⁴ at the 2- or 3-position on the ring. Still further preferred are those compounds in which R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

In some related embodiments, the compounds have formula I in which Z¹, Z² and Z³ are CR¹, CR² and CR⁴, respectively; R⁵ is —X⁷R²⁷ wherein X⁷ is lower alkylene and R²⁷ is phenyl, and wherein the phenyl is optionally substituted with one to three R²⁹; R⁶ is subformula (d); and R⁷ is —X⁸R³⁰, where X⁸ is —CH₂—, —C(CH₃)₂—, —C(CH₂OH)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring. Preferred compounds in this group of embodiments are those in which R⁷ is —X⁸R³⁰, where X⁸ is —C(CH₃)₂—, —C(CH₂OH)₂—, or cycloprop-1,1-ylene, and R³⁰ is phenyl, optionally substituted with one to two R³³ at any position on the ring, or with one R³⁴ at the 2- or 3-position on the ring. Still further preferred are those compounds in which R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

In a related aspect, the present invention provides compounds of Formula IV: 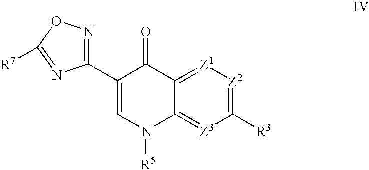
in which:

Z¹, Z² and Z³ are CR¹, N and N, respectively, or

Z¹, Z² and Z³ are N, CR² and N, respectively, or

Z¹, Z² and Z³ are CR¹, N and CR⁴, respectively, or

Z¹, Z² and Z³ are CR¹, CR² and N, respectively, or

Z¹, Z² and Z³ are CR¹, CR² and CR⁴, respectively;

R¹ is halo, —R⁸ or —OR⁸, where R⁸ is hydrogen, alkyl or halo-alkyl, or R¹ together with R² form methylenedioxy or ethylenedioxy;

R² is as defined above or is hydrogen, halo, alkyl, halo-alkyl, —OR⁹, —X²COOR⁹, —NR¹⁰C(O)NR⁹R¹⁰ or —X²C(O)NR⁹R¹⁰, where X² is a bond or alkylene, R⁹ is hydrogen, alkyl, halo-alkyl or heterocycloalkylalkyl and R¹⁰ is hydrogen or alkyl, or R² and R³ together form methylenedioxy, ethylenedioxy, 1,3-diazaprop-1-enylene, 1,3-diazaprop-2-enylene, 1-azaprop-2-enylene or 3-azaprop-1-enylene;

R³ is as defined above or is halo, —X³R¹¹, —X³OR¹², —X³C(O)R¹³, —X³C(O)OR¹², —X³OC(O)R¹³, X³C(O)OR¹², —X³NR¹⁴R¹², —X³NR¹⁵R¹⁶, —X³OR¹⁵, —X³SR¹⁵, —X³NR¹⁴C(O)R¹³, —X³C(O)NR¹²R¹⁴, —X³S(O)₀₋₂R¹², —X³S(O)₂NR¹²R¹⁴, —X³NR¹⁴C(O)NR¹²R¹⁴ or —X³NR¹⁴C(O)OR¹², wherein X³ is a bond or alkylene, R¹² is hydrogen, alkyl or —X⁴R¹¹, R¹³ is alkyl or —X⁴R¹¹, R¹⁴ is hydrogen or alkyl and R¹⁵ is hydrogen, alkyl or —X⁵R¹⁷ and R¹⁶ is —X⁵R¹⁷, wherein X⁴ is a bond or alkylene, X⁵ is lower alkylene, R¹¹ is aryl, heteroaryl, cycloalkyl or heterocycloalkyl and R¹⁷ is —NR¹⁸R¹⁹, —C(O)OR¹⁸, —C(O)NR¹⁸R¹⁸, —OR¹⁹ or —SR¹⁹, wherein R¹⁸ is hydrogen or alkyl and R¹⁹ is hydrogen, alkyl or —NR²⁰R²¹, —OR²⁰ or —SR²⁰, wherein R²⁰ and R²¹ independently are hydrogen or alkyl, or R³ and R⁴ together form methylenedioxy or ethylenedioxy, wherein within R¹¹ any aromatic or alicyclic ring may be substituted with one R²² and within R³ any aliphatic moiety may be substituted with one to three halo, any alicyclic ring may be substituted with one to three R²³ and any aromatic ring may be substituted with one to three R²⁴;

R⁴ is as defined above or is halo, —R²⁵ or —OR²⁵, where R²⁵ is hydrogen, alkyl or halo-alkyl, or R⁴ together with R⁵ form ethylene, trimethylene or heteroalkylene which may be substituted with one R²⁶;

R⁵ is as defined above or is alkyl or —X⁷R²⁷ where X⁷ is —NH—, lower alkylene or cycloprop-1,1-ylene and R²⁷ is cycloalkyl, heterocycloalkyl, heteroaryl or aryl, where within R⁵ any aliphatic moiety may be substituted with one to three halo, any alicyclic ring may be substituted with one to three R²⁸ and any aromatic ring may be substituted with one to three R²⁹;

R⁷ is isopropyl, tert-butyl, pentyl or —X⁸R³⁰, where X⁸ is methylene, —C(CH₃)₂—, —C(CH₂OH)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring and wherein within R⁷ any aliphatic moiety may be substituted with one to three halo;

R²² is aryl, heteroaryl, cycloalkyl or heterocycloalkyl;

R²³ and R²⁸ at each occurrence independently are alkyl, halo-alkyl, hydroxy, alkoxy, acetyl or oxo;

R²⁴ and R²⁹ at each occurrence independently are alkyl, halo-alkyl, hydroxy, alkoxy or acetyl;

R²⁶ is —X⁷R²⁷ where X⁷ and R²⁷ are as defined above;

R³³ at each occurrence independently are methyl or halo; and

R³⁴ is lower alkyl, —CH₂COOR³⁵, —CH₂CONHR³⁵ or —CH₂NH₂ wherein R³⁵ is hydrogen or alkyl; or a pharmaceutically acceptable salt thereof.

Some preferred compounds of Formula IV, are those in which R¹ and R⁴ are hydrogen; R² is hydrogen, halo, alkyl, halo-alkyl, —OR⁹, —X²COOR⁹, —NR¹⁰C(O)NR⁹R¹⁰ or —C(O)NR⁹R¹⁰, where X² is a bond or alkylene, R⁹ is hydrogen, alkyl, halo-alkyl or heterocycloalkylalkyl and R¹⁰ is hydrogen or alkyl; R³ is hydrogen, halo, —NHR³⁶ or —N(R³⁷)₂, wherein R³⁶ is alkyl, alkyloxycarbonylalkyl, carbamoylalkyl, heterocylcoalkylalkyl, hydroxyalkoxyalkyl, aminoalkyl, hydroxyalkyl and R³⁷ is hydrogen, alkyl or hydroxyalkyl, or each R³⁷ together with the nitrogen atom to which they are attached form heterocycloalkyl or heteroaryl, wherein heterocycloalkyl may be substituted with one to three groups independently selected from alkyl, hydroxy, acetyl, heteroaryl, carboxy and oxo and heteroaryl may be substituted with one to three groups independently selected from alkyl, hydroxy, acetyl, heteroaryl and carboxy; R⁵ is alkyl or —X⁶R²⁷ where X⁶ is lower alkylene or cycloprop-1,1-ylene and R²⁷ is cycloalkyl, heterocycloalkyl, heteroaryl or aryl, wherein aryl and heteroaryl may be substituted with one to three halo or halo-alkyl; and R⁷ is isopropyl, tert-butyl, pentyl or —X⁸R³⁰, where X⁸ is methylene, —C(CH₂OH)₂—, cycloprop-1,1-ylene or —CHR³¹—, wherein R³¹ is —X⁹R³² wherein X⁹ is alkylene and R³² is carboxy, carbamoyl, cyano, aminoacetylamino or tert-butoxycarbonylamino and R³⁰ is cycloalkyl, heterocycloalkyl, phenyl or heteroaryl, wherein within R³⁰ phenyl may be substituted with one to two groups independently selected from halo and methyl at any position on the ring, cycloalkyl may be substituted with one —CH₂COOR³⁵ at the 1-, 2- or 3-position on the ring and phenyl with one lower alkyl, halo, —CH₂COOR³⁵ or —CH₂NH₂ at the 2- or 3-position on the ring, wherein R³⁵ is hydrogen or alkyl; or a pharmaceutically acceptable salt thereof.

Still further preferred are those compounds of Formula IV in which R¹ is hydrogen; R² is carboxy, carboxymethyl, fluoro, hydrogen, hydroxy, methoxy, N-(2-morpholin-4-ylethyl)carbamoyl or ureido; R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl or 4-pyrid-2-ylpiperazin-1-yl; R⁴ is hydrogen; R⁵ is 4-chlorobenzyl, 2-chloro-6-fluorobenzyl, 4-chloro-2-fluorobenzyl, 2-chloropyrid-5-ylmethyl, cyclohexylmethyl, 2,4-difluorobenzyl, 2-ethylbutyl, 2-fluorobenzyl, 2-fluoro-4-trifluoromethylbenzyl, 2-morpholin-4-ylethyl, phenethyl, 3-trifluoromethylbenzyl 4-trifluoromethylbenzyl or 1-(4-trifluoromethylphenyl)cyclopropyl; and R⁷ is 5-acetylamino-1-phenylpentyl, 2-aminomethylbenzyl, 5-amino-1-phenylpentyl, benzyl, 2-(tert-butoxycarbonylaminomethyl)benzyl, 5-tert-butoxycarbonylamino-1-phenylpentyl, tert-butylmethyl, (1-carbamoylmethylcyclohexyl)methyl, 2-carbamoyl-1-phenylethyl, 3-carbamoyl-1-phenylpropyl, 4-carbamoyl-1-phenylbutyl, 2-carboxymethylbenzyl, 4-carboxy-1-phenylbutyl, 2-cyano-1-phenylethyl, 4-cyano-1-phenylbutyl, 5-cyano-1-phenylpentyl, cyclohexylmethyl, cyclopentylmethyl, cyclopropylmethyl, 2,4-difluorobenzyl, 2,6-difluorobenzyl, 3,5-difluorobenzyl, 4-fluorobenzyl, isobutyl, 2-methylbenzyl, 3-methylbenzyl, pentyl, 1-methyl-1-phenylethyl, 1-phenylcyclopropyl, piperidin-1-ylmethyl, pyrid-2-ylmethyl, pyrid-3-ylmethyl, pyrrolidin-1-ylmethyl or thien-3-ylmethyl; or a pharmaceutically acceptable salt thereof.

Other preferred compounds of Formula IV are those in which R¹ and R⁴ are hydrogen; R² is fluoro; R³ is —N(R³⁷)₂, wherein each R³⁷ together with the nitrogen atom to which they are attached form heterocycloalkyl wherein heterocycloalkyl may be substituted with alkyl, hydroxy, acetyl, heteroaryl, carboxy or oxo; R⁵ is 4-chlorobenzyl, 4-chloro-2-fluorobenzyl, 2-chloropyrid-5-ylmethyl, 2-fluoro-4-trifluoromethylbenzyl, 4-trifluoromethylbenzyl or 1-(4-trifluoromethylphenyl)cyclopropyl; and R⁷ is 5-acetyl-1-phenylpentyl, 5-amino-1-phenylpentyl, benzyl, 4-carbamoyl-1-phenylbutyl, 4-carboxy-1-phenylbutyl, 4-cyano-1-phenylbutyl, cyclopentylmethyl, 4-fluorobenzyl or 1-methyl-1-phenylethyl; or a pharmaceutically acceptable salt thereof.

A further aspect of the invention is a compound of the following structure:

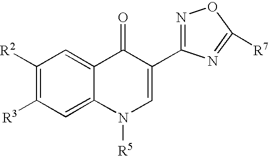

wherein R², R³, R⁵ and R⁷ are as set forth in the following Table I.

TABLE I

| R² | R³ | R⁵ | R⁷ |
|---|---|---|---|
| •OCH₃ | •OCH₃ | 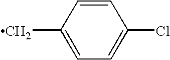 | 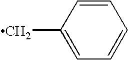 |
| •OCH₃ | •OCH₃ | 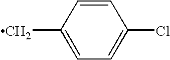 | 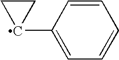 |
| •OCH₃ | •OCH₃ | 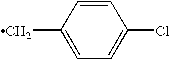 | 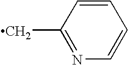 |
| •OCH₃ | •OCH₃ | 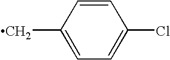 | 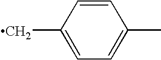 |
| •OCH₃ | •OCH₃ | 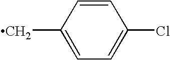 | 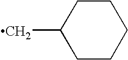 |
| •OCH₃ | •OCH₃ | 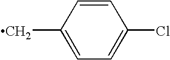 | 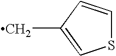 |
| •OCH₃ | •OCH₃ | 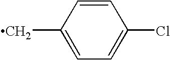 | 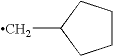 |
| •F | 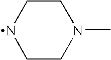 | 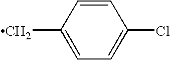 | 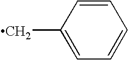 |
| •F | 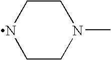 | 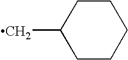 | 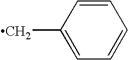 |
| •OCH₃ | •H | 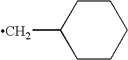 | 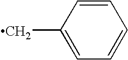 |
| •OCH₃ | •H | 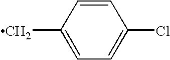 | 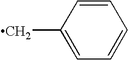 |
| •OH | •H | 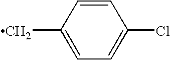 | 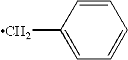 |
| •F | 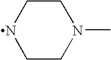 | 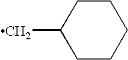 | 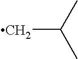 |
| •F | 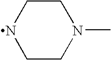 | 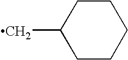 | 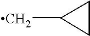 |
| •F | 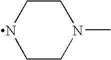 | 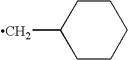 | 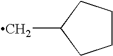 |
| •F | 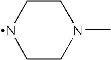 | 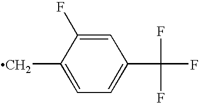 | 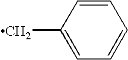 |
| •F | 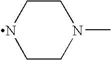 | 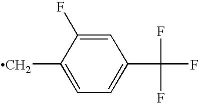 | 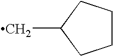 |
| •F | 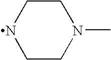 | 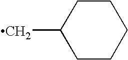 | 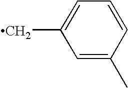 |
| •F | 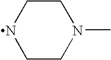 | 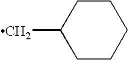 | 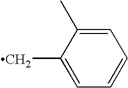 |
| •F | 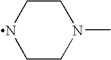 | 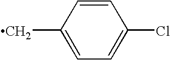 | 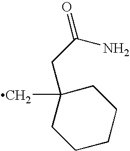 |
| •F | 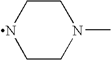 | 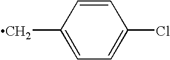 | 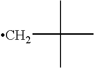 |
| •F | 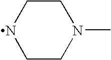 | 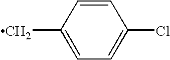 | 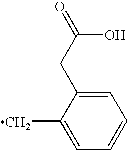 |
| •F | 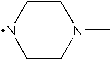 | 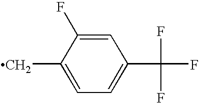 | 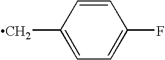 |
| •OH | •H | 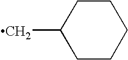 | 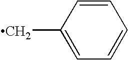 |
| •F | 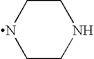 | 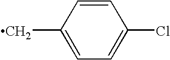 | 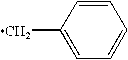 |
| •F | 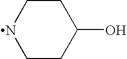 | 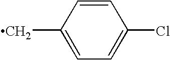 | 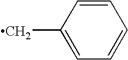 |
| •F | 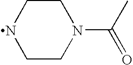 | 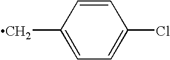 | 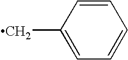 |
| •F | 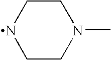 | 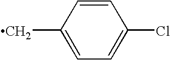 | 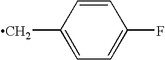 |
| •F | 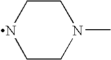 | 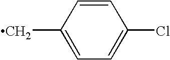 | 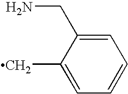 |
| •F | 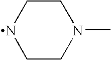 | 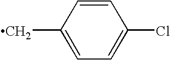 | 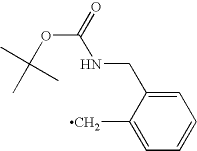 |
| •F | 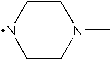 | 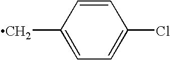 | 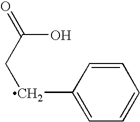 |
| •F | 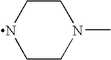 | 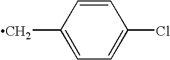 | 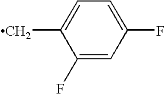 |
| •F | 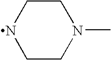 | 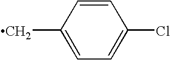 | 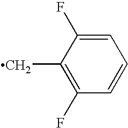 |
| •F | 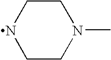 | 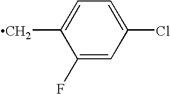 | 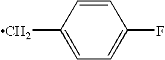 |
| •F | 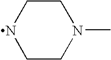 | 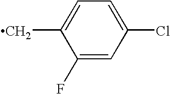 | 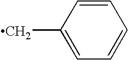 |
| •F | 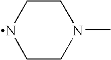 | 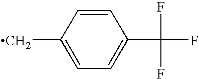 | 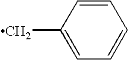 |
| •F | 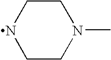 | 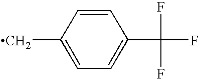 | 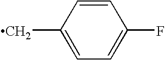 |
| •F | 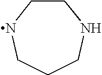 | 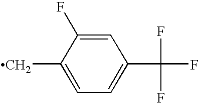 | 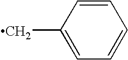 |
| •F | 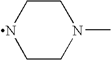 | 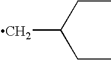 | 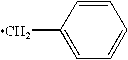 |
| •F | 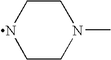 | 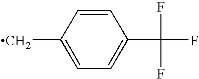 | 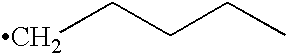 |
| •F | 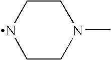 | 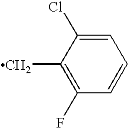 | 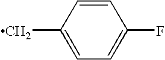 |
| •F | 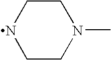 | 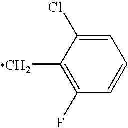 | 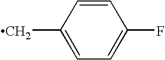 |
| •COOH | •H | 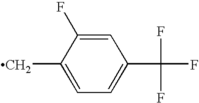 | 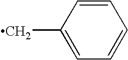 |
| •F | 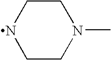 | 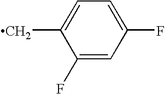 | 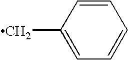 |
| •F | 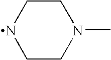 | 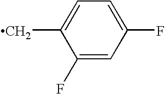 | 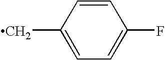 |
| •F | 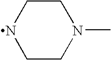 | 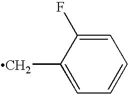 | 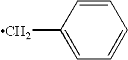 |
| •F | 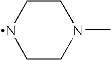 | 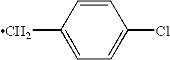 | 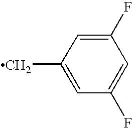 |
| •F | 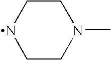 | 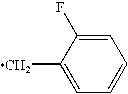 | 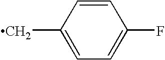 |
| •F | 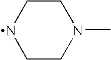 | 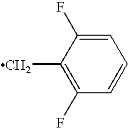 | 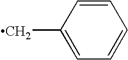 |
| •F | 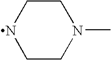 | 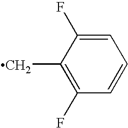 | 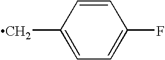 |
| 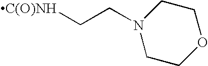 | •H | 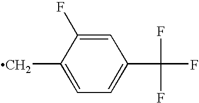 | 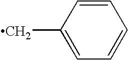 |
| •F | 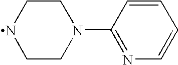 | 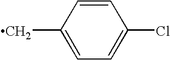 | 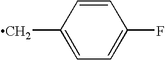 |
| •F | 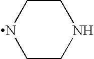 | 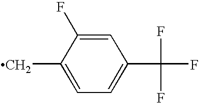 | 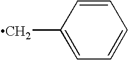 |
| •F | •N(CH₃)₂ | 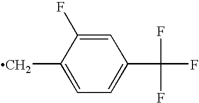 | 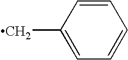 |
| •F | 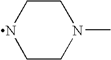 | 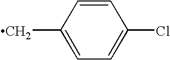 | 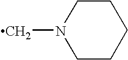 |
| •F | 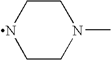 | 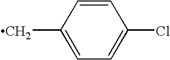 | 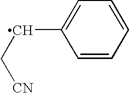 |
| •F | 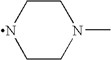 | 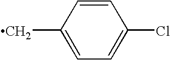 | 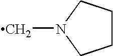 |
| •F | •NHCH₃ | 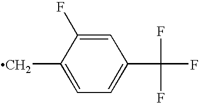 | 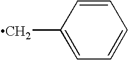 |
| •F | 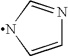 | 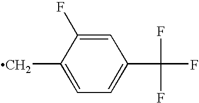 | 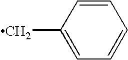 |
| •F | 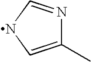 | 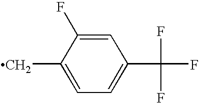 | 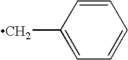 |
| •F | 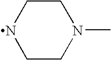 | 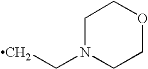 | 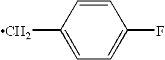 |
| •F | 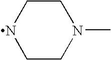 | 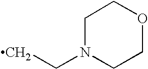 | 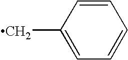 |
| •F | 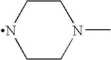 | 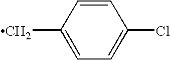 | 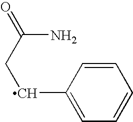 |
| •F | 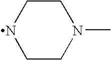 | 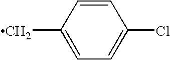 | 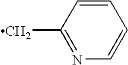 |
| •F | 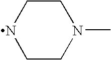 | 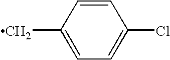 | 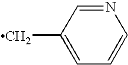 |
| •F | 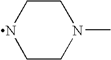 | 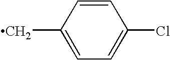 | 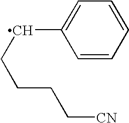 |
| •F | 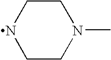 | 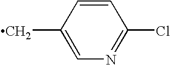 | 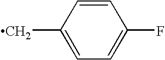 |
| •F | 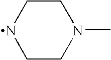 | 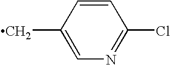 | 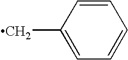 |
| •F | 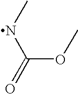 | 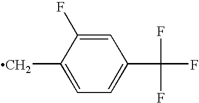 | 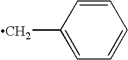 |
| •F | 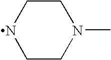 | 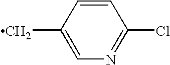 | 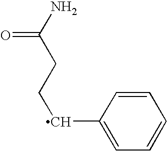 |
| •H | •OCH₃ | 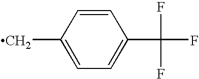 | 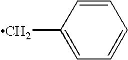 |
| •F | 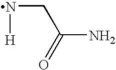 | 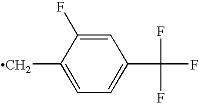 | 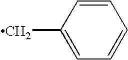 |
| •F | 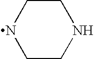 | 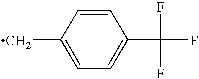 | 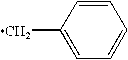 |
| •F | 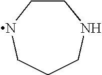 | 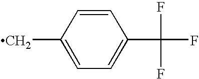 | 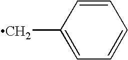 |
| •F | 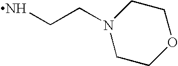 | 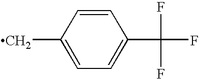 | 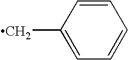 |
| •F | 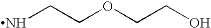 | 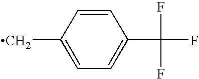 | 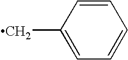 |
| •F | •N(CH₃)₂ | 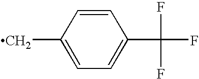 | 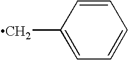 |
| •F | 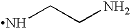 | 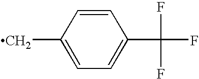 | 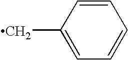 |
| 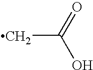 | •H | 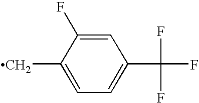 | 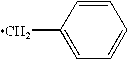 |
| 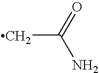 | •H | 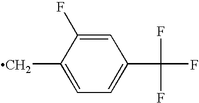 | 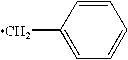 |
| •F | 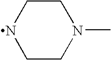 | 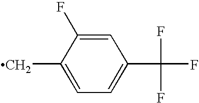 | 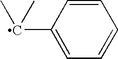 |
| •F | 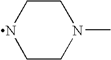 | 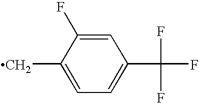 | 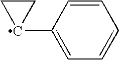 |
| •F | 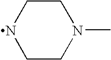 | 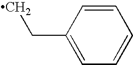 | 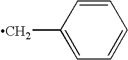 |
| •F | 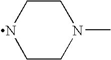 | 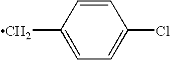 | 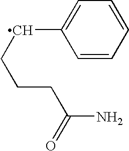 |
| •F | 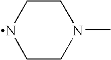 | 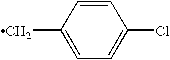 | 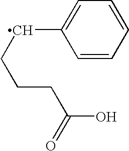 |
| •F | 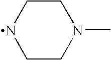 | 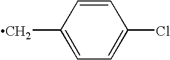 | 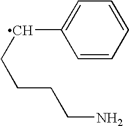 |
| •F | •N(CH₂CH₂OH)₂ | 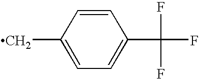 | 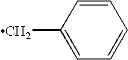 |
| •F | 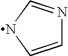 | 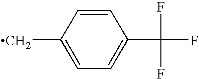 | 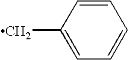 |
| •F | 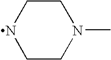 | 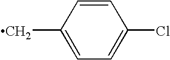 | 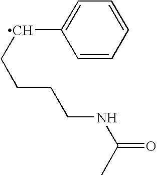 |
| •F | 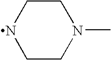 | 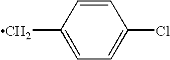 | 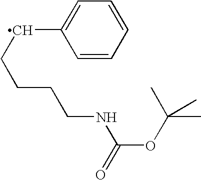 |
| •F | 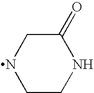 | 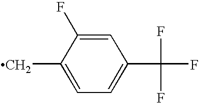 | 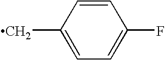 |
| •F | 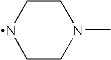 | 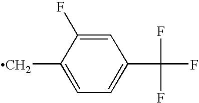 | 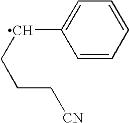 |
| •F | 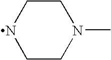 | 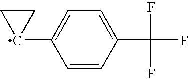 | 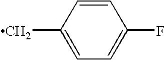 |
| •F | 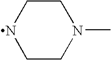 | 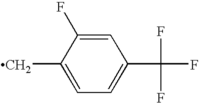 | 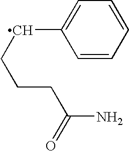 |
| •F | 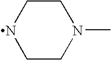 | 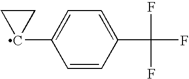 | 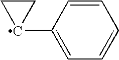 |
| •F | 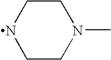 | 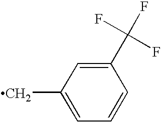 | 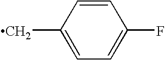 |
| •F | 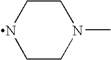 | 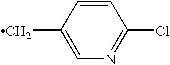 | 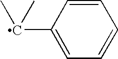 |
| •F | 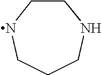 | 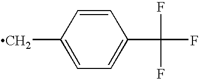 | 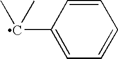 |
| •H | •H | 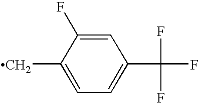 | 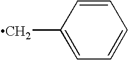 |
| •H | •H | 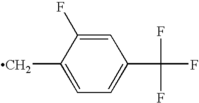 | 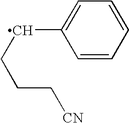 |
| •F | 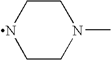 | 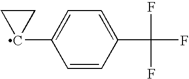 | 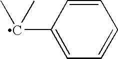 |
| •F | 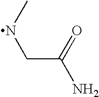 | 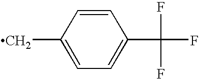 | 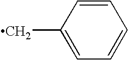 |
| •F | 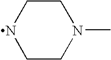 | 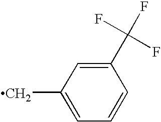 | 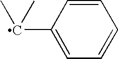 |
| •H | •H | 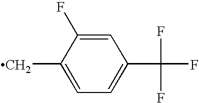 | 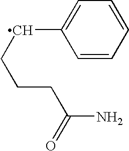 |
| •F | 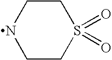 | 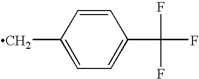 | 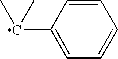 |
| •F | 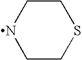 | 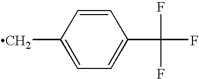 | 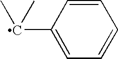 |
| •F | 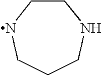 | 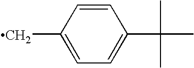 | 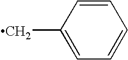 |
| •F | 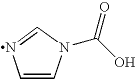 | 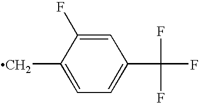 | 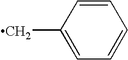 |
| •H | •H | 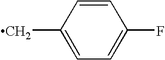 | 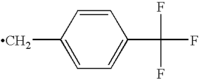 |
| •F | 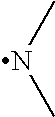 | 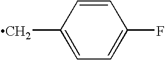 | 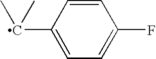 |
| •H | •H | 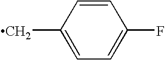 | 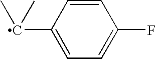 |
| •F | •F | 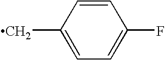 | 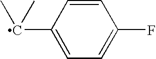 |
| •F | 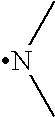 | 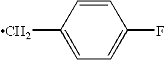 | 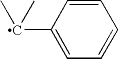 |
| •F | 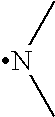 | 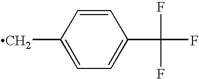 | 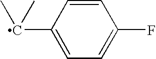 |
| •F | 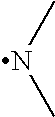 | 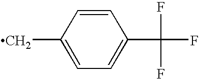 | 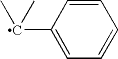 |
| •F | 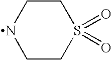 | 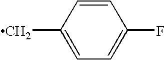 | 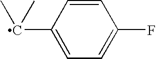 |
| •F | 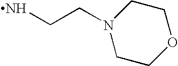 | 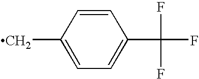 | 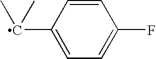 |
| •H | •H | 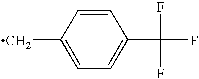 | 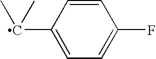 |
| •F | 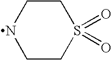 | 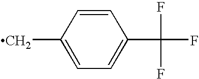 | 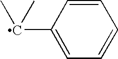 |
| •H | •H | 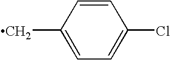 | 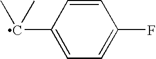 |
| •F | 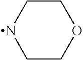 | 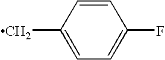 | 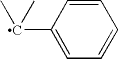 |
| •F | 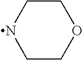 | 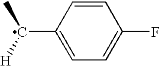 | 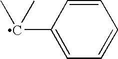 |
| •F | 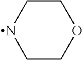 | 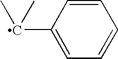 | 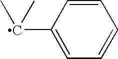 |
| •F | 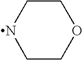 | 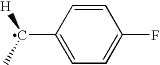 | 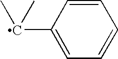 |
| •F | 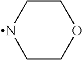 | 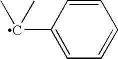 | 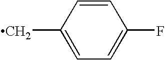 |
| •H | •H | 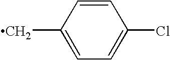 | 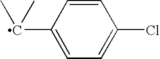 |
| •F | 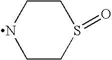 | 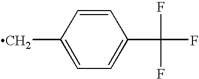 | 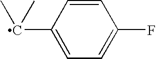 |
| •F | 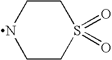 | 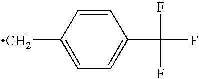 | 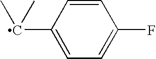 |
| •F | 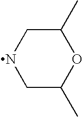 | 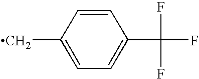 | 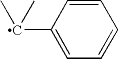 |
| •F | 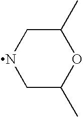 | 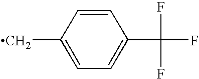 | 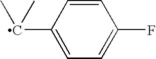 |
| •H | •H | 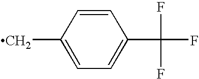 | 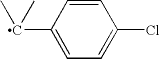 |
| •H | •H | 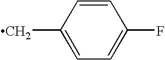 | 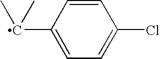 |
| •F | 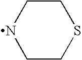 | 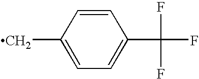 | 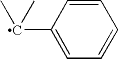 |
| •F | 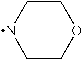 | 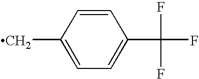 | 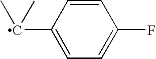 |
| •F | 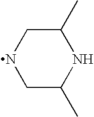 | 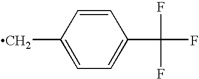 | 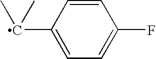 |
| •F | 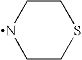 | 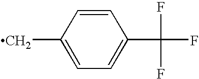 | 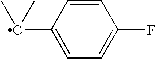 |
| •F | 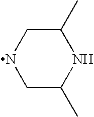 | 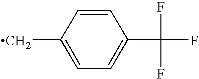 | 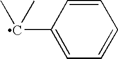 |
| •F | 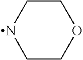 | 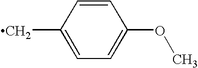 | 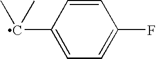 |
| •F | 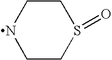 | 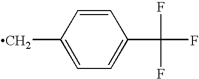 | 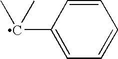 |
| •F | 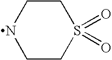 | 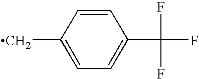 | 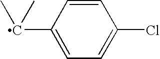 |
| •F | 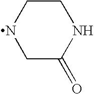 | 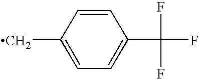 | 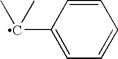 |
| •F | 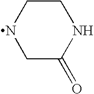 | 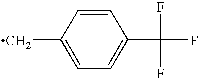 | 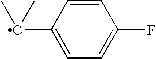 |
| •H | 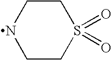 | 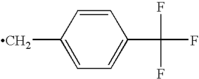 | 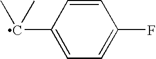 |
| •F | 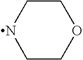 | 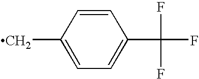 | 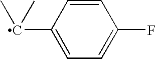 |
| •OCH₃ | •OCH₃ | 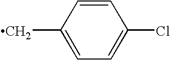 | 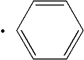 |
| •F | 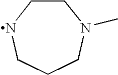 | 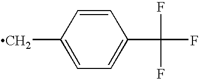 | 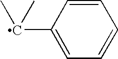 |
| •F | 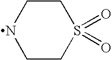 | 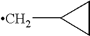 | 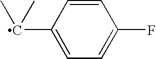 |
| •F | 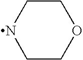 | 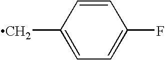 | 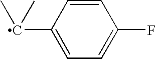 |
| •F | •F | 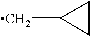 | 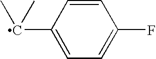 |
| •F | 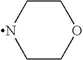 | 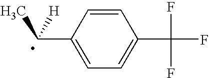 | 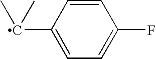 |
| •F | 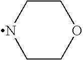 | 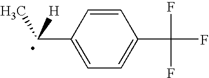 | 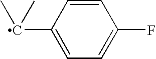 |
| •F | 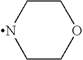 | 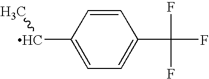 | 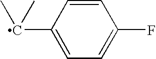 |
| •F | 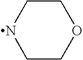 | 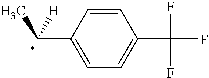 | 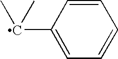 |
| •F | 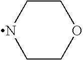 | 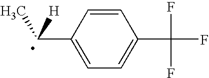 | 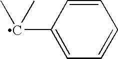 |
| •F | 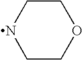 | 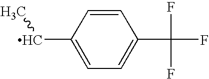 | 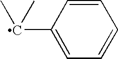 |
| •F | 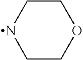 | 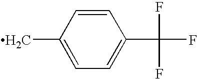 | 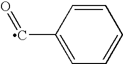 |
| •F | Cl | 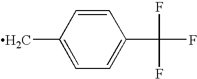 | 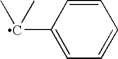 |
| •F | 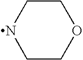 | 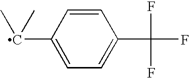 | 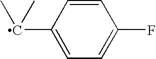 |
| •F | OMe | 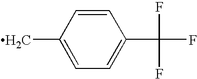 | 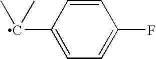 |
| •F | 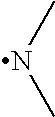 | 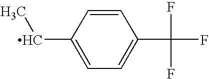 | 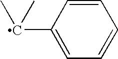 |
| •F | 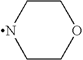 | 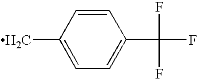 | 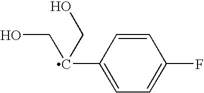 |
| •F | 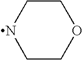 | 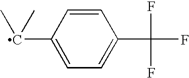 | 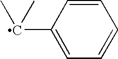 |
| •F | 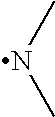 | 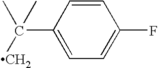 | 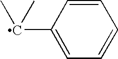 |
| •F | •OEt | 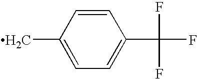 | 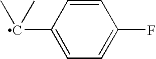 |
| •F | 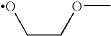 | 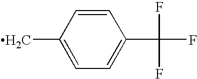 | 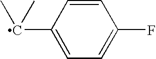 |
| •F | 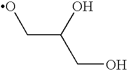 | 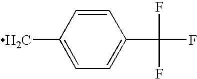 | 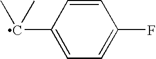 |
| •F | 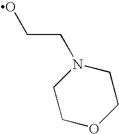 | 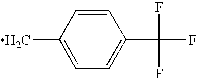 | 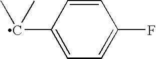 |

A further aspect of the invention is a compound of the following structure:

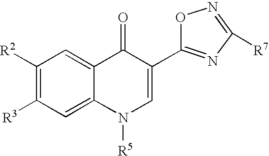

wherein R², R³, R⁵ and R⁷ are as set forth in the following Table II.

TABLE II

| R² | R³ | R⁵ | R⁷ |
|---|---|---|---|
| •OCH₃ | •OCH₃ | 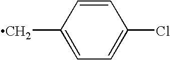 | 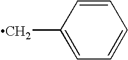 |
| •OCH₃ | •OCH₃ | 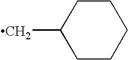 | 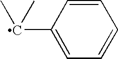 |
| •F | 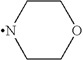 | 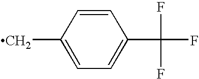 | 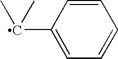 |
| •F | 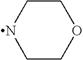 | 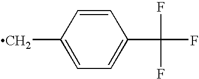 | 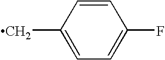 |
| •F | 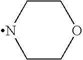 | 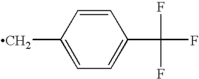 | 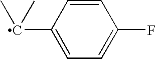 |
| •OCH₃ | •OCH₃ | 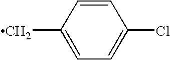 | 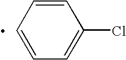 |

A further aspect of the invention is a compound of the following structure:

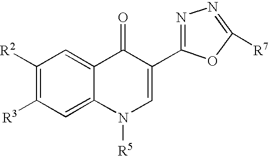

wherein R², R³, R⁵ and R⁷ are as set forth in the following Table III.

TABLE III

| R² | R³ | R⁵ | R⁷ |
|---|---|---|---|
| •OCH₃ | •OCH₃ | 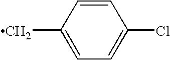 | 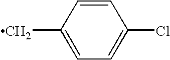 |
| •OCH₃ | •OCH₃ | 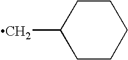 | 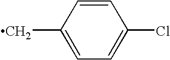 |
| •F | 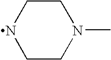 | 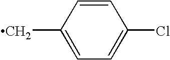 | 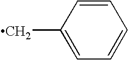 |
| •F | 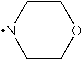 | 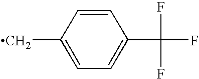 | 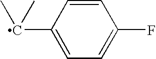 |
| •F | 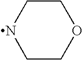 | 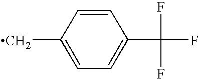 | 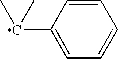 |
| •F | 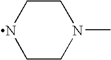 | 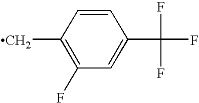 | 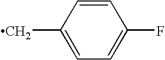 |

A further aspect of the invention is a compound of the following structure:

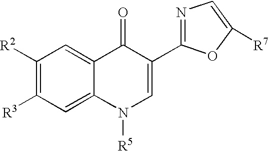

wherein R², R³, R⁵ and R⁷ are as set forth in the following Table IV.

TABLE IV

| R² | R³ | R⁵ | R⁷ |
|---|---|---|---|
| •OCH₃ | •H | 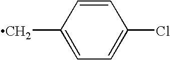 | 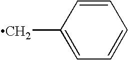 |
| •OH | •H | 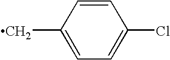 | 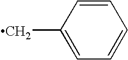 |
| 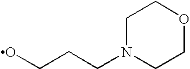 | •H | 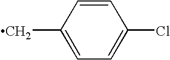 | 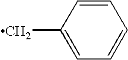 |

A further aspect of the invention is a compound of the following structure:

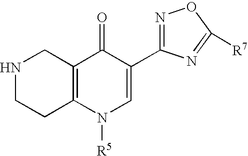

wherein R², R³, R⁵ and R⁷ are as set forth in the following Table V.

TABLE V

| R⁵ | R⁷ |
|---|---|
| 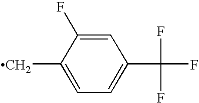 | 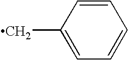 |
| 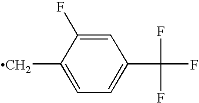 | 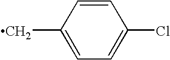 |

A further aspect of the invention is a compound of the following structure:

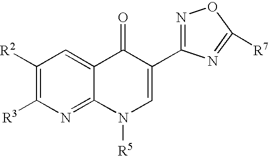

wherein R⁵ and R⁷ are as set forth in the following Table VI.

TABLE VI

| R² | R³ | R⁵ | R⁷ |
|---|---|---|---|
| •F | 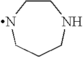 | 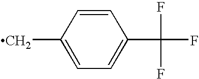 | 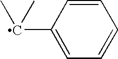 |

A further aspect of the present invention is a compound of Formula IV in which:

R¹ hydrogen; R² is hydrogen, halo, alkyl, halo-alkyl, —OR⁹, —X²COOR⁹, —NR¹⁰C(O)NR⁹R¹⁰ or —C(O)NR⁹R¹⁰, where X² is a bond or alkylene, R⁹ is hydrogen, alkyl, halo-alkyl or heterocycloalkylalkyl and R¹⁰ is hydrogen or alkyl; R³ is hydrogen, halo, —NHR³⁶ or —N(R³⁷)₂, wherein R³⁶ is alkyl, alkyloxycarbonylalkyl, carbamoylalkyl, heterocylcoalkylalkyl, hydroxyalkoxyalkyl, aminoalkyl, hydroxyalkyl and R³⁷ is hydrogen, alkyl or hydroxyalkyl, or each R³⁷ together with the nitrogen atom to which they are attached form heterocycloalkyl or heteroaryl, wherein heterocycloalkyl may be substituted with one to three groups independently selected from alkyl, hydroxy, acetyl, heteroaryl, carboxy and oxo and heteroaryl may be substituted with one to three groups independently selected from alkyl, hydroxy, acetyl, heteroaryl and carboxy; R⁴ is hydrogen; R⁵ is alkyl or —X⁷R²⁷ where X⁷ is lower alkylene or cycloprop-1,1-ylene and R²⁷ is cycloalkyl, heterocycloalkyl, heteroaryl or aryl, wherein aryl and heteroaryl may be substituted with one to three halo or halo-alkyl; and R⁷ is isopropyl, tert-butyl, pentyl or —X⁸R³⁰, where X⁸ is methylene, —C(CH₂OH)₂—, cycloprop-1,1-ylene or —CHR³¹—, wherein R³¹ is —X⁹R³² wherein X⁹ is alkylene and R³² is carboxy, carbamoyl, cyano, aminoacetylamino or tert-butoxycarbonylamino and R³⁰ is cycloalkyl, heterocycloalkyl, phenyl or heteroaryl, wherein within R³⁰ phenyl may be substituted with one to two groups independently selected from halo and methyl at any position on the ring, cycloalkyl may be substituted with one —CH₂COOR³⁵ at the 1-, 2- or 3-position on the ring and phenyl with one lower alkyl, halo, —CH₂COOR³⁵ or —CH₂NH₂ at the 2- or 3-position on the ring, wherein R³⁵ is hydrogen or alkyl; or a pharmaceutically acceptable salt thereof.

A further a aspect of the present invention is a compound of Formula IV in which R¹ is hydrogen; R² is carboxy, carboxymethyl, fluoro, hydrogen, hydroxy, methoxy, N-(2-morpholin-4-ylethyl)carbamoyl or ureido; R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl or 4-pyrid-2-ylpiperazin-1-yl; R⁴ is hydrogen; R⁵ is 4-chlorobenzyl, 2-chloro-6-fluorobenzyl, 4-chloro-2-fluorobenzyl, 2-chloropyrid-5-ylmethyl, cyclohexylmethyl, 2,4-difluorobenzyl, 2-ethylbutyl, 2-fluorobenzyl, 2-fluoro-4-trifluoromethylbenzyl, 2-morpholin-4-ylethyl, phenethyl, 3-trifluoromethylbenzyl 4-trifluoromethylbenzyl or 1-(4-trifluoromethylphenyl)cyclopropyl; and R⁷ is 5-acetylamino-1-phenylpentyl, 2-aminomethylbenzyl, 5-amino-1-phenylpentyl, benzyl, 2-(tert-butoxycarbonylaminomethyl)benzyl, 5-tert-butoxycarbonylamino-1-phenylpentyl, tert-butylmethyl, (1-carbamoylmethylcyclohexyl)methyl, 2-carbamoyl-1-phenylethyl, 3-carbamoyl-1-phenylpropyl, 4-carbamoyl-1-phenylbutyl, 2-carboxymethylbenzyl, 4-carboxy-1-phenylbutyl, 2-cyano-1-phenylethyl, 4-cyano-1-phenylbutyl, 5-cyano-1-phenylpentyl, cyclohexylmethyl, cyclopentylmethyl, cyclopropylmethyl, 2,4-difluorobenzyl, 2,6-difluorobenzyl, 3,5-difluorobenzyl, 4-fluorobenzyl, isobutyl, 2-methylbenzyl, 3-methylbenzyl, pentyl, 1-methyl-1-phenylethyl, 1-phenylcyclopropyl, piperidin-1-ylmethyl, pyrid-2-ylmethyl, pyrid-3-ylmethyl, pyrrolidin-1-ylmethyl or thien-3-ylmethyl; or a pharmaceutically acceptable salt thereof.

A further aspect of the present invention is a compound of Formula IV in which R¹ and R⁴ are hydrogen; R² is fluoro; R³ is —N(R³⁷)₂, wherein each R³⁷ together with the nitrogen atom to which they are attached form heterocycloalkyl wherein heterocycloalkyl may be substituted with alkyl, hydroxy, acetyl, heteroaryl, carboxy or oxo; R⁵ is 4-chlorobenzyl, 4-chloro-2-fluorobenzyl, 2-chloropyrid-5-ylmethyl, 2-fluoro-4-trifluoromethylbenzyl, 4-trifluoromethylbenzyl or 1-(4-trifluoromethylphenyl)cyclopropyl; and R⁷ is 5-acetyl-1-phenylpentyl, 5-amino-1-phenylpentyl, benzyl, 4-carbamoyl-1-phenylbutyl, 4-carboxy-1-phenylbutyl, 4-cyano-1-phenylbutyl, cyclopentylmethyl, 4-fluorobenzyl or 1-methyl-1-phenylethyl; or a pharmaceutically acceptable salt thereof.

A further aspect of the present invention is that in which R¹ is halo, —R⁸ or —OR⁸, where R⁸ is hydrogen, alkyl or halo-alkyl and specifically where R¹ is hydrogen.

A further aspect of the present invention is that in which R² is hydrogen, halo, alkyl, halo-alkyl, —OR⁹, —X²COOR⁹, —NR¹⁰C(O)NR⁹R¹⁰ or —C(O)NR⁹R¹⁰, where X² is a bond or alkylene, R⁹ is hydrogen, alkyl, halo-alkyl or heterocycloalkylalkyl and R¹⁰ is hydrogen or alkyl; and specifically where R² is carboxy, carboxymethyl, fluoro, hydrogen, hydroxy, methoxy, N-(2-morpholin-4-ylethyl)carbamoyl or ureido. A particular aspect of the invention is that in which R² is fluoro.

A further aspect of the invention is that in which R³ is halo, —X³R¹¹, —X³OR¹², —X³C(O)R¹³, —X³C(O)OR¹², —X³OC(O)R¹³, —X³C(O)OR¹², —X³NR¹⁴R¹², —X³NR¹⁵R¹⁶, —X³OR¹⁵, —X³SR¹⁵, —X³NR¹⁴C(O)R¹³, —X³C(O)NR¹²R¹⁴, —X³S(O)₀₋₂R¹², —X³S(O)₂NR¹²R¹⁴, —X³NR¹⁴C(O)NR¹²R¹⁴ or —X³NR¹⁴C(O)OR¹², wherein X³ is a bond or alkylene, R¹² is hydrogen, alkyl or —X⁴R¹¹, R¹³ is alkyl or —X⁴R¹¹, R¹⁴ is hydrogen or alkyl and R¹⁵ is hydrogen, alkyl or —X⁵R¹⁷ and R¹⁶ is —X⁵R¹⁷, wherein X⁴ is a bond or alkylene, X⁵ is lower alkylene, R¹¹ is aryl, heteroaryl, cycloalkyl or heterocycloalkyl and R¹⁷ is —NR¹⁸R¹⁹, —C(O)OR¹⁸, —C(O)NR¹⁸R¹⁸, —OR¹⁹ or —SR¹⁹, wherein R¹⁸ is hydrogen or alkyl and R¹⁹ is hydrogen, alkyl or —NR²⁰R²¹, —OR²⁰ or —SR²⁰, wherein R²⁰ and R²¹ independently are hydrogen or alkyl and wherein within R¹¹ any alicyclic or aromatic ring may be substituted with one aryl, heteroaryl, cycloalkyl or heterocycloalkyl and within R³ any aliphatic moiety may be substituted with one to three halo, any alicyclic ring may be substituted with one to three groups independently selected from alkyl, halo-alkyl, hydroxy, alkoxy, acetyl and oxo and any aromatic ring may be substituted with one to three groups independently selected from alkyl, halo-alkyl, hydroxy, alkoxy and acetyl.

A further aspect of the invention is that in which R³ is hydrogen, halo, —NHR³⁶ or —N(R³⁷)₂, wherein R³⁶ is alkyl, alkyloxycarbonylalkyl, carbamoylalkyl, heterocylcoalkylalkyl, hydroxyalkoxyalkyl, aminoalkyl, hydroxyalkyl and R³⁷ is hydrogen, alkyl or hydroxyalkyl, or each R³⁷ together with the nitrogen atom to which they are attached form heterocycloalkyl or heteroaryl, wherein heterocycloalkyl may be substituted with alkyl, hydroxy, acetyl, heteroaryl, carboxy or oxo and heteroaryl may be substituted with alkyl, hydroxy, acetyl, heteroaryl or carboxy.

A further aspect of the invention is that in which R³ is —N(R³⁷)₂, wherein each R³⁷ together with the nitrogen atom to which they are attached form heterocycloalkyl wherein heterocycloalkyl may be substituted with alkyl, hydroxy, acetyl, heteroaryl, carboxy or oxo.

A further aspect of the invention is that in which R⁶ is selected from Formula (a), (b), (c) or (d): 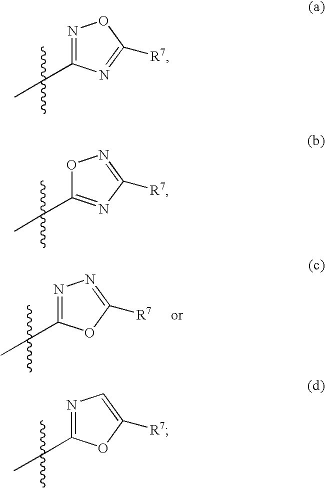
where R⁷ is as defined in the Summary of the Invention. More preferably R⁶ is Formula (a): 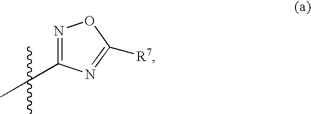
where R⁷ is as defined in the Summary of the Invention.

A further aspect of the invention is a compound selected from: 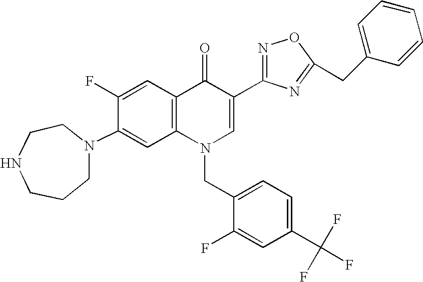
3-(5-benzyl-[1,2,4]oxadiazol-3-yl)-7-[1,4]diazepan-1-yl-6-fluoro-1-(2-fluoro-4-trifluoromethyl-benzyl)-1H-quinolin-4-one; 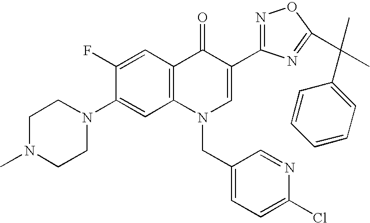
1-(6-chloro-pyridin-3-ylmethyl)-6-fluoro-3-[5-(1-methyl-1-phenyl-ethyl)-[1,2,4]oxadiazol-3-yl]-7-(4-methyl-piperazin-1-yl)-1H-quinolin-4-one; 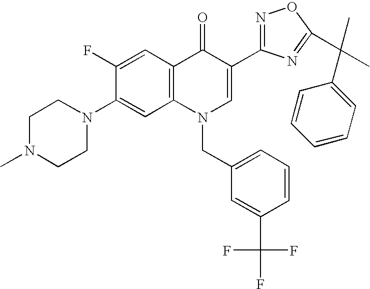
6-fluoro-3-[5-(1-methyl-1-phenyl-ethyl)-[1,2,4]oxadiazol-3-yl]-7-(4-methyl-piperazin-1-yl)-1-(3-trifluoromethyl-benzyl)-1H-quinolin-4-one; 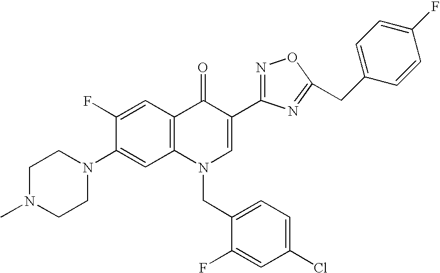
1-(4-chloro-2-fluoro-benzyl)-6-fluoro-3-[5-(4-fluoro-benzyl)-[1,2,4]oxadiazol-3-yl]-7-(4-methyl-piperazin-1-yl)-1H-quinolin-4-one; 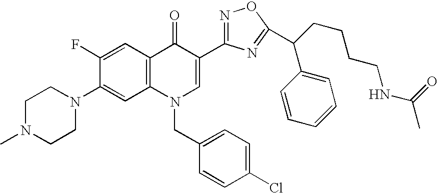
N-(5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentyl)-acetamide; 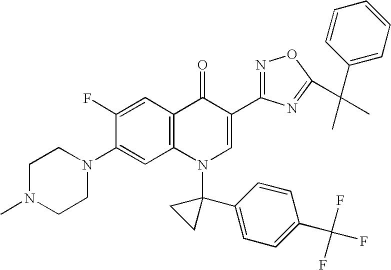
6-fluoro-3-[5-(1-methyl-1-phenyl-ethyl)-[1,2,4]oxadiazol-3-yl]-7-(4-methyl-piperazin-1-yl)-1-[1-(4-trifluoromethyl-phenyl)-cyclopropyl]-1H-quinolin-4-one; 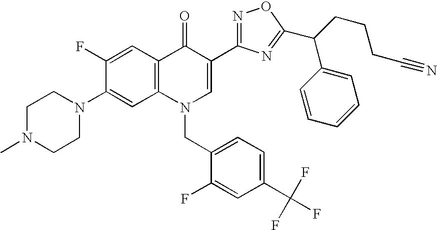
5-{3-[6-fluoro-1-(2-fluoro-4-trifluoromethyl-benzyl)-7-(4-methyl-piperazin-1-yl)-4-oxo-1,4-dihydro-quinolin-3-yl]-[1,2,4]oxadiazol-5-yl}-5-phenyl-pentanenitrile; 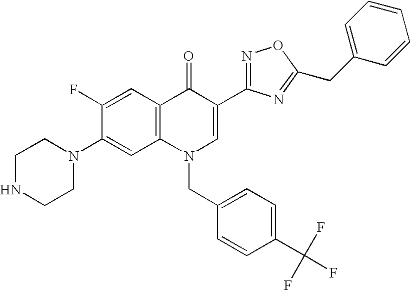
3-(5-benzyl-[1,2,4]oxadiazol-3-yl)-6-fluoro-7-piperazin-1-yl-1-(4-trifluoromethyl-benzyl)-1H-quinolin-4-one; 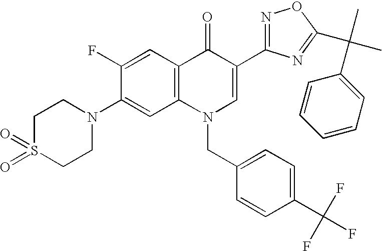
7-(1,1-dioxo-thiomorpholin-4-yl)-6-fluoro-3-[5-(1-methyl-1-phenyl-ethyl)-[1,2,4]oxadiazol-3-yl]-1-(4-trifluoromethyl-benzyl)-1H-quinolin-4-one; 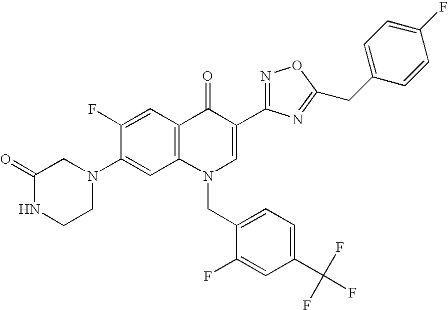
6-fluoro-3-[5-(4-fluoro-benzyl)-[1,2,4]oxadiazol-3-yl]-1-(2-fluoro-4-trifluoromethyl-benzyl)-7-(3-oxo-piperazin-1-yl)-1H-quinolin-4-one; 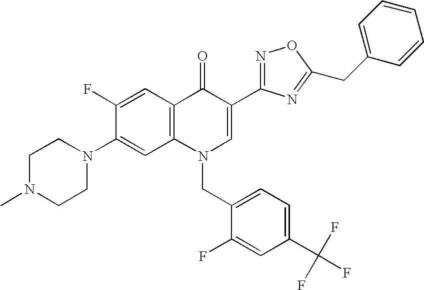
3-(5-benzyl-[1,2,4]oxadiazol-3-yl)-6-fluoro-1-(2-fluoro-4-trifluoromethyl-benzyl)-7-(4-methyl-piperazin-1-yl)-1H-quinolin-4-one; 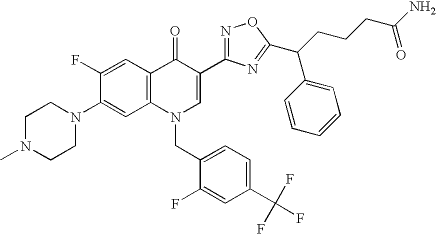
5-{3-[6-fluoro-1-(2-fluoro-4-trifluoromethyl-benzyl)-7-(4-methyl-piperazin-1-yl)-4-oxo-1,4-dihydro-quinolin-3-yl]-[1,2,4]oxadiazol-5-yl}-5-phenyl-pentanamide; 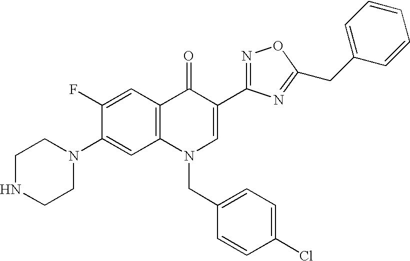
3-(5-benzyl-[1,2,4]oxadiazol-3-yl)-1-(4-chloro-benzyl)-6-fluoro-7-piperazin-1-yl-1H-quinolin-4-one; 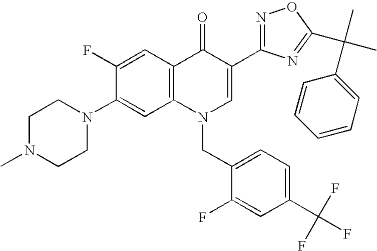
6-fluoro-1-(2-fluoro-4-trifluoromethyl-benzyl)-3-[5-(1-methyl-1-phenyl-ethyl)-[1,2,4]oxadiazol-3-yl]-7-(4-methyl-piperazin-1-yl)-1H-quinolin-4-one; 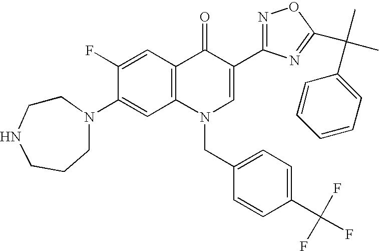
7-[1,4]diazepan-1-yl-6-fluoro-3-[5-(1-methyl-1-phenyl-ethyl)-[1,2,4]oxadiazol-3-yl]-1-(4-trifluoromethyl-benzyl)-1H-quinolin-4-one; 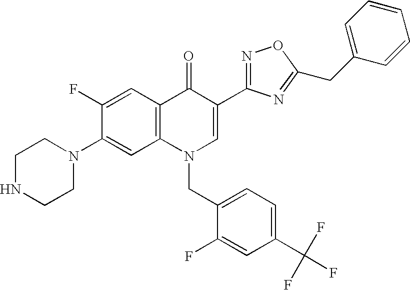
3-(5-benzyl-[1,2,4]oxadiazol-3-yl)-6-fluoro-1-(2-fluoro-4-trifluoromethyl-benzyl)-7-piperazin-1-yl-1H-quinolin-4-one; 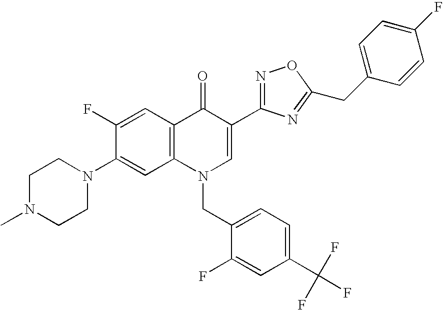
6-fluoro-3-[5-(4-fluoro-benzyl)-[1,2,4]oxadiazol-3-yl]-1-(2-fluoro-4-trifluoromethyl-benzyl)-7-(4-methyl-piperazin-1-yl)-1H-quinolin-4-one; 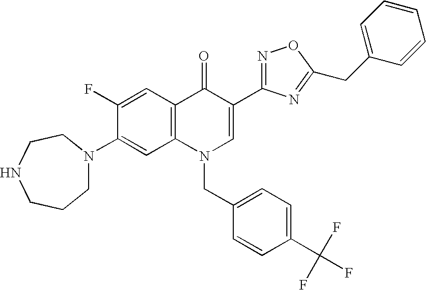
3-(5-benzyl-[1,2,4]oxadiazol-3-yl)-7-[1,4]diazepan-1-yl-6-fluoro-1-(4-trifluoromethyl-benzyl)-1H-quinolin-4-one; 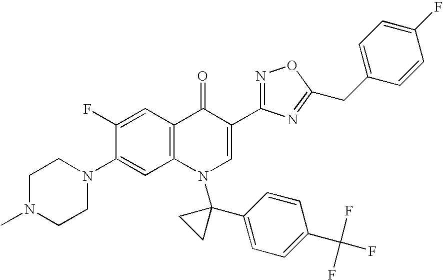
6-fluoro-3-[5-(4-fluoro-benzyl)-[1,2,4]oxadiazol-3-yl]-7-(4-methyl-piperazin-1-yl)-1-[1-(4-trifluoromethyl-phenyl)-cyclopropyl]-1H-quinolin-4-one; 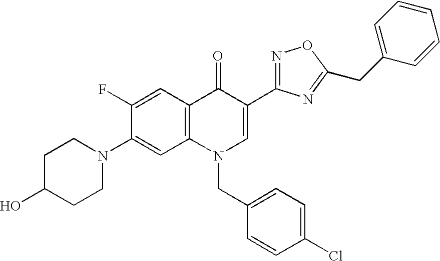
3-(5-benzyl-[1,2,4]oxadiazol-3-yl)-1-(4-chloro-benzyl)-6-fluoro-7-(4-hydroxy-piperidin-1-yl)-1H-quinolin-4-one; 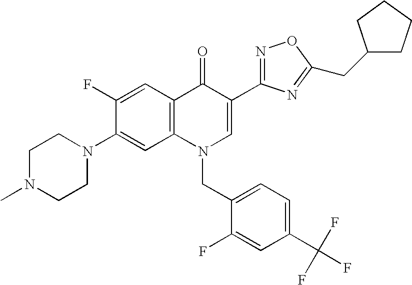
3-(5-cyclopentylmethyl-[1,2,4]oxadiazol-3-yl)-6-fluoro-1-(2-fluoro-4-trifluoromethyl-benzyl)-7-(4-methyl-piperazin-1-yl)-1H-quinolin-4-one; 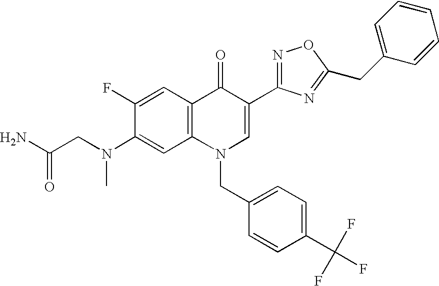
2-{[3-(5-benzyl-[1,2,4]oxadiazol-3-yl)-6-fluoro-4-oxo-1-(4-trifluoromethyl-benzyl)-1,4-dihydro-quinolin-7-yl]-methyl-amino}-acetamide; 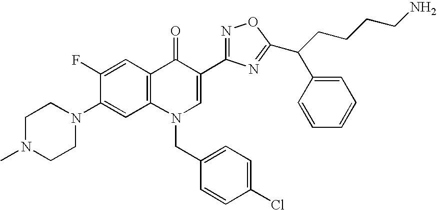
3-[5-(5-amino-1-phenyl-pentyl)-[1,2,4]oxadiazol-3-yl]-1-(4-chloro-benzyl)-6-fluoro-7-(4-methyl-piperazin-1-yl)-1H-quinolin-4-one; 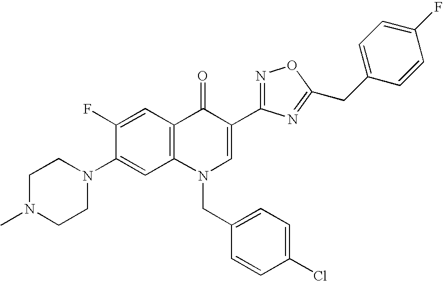
1-(4-chloro-benzyl)-6-fluoro-3-[5-(4-fluoro-benzyl)-[1,2,4]oxadiazol-3-yl]-7-(4-methyl-piperazin-1-yl)-1H-quinolin-4-one; 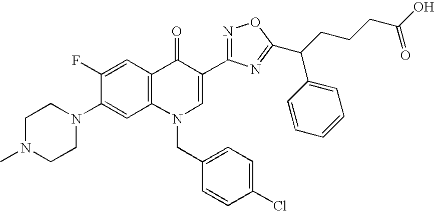
5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentanoic acid. 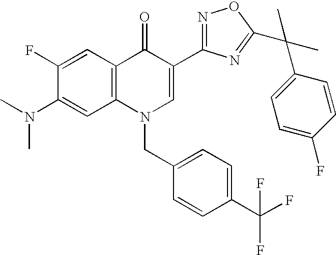
7-(dimethylamino)-6-fluoro-3-(5-(2-(4-fluorophenyl)propan-2-yl)-1,2,4-oxadiazol-3-yl)-1-(4-(trifluoromethyl)benzyl)quinolin-4(1H)-one; 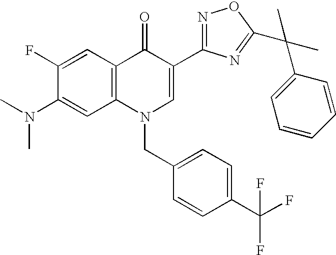
7-(dimethylamino)-6-fluoro-3-(5-(2-phenylpropan-2-yl)-1,2,4-oxadiazol-3-yl)-1-(4-(trifluoromethyl)benzyl)quinolin-4(1H)-one; 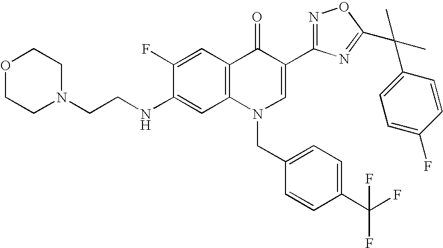
6-fluoro-3-(5-(2-(4-fluorophenyl)propan-2-yl)-1,2,4-oxadiazol-3-yl)-7-(2-morpholinoethylamino)-1-(4-(trifluoromethyl)benzyl)quinolin-4(1H)-one; 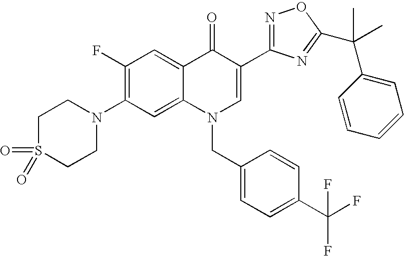
6-fluoro-3-(5-(2-phenylpropan-2-yl)-1,2,4-oxadiazol-3-yl)-7-(1,1-dioxo-thiomorpholino-1-(4-(trifluoromethyl)benzyl)quinolin-4(1H)-one; 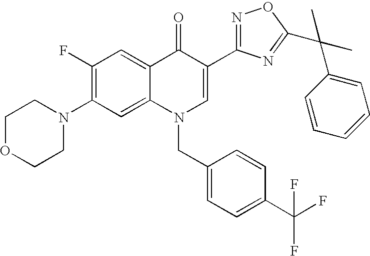
6-fluoro-7-morpholino-3-(5-(2-phenylpropan-2-yl)-1,2,4-oxadiazol-3-yl)-1-(4-(trifluoromethyl)benzyl)quinolin-4(1H)-one; 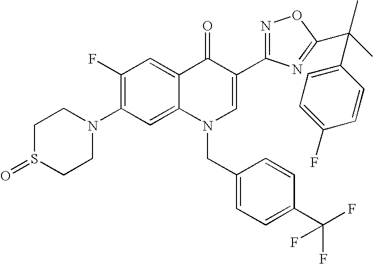
6-fluoro-3-(5-(2-(4-fluorophenyl)propan-2-yl)-1,2,4-oxadiazol-3-yl)-7-(1-oxo-thiomorpholino-1-(4-(trifluoromethyl)benzyl)quinolin-4(1H)-one; 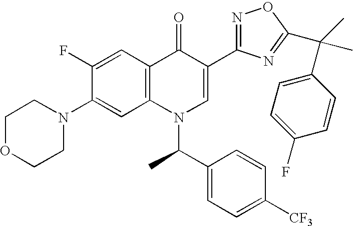
(R)-6-fluoro-3-(3-(2-(4-fluorophenyl)propan-2-yl)-1,2,4-oxadiazol-5-yl)-7-morpholino-1-(1-(4-(trifluoromethyl)phenyl)ethyl)quinolin-4(1H)-one 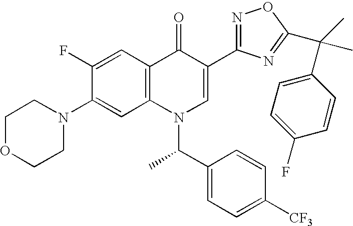
(S)-6-fluoro-3-(3-(2-(4-fluorophenyl)propan-2-yl)-1,2,4-oxadiazol-5-yl)-7-morpholino-1-(1-(4-(trifluoromethyl)phenyl)ethyl)quinolin-4(1H)-one 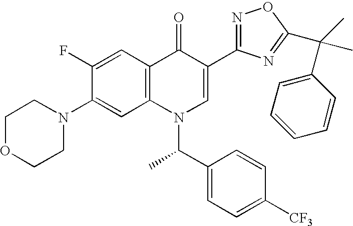
(R)-6-fluoro-7-morpholino-3-(3-(2-phenylpropan-2-yl)-1,2,4-oxadiazol-5-yl)-1-(1-(4-(trifluoromethyl)phenyl)ethyl)quinolin-4(1H)-one 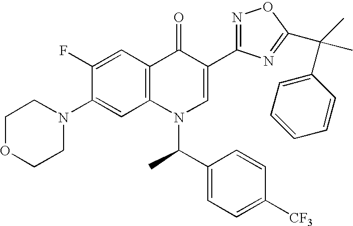
(S)-6-fluoro-7-morpholino-3-(3-(2-phenylpropan-2-yl)-1,2,4-oxadiazol-5-yl)-1-(1-(4-(trifluoromethyl)phenyl)ethyl)quinolin-4(1H)-one 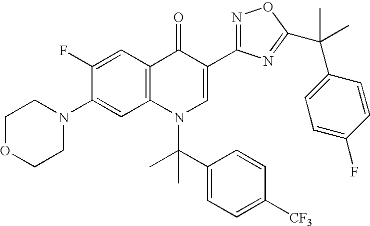
6-fluoro-3-(3-(2-(4-fluorophenyl)propan-2-yl)-1,2,4-oxadiazol-5-yl)-7-morpholino-1-(2-(4-(trifluoromethyl)phenyl)propan-2-yl)quinolin-4(1H)-one 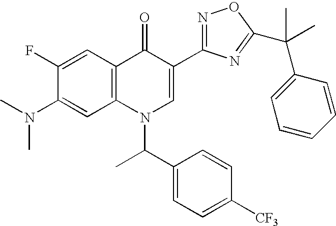
7-(dimethylamino)-6-fluoro-3-(5-(2-phenylpropan-2-yl)-1,2,4-oxadiazol-3-yl)-1-(1-(4-(trifluoromethyl)phenyl)ethyl)quinolin-4(1H)-one; and 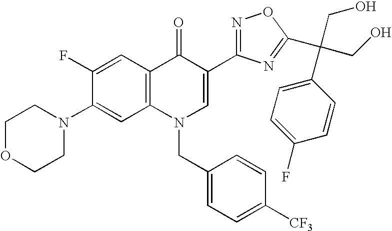
6-fluoro-3-(5-(2-(4-fluorophenyl)-1,3-dihydroxypropan-2-yl)-1,2,4-oxadiazol-3-yl)-7-morpholino-1-(4-(trifluoromethyl)benzyl)quinolin-4(1H)-one.
   Pharmacology and Utility:

The compounds of the present invention inhibit replication of HCV and are therefore useful in treating hepatitis C. The inhibitory activities of the compounds of Formula I, II and III can be determined by methods known to those of ordinary skill in the art. Details of assays for measuring inhibition of HCV replication are set forth in Biological Example 1, Biological Example 2, infra.

Administration and Pharmaceutical Compositions:

In general, compounds of Formula I, II and III will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula I, II, III or IV may range from about 10 micrograms per kilogram body weight (μ/kg) per day to about 100 milligram per kilogram body weight (mg/kg) per day, typically from about 100 μg/kg/day to about 10 mg/kg/day. Therefore, a therapeutically effective amount for an 80 kg human patient may range from about 1 mg/day to about 8 g/day, typically from about 1 mg/day to about 800 mg/day. In general, one of ordinary skill in the art, acting in reliance upon personal knowledge and the disclosure of this application, will be able to ascertain a therapeutically effective amount of a compound of Formula I, II, III or IV for treating a given disease.

The compounds of Formula I, II, III and IV can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula I, II, III or IV in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula I, II, III or IV in the composition may vary widely depending upon the type of formulation, size of a unit dosage, kind of excipients and other factors known to those of skill in the art of pharmaceutical sciences. In general, a composition of a compound of Formula I, II, III or IV for treating a given disease will comprise from 0.01% w to 90% w, preferably 5% w to 50% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula I, II and III are described below.

### EXAMPLES

General and specific schemes for preparing the compounds of the invention are provided in the Figures and the discussion below.

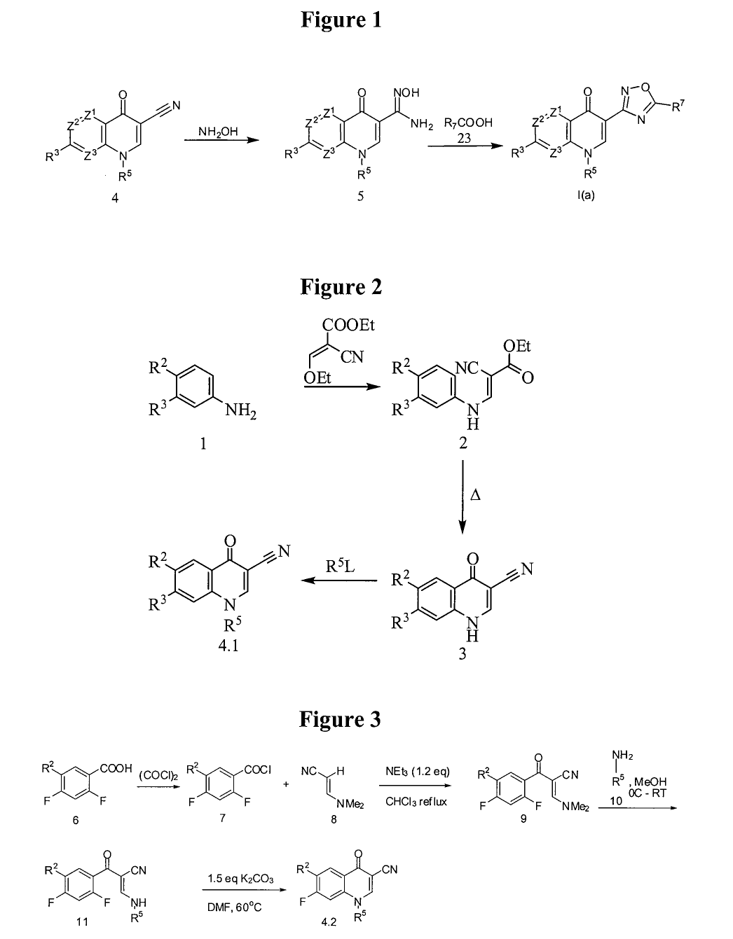

Compounds of Formula I in which R⁶ is Formula (a): 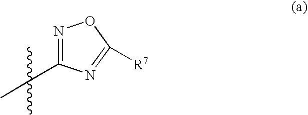
can be prepared by the reaction scheme provided in FIG. 1, in which each Z¹, Z², Z³, R³, R⁵ and R⁷ are as defined in the Summary of the Invention. Briefly, compounds of Formula I in which R⁶ is Formula (a) can be prepared by reacting a hydroxyamidine of Formula 5 with an acid of the formula R⁷COOH. The reaction is carried out in a suitable solvent, e.g., DMF. Hydroxamidines of Formula 5 can be prepared by reacting a nitrile of Formula 4 with hydroxylamine in a suitable solvent, e.g., ethanol, or the like, at 60 to 90° C. and typically requires 2 to 3 hours to complete. Compounds of formula R⁷COOH can be prepared by methods described in the literature.

Compounds of Formula 4 in which Z¹, Z², Z³ are CH can be prepared by the reaction scheme provided in FIG. 2, in which L is a leaving group and each R¹, R², R³ and R⁵ are as defined in the Summary of the Invention. Briefly, nitriles of Formula 4.1 can be prepared by reacting a nitrile of Formula 3 with a compound of R⁵L, where L is a leaving group, e.g., chloro, bromo, or the like. The reaction is carried out in the presence of an appropriate strong base, e.g., sodium hydride, potassium carbonate, or the like, and in a suitable solvent, e.g., DMF, DMA, or the like, at 50 to 60° C. and requires 2 to 4 hours to complete.

Compounds of Formula 3 can be prepared by reacting a compound of Formula 1 with ethyl 2-cyano-3-ethoxy-acrylate (available from Aldrich) to give the corresponding compound of Formula 2 and then cyclizing to give a compound of Formula 3. The reaction with the acrylate is carried out in a suitable solvent, e.g., isopropanol, ethanol, or the like, at 90 to 100° C. and typically requires 4 to 6 hours to complete. The cyclization is carried out via intramolecular thermal cyclization at 250° C. in the solvents such as diphenyl ether, dowtherm or the like.

Compounds of Formula IV in which Z¹, Z², Z³ are CH, CR² and CH, respectively, can also be prepared by the reaction scheme shown in FIG. 3, wherein R³ is shown as F, and R² and R⁵ are as defined in the Summary of the Invention.

Nitriles of Formula 4.2 can be formed by reacting amines of formula 11 in a suitable solvent, e.g., DMF with a base such as K₂CO₃ at elevated temperatures such as 60° C. or in a solvent such as CH₃CN carried out in a sealed tube heated with an oil bath set at 150° C. Amines of the formula 11 can be formed by reacting amines formula 10 with trisubstituted amines of the formula 9 in a suitable solvent. Trisubstituted amines of formula 9 can be formed by the reaction nitriles of the formula 8 with acid chlorides of the formula 7 in the presence of base. Acid chlorides of the formula 6 can be formed from the substituted benzoic acids of the formula 6 by reacting with oxalyl chloride, P(O)Cl₃ or a suitable chlorinating agent in the presence of a suitable solvent such as CH₂Cl₂ or the absence of solvent.

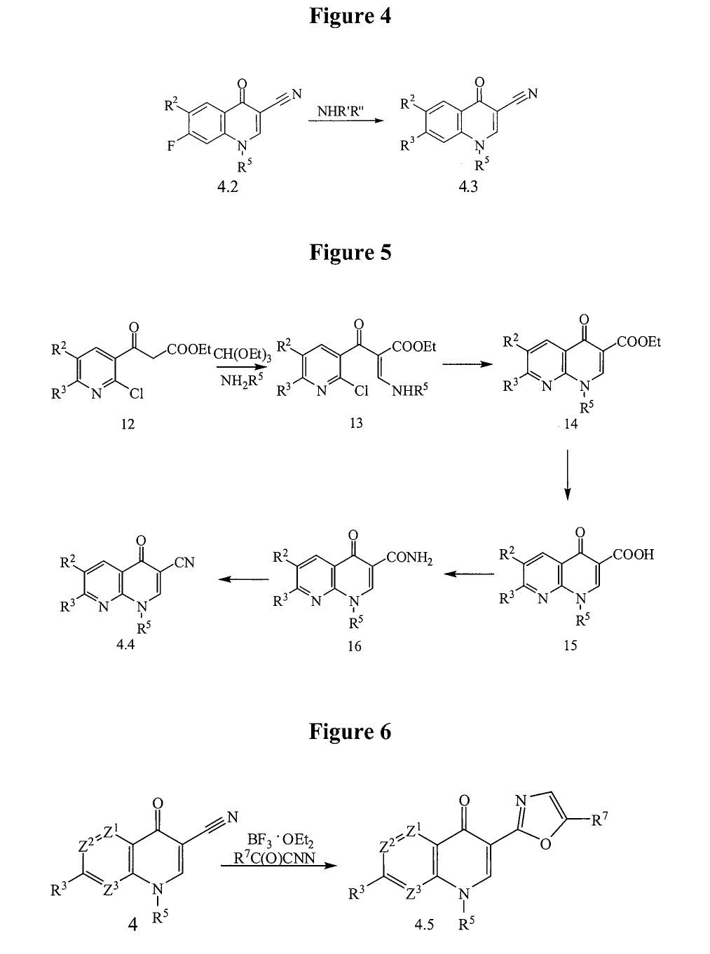

Nitriles of Formula 4.3 in which R³ is —NHR³⁶ or —N(R³⁷)₂ (Formula 4.3) can be prepared by the reaction scheme shown in FIG. 4, in which R² and R⁵ are as defined in the Summary of the Invention and R³ is —NHR³⁶ or —N(R³⁷)₂, wherein R³⁶ is alkyl, alkyloxycarbonylalkyl, carbamoylalkyl, heterocycloalkylalkyl, hydroxyalkoxyalkyl, aminoalkyl, hydroxyalkyl and R³⁷ is hydrogen, alkyl or hydroxyalkyl, or each R³⁷ together with the nitrogen atom to which they are attached form heterocycloalkyl or heteroaryl, wherein heterocycloalkyl may be substituted with one to three groups independently selected from alkyl, hydroxy, acetyl, heteroaryl, carboxy and oxo and heteroaryl may be substituted with one to three groups independently selected from alkyl, hydroxy, acetyl, heteroaryl and carboxy. Briefly, nitriles of Formula 4.3 can be prepared by reacting a nitrile of Formula 4.2 with NH₂R³⁶ or NH(R³⁷)₂ in a suitable solvent, e.g., acetonitrile, DMF or the like at temperatures between 60° C. and 100° C. in the presence or absence of inorganic base for up to 12 to 14 hours.

Compounds of Formula 4.4 in (Formula I in which Z¹ is CH, Z² is CR² and Z³ is N) can be prepared by the reaction scheme shown in FIG. 5, wherein each R², R³ and R⁵ are as defined in the Summary of the Invention. In which some groups R³ is —NHR³⁶ or —N(R³⁷)₂, wherein R³⁶ is alkyl, alkyloxycarbonylalkyl, carbamoylalkyl, heterocycloalkylalkyl, hydroxyalkoxyalkyl, aminoalkyl, hydroxyalkyl and R³⁷ is hydrogen, alkyl or hydroxyalkyl, or each R³⁷ together with the nitrogen atom to which they are attached form heterocycloalkyl or heteroaryl, wherein heterocycloalkyl may be substituted with one to three groups independently selected from alkyl, hydroxy, acetyl, heteroaryl, carboxy and oxo and heteroaryl may be substituted with one to three groups independently selected from alkyl, hydroxy, acetyl, heteroaryl and carboxy.

Nitriles of the Formula 4.4 can be prepared by reacting diazepam with ethyl 7-chloro-6-fluoro-4-oxo-1-(4-trifluoromethylbenzyl)-1,4-dihydro-[1,8]naphthyridine-3-carboxylate in a suitable solvent like DMA in the presence of organic base. The intermediate can be hydrolyzed by treating with a strong base such as KOH in a solvent such as EtOH. The resulting carboxylic acid can be activated with a coupling agent such as a mixed anhydride and treated with ammonia. The primary amide can then be converted to the nitrile by treating with PPE at 60° C.

Compounds of Formula I in which R⁶ is Formula (d): 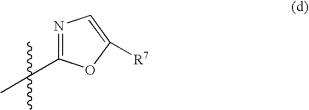
can be prepared by the reaction scheme provided in FIG. 6. Briefly, compounds of Formula I in which R⁶ is Formula (d) can be prepared by reacting a compound of Formula 4 with a diazoketone having the formula R⁷C(O)CNN in which Z¹, Z², Z³, R³, R⁵, R⁷ are defined in the Summary of the Invention.

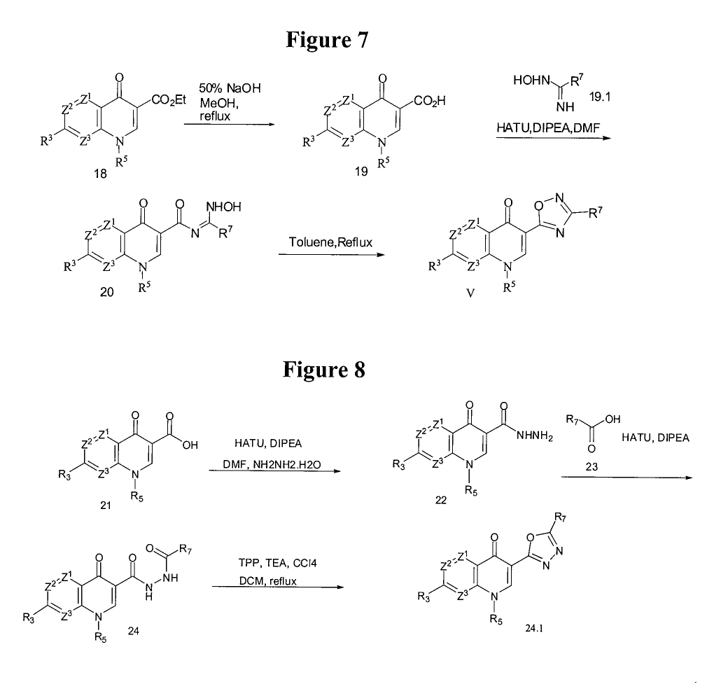

Compounds of Formula I in which R⁶ is Formula (b): 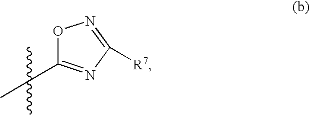
can be prepared by the reaction scheme shown in FIG. 7, in which each Z¹, Z², Z³, R³, R⁵ and R⁷ are as defined in the Summary of the Invention. Here, compounds of Formula I in which R⁶ is Formula (b) can be prepared by reacting a carboxylic acid of Formula 19 with a hydroxyamidine of the formula R⁷C(NH)NHOH. The reaction is carried out in a suitable solvent, e.g., DMF, DMA. Hydroxyamidines of the formula R⁷C(NH)NHOH can be prepared by reacting R⁷CN with hydroxylamine in a suitable solvent, e.g., ethanol, or the like, at 60 to 90° C. and typically requires 2 to 3 hours to complete. Carboxylic acids of the formula 19 can be synthesized as described for the synthesis of the compound of formula 15, FIG. 5. In the synthesis of compounds of the formula 19 (Z³=CR³), the chlorine in compounds of formula 12-13 can be replaced by other halogens, e.g. fluorine.

Compounds of Formula I in which R⁶ is Formula (c): 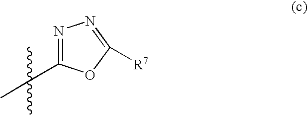
can be prepared by the reaction scheme shown in FIG. 8, in which each Z¹, Z², Z³, R³, R⁵ and R⁷ are as defined in the Summary of the Invention. These compounds of Formula I in which R⁶ is Formula (c) can be prepared by coupling a carboxylic acid of Formula 23 with hydrazine hydrate. The reaction is carried out in a suitable solvent, e.g., DMF. The coupling of the hydrazine amide with a substituted phenylacetic acid followed by cyclization in the presence of triphenylphosphine, CCl₄ and triethylamine affords the 1,3,4 oxadiazole of the formula 24.1, in which each Z¹, Z², Z³, R³, R⁵ and R⁷ are as defined in the Summary of the Invention.

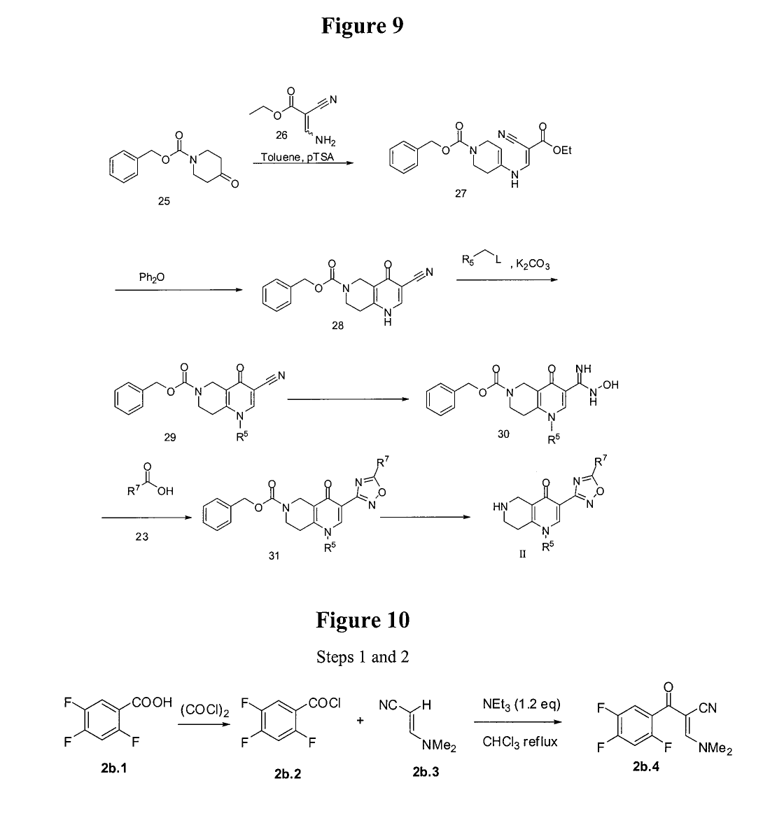

Compounds of Formula II in which R⁶ is Formula (a) can be prepared as shown in FIG. 9, in which each R⁵ and R⁷ are as defined in the Summary of the Invention.

Compounds of Formula II in which R⁶ is Formula (b) can be prepared by condensing a piperdone Formula 25 with ethyl 3-amino-2-cyanoacrylate. The reaction is carried out in a suitable solvent, e.g., toluene. The cyclization to the 4-pyridone in Ph₂O at elevated temperatures. Hydroxamidines of the formula 30 can be prepared by reacting 29 with hydroxylamine in a suitable solvent, e.g., ethanol, or the like, at 60 to 90° C. and requires 2 to 3 hours to complete. Subsequent coupling of hydroxyamidine of the formula 30 with a substituted phenylacetic acid of the Formula 25 followed by cyclization affords the 1,3,4 oxadiazole containing structures of Formula II.

### Reference 1

### 6,7-Dimethoxy-4-oxo-1,4-dihydro-quinoline-3-carbonitrile

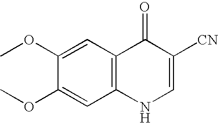

Ethyl 2-cyano-3-ethoxy-acrylate (5.5 gm, 32.7 mmol) was combined with 3,4-dimethoxy-phenylamine (5.0 gm, 32.7 mmol) in isopropanol (100 mL) and the mixture was heated at reflux for approximately 5 hours. The solvent was evaporated on the rotovap and the residue was purified by silica gel column chromatography to give ethyl 2-cyano-3-(3,4-dimethoxy-phenylamino)-acrylate (6.8 gm, 24.6 mmols). Mass Spec: m/z 275 (M−1), 277 (M+1).

A solution of ethyl 2-cyano-3-(3,4-dimethoxy-phenylamino)-acrylate (6.8 gm, 24.6 mmols) in diphenyl ether (8 mL) was added slowly to diphenyl ether heated at 250° C. The reaction mixture was heated at 250° C. for 30 to 60 min and the reaction was monitored by mass spec and HPLC. The mixture was cooled to room temperature and solid material was collected by filtration and washed with hexane to give 6,7-dimethoxy-4-oxo-1,4-dihydro-quinoline-3-carbonitrile (3.9 gm, 16.9 mmol). Mass Spec: m/z 229 (M−1), 231 (M+1).

Proceeding as in Reference 1, but substituting 4-methoxy-phenylamine, gave 6-methoxy-4-oxo-1,4-dihydro-quinoline-3-carbonitrile.

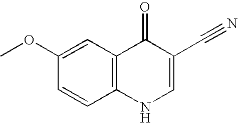

Proceeding as in Reference 1, but substituting 4-amino-phenol gave 6-hydroxy-4-oxo-1,4-dihydro-quinoline-3-carbonitrile.

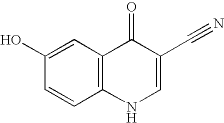

Proceeding as in Reference 1, but substituting 4-amino-benzoic acid gave 3-cyano-4-oxo-1,4-dihydro-quinoline-6-carboxylic acid.

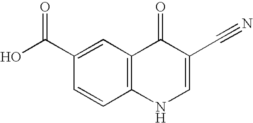

Proceeding as in Reference 1, but substituting 3,4-difluoro-phenylamine, gave 6,7-difluoro-4-oxo-1,4-dihydro-quinoline-3-carbonitrile. TLC system: 50% Ethyl acetate/Pet ether. (R_{f} value: 0.3); ¹H NMR (DMSO-d₆; 300 MHz): 12.95 (broad, 1H), 8.8-8.78 (d, J=6, 1H), 8.06-8.03 (t, 1H), 7.67-7.64 (m, 1H), 7.44-7.42 (t, 1H), 7.01 (t d, 1H); Mass: (M+H)⁺ 206; Brown solid; 1.1 gm (50%)

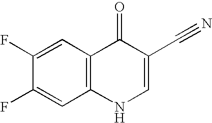

Proceeding as in Reference 1, but substituting 3-methoxy-phenylamine gave 7-methoxy-4-oxo-1,4-dihydro-quinoline-3-carbonitrile.

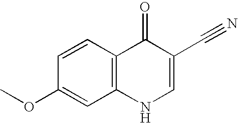

Proceeding as in Reference 1, but substituting 3-fluoro-phenylamine gave 7-fluoro-4-oxo-1,4-dihydro-quinoline-3-carbonitrile.

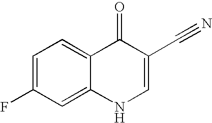

Proceeding as in Reference 1, but substituting (4-amino-phenyl)-acetic acid gave (3-cyano-4-oxo-1,4-dihydro-quinolin-6-yl)-acetic acid.

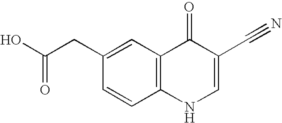

Proceeding as in Reference 1, but substituting phenylamine, gave 4-oxo-1,4-dihydro-quinoline-3-carbonitrile. TLC system: 50% Ethyl Acetate/Pet ether; (Rf value: 0.3); ¹H NMR (DMSO-d₆; 300 MHz): 12.65 (broad, 1H), 8.5 (s, 1H), 7.70-7.66 (m, 2H), 7.62 (m, 2H); Mass: (M+H)⁺ 171; Brown Solid; 6.2 gm, (65%)

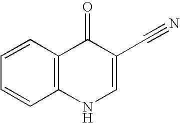

### Reference 2

### 1-(4-Chloro-benzyl)-6,7-dimethoxy-4-oxo-1,4-dihydro-quinoline-3-carbonitrile

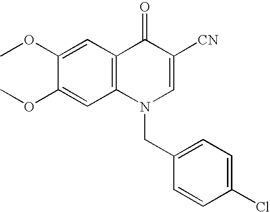

Dry potassium carbonate (1.2 gm, 8.6 mmol) and 4-chlorobenzyl bromide (1.3 gm, 6.45 mmol) was added to a solution of 6,7-dimethoxy-4-oxo-1,4-dihydro-quinoline-3-carbonitrile (1 gm, 4.3 mmol), prepared as in Reference 1, in DMF (25 mL) and the mixture was heated to 60° C. for 2 to 4 hours. The solvent was removed by rotoevaporation and the residue was purified by silica gel column chromatography using ethylacetate and hexane as eluents to give 1-(4-chloro-benzyl)-6,7-dimethoxy-4-oxo-1,4-dihydro-quinoline-3-carbonitrile (1.1 gm, 3.1 mmol). Mass Spec: m/z 353 (M−1), 355 (M+1).

Proceeding as in Reference 2, but substituting 6,7-difluoro-4-oxo-1,4-dihydro-quinoline-3-carbonitrile, gave 1-(4-chloro-benzyl)-6,7-difluoro-4-oxo-1,4-dihydro-quinoline-3-carbonitrile.

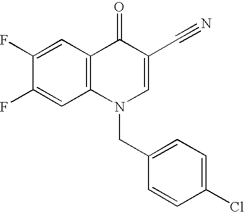

Proceeding as in Reference 2, but substituting 6,7-difluoro-4-oxo-1,4-dihydro-quinoline-3-carbonitrile and cyclohexylmethyl chloride, gave 1-cyclohexylmethyl-6,7-difluoro-4-oxo-1,4-dihydro-quinoline-3-carbonitrile.

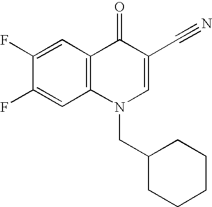

Proceeding as in Reference 2, but substituting 6-methoxy-4-oxo-1,4-dihydro-quinoline-3-carbonitrile and cyclohexylmethyl chloride, gave 1-cyclohexylmethyl-6-methoxy-4-oxo-1,4-dihydro-quinoline-3-carbonitrile.

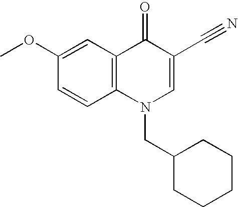

Proceeding as in Reference 2, but substituting 6-methoxy-4-oxo-1,4-dihydro-quinoline-3-carbonitrile, gave 1-(4-chloro-benzyl)-6-methoxy-4-oxo-1,4-dihydro-quinoline-3-carbonitrile.

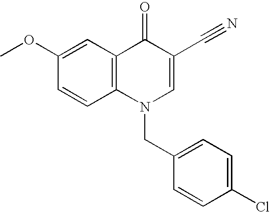

Proceeding as in Reference 2, but substituting 6-hydroxy-4-oxo-1,4-dihydro-quinoline-3-carbonitrile, gave 1-(4-chloro-benzyl)-6-hydroxy-4-oxo-1,4-dihydro-quinoline-3-carbonitrile.

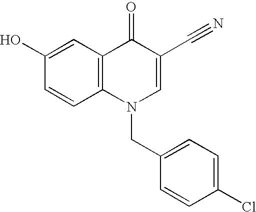

Proceeding as in Reference 2, but substituting 6,7-difluoro-4-oxo-1,4-dihydro-quinoline-3-carbonitrile and 4-trifluoromethylbenzyl chloride, gave 6,7-difluoro-4-oxo-1-(4-trifluoromethyl-benzyl)-1,4-dihydro-quinoline-3-carbonitrile. TLC system: 50% Ethyl Acetate/Pet-ether; (R_{f} value: 0.5); ¹H NMR (DMSO-d₆; 300 MHz): 9.17 (s, 1H), 8.1 (t, 1H), 7.88-7.94 (m, 1H), 7.7 (d, J=8.1 Hz, 2H), 7.5 (d, J=8.1 Hz, 2H), 5.73 (s, 2H); Mass: (M+H)⁺ 365; Brown solid; 950 mg (50%).

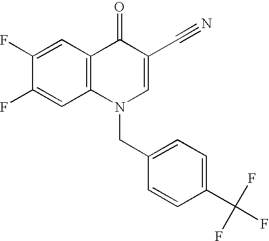

Proceeding as in Reference 2, but substituting 6,7-difluoro-4-oxo-1,4-dihydro-quinoline-3-carbonitrile and 2-fluoro-4-trifluoromethyl-benzyl bromide, gave 6,7-difluoro-1-(2-fluoro-4-trifluoromethyl-benzyl)-4-oxo-1,4-dihydro-quinoline-3-carbonitrile. Mass Spec: m/z 382.8 (M+1).

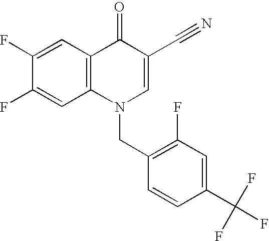

Proceeding as in Reference 2, but substituting 6-hydroxy-4-oxo-1,4-dihydro-quinoline-3-carbonitrile and cyclohexylmethyl chloride, gave 1-cyclohexylmethyl-6-hydroxy-4-oxo-1,4-dihydro-quinoline-3-carbonitrile.

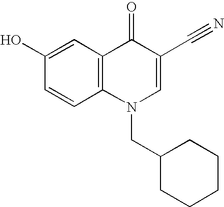

Proceeding as in Reference 2, but substituting 6,7-difluoro-4-oxo-1,4-dihydro-quinoline-3-carbonitrile and 2-fluoro-4-trifluoromethyl-benzyl bromide and 4-chloro-2-fluorobenzyl chloride, gave 1-(4-chloro-2-fluoro-benzyl)-6,7-difluoro-4-oxo-1,4-dihydro-quinoline-3-carbonitrile.

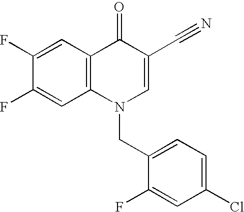

Proceeding as in Reference 2, but substituting 6,7-difluoro-4-oxo-1,4-dihydro-quinoline-3-carbonitrile and 2-ethyl-butyl chloride, gave 1-(2-ethyl-butyl)-6,7-difluoro-4-oxo-1,4-dihydro-quinoline-3-carbonitrile.

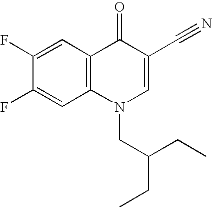

Proceeding as in Reference 2, but substituting 6,7-difluoro-4-oxo-1,4-dihydro-quinoline-3-carbonitrile and 2-chloro-6-fluoro-benzyl chloride, gave 1-(2-chloro-6-fluoro-benzyl)-6,7-difluoro-4-oxo-1,4-dihydro-quinoline-3-carbonitrile.

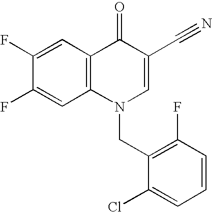

Proceeding as in Reference 2, but substituting 6,7-difluoro-4-oxo-1,4-dihydro-quinoline-3-carbonitrile and 2,4-difluoro-benzyl chloride, gave 1-(2,4-difluoro-benzyl)-6,7-difluoro-4-oxo-1,4-dihydro-quinoline-3-carbonitrile.

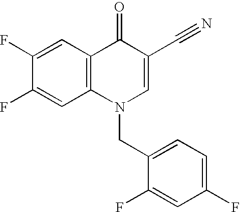

Proceeding as in Reference 2, but substituting 6,7-difluoro-4-oxo-1,4-dihydro-quinoline-3-carbonitrile and 2-fluoro-benzyl chloride, gave 6,7-difluoro-1-(2-fluoro-benzyl)-4-oxo-1,4-dihydro-quinoline-3-carbonitrile.

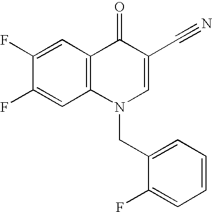

Proceeding as in Reference 2, but substituting 6,7-difluoro-4-oxo-1,4-dihydro-quinoline-3-carbonitrile and 2,6-difluoro-benzyl chloride, gave 1-(2,6-difluoro-benzyl)-6,7-difluoro-4-oxo-1,4-dihydro-quinoline-3-carbonitrile.

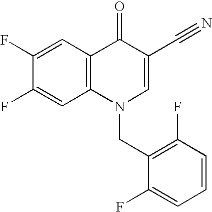

Proceeding as in Reference 2, but substituting 3-cyano-4-oxo-1,4-dihydro-quinoline-6-carboxylic acid and 2-fluoro-4-trifluoromethylbenzyl chloride, gave 3-cyano-1-(2-fluoro-4-trifluoromethyl-benzyl)-4-oxo-1,4-dihydro-quinoline-6-carboxylic acid.

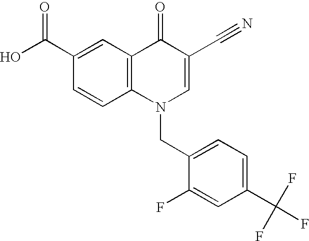

Proceeding as in Reference 2, but substituting 6-fluoro-7-(4-methyl-piperazin-1-yl)-4-oxo-1,4-dihydro-quinoline-3-carbonitrile and 2,6-difluorobenzyl chloride, gave 1-(2,6-difluoro-benzyl)-6-fluoro-7-(4-methyl-piperazin-1-yl)-4-oxo-1,4-dihydro-quinoline-3-carbonitrile.

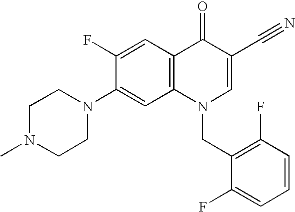

Proceeding as in Reference 2, but substituting 6,7-difluoro-4-oxo-1,4-dihydro-quinoline-3-carbonitrile and 4-(2-chloro-ethyl)-morpholine, gave 6,7-difluoro-1-(2-morpholin-4-yl-ethyl)-4-oxo-1,4-dihydro-quinoline-3-carbonitrile.

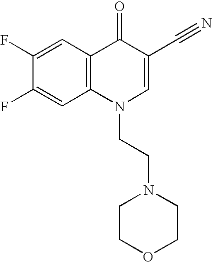

Proceeding as in Reference 2, but substituting 6,7-difluoro-4-oxo-1,4-dihydro-quinoline-3-carbonitrile and 2-chloro-pyrid-5-ylmethyl chloride, gave 1-(6-chloro-pyridin-3-ylmethyl)-6,7-difluoro-4-oxo-1,4-dihydro-quinoline-3-carbonitrile.

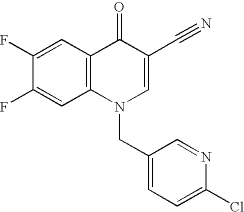

Proceeding as in Reference 2, but substituting 7-methoxy-4-oxo-1,4-dihydro-quinoline-3-carbonitrile and 4-trifluoromethyl-benzyl chloride gave 7-methoxy-4-oxo-1-(4-trifluoromethyl-benzyl)-1,4-dihydro-quinoline-3-carbonitrile.

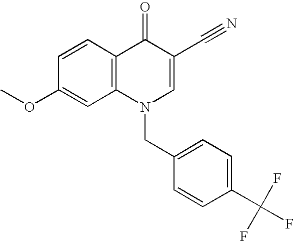

Proceeding as in Reference 2, but substituting (3-cyano-4-oxo-1,4-dihydro-quinolin-6-yl)-acetic acid and 2-fluoro-4-trifluoromethyl-benzyl chloride, gave [3-cyano-1-(2-fluoro-4-trifluoromethyl-benzyl)-4-oxo-1,4-dihydro-quinolin-6-yl]-acetic acid.

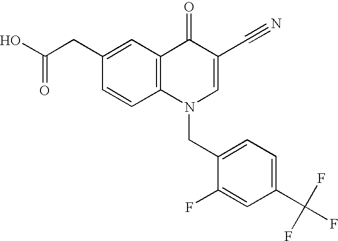

Proceeding as in Reference 2, but substituting 6,7-difluoro-4-oxo-1,4-dihydro-quinoline-3-carbonitrile and phenethyl chloride, gave 6,7-difluoro-4-oxo-1-phenethyl-1,4-dihydro-quinoline-3-carbonitrile.

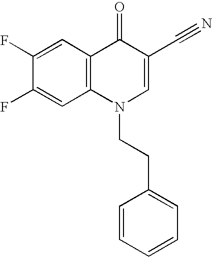

Proceeding as in Reference 2, but substituting 6,7-difluoro-4-oxo-1,4-dihydro-quinoline-3-carbonitrile and 1-(4-trifluoromethylphenyl)cyclopropyl chloride, gave 6,7-difluoro-4-oxo-1-[1-(4-trifluoromethyl-phenyl)-cyclopropyl]-1,4-dihydro-quinoline-3-carbonitrile.

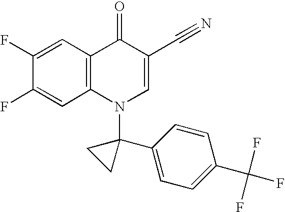

Proceeding as in Reference 2, but substituting 6,7-difluoro-4-oxo-1,4-dihydro-quinoline-3-carbonitrile and 3-trifluoromethyl-benzyl chloride, gave 6,7-difluoro-4-oxo-1-(3-trifluoromethyl-benzyl)-1,4-dihydro-quinoline-3-carbonitrile.

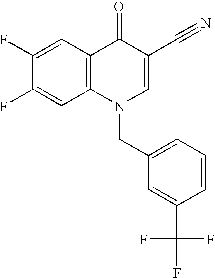

Proceeding as in Reference 2, but substituting 4-oxo-1,4-dihydro-quinoline-3-carbonitrile and 2-fluoro-4-trifluoromethyl-benzyl chloride, gave 1-(2-fluoro-4-trifluoromethyl-benzyl)-4-oxo-1,4-dihydro-quinoline-3-carbonitrile.

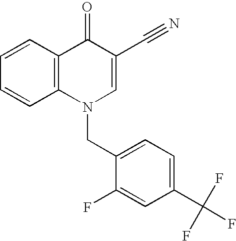

Proceeding as in Reference 2, but substituting 6,7-difluoro-4-oxo-1,4-dihydro-quinoline-3-carbonitrile and 4-tert-butyl-benzyl chloride, gave 1-(4-tert-butyl-benzyl)-6,7-difluoro-4-oxo-1,4-dihydro-quinoline-3-carbonitrile.

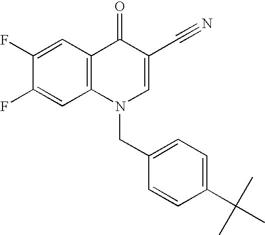

Proceeding as in Reference 2, but substituting benzyl 3-cyano-1,5,7,8-tetrahydro-4H-[1,6]naphthyridine-6-carboxylate and 2-fluoro-4-trifluoromethyl-benzyl bromide, gave benzyl 3-cyano-1-(2-fluoro-4-(trifluoromethyl)benzyl)-4-oxo-4,5,7,8-tetrahydro-1,6-naphthyridine-6(1H)-carboxylate

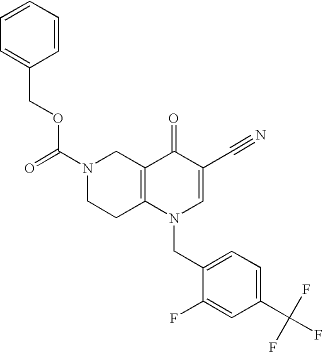

Proceeding as in Reference 2 but substituting 6,7-difluoro-4-oxo-1,4-dihydro-quinoline-3-carbonitrile and 4-trifluormethyl-benzyl chloride, gave 7-chloro-6-fluoro-4-oxo-1-(4-(trifluoromethyl)benzyl)-1,4-dihydroquinoline-3-carbonitrile.

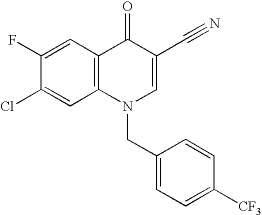

Proceeding as in Reference 2, but substituting 6,7-difluoro-4-oxo-1,4-dihydro-quinoline-3-carbonitrile and 4-fluoro-benzyl chloride, gave 1-(4-fluoro-butyl-benzyl)-6,7-difluoro-4-oxo-1,4-dihydro-quinoline-3-carbonitrile. TLC system: 50% Ethyl Acetate/Pet-ether; (R_{f} value: 0.5); ¹H NMR (DMSO-d₆; 300 MHz): 9.17 (s, 1H), 8.1 (t, 1H), 7.88-7.94 (m, 1H), 7.7 (d, J=8.1 Hz, 2H), 7.5 (d, J=8.1 Hz, 2H), 5.73 (s, 2H); Mass: (M+H)⁺ 315; Brown solid; 762 mg (50%).

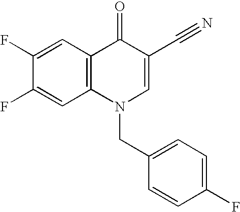

### Reference 2b

### 6,7-difluoro-4-oxo-1-(1-(4-(trifluoromethyl)phenyl)ethyl)-1,4-dihydroquinoline-3-carbonitrile

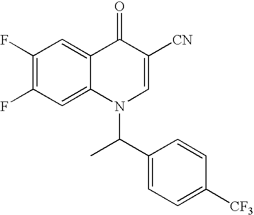

### See FIG. 10

2,4,5-trifluorobenzoic acid (20 g, 113.6 mmol) was dissolved in thionyl chloride (15 ml, 170.4 mmol) at room temperature under nitrogen atmosphere. The reaction mixture was heated to reflux for 4 h and then thionyl chloride was removed under reduced pressure to provide 2,4,5-trifluorobenzoyl chloride 2b.2 (20 g, 103.8 mmol). The acid chloride 2b.2 was dissolved in DCM, cooled to 0° C., and triethyl amine (17.2 ml, 126.2 mmol) followed by 3-dimethyl amino acrylonitrile 2b.3 (11.3 ml, 103.8 mmol) were added. The ice bath was removed and the reaction mixture was stirred at room temperature overnight. The reaction mixture was portioned between water and DCM. The organic layer was separated and dried over Na₂SO₄, filtered and concentrated. The residue was purified by flash column chromatography eluting with 30% ethyl acetate in n-hexane to provide compound 2b.4 (12.2 g, 46.4%). ¹H-NMR (DMSO-d₆) δ 7.81 (1H, s, —CH), 7.78-7.56 (2H, m, Ar—H), 3.31 (3H, s, —CH₃), 3.27 (3H, s, —CH₃). EIMS (m/z): 255.21 (M+H)⁺.

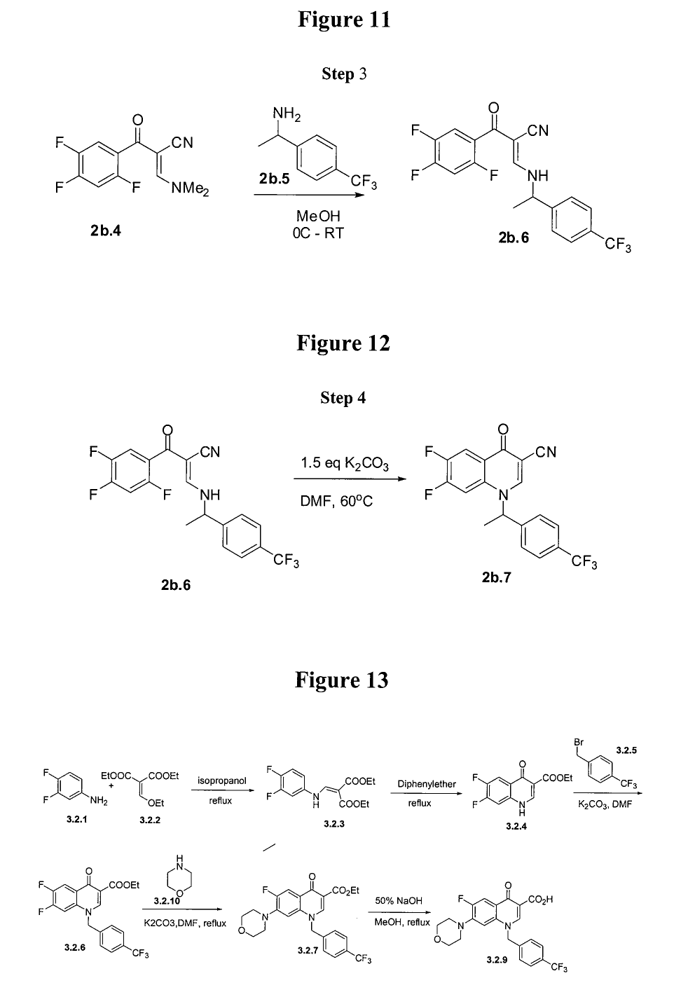

### See FIG. 11

To a stirred solution of compound 2b.4 (2.0 g, 7.8 mmol) in methanol (60 mL) was added 4-trifluoromethyl-α-methyl benzyl amine 2b.5 (1.6 g, 9.4 mmol) under nitrogen atmosphere. The reaction mixture stirred at room temperature for 4 to 5 days. Once the reaction was completed then solvent was removed under reduced pressure. The residue was purified by flash column chromatography eluting with 15% ethyl acetate in n-hexane to provide compound 2b.6 (2.6 g, 92%). ¹H-NMR (DMSO-d₆) δ 10.98 (½H, m, Ar—H), 9.94 (½H, m, Ar—H), 8.24-7.98 (1H, m, Ar—H), 7.80-7.56 (6H, m, Ar—H), 5.02 (1H, m, —CH), 1.72-1.47 (3H, 2×d, —CH₃). EIMS (m/z): 399.4 (M+H)⁺.

### See FIG. 12

To a stirred solution of compound 2b.6 (2.6 g, 7.2 mmol) in DMF (40 mL) was added potassium carbonate (1.4 g, 10.8 mmol) under nitrogen atmosphere. The reaction mixture was stirred at 60° C. for 1 h then water added to the reaction mixture, the resulting precipitate was filtered, washed diethyl ether and dried under reduced pressure at 60° C. for 1 h to provide compound 2b.7 (2.1 g, 76%). ¹H-NMR (DMSO-d₆) δ 9.0 (1H, s, Ar—H), 8.2-8.02 (2H, m, Ar—H), 7.88-7.69 (4H, ABq, Ar—H), 6.24 (1H, q, —CH), 1.98 (3H, d, —CH₃). EIMS (m/z): 379.6 (M+H)⁺.

Proceeding as in Reference 2b but substituting 2-(4-(trifluoromethyl)phenyl)propan-2-amine and 2-(3,4-difluorobenzoyl)-3-(dimethylamino)acrylonitrile gave 6,7-difluoro-4-oxo-1-(2-(4-(trifluoromethyl)phenyl)propan-2-yl)-1,4-dihydroquinoline-3-carbonitrile TLC system: 50% Ethyl Acetate/Pet ether; (Rf value: 0.3); ¹H NMR (DMSO-d₆; 300 MHz): 9.01 (s, 1H); 8.16 (t, 1H); 7.77-7.74 (d, J=9 Hz, 2H); 7.60-7.57 (d, J=9 Hz; 2H); 6.98-6.96 (q, 1H); 2.08 (s, 6H); Mass: (M+H)⁺ 393; Pale Yellow Solid; 530 mg; (84.3%)

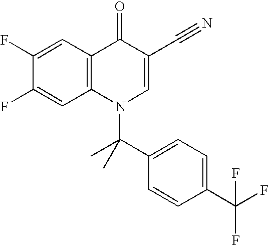

Proceeding as in Reference 2b, but in step 3 substituting 2-methyl-2-(4-(trifluoromethyl)phenyl)propan-1-amine, 6,7-difluoro-1-(2-methyl-2-(4-(trifluoromethyl)phenyl)propyl)-4-oxo-1,4-dihydroquinoline-3-carbonitrile

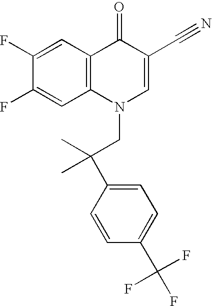

Proceeding as in Reference 2b, but in step 3 substituting 2-(phenyl)propan-2-amine, gave 6,7-difluoro-4-oxo-1-(2-phenylpropan-2-yl)-1,4-dihydroquinoline-3-carbonitrile

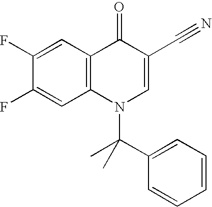

Proceeding as in Reference 2b, but in step 3 substituting p-methoxybenzyl amine, gave 6,7-difluoro-1-(4-methoxybenzyl)-4-oxo-1,4-dihydroquinoline-3-carbonitrile.

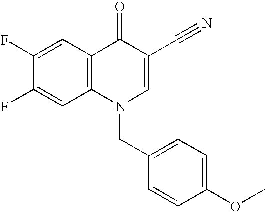

Proceeding as in Reference 2b but substituting 1-(4-(trifluoromethyl)phenyl)ethanamine and 2-(3,4-difluorobenzoyl)-3-(dimethylamino)acrylonitrile gave 6,7-difluoro-4-oxo-1-(1-(4-(trifluoromethyl)phenyl)ethyl)-1,4-dihydroquinoline-3-carbonitrile. ¹H-NMR (DMSO-d₆) δ 9.0 (1H, s, Ar—H), 8.2-8.02 (2H, m, Ar—H), 7.88-7.69 (4H, ABq, Ar—H), 6.24 (1H, q, —CH), 1.98 (3H, d, —CH₃). EIMS (m/z): 379.6 (M+H)⁺

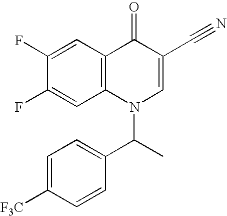

### Reference 2c

### Ethyl 2-(2,6-dichloro-5-fluoropyridine-3-carbonyl)-3-(4-trifluoromethyl-benzylamino)acrylate

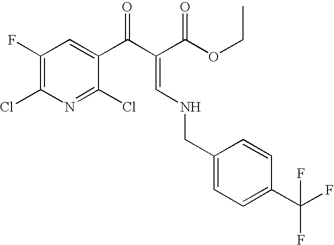

A mixture of ethyl 2,6-dichloro-5-fluoro-pyrid-3-yl-3-oxo-propionate (3.0 g, 10.7 mmol), triethylorthoformate (2.4 g, 2.68 mL, 16.1 mmol) and acetic anhydride (2.73 g, 2.38 mL, 26.7 mmol) was heated to reflux for 1 hour at 140° C. The mixture was cooled and then concentrated. The residue was diluted with ethanol (5 mL) and 4-trifluoromethylbenzyl amine (2.25 g, 1.83 mL, 12.84 mmol) was added. The mixture was stirred for approximately 12 hours and was filtered to collect a solid material (2.2 g). The filtrate was concentrated and product was purified from the residue by chromatography over silica to give an additional solid material (0.8 g). The solid material was combined to give ethyl 2-(2,6-dichloro-5-fluoropyridine-3-carbonyl)-3-(4-trifluoromethyl-benzylamino)acrylate (3.0 g, 60%). Rₚ=0.55 (20% EtOAc/hexane).

### Reference 3

### 6-Fluoro-1-(2-fluoro-4-trifluoromethyl-benzyl)-7-(4-methyl-piperazin-1-yl)-4-oxo-1,4-dihydro-quinoline-3-carbonitrile

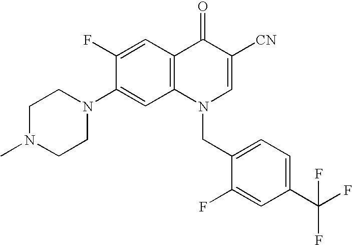

1-Methyl-piperazine (220 mg, 0.24 mL, 2.2 mmol) was added to a suspension of 6,7-difluoro-1-(2-fluoro-4-trifluoromethyl-benzyl)-4-oxo-1,4-dihydro-quinoline-3-carbonitrile (302 mg, 1 mmol), prepared as in Reference 2, in acetonitrile (10 mL). The reaction mixture was heated in a sealed tube at 150° C. for 16 hours and then cooled to room temperature. Precipitate was collected by filtration and then dried on high vacuum to give 6-fluoro-1-(2-fluoro-4-trifluoromethyl-benzyl)-7-(4-methyl-piperazin-1-yl)-4-oxo-1,4-dihydro-quinoline-3-carbonitrile (370 mg, 0.8 mmol, 80%) as a tan solid. Mass Spec: m/z 463.0 (M+1)⁺.

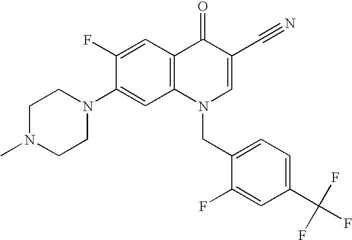

Proceeding as in Reference 3, but substituting 1-(4-chloro-benzyl)-6,7-difluoro-4-oxo-1,4-dihydro-quinoline-3-carbonitrile, gave 1-(4-chloro-benzyl)-6-fluoro-7-(4-methyl-piperazin-1-yl)-4-oxo-1,4-dihydro-quinoline-3-carbonitrile.

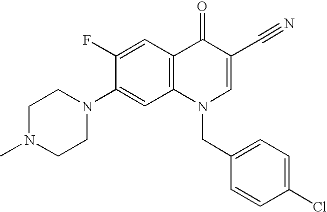

Proceeding as in Reference 3, but substituting 1-cyclohexylmethyl-6,7-difluoro-4-oxo-1,4-dihydro-quinoline-3-carbonitrile, gave 1-cyclohexylmethyl-6-fluoro-7-(4-methyl-piperazin-1-yl)-4-oxo-1,4-dihydro-quinoline-3-carbonitrile.

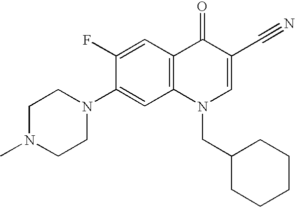

Proceeding as in Reference 3, but substituting 1-(4-chloro-benzyl)-6,7-difluoro-4-oxo-1,4-dihydro-quinoline-3-carbonitrile and piperazine, gave 1-(4-chloro-benzyl)-6-fluoro-4-oxo-7-piperazin-1-yl-1,4-dihydro-quinoline-3-carbonitrile.

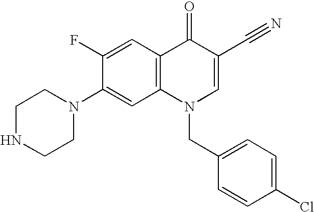

Proceeding as in Reference 3, but substituting 1-(4-chloro-benzyl)-6,7-difluoro-4-oxo-1,4-dihydro-quinoline-3-carbonitrile and 4-hydroxypiperidine, gave 1-(4-chloro-benzyl)-6-fluoro-7-(4-hydroxy-piperidin-1-yl)-4-oxo-1,4-dihydro-quinoline-3-carbonitrile.

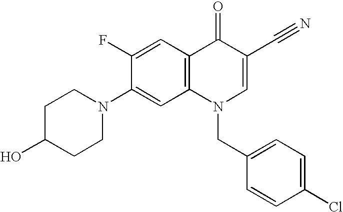

Proceeding as in Reference 3, but substituting 1-(4-chloro-benzyl)-6,7-difluoro-4-oxo-1,4-dihydro-quinoline-3-carbonitrile and 4-acetylpiperazine, gave 7-(4-acetyl-piperazin-1-yl)-1-(4-chloro-benzyl)-6-fluoro-4-oxo-1,4-dihydro-quinoline-3-carbonitrile.

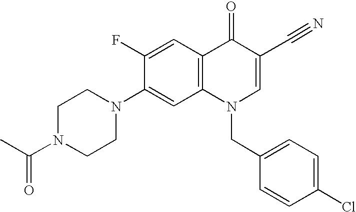

Proceeding as in Reference 3, but substituting 1-(4-chloro-2-fluoro-benzyl)-6,7-difluoro-4-oxo-1,4-dihydro-quinoline-3-carbonitrile, gave 1-(4-chloro-2-fluoro-benzyl)-6-fluoro-7-(4-methyl-piperazin-1-yl)-4-oxo-1,4-dihydro-quinoline-3-carbonitrile.

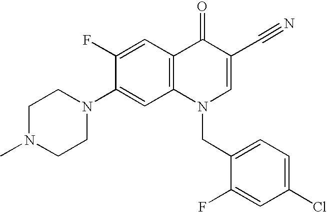

Proceeding as in Reference 3, but substituting 6,7-difluoro-4-oxo-1-(4-trifluoromethyl-benzyl)-1,4-dihydro-quinoline-3-carbonitrile, gave 6-fluoro-7-(4-methyl-piperazin-1-yl)-4-oxo-1-(4-trifluoromethyl-benzyl)-1,4-dihydro-quinoline-3-carbonitrile.

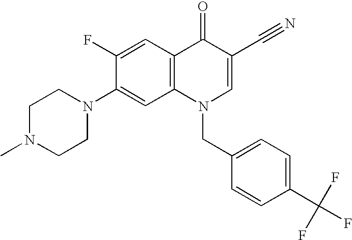

Proceeding as in Reference 3, but substituting 6,7-difluoro-1-(2-fluoro-4-trifluoromethyl-benzyl)-4-oxo-1,4-dihydro-quinoline-3-carbonitrile and [1,4]diazepane, gave 7-[1,4]diazepan-1-yl-6-fluoro-1-(2-fluoro-4-trifluoromethyl-benzyl)-4-oxo-1,4-dihydro-quinoline-3-carbonitrile.

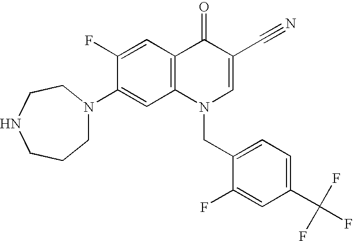

Proceeding as in Reference 3, but substituting 1-(2-ethyl-butyl)-6,7-difluoro-4-oxo-1,4-dihydro-quinoline-3-carbonitrile, gave 1-(2-ethyl-butyl)-6-fluoro-7-(4-methyl-piperazin-1-yl)-4-oxo-1,4-dihydro-quinoline-3-carbonitrile.

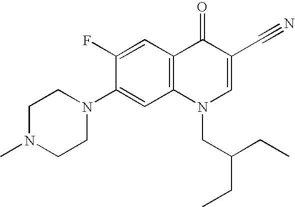

Proceeding as in Reference 3, but substituting 1-(2-chloro-6-fluoro-benzyl)-6,7-difluoro-4-oxo-1,4-dihydro-quinoline-3-carbonitrile, gave 1-(2-chloro-6-fluoro-benzyl)-6-fluoro-7-(4-methyl-piperazin-1-yl)-4-oxo-1,4-dihydro-quinoline-3-carbonitrile.

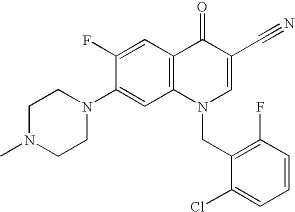

Proceeding as in Reference 3, but substituting 1-(2,4-difluoro-benzyl)-6,7-difluoro-4-oxo-1,4-dihydro-quinoline-3-carbonitrile, gave 1-(2,4-difluoro-benzyl)-6-fluoro-7-(4-methyl-piperazin-1-yl)-4-oxo-1,4-dihydro-quinoline-3-carbonitrile.

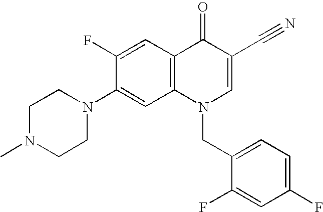

Proceeding as in Reference 3, but substituting 6,7-difluoro-1-(2-fluoro-benzyl)-4-oxo-1,4-dihydro-quinoline-3-carbonitrile, gave 6-fluoro-1-(2-fluoro-benzyl)-7-(4-methyl-piperazin-1-yl)-4-oxo-1,4-dihydro-quinoline-3-carbonitrile.

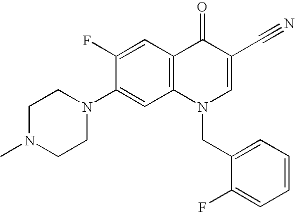

Proceeding as in Reference 3, but substituting 1-(2,6-difluoro-benzyl)-6,7-difluoro-4-oxo-1,4-dihydro-quinoline-3-carbonitrile, gave 1-(2,6-difluoro-benzyl)-6-fluoro-7-(4-methyl-piperazin-1-yl)-4-oxo-1,4-dihydro-quinoline-3-carbonitrile.

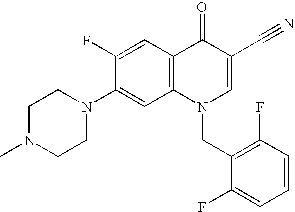

Proceeding as in Reference 3, but substituting 1-(4-chloro-benzyl)-6,7-difluoro-4-oxo-1,4-dihydro-quinoline-3-carbonitrile and 4-pyridin-2-yl-piperazine, gave 1-(4-chloro-benzyl)-6-fluoro-4-oxo-7-(4-pyridin-2-yl-piperazin-1-yl)-1,4-dihydro-quinoline-3-carbonitrile.

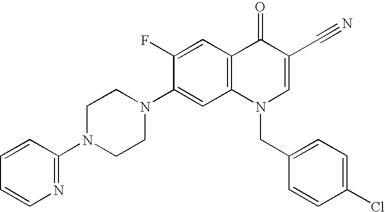

Proceeding as in Reference 3, but substituting 6,7-difluoro-1-(2-fluoro-4-trifluoromethyl-benzyl)-4-oxo-1,4-dihydro-quinoline-3-carbonitrile, gave 6-fluoro-1-(2-fluoro-4-trifluoromethyl-benzyl)-4-oxo-7-piperazin-1-yl-1,4-dihydro-quinoline-3-carbonitrile.

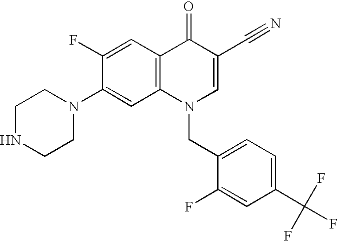

Proceeding as in Reference 3, but substituting 6,7-difluoro-1-(2-fluoro-4-trifluoromethyl-benzyl)-4-oxo-1,4-dihydro-quinoline-3-carbonitrile and dimethylamine gave 7-dimethylamino-6-fluoro-1-(2-fluoro-4-trifluoromethyl-benzyl)-4-oxo-1,4-dihydro-quinoline-3-carbonitrile.

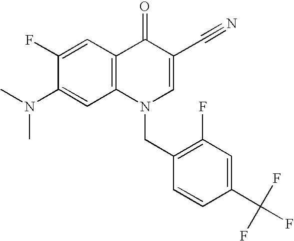

Proceeding as in Reference 3, but substituting 1-(4-chloro-benzyl)-6-fluoro-7-(4-methyl-piperazin-1-yl)-4-oxo-1,4-dihydro-quinoline-3-carbonitrile and 3-cyano-2-phenyl-propionic acid, gave 3-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-3-phenyl-propionitrile.

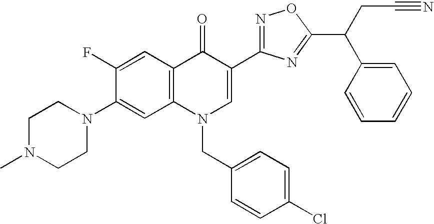

Proceeding as in Reference 3, but substituting 6,7-difluoro-1-(2-fluoro-4-trifluoromethyl-benzyl)-4-oxo-1,4-dihydro-quinoline-3-carbonitrile and methylamine, gave 6-fluoro-1-(2-fluoro-4-trifluoromethyl-benzyl)-7-methylamino-4-oxo-1,4-dihydro-quinoline-3-carbonitrile.

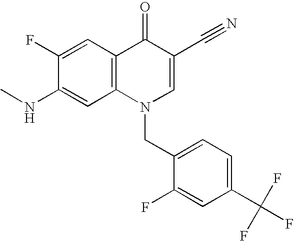

Proceeding as in Reference 3, but substituting 6,7-difluoro-1-(2-fluoro-4-trifluoromethyl-benzyl)-4-oxo-1,4-dihydro-quinoline-3-carbonitrile and 1H-imidazole, gave 6-fluoro-1-(2-fluoro-4-trifluoromethyl-benzyl)-7-imidazol-1-yl-4-oxo-1,4-dihydro-quinoline-3-carbonitrile.

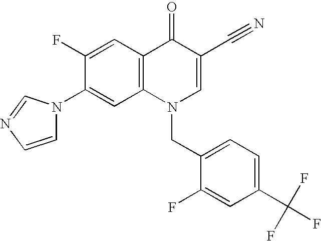

Proceeding as in Reference 3, but substituting 6,7-difluoro-1-(2-fluoro-4-trifluoromethyl-benzyl)-4-oxo-1,4-dihydro-quinoline-3-carbonitrile and 4-methyl-1H-imidazole, gave 6-fluoro-1-(2-fluoro-4-trifluoromethyl-benzyl)-7-(4-methyl-imidazol-1-yl)-4-oxo-1,4-dihydro-quinoline-3-carbonitrile.

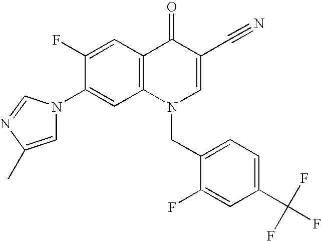

Proceeding as in Reference 3, but substituting 6,7-difluoro-1-(2-morpholin-4-yl-ethyl)-4-oxo-1,4-dihydro-quinoline-3-carbonitrile gave 6-fluoro-7-(4-methyl-piperazin-1-yl)-1-(2-morpholin-4-yl-ethyl)-4-oxo-1,4-dihydro-quinoline-3-carbonitrile.

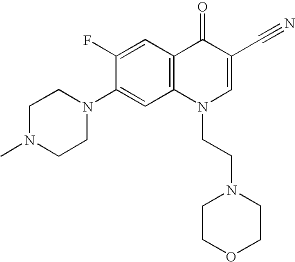

Proceeding as in Reference 3, but substituting 1-(6-chloro-pyridin-3-ylmethyl)-6,7-difluoro-4-oxo-1,4-dihydro-quinoline-3-carbonitrile, gave 1-(6-chloro-pyridin-3-ylmethyl)-6-fluoro-7-(4-methyl-piperazin-1-yl)-4-oxo-1,4-dihydro-quinoline-3-carbonitrile.

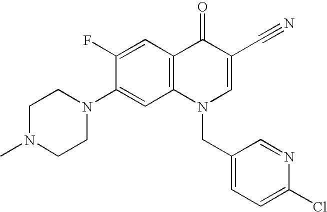

Proceeding as in Reference 3, but substituting 6,7-difluoro-1-(2-fluoro-4-trifluoromethyl-benzyl)-4-oxo-1,4-dihydro-quinoline-3-carbonitrile and methyl amino-acetate, gave methyl[3-cyano-6-fluoro-1-(2-fluoro-4-trifluoromethyl-benzyl)-4-oxo-1,4-dihydro-quinolin-7-ylamino]-acetate.

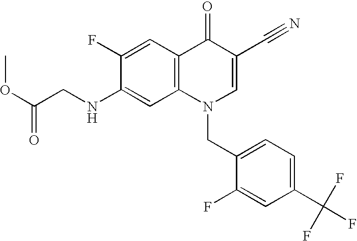

Proceeding as in Reference 3, but substituting 6,7-difluoro-1-(2-fluoro-4-trifluoromethyl-benzyl)-4-oxo-1,4-dihydro-quinoline-3-carbonitrile and 2-amino-acetamide gave 2-[3-cyano-6-fluoro-1-(2-fluoro-4-trifluoromethyl-benzyl)-4-oxo-1,4-dihydro-quinolin-7-ylamino]-acetamide.

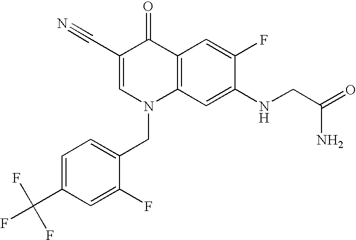

Proceeding as in Reference 3, but substituting 6,7-difluoro-4-oxo-1-(4-trifluoromethyl-benzyl)-1,4-dihydro-quinoline-3-carbonitrile and 2-oxopiperazine, gave 6-fluoro-4-oxo-7-(3-oxo-piperazin-1-yl)-1-(4-trifluoromethyl-benzyl)-1,4-dihydro-quinoline-3-carbonitrile.

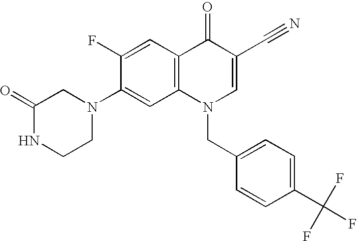

Proceeding as in Reference 3, but substituting 6,7-difluoro-4-oxo-1-(4-trifluoromethyl-benzyl)-1,4-dihydro-quinoline-3-carbonitrile and [1,4]diazepane gave 7-[1,4]diazepan-1-yl-6-fluoro-4-oxo-1-(4-trifluoromethyl-benzyl)-1,4-dihydro-quinoline-3-carbonitrile.

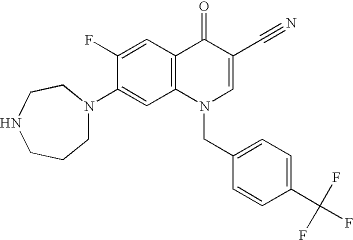

Proceeding as in Reference 3, but substituting 6,7-difluoro-4-oxo-1-(4-trifluoromethyl-benzyl)-1,4-dihydro-quinoline-3-carbonitrile and 2-morpholin-4-yl-ethylamine gave 6-fluoro-7-(2-morpholin-4-yl-ethylamino)-4-oxo-1-(4-trifluoromethyl-benzyl)-1,4-dihydro-quinoline-3-carbonitrile.

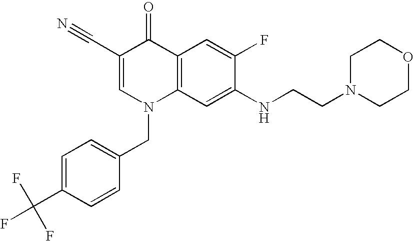

Proceeding as in Reference 3, but substituting 6,7-difluoro-4-oxo-1-(4-trifluoromethyl-benzyl)-1,4-dihydro-quinoline-3-carbonitrile and 2-(2-amino-ethoxy)-ethanol, gave 6-fluoro-7-[2-(2-hydroxy-ethoxy)-ethylamino]-4-oxo-1-(4-trifluoromethyl-benzyl)-1,4-dihydro-quinoline-3-carbonitrile.

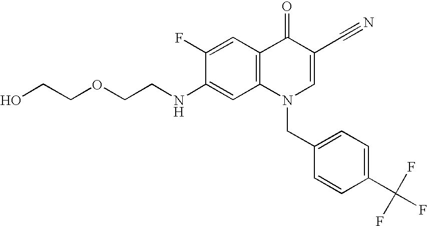

Proceeding as in Reference 3, but substituting 6,7-difluoro-4-oxo-1-(4-trifluoromethyl-benzyl)-1,4-dihydro-quinoline-3-carbonitrile, gave 7-dimethylamino-6-fluoro-4-oxo-1-(4-trifluoromethyl-benzyl)-1,4-dihydro-quinoline-3-carbonitrile.

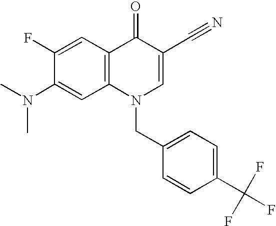

Proceeding as in Reference 3, but substituting 6,7-difluoro-4-oxo-1-(4-trifluoromethyl-benzyl)-1,4-dihydro-quinoline-3-carbonitrile and ethane-1,2-diamine, gave 7-(2-amino-ethylamino)-6-fluoro-4-oxo-1-(4-trifluoromethyl-benzyl)-1,4-dihydro-quinoline-3-carbonitrile.

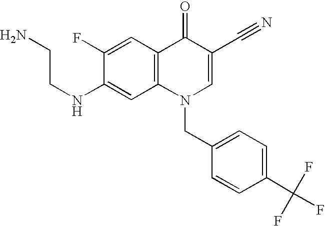

Proceeding as in Reference 3, but substituting 6,7-difluoro-4-oxo-1-phenethyl-1,4-dihydro-quinoline-3-carbonitrile, gave 6-fluoro-7-(4-methyl-piperazin-1-yl)-4-oxo-1-phenethyl-1,4-dihydro-quinoline-3-carbonitrile.

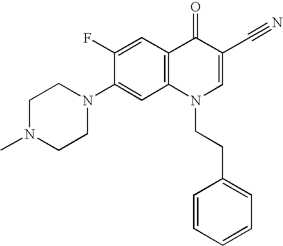

Proceeding as in Reference 3, but substituting 6,7-difluoro-4-oxo-1-(4-trifluoromethyl-benzyl)-1,4-dihydro-quinoline-3-carbonitrile, gave 7-[bis-(2-hydroxy-ethyl)-amino]-6-fluoro-4-oxo-1-(4-trifluoromethyl-benzyl)-1,4-dihydro-quinoline-3-carbonitrile.

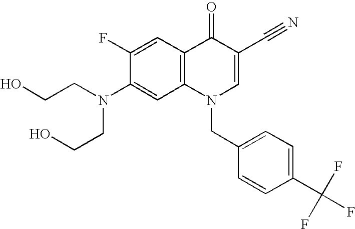

Proceeding as in Reference 3, but substituting 6,7-difluoro-4-oxo-1-(4-trifluoromethyl-benzyl)-1,4-dihydro-quinoline-3-carbonitrile and imidazole, gave 6-fluoro-7-imidazol-1-yl-4-oxo-1-(4-trifluoromethyl-benzyl)-1,4-dihydro-quinoline-3-carbonitrile.

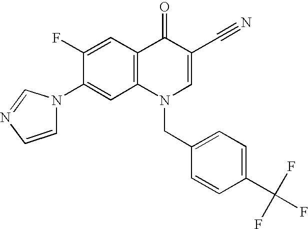

Proceeding as in Reference 3, but substituting 6,7-difluoro-1-(2-fluoro-4-trifluoromethyl-benzyl)-4-oxo-1,4-dihydro-quinoline-3-carbonitrile and piperazin-2-one, gave 6-fluoro-1-(2-fluoro-4-trifluoromethyl-benzyl)-4-oxo-7-(3-oxo-piperazin-1-yl)-1,4-dihydro-quinoline-3-carbonitrile.

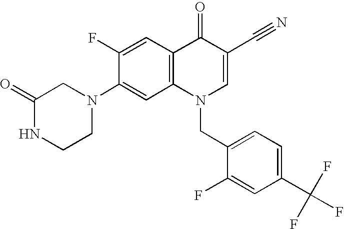

Proceeding as in Reference 3, but substituting 6,7-difluoro-4-oxo-1-[1-(4-trifluoromethyl-phenyl)-cyclopropyl]-1,4-dihydro-quinoline-3-carbonitrile, gave 6-fluoro-7-(4-methyl-piperazin-1-yl)-4-oxo-1-[1-(4-trifluoromethyl-phenyl)-cyclopropyl]-1,4-dihydro-quinoline-3-carbonitrile.

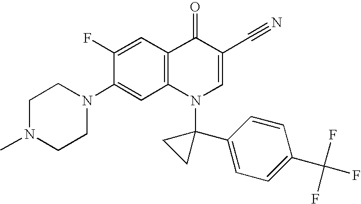

Proceeding as in Reference 3, but substituting 6,7-difluoro-4-oxo-1-(3-trifluoromethyl-benzyl)-1,4-dihydro-quinoline-3-carbonitrile, gave 6-fluoro-7-(4-methyl-piperazin-1-yl)-4-oxo-1-(3-trifluoromethyl-benzyl)-1,4-dihydro-quinoline-3-carbonitrile.

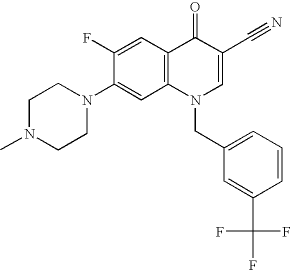

Proceeding as in Reference 3, but substituting 6,7-difluoro-4-oxo-1-(4-trifluoromethyl-benzyl)-1,4-dihydro-quinoline-3-carbonitrile, gave 2-{[3-cyano-6-fluoro-4-oxo-1-(4-trifluoromethyl-benzyl)-1,4-dihydro-quinolin-7-yl]-methyl-amino}-acetamide.

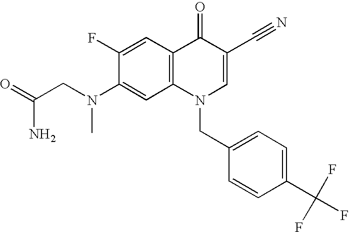

Proceeding as in Reference 3, but substituting 6,7-difluoro-4-oxo-1-(4-trifluoromethyl-benzyl)-1,4-dihydro-quinoline-3-carbonitrile and 1,1-dioxothiomorpholine, gave 7-(1,1-dioxo-thiomorpholin-4-yl)-6-fluoro-4-oxo-1-(4-trifluoromethyl-benzyl)-1,4-dihydro-quinoline-3-carbonitrile.

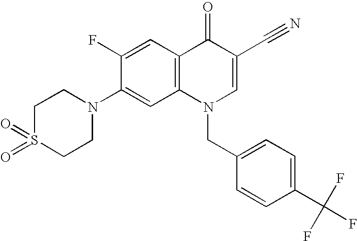

Proceeding as in Reference 3, but substituting 6,7-difluoro-4-oxo-1-(4-trifluoromethyl-benzyl)-1,4-dihydro-quinoline-3-carbonitrile and thiomorpholine, gave 6-fluoro-4-oxo-7-thiomorpholin-4-yl-1-(4-trifluoromethyl-benzyl)-1,4-dihydro-quinoline-3-carbonitrile.

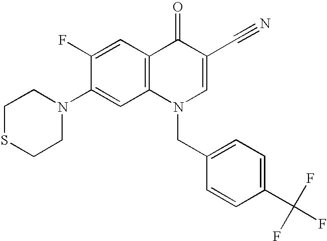

Proceeding as in Reference 3, but substituting 6,7-difluoro-4-oxo-1-(4-trifluoromethyl-benzyl)-1,4-dihydro-quinoline-3-carbonitrile and 1H-imidazole-4-carboxylic acid, gave 1-(3-cyano-6-fluoro-4-oxo-1-(4-(trifluoromethyl)benzyl)-1,4-dihydroquinolin-7-yl)-1H-imidazole-4-carboxylic acid.

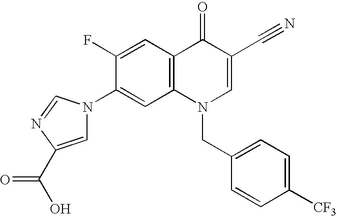

Proceeding as in Reference 3, but substituting 1-(4-tert-butyl-benzyl)-6,7-difluoro-4-oxo-1,4-dihydro-quinoline-3-carbonitrile and [1,4]diazepane gave 1-(4-tert-butyl-benzyl)-7-[1,4]diazepan-1-yl-6-fluoro-4-oxo-1,4-dihydro-quinoline-3-carbonitrile.

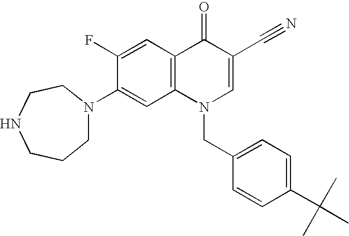

Proceeding as in Reference 3, but substituting 6,7-difluoro-4-oxo-1-(4-(trifluoromethyl)benzyl)-1,4-dihydroquinoline-3-carbonitrile gave 6-fluoro-7-(4-methyl-1,4-diazepan-1-yl)-4-oxo-1-(4-(trifluoromethyl)benzyl)-1,4-dihydroquinoline-3-carbonitrile.

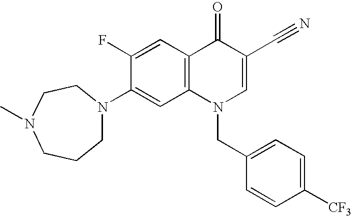

### Reference 3b

### 6-fluoro-7-morpholino-4-oxo-1-(1-(4-(trifluoromethyl)phenyl)ethyl)-1,4-dihydroquinoline-3-carbonitrile

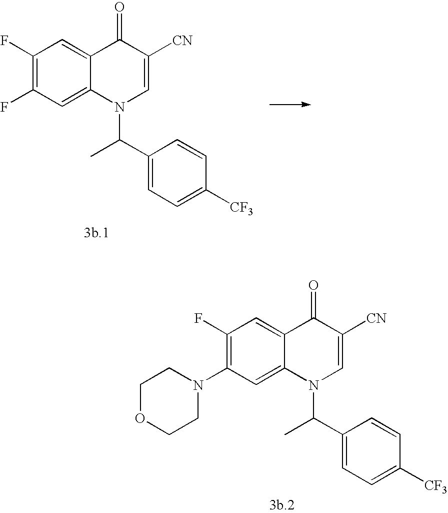

To stirred solution of compound 3.1 (2.1 g, 5.7 mmol) in DMF (30 mL) was treated with potassium carbonate (1.1 g, 8.6 mmol) followed by morpholine (750 g, 8.6 mmol) under nitrogen atmosphere. The reaction mixture was stirred at 60° C. for 4 h. Once the reaction was completed then treated with water to form a precipitate, filtered, washed with pentane and dried under high vacuum at 60° C. for 1 hr to provide compound 3b.2 (1.6 g, 65.2%). ¹H-NMR (DMSO-d₆) δ 8.9 (1H, s, Ar—H), 7.91-7.58 (5H, m, Ar—H), 7.03 (1H, d, Ar—CH), 6.36 (1H, q, Ar—H), 3.73 (4H, bs, OCH₂), 3.24-2.97 (4H, m, NCH₂), 1.9 (3H, d, —CH₃). EIMS (m/z): 446.4 (M+H)⁺.

Proceeding as in Reference 3b, but substituting 6,7-difluoro-1-(4-fluorobenzyl)-4-oxo-1,4-dihydroquinoline-3-carbonitrile and dimethyl amine gave 7-(dimethylamino)-6-fluoro-1-(4-fluorobenzyl)-4-oxo-1,4-dihydroquinoline-3-carbonitrile. TLC system: 50% Ethyl acetate/pet-ether; (R_{f} value: 0.5) ¹H NMR (DMSO-d₆; 300 MHz): 9.01 (s, 1H); 7.73-7.8 (m, 3H); 7.6 (d, J=9 Hz, 2H); 5.68 (s, 2H); 2.86 (s, 6H). Mass: (M+H)⁺ 340. Brown solid, 180 mg (33.33%)

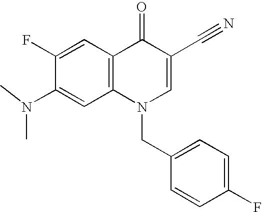

Proceeding as in Reference 3b, but substituting 6,7-difluoro-1-(4-fluorobenzyl)-4-oxo-1,4-dihydroquinoline-3-carbonitrile and thiomorpholine gave 6-fluoro-1-(4-fluorobenzyl)-4-oxo-7-thiomorpholino-1,4-dihydroquinoline-3-carbonitrile. TLC system: 5% MeOH/DCM; (R_{f} value: 0.3); ¹H NMR (DMSO-d₆; 300 MHz): 9.06 (s, 1H); 7.77-7.75 (m, 3H); 7.59-7.56 (d, J=9 Hz, 2H); 6.91-6.89 (d, J=6 Hz, 2H); 5.72 (s, 2H); 3.35 (m, 4H); 2.66-2.64 (m, 4H); Mass: (M+H)⁺ 398; Yellow Solid; 600 mg (62.1%).

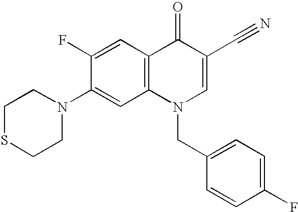

Proceeding as in Reference 3b but substituting morpholine and 6,7-difluoro-4-oxo-1-(2-(4-(trifluoromethyl)phenyl)propan-2-yl)-1,4-dihydroquinoline-3-carbonitrile gave 6-fluoro-7-morpholino-4-oxo-1-(2-(4-(trifluoromethyl)phenyl)propan-2-yl)-1,4-dihydroquinoline-3-carbonitrile. TLC system: 10% MeOH/CHCl₃; (Rf value: 0.4); ¹H NMR (DMSO-d₆; 300 MHz): 8.89 (s, 1H); 7.77-7.74 (m, 3H); 7.72-7.69 (d, J=9 Hz; 2H); 6.25-6.23 (d, J=6 Hz; 1H); 3.58 (s, 4H); 2.6 (s, 4H); 2.06 (s, 6H) Mass: (M+H)⁺ 460; Pale Yellow Solid; 520 mg (98.1%)

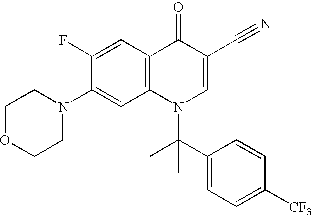

Proceeding as in Reference 3b, but substituting 6,7-difluoro-4-oxo-1-(4-trifluoromethyl-benzyl)-1,4-dihydro-quinoline-3-carbonitrile and 2-morpholinoethylamine gave 6-fluoro-7-(2-morpholinoethylamino)-4-oxo-1-(4-(trifluoromethyl)benzyl)-1,4-dihydroquinoline-3-carbonitrile. TLC system: 5% MeOH/DCM. (R_{f} value: 0.4); ¹H NMR (DMSO-d₆; 300 MHz): 8.91 (s, 1H); 7.75 (m, 3H); 7.4 (d, J=9 Hz, 2H); 6.5 (m, 1H); 6.40 (d, J=12 Hz, 1H); 5.67 (s, 2H); 3.51 (s, 4H); 3.2 (m, 2H); 2.2 (m, 6H); Mass: (M+H)⁺ 475; Off-white Solid; 400 mg (30%)

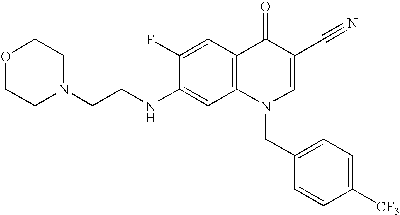

Proceeding as in Reference 3b, but substituting 6,7-difluoro-4-oxo-1-(1-phenylethyl)-1,4-dihydroquinoline-3-carbonitrile and morpholine gave 6-fluoro-7-morpholino-4-oxo-1-(1-phenylethyl)-1,4-dihydroquinoline-3-carbonitrile. ¹H-NMR (DMSO-d₆) δ 8.9 (1H, s, Ar—H), 7.91-7.58 (5H, m, Ar—H), 7.03 (1H, d, Ar—CH), 6.36 (1H, q, Ar—H), 3.73 (4H, bs, OCH₂), 3.24-2.97 (4H, m, NCH₂), 1.9 (3H, d, —CH₃). EIMS (m/z): 446.4 (M+H)⁺.

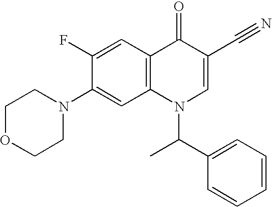

Proceeding as in Reference 3b, but substituting 6,7-difluoro-4-oxo-1-(2-phenylpropan-2-yl)-1,4-dihydroquinoline-3-carbonitrile and morpholine gave 6-fluoro-7-morpholino-4-oxo-1-(2-phenylpropan-2-yl)-1,4-dihydroquinoline-3-carbonitrile.

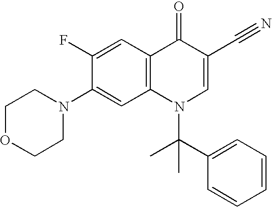

Proceeding as in Reference 3b, but substituting 6,7-difluoro-4-oxo-1-(4-(trifluoromethyl)benzyl)-1,4-dihydroquinoline-3-carbonitrile and 2,6-dimethylmorpholine gave 7-(2,6-dimethylmorpholino)-6-fluoro-4-oxo-1-(4-(trifluoromethyl)benzyl)-1,4-dihydroquinoline-3-carbonitrile. TLC system: 5% MeOH/DCM; (R_{f} value: 0.3) ¹H NMR (DMSO-d₆; 300 MHz): 9.2 (s, 1H); 8.6 (s, 1H); 7.8 (m, 3H); 7.46 (d, J=9 Hz, 2H); 6.83 (d, J=9 Hz, 1H); 6.36 (s, 1H); 5.87 (s, 1H); 3.61 (t, J=7.5 Hz, 2H); 3.28 (t, 2H); 2.3 (t, J=10.5 Hz, 2H); 1.08 (d, J=6 Hz, 6H); Mass: (M+H)⁺ 493; Yellow solid; 825 mg; (69.9%).

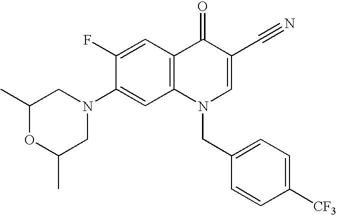

Proceeding as in Reference 3b, but substituting 6,7-difluoro-4-oxo-1-(4-(trifluoromethyl)benzyl)-1,4-dihydroquinoline-3-carbonitrile and morpholine gave 7-(morpholino)-6-fluoro-4-oxo-1-(4-(trifluoromethyl)benzyl)-1,4-dihydroquinoline-3-carbonitrile. TLC system: 30% Ethyl Acetate/Pet-ether; (R_{f} value: 0.3); ¹H NMR (DMSO-d₆; 300 MHz): 9.0 (s, 1H); 7.7 (t, 3H); 7.5 (d, J=6 Hz, 2H); 6.9 (d, J=6 Hz, 1H); 5.7 (s, 2H); 3.6 (s, 4H); 3.0 (s, 4H); Mass: (M+H)⁺ 432; Yellow solid; 460 mg (41%).

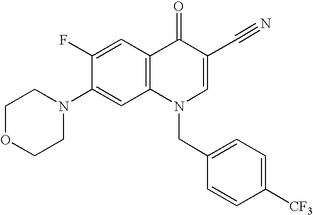

Proceeding as in Reference 3b, but substituting 7-fluoro-4-oxo-1-(4-(trifluoromethyl)benzyl)-1,4-dihydroquinoline-3-carbonitrile and morpholine gave 7-(morpholino)-4-oxo-1-(4-(trifluoromethyl)benzyl)-1,4-dihydroquinoline-3-carbonitrile.

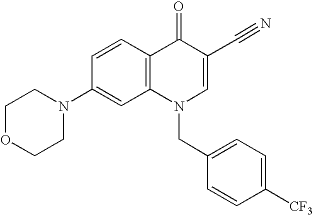

Proceeding as in Reference 3b, but substituting 6,7-difluoro-4-oxo-1-(4-(trifluoromethyl)benzyl)-1,4-dihydroquinoline-3-carbonitrile and (2S,6R)-2,6-dimethylpiperazine gave 7-((3S,5R)-3,5-dimethylpiperazin-1-yl)-6-fluoro-4-oxo-1-(4-(trifluoromethyl)benzyl)-1,4-dihydroquinoline-3-carbonitrile. TLC system: 10% Methanol/Chloroform; (R_{f} value: 0.5); ¹H NMR (DMSO-d₆; 300 MHz): 9.0 (s, 1H); 7.67-7.74 (t, 3H); 7.55-7.57 (d, J=6 Hz, 2H); 6.71-6.73 (d, J=6 Hz, 1H); 5.69 (s, 2H); 3.16-3.20 (d, J=12 Hz, 4H); 2.61 (m, 2H); 2.18 (t, 2H); 0.93-0.91 (d, J=6 Hz, 6H); Mass: (M+H)⁺ 459; Brown solid; 400 mg; (63.5%).

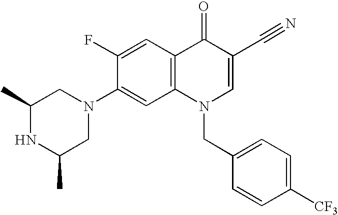

Proceeding as in Reference 3b, but substituting 6,7-difluoro-1-(4-methoxybenzyl)-4-oxo-1,4-dihydroquinoline-3-carbonitrile and morpholine gave 6-fluoro-1-(4-methoxybenzyl)-7-morpholino-4-oxo-1,4-dihydroquinoline-3-carbonitrile.

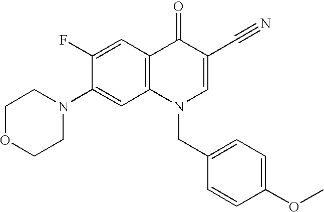

Proceeding as in Reference 3b but substituting piperazin-2-one and 6,7-difluoro-4-oxo-1-(4-(trifluoromethyl)benzyl)-1,4-dihydroquinoline-3-carbonitrile gave 6-fluoro-4-oxo-7-(3-oxopiperazin-1-yl)-1-(4-(trifluoromethyl)benzyl)-1,4-dihydroquinoline-3-carbonitrile. TLC system: 5% MeOH/DCM; (R_{f} value: 0.4); ¹H NMR (DMSO-d₆; 300 MHz): 9.06 (s, 1H); 7.75-7.77 (m, 4H); 7.54-7.51 (d, J=9 Hz, 2H); 6.92-6.90 (d, J=6 Hz, 1H); 5.72 (s, 2H); 3.58 (s, 2H); 3.31 (s, 2H); 3.2 (s, 2H); Mass: (M+H)⁺ 445; Brown solid; 660 mg; (56%)

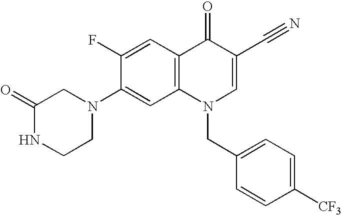

Proceeding as in Reference 3b but substituting morpholine and 6,7-difluoro-1-(4-fluorobenzyl)-4-oxo-1,4-dihydroquinoline-3-carbonitrile gave 6-fluoro-1-(4-fluorobenzyl)-7-morpholino-4-oxo-1,4-dihydroquinoline-3-carbonitrile. TLC system: 5% MeOH/DCM; (R_{f} value: 0.3); ¹H NMR (DMSO-d₆; 300 MHz): 9.06 (s, 1H); 7.77-7.75 (m, 3H); 7.59-7.56 (d, J=9 Hz, 2H); 6.91-6.89 (d, J=6 Hz, 2H); 5.72 (s, 2H); 3.35 (m, 4H); 2.66-2.64 (m, 4H); Mass: (M+H)⁺ 382; Yellow Solid; 300 mg (50%)

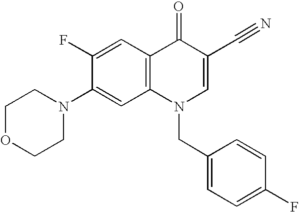

Proceeding as in Reference 3b but substituting dimethylamine and 6,7-difluoro-4-oxo-1-(1-(4-(trifluoromethyl)phenyl)ethyl)-1,4-dihydroquinoline-3-carbonitrile gave 7-(dimethylamino)-6-fluoro-4-oxo-1-(1-(4-(trifluoromethyl)phenyl)ethyl)-1,4-dihydroquinoline-3-carbonitrile

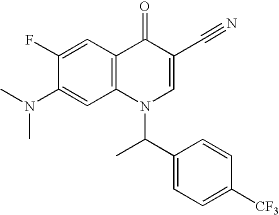

Proceeding as in Reference 3b but substituting dimethylamine and 6,7-difluoro-1-(2-methyl-2-(4-(trifluoromethyl)phenyl)propyl)-4-oxo-1,4-dihydroquinoline-3-carbonitrile gave 7-(dimethylamino)-6-fluoro-1-(2-methyl-2-(4-(trifluoromethyl)phenyl)propyl)-4-oxo-1,4-dihydroquinoline-3-carbonitrile

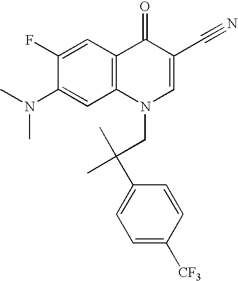

Proceeding as in Reference 3b but substituting thiomorpholine and 1-(cyclopropylmethyl)-6,7-difluoro-4-oxo-1,4-dihydroquinoline-3-carbonitrile gave 1-(cyclopropylmethyl)-6-fluoro-4-oxo-7-thiomorpholino-1,4-dihydroquinoline-3-carbonitrile. TLC system: 30% Ethyl acetate/Pet-ether; (R_{f} value: 0.3); ¹H NMR (DMSO-d₆; 300 MHz): 9.0 (s, 1H); 7.7 (t, 3H); 7.5 (d, J=6 Hz, 2H); 6.9 (d, J=6 Hz, 1H); 5.70 (s, 2H); 3.67 (s, 4H); 3.04 (s, 4H); Mass: (M+H)⁺ 344; Yellow solid; 650 mg (65%)

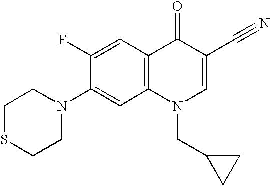

Proceeding as in Reference 3b but substituting thiomorpholine and 6,7-difluoro-4-oxo-1-(4-(trifluoromethyl)benzyl)-1,4-dihydroquinoline-3-carbonitrile gave 6-fluoro-4-oxo-7-thiomorpholino-1-(4-(trifluoromethyl)benzyl)-1,4-dihydroquinoline-3-carbonitrile

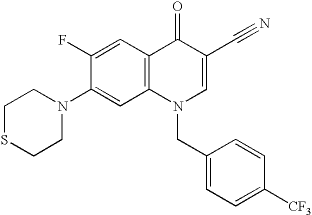

Proceeding as in Reference 3b but substituting dimethylamine and 6,7-difluoro-4-oxo-1-(4-(trifluoromethyl)benzyl)-1,4-dihydroquinoline-3-carbonitrile gave 7-(dimethylamino)-6-fluoro-4-oxo-1-(4-(trifluoromethyl)benzyl)-1,4-dihydroquinoline-3-carbonitrile. TLC system: 50% Ethyl acetate/pet-ether; (R_{f} value: 0.4) ¹H NMR (DMSO-d₆; 300 MHz): 9.01 (s, 1H); 7.73-7.8 (m, 3H); 7.6 (d, J=9 Hz, 2H); 5.68 (s, 2H); 2.86 (s, 6H). Mass: (M+H)⁺ 390. Brown solid; 150 mg (28%).

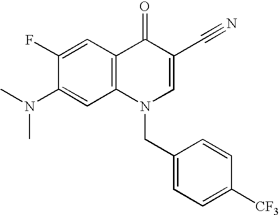

### Reference 3.1

### 6-Fluoro-7-(4-methyl[1,4]diazepam-1-yl)-4-oxo-1-(4-trifluoro-methylbenzyl) 1,4-dihydro[1,8]naphthyridine-3-carbonitrile

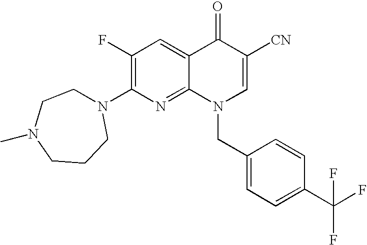

Ethyl 2-(2,6-dichloro-5-fluoropyridine-3-carbonyl)-3-(4-trifluoromethyl-benzylamino)acrylate (3.0 g, 6.45 mmol), prepared as in Reference 2c, was dissolved acetonitrile (50 mL) and the solution was cooled in an ice bath. Anhydrous potassium carbonate (7.24 mmol, 1.0 g) was added to the solution and the mixture was heated under reflux until the reaction was completed. The mixture was cooled and extracted with methylene chloride. The extract was filtered and then concentrated by evaporation under reduced pressure. Product was purified from the residue by chromatography over silica eluting with 2% MeOH/CH₂Cl₂. Solvents were removed by evaporation under reduced pressure and the residue was pumped down under vacuum to give ethyl 7-chloro-6-fluoro-4-oxo-1-(4-trifluoromethylbenzyl)-1,4-dihydro-[1,8]naphthyridine-3-carboxylate as an oil (2.24 g, 81%). R_{f}=0.43 (5% MeOH/DCM). LCMS (MH+)=429.9; (M+23)=452.1; actual=428.77.

4-methyl[1,4]diazepam (2.8 mmol, 0.35 mL) and TEA (1.30 mL, 9.3 mmol) were added to a solution of ethyl 7-chloro-6-fluoro-4-oxo-1-(4-trifluoromethylbenzyl)-1,4-dihydro-[1,8]naphthyridine-3-carboxylate (1.0 g, 2.33 mmol) in DMA (20 mL) and the mixture was heated at 65° C. for 1 hour. The solvent was removed by evaporation and the residue was partitioned between ethyl acetate and water. The aqueous layer was brought to pH 10 and extracted with methylene chloride. The combined organic layers were dried and concentrated to give ethyl 6-fluoro-7-(4-methyl[1,4]diazepam-1-yl)-4-oxo-1-(4-trifluoro-methylbenzyl)1,4-dihydro[1,8]naphthyridine-3-carboxylate (1.15 g, 97%) as a nearly pure crude oil. LCMS: (MH+)=506.9; (MH−)=505.4; (M+23)=529.3; actual=506.49.

6-Fluoro-7-(4-methyl[1,4]diazepam-1-yl)-4-oxo-1-(4-trifluoro-methylbenzyl) 1,4-dihydro[1,8]naphthyridine-3-carboxylate (1.15 g, 2.2 mmol) was dissolved in ethanol and potassium hydroxide (10 mmol in water) was added to the solution and the reaction was allowed to proceed for one hour. Solvents were removed by evaporation and the residue was combined with water. The mixture was brought to pH 7 with 1N hydrochloric acid and then stirred for approximately 12 hours. A granular precipitate was collected by filtration and then dried under vacuum to give 6-fluoro-7-(4-methyl[1,4]diazepam-1-yl)-4-oxo-1-(4-trifluoro-methylbenzyl)1,4-dihydro[1,8]naphthyridine-3-carboxylic acid (0.96 g, 91%). LCMS: (MH+)=478.8) (MH−)=477.3; (M+23)=501.3; actual=478.36.

6-Fluoro-7-(4-methyl[1,4]diazepam-1-yl)-4-oxo-1-(4-trifluoro-methylbenzyl) 1,4-dihydro[1,8]naphthyridine-3-carboxylic acid (0.96 g, 2.0 mmol) was dissolved in DMA (10 mL, evaporated×2 to remove residual water) and the solution was treated with TEA (10 mmol 1.012 g, 1.39 mL) and isobutylchloroformate (2.4 mmol, 0.328 g, 0.311 mL). The mixture was stirred for 2 hours and then ammonia (21.0 mmol, 7N in methanol) was added and the mixture was stirred for 1 hour. Solvents were removed by evaporation and the residue was combined with water. The mixture was stirred for approximately 12 hours. A granular precipitate was collected by filtration and pumped dry under vacuum to give solid material (0.73 g). The filtrate was concentrated and further solid material was obtained (0.105 g). The solid materials were combined to give 6-fluoro-7-(4-methyl[1,4]diazepam-1-yl)-4-oxo-1-(4-trifluoro-methylbenzyl) 1,4-dihydro[1,8]naphthyridine-3-carboxamide (0.835 g, 81%). LCMS (MH+)=478.0; (M+23)=500.5; actual=477.48.

6-Fluoro-7-(4-methyl[1,4]diazepam-1-yl)-4-oxo-1-(4-trifluoro-methylbenzyl) 1,4-dihydro[1,8]naphthyridine-3-carboxamide (700 mg, 1.47 mmol) was treated with PPE (7 g). The mixture was stirred and heated to 60° C. until the reaction was completed. The mixture was cooled and then treated with 30% sodium carbonate solution (14 mL). The mixture was stirred for several hours and then a granular precipitate was collected by filtration and dried to give 6-fluoro-7-(4-methyl[1,4]diazepam-1-yl)-4-oxo-1-(4-trifluoro-methylbenzyl) 1,4-dihydro[1,8]naphthyridine-3-carbonitrile (598 mg, 89%). LCMS: (MH+)=460.5; (M+23)=482.3; actual 459.47.

### Reference 3.2

### See FIG. 13

### Compound 3.2.3

### Synthesis of diethyl 2-(3,4diflorophenylamino)methylen) malonate

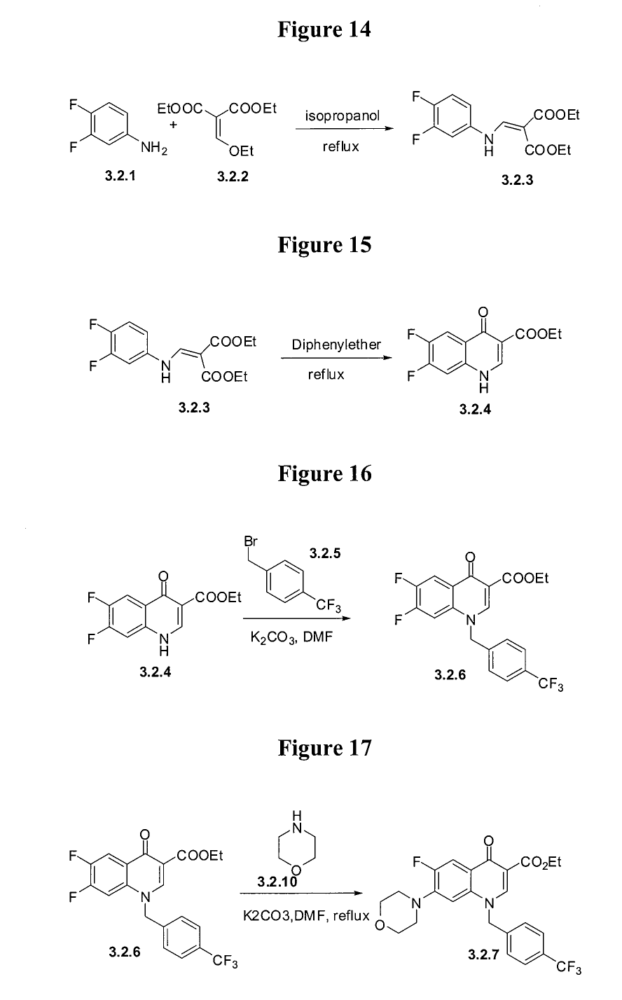

### See FIG. 14

To a stirred solution of 2-ethoxymethylenmalonic acid diethyl ester 3.2.2 (25 g, 19.4 mol) in 2-propanol (300 mL) under nitrogen was added 3,4-difluoroaniline 3.2.1 (41.9 g, 19.38 mol) and the reaction mixture was refluxed for 3 h. The reaction was monitored by TLC and after complete disappearance of the starting material, the reaction mixture was cooled to room temperature, solvent was evaporated under reduced pressure and the residue was purified by silica gel column chromatography eluting with 5% ethyl acetate in hexane to provide compound 3.2.3 (49.9 g, 86%) as a solid. ¹H-NMR (DMSO-d₆) δ 10.62 (1H, d, —NH), 8.31 (1H, d, Ar—H), 7.63 (1H, m, Ar—H), 7.44 (1H, m, Ar—H), 7.25 (1H, s, Olefin-CH) 4.19 (4H, q, —CH₂CH₃), 1.29 (6H, t, —CH₃). EIMS (m/z): 299.9 (M+H)⁺.

### Compound 3.2.4

### Synthesis of ethyl 6,7-difluoro-4-oxo-1,4-dihydroquinoline-3 carboxylate

### See FIG. 15

To refluxing diphenyl ether (280 mL) was added drop-wise a solution compound-3.2.3 (49.96 g, 167 mmol) in diphenyl ether (100 mL) and the reaction mixture was allowed to reflux for 1 h. The reaction was monitored by TLC and after complete disappearance of the starting material, the reaction mixture was cooled to room temperature, the formed solid compound was filtered and washed with diethyl ether and dried under reduced pressure to provide compound 3.2.4 (21.4 g, 61% yield). ¹H-NMR (DMSO-d₆) δ 12.5 (1H, br, —NH), 8.63 (1H, s, NCH), 8.06 (1H, m, Ar—H), 7.67 (1H, m, Ar—H), 4.28 (2H, q, —CH₂), 1.29 (3H, t, —CH₃). EIMS (m/z): 254 (M⁺+1)⁺.

### Compound 3.2.6

### Synthesis of ethyl 1-(4-(trifluoromethyl)benzyl) 6,7-difluoro-1,4 dihydro-4-oxoquinoline-3 carboxylate

### See FIG. 16

To a stirred solution of 6,7-difluoro-1,4 dihydro-4-oxoquinoline-3 carboxylic acid ethyl ester (10 g, 39.5 mmol) in anhydrous dimethyl formamide (100 mL) was added potassium carbonate (13.60 g, 98.8 mmol), compound 3.2.5 (11.33 g, 47.43 mmol) and the reaction mixture was heated at 80° C. for 2 hrs. Once the reaction was complete, the solvent was evaporated under the reduced pressure and the residue was treated with water and extracted with ethyl acetate (2×250 mL). The combined organic layer were washed with brine (200 ml), dried over NaSO₄ and concentrated to give the desired product 3.2.6 (14.2 g, 87% yield) as a solid. ¹H-NMR (DMSO-d₆) δ 8.92 (1H, s, Ar—H), 8.09 (1H, m, Ar—H), 7.82-7.67 (3H, m, Ar—H), 7.45 (2H, d, Ar—H), 5.69 (2H, s, —CH₂Ph), 4.24 (2H, q, —CH₂), 1.28 (3H, t, —CH₃). EIMS (m/z): 412 (M+H)⁺.

### Compound 3.2.7

### Synthesis of ethyl 1-(4-(trifluoromethyl)benzyl) 6-fluoro-1,4 dihydro-7-morpholino-4-oxoquinoline-3 carboxylate

### See FIG. 17

To a stirred solution of ethyl 1-(4-(trifluoromethyl)benzyl) 6-fluoro-1,4 dihydro-7-morpholino-4-oxoquinoline-3 carboxylate 3.2.6 (10 g, 24.33 mmol) in anhydrous dimethyl formamide (120 mL) was added potassium carbonate (10.07 g, 72.3 mmol) and morpholine 3.2.10 (5.29 g, 60.82 mmol). The reaction mixture was heated at 60° C. overnight. The solvent was evaporated under reduced pressure and the residue was treated with water and extracted with ethyl acetate (2×200 mL). The combined organic layers were washed with brine (150 ml), dried over NaSO₄, filtered and concentrated and the crude compound was purified by silica gel chromatography eluting with 1% methanol in dichloromethane to provide the desired product 3.2.7 (11.1 g, 95.7%) as a solid. ¹H-NMR (DMSO-d₆) δ 8.89 (1H, s, Ar—H), 7.81-7.69 (3H, m, Ar—H), 7.58 (2H, d, Ar—H), 6.88 (1H, d, Ar—H), 5.88 (2H, s, —CH₂Ph), 4.30 (2H, q, —CH₂), 3.66 (4H, m, OCH₂), 3.02 (4H, m, NCH₂), 1.27 (3H, t, —CH₃). EIMS (m/z): 479 (M+H)⁺.

### Compound 3.2.9

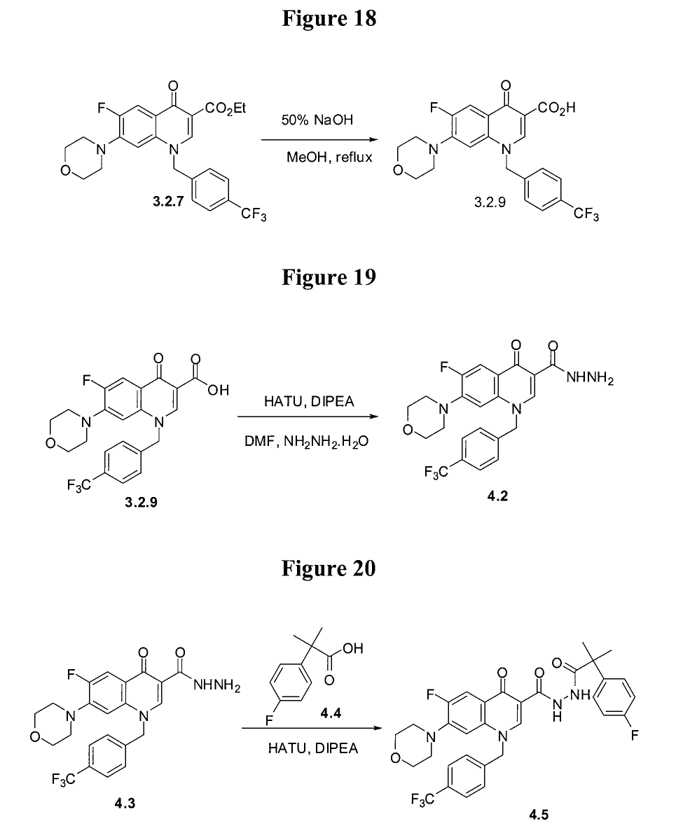

### Hydrolysis of 1-(4-(trifluoromethyl)benzyl) 6-fluoro-1,4 dihydro-7-morpholino-4-oxoquinoline-3 carboxylic acid ethyl ester. See FIG. 18

To a stirred solution of 1-(4-(trifluoromethyl)benzyl) 6-fluoro-1,4 dihydro-7-morpholino-4-oxoquinoline-3 carboxylic acid ethyl ester 3.2.7 (13.0 g, 27.19 mmol) in methanol (50 mL) was added 50% aq. NaOH (50 mL) and the reaction mixture was heated to reflux for 2 h. The reaction was monitored by TLC and after complete disappearance of the starting material the reaction mixture was cooled to room temperature, and solvent was evaporated under reduced pressure to give the sodium salt of 1-(4-(trifluoromethyl)benzyl) 6-fluoro-1,4 dihydro-7-morpholino-4-oxoquinoline-3 carboxylic acid as a white solid. The resulting white solid compound was stirred in 4 M HCl (60 ml) for 15 min and filtered, washed with ethyl ether and dried to provide 1-(4-(trifluoromethyl)benzyl) 6-fluoro-1,4 dihydro-7-morpholino-4-oxoquinoline-3 carboxylic acid 3.2.9 (11.9 g, 97.1%). ¹H-NMR (DMSO-d₆) δ 9.24 (1H, s, Ar—H), 7.98 (1H, d, Ar—H), 7.77 (2H, d, Ar—H), 7.58 (2H, d, Ar—H), 7.04 (1H, d, Ar—H), 5.97 (2H, s, —CH₂Ph), 3.72 (4H, m, OCH₂), 3.14 (4H, m, NCH₂). EIMS (m/z): 451 (M+H)⁺.

### Reference 4

### 6-fluoro-N′-(2-(4-fluorophenyl)-2-methylpropanoyl)-7-morpholino-4-oxo-1-(4-(trifluoromethyl)benzyl)-1,4-dihydroquinoline-3-carbohydrazide

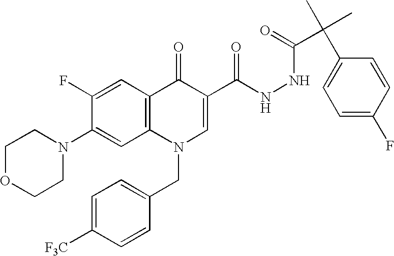 Step 1

### See FIG. 19

To a stirred solution of 3.2.9 (250 mg, 0.5 mmol) in DMF (15 mL) was added HATU (253 mg, 0.6 mmol) and DIPEA (358 mg, 2.77 mmol). After 1 h, hydrazine hydrate (277 mg, 5.5 mmol) was added and the reaction mixture was stirred under nitrogen atmosphere at room temperature overnight. Once the reaction was complete, water was added to the reaction mixture, and a precipitate formed. The solid was filtered and washed with diethyl ether to provide compound 4.2 (241 mg, 93.8%) as white solid.

Step 2

### See FIG. 20

To a stirred solution of 4-Fluoro-α,α-dimethylphenyl acetic acid 4.4 (70 mg, 0.38 mmol) in DMF (8 mL) was added HATU (147 mg, 0.38 mmol) and DIPEA (208 mg, 1.61 mmol). The reaction mixture stirred for 1 h under nitrogen atmosphere at room temperature, compound 4.3 (150 mg, 0.32 mmol) was added, and the reaction mixture stirred overnight. The reaction mixture was partitioned between water and ethyl acetate. The organic layer was washed with brine solution, dried over MgSO₄, filtered and concentrated. The crude residue was purified by silica gel flash column chromatography eluting with 2% methanol in dichloromethane to provide compound 4.5 (132 mg, 65%). ¹H-NMR (DMSO-d₆) δ 11.28 (1H, s, CONH), 9.89 (1H, s, CONH), 9.09 (1H, s, NCH), 7.88 (1H, d, Ar—H), 7.78 (2H, d, Ar—H), 7.46 (4H, m, Ar—H), 7.46 (2H, m, Ar—H), 6.99 (1H, d, Ar—H), 5.97 (2H, s, CH₂), 4.62 (2H, bs, NH₂), 3.71 (4H, m, OCH₂), 3.07 (4H, m, NCH₂). EIMS (m/z): 629.7 (M+H)+.

### Reference 5

### Benzyl 3-(5-benzyl-1,2,4-oxadiazol-3-yl)-1-(2-fluoro-4-(trifluoromethyl)benzyl)-4-oxo-4,5,7,8-tetrahydro-1,6-naphthyridine-6(1H)-carboxylate

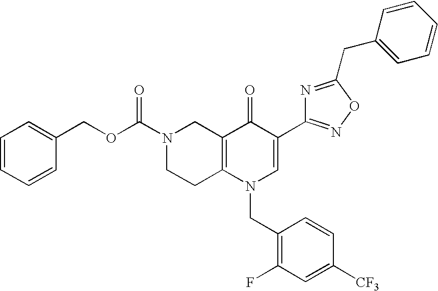

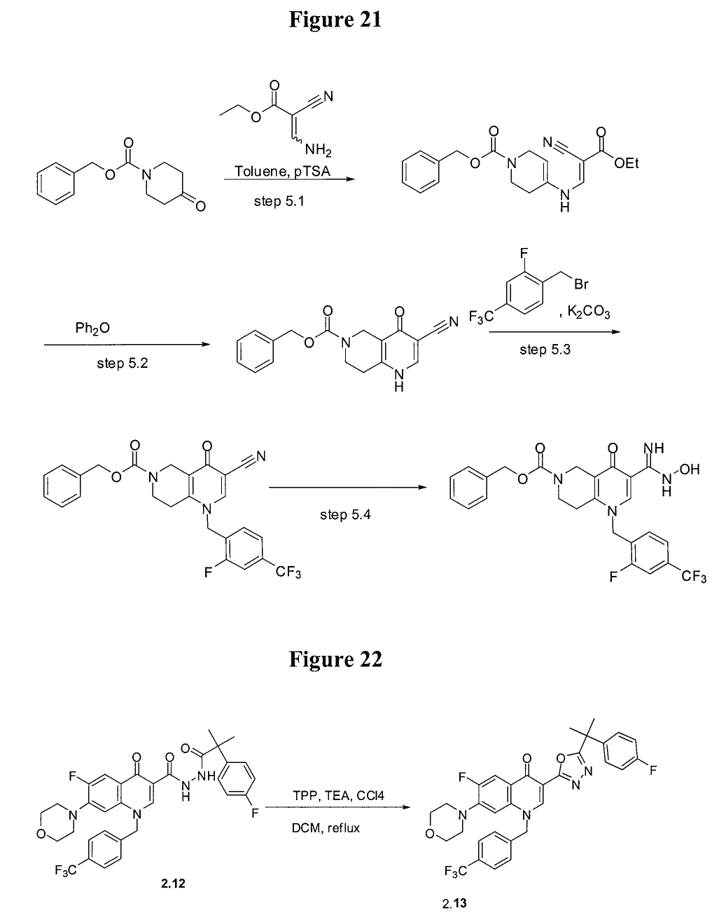

### See FIG. 21

Step 5.1

A solution of benzyl 4-oxopiperidine-1-carboxylate (3.4 g; 14.6 mmol), ethyl 3-amino-2-cyanoacrylate (2.04 g; 14.6 mmol), pTSA (0.25 g) in toluene was heated to reflux overnight in a flask fitted with a Dean-Stark trap. The reaction was evaporated to dryness and purified by silica gel chromatography to afford (E)-benzyl 4-(2-cyano-3-ethoxy-3-oxoprop-1-enylamino)-5,6-dihydropyridine-1(2H)-carboxylate (2.58 g)

Step 5.2

A solution of (E)-benzyl 4-(2-cyano-3-ethoxy-3-oxoprop-1-enylamino)-5,6-dihydropyridine-1(2H)-carboxylate (2.58 g) in Ph₂O (75 mL) was heated to reflux for 1 h. The reaction was evaporated under reduced pressure to afford benzyl 3-cyano-4-oxo-4,5,7,8-tetrahydro-1,6-naphthyridine-6(1H)-carboxylate.

Step 5.3

To a solution of benzyl 3-cyano-4-oxo-4,5,7,8-tetrahydro-1,6-naphthyridine-6(1H)-carboxylate (0.39 g; 1.26 mmol) in DMF was added K₂CO₃ (0.5 g; 3.8 mmol) and 1-(bromomethyl)-2-fluoro-4-(trifluoromethyl)benzene (0.32 g; 1.26 mmol). The reaction mixture was heated to 60° C. until the reaction was complete. The reaction was evaporated to dryness and the residue was purified by silica gel chromatography to afford benzyl 3-cyano-1-(2-fluoro-4-(trifluoromethyl)benzyl)-4-oxo-4,5,7,8-tetrahydro-1,6-naphthyridine-6(1H)-carboxylate (0.236 g)

Step 5.4

Proceeding as Example 1, but substituting benzyl 3-cyano-1-(2-fluoro-4-(trifluoromethyl)benzyl)-4-oxo-4,5,7,8-tetrahydro-1,6-naphthyridine-6(1H)-carboxylate and 2-phenylacetic acid to afford benzyl 3-(5-benzyl-1,2,4-oxadiazol-3-yl)-1-(2-fluoro-4-(trifluoromethyl)benzyl)-4-oxo-4,5,7,8-tetrahydro-1,6-naphthyridine-6(1H)-carboxylate.

Proceeding as in Reference 5 but substituting 2-(4-chlorophenyl)acetic acid in step 5.4 gave benzyl 3-(5-(4-chlorobenzyl)-1,2,4-oxadiazol-3-yl)-1-(2-fluoro-4-(chloro)benzyl)-4-oxo-4,5,7,8-tetrahydro-1,6-naphthyridine-6(1H)-carboxylate.

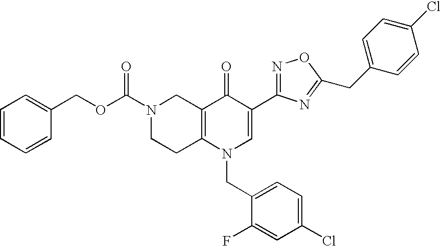

### Example 1

### 3-(5-Benzyl-[1,2,4]oxadiazol-3-yl)-1-(4-chloro-benzyl)-6,7-dimethoxy-1H-quinolin-4-one Compound 1

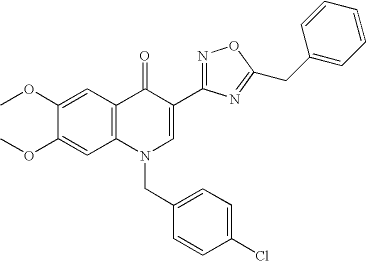

1-(4-Chloro-benzyl)-6,7-dimethoxy-4-oxo-1,4-dihydro-quinoline-3-carbonitrile (100 mg, 0.28 mmol), prepared as in Reference 2, was dissolved in ethanol (10 mL) and then 50% hydroxyamine (0.5 mL) was added to the solution. The mixture was heated at reflux and then concentrated. The residue was dried in a vacuum for 2 hours to give 1-(4-chloro-benzyl)-N-hydroxy-6,7-dimethoxy-4-oxo-1,4-dihydro-quinoline-3-carboxamidine. Mass Spec: m/z 386 (M−1), 388 (M+1).

O-Benzotriazol-1-yl-N,N,N′,N′-tetramethyluroniumhexafluorophosphate (106 mg, 0.28 mmol), diisopropyldiethylamine (180 mg, 1.4 mmol) and the 1-(4-chloro-benzyl)-N-hydroxy-6,7-dimethoxy-4-oxo-1,4-dihydro-quinoline-3-carboxamidine were added sequentially to a stirring solution of phenylacetic acid (40 mg, 0.28 mmol) in DMF (7 mL). The mixture was microwaved to 200° C. for 180 seconds or and then concentrated. The product was purified from the residue by reverse phase HPLC employing 10 mM HCl and acetonitrile as eluents. Fractions were collected and evaporated to give 3-(5-benzyl-[1,2,4]oxadiazol-3-yl)-1-(4-chloro-benzyl)-6,7-dimethoxy-1H-quinolin-4-one (Compound 1; +). (68 mg, 0.14 mmol). +. Mass Spec: m/z 486 (M−1), 488 (M+1). ¹H NMR (400 MHz, DMSO): 8.88 (1H, s), 7.65 (1H, s), 7.45-7.33 (9H, m), 7.0 (1H, s), 5.75 (2H, s), 4.42 (2H, s), 3.87 (3H, s) and 3.79 (3H, s).

Proceeding as in Example 1, but substituting 1-phenyl-cyclopropanecarboxylic acid, gave 1-(4-chloro-benzyl)-6,7-dimethoxy-3-[5-(1-phenyl-cyclopropyl)-[1,2,4]oxadiazol-3-yl]-1H-quinolin-4-one (Compound 2; +). Mass Spec: 515 (M+1). ¹H NMR: 8.74 (1H, s), 7.62 (1H, s), 7.5-7.25 (9H, m), 7.0 (1H, s), 5.72 (1H, s), 3.84 (1H, s), 3.76 (1H, s), 1.8-1.75 (2H, m), 1.62-1.55 (2H, m).

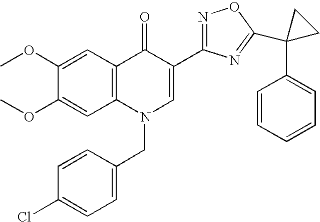

Proceeding as in Example 1, but substituting pyrid-2-ylacetic acid, gave 1-(4-chloro-benzyl)-6,7-dimethoxy-3-(5-pyridin-2-ylmethyl-[1,2,4]oxadiazol-3-yl)-1H-quinolin-4-one (Compound 3; +). Mass Spec: 490 (M+1).

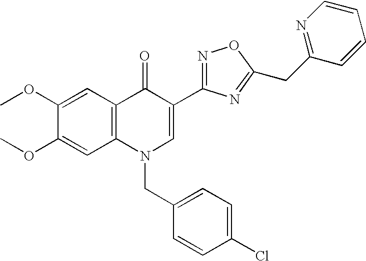

Proceeding as in Example 1, but substituting 4-methylphenylacetic acid, gave 1-(4-chloro-benzyl)-6,7-dimethoxy-3-[5-(4-methyl-benzyl)-[1,2,4]oxadiazol-3-yl]-1H-quinolin-4-one (Compound 4; +). Mass Spec: 503 (M+1). ¹H NMR: 8.87 (1H, s), 7.65 (1H, s), 7.44 (2H, d, J=8.4 Hz), 7.34 (2H, d, J=8.4 Hz), 7.28 (2H, d, J=8.0 Hz), 7.20 (2H, d, J=8.0 Hz), 7.05 (1H, s), 5.76 (2H, s), 4.36 (2H, s), 3.87 (3H, s), 3.79 (3H, s), 2.3 (3H, s).

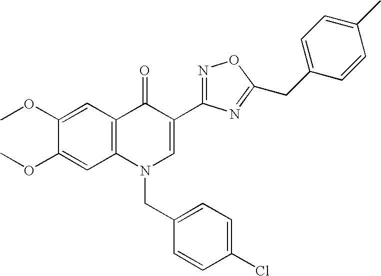

Proceeding as in Example 1, but substituting cyclohexylacetic acid, gave 1-(4-chloro-benzyl)-3-(5-cyclohexylmethyl-[1,2,4]oxadiazol-3-yl)-6,7-dimethoxy-1H-quinolin-4-one (Compound 5: +). Mass Spec: 495 (M+1). ¹H NMR: 8.86 (1H, s), 7.64 (1H, s), 7.43 (2H, d, J=8.4 Hz), 7.32 (2H, d, J=8.4 Hz), 7.03 (1H, s), 5.74 (2H, s), 3.85 (3H, s), 3.78 (3H, s), 2.86 (2H, d, J=7.2 Hz), 1.89-1.55 (5H, m), 1.3-1.0 (6H, m).

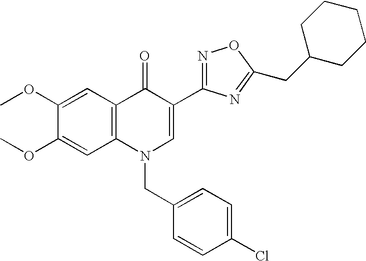

Proceeding as in Example 1, but substituting thien-3-ylacetic acid, gave 1-(4-chloro-benzyl)-6,7-dimethoxy-3-(5-thiophen-3-ylmethyl-[1,2,4]oxadiazol-3-yl)-1H-quinolin-4-one (Compound 6; ++). Mass Spec: 495 (M+1). ¹H NMR: 8.87 (1H, s), 7.64 (1H, s), 7.59-7.55 (1H, s), 7.50 (1H, br m), 7.43 (2H, d, J=8.2 Hz), 7.32 (2H, d, J=8.4 Hz), 7.24 (1H, dd, J=4.8 and 1.2 Hz), 7.03 (1H, s), 5.74 (2H, s), 4.42 (2H, s), 3.85 (3H, s), 3.77 (3H, s).

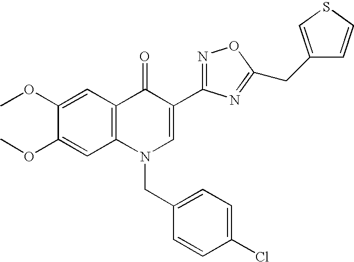

Proceeding as in Example 1, but substituting cyclopentylacetic acid, gave 1-(4-chloro-benzyl)-3-(5-cyclopentylmethyl-[1,2,4]oxadiazol-3-yl)-6,7-dimethoxy-1H-quinolin-4-one (Compound 7: +). Mass Spec: 481 (M+1). ¹H NMR: 8.86 (1H, s), 7.64 (1H, s), 7.43 (2H, d, J=8.4 Hz), 7.32 (2H, d, J=8.2 Hz), 7.04 (1H, s), 5.75 (1H, s), 3.85 (3H, s), 3.78 (3H, s), 2.97 (2H, d, J=7.2 Hz), 1.87-1.20 (9H, m).

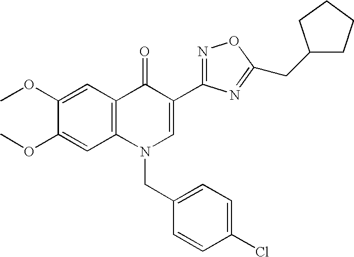

Proceeding as in Example 1, but substituting 1-(4-chloro-benzyl)-6-fluoro-7-(4-methyl-piperazin-1-yl)-4-oxo-1,4-dihydro-quinoline-3-carbonitrile, gave 3-(5-benzyl-[1,2,4]oxadiazol-3-yl)-1-(4-chloro-benzyl)-6-fluoro-7-(4-methyl-piperazin-1-yl)-1H-quinolin-4-one (Compound 8; +++).

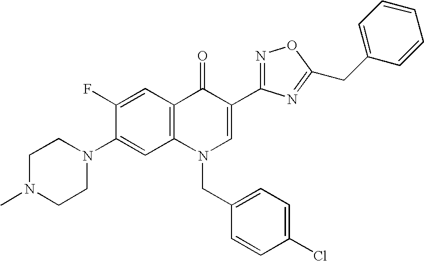

Proceeding as in Example 1, but substituting 1-cyclohexylmethyl-6-fluoro-7-(4-methyl-piperazin-1-yl)-4-oxo-1,4-dihydro-quinoline-3-carbonitrile, gave 3-(5-benzyl-[1,2,4]oxadiazol-3-yl)-1-cyclohexylmethyl-6-fluoro-7-(4-methyl-piperazin-1-yl)-1H-quinolin-4-one (Compound 9; +++).

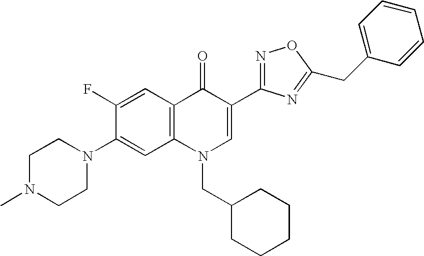

Proceeding as in Example 1, but substituting 1-cyclohexylmethyl-6-methoxy-4-oxo-1,4-dihydro-quinoline-3-carbonitrile, gave 3-(5-benzyl-[1,2,4]oxadiazol-3-yl)-1-cyclohexylmethyl-6-methoxy-1H-quinolin-4-one (Compound 10: +).

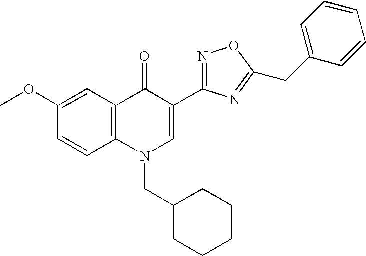

Proceeding as in Example 1, but substituting 1-(4-chloro-benzyl)-6-methoxy-4-oxo-1,4-dihydro-quinoline-3-carbonitrile, gave 3-(5-benzyl-[1,2,4]oxadiazol-3-yl)-1-(4-chloro-benzyl)-6-methoxy-1H-quinolin-4-one (Compound 11: +).

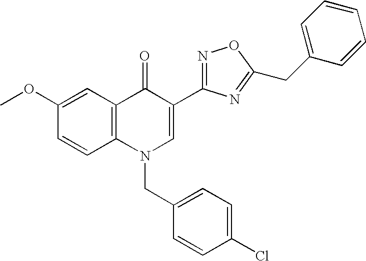

Proceeding as in Example 1, but substituting 1-(4-chloro-benzyl)-6-hydroxy-4-oxo-1,4-dihydro-quinoline-3-carbonitrile, gave 3-(5-benzyl-[1,2,4]oxadiazol-3-yl)-1-(4-chloro-benzyl)-6-hydroxy-1H-quinolin-4-one (Compound 12; ++).

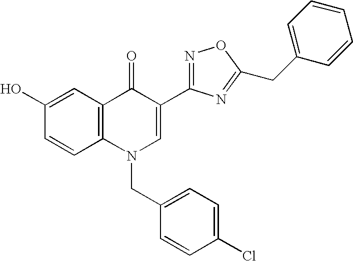

Proceeding as in Example 1, but substituting 1-cyclohexylmethyl-6-fluoro-7-(4-methyl-piperazin-1-yl)-4-oxo-1,4-dihydro-quinoline-3-carbonitrile and 3-methyl-butyric acid, gave 1-cyclohexylmethyl-6-fluoro-3-(5-isobutyl-[1,2,4]oxadiazol-3-yl)-7-(4-methyl-piperazin-1-yl)-1H-quinolin-4-one (Compound 13: +).

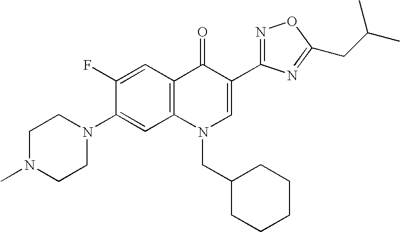

Proceeding as in Example 1, but substituting 1-cyclohexylmethyl-6-fluoro-7-(4-methyl-piperazin-1-yl)-4-oxo-1,4-dihydro-quinoline-3-carbonitrile and cyclopropylacetic acid, gave 1-cyclohexylmethyl-3-(5-cyclopropylmethyl-[1,2,4]oxadiazol-3-yl)-6-fluoro-7-(4-methyl-piperazin-1-yl)-1H-quinolin-4-one (Compound 14: ++).

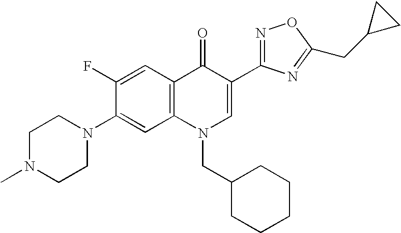

Proceeding as in Example 1, but substituting 1-cyclohexylmethyl-6-fluoro-7-(4-methyl-piperazin-1-yl)-4-oxo-1,4-dihydro-quinoline-3-carbonitrile and cyclopentylacetic acid, gave 1-cyclohexylmethyl-3-(5-cyclopentylmethyl-[1,2,4]oxadiazol-3-yl)-6-fluoro-7-(4-methyl-piperazin-1-yl)-1H-quinolin-4-one (Compound 15: ++).

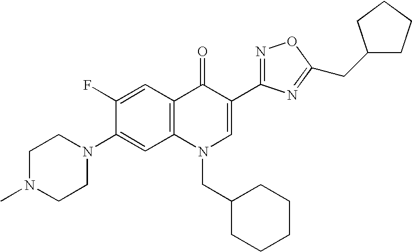

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile, gave 3-(5-benzyl-[1,2,4]oxadiazol-3-yl)-6-fluoro-1-(2-fluoro-4-trifluoromethyl-benzyl)-7-(4-methyl-piperazin-1-yl)-1H-quinolin-4-one (Compound 16: ++++). Mass Spec: 614.3 (M+1). ¹H NMR (DMSO-d⁶): 8.88 (1H, s), 7.88 (1H, d, J=13.2 Hz), 7.77 (1H, d, J=10.2 Hz), 7.56 (1H, d, J=7.3 Hz), 7.41 (3H, m), 7.20 (2H, m), 7.04 (1H, d, J=7.1 Hz), 5.90 (2H, s), 4.40 (2H, s), 3.14-3.63 (8H, m), 2.79 (3H, s).

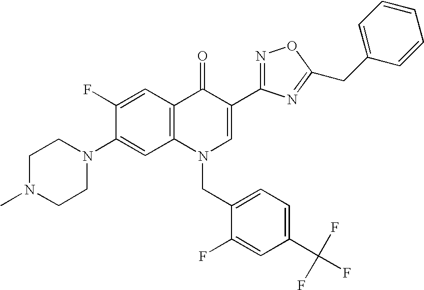

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and cyclopentylacetic acid, gave 3-(5-cyclopentylmethyl-[1,2,4]oxadiazol-3-yl)-6-fluoro-1-(2-fluoro-4-trifluoromethyl-benzyl)-7-(4-methyl-piperazin-1-yl)-1H-quinolin-4-one (Compound 17; +++). Mass Spec: 588.0 (M+1). ¹H NMR (DMSO-d⁶): 9.91 (1H, bs), 8.90 (1H, s), 7.90 (1H, d, J=13.1 Hz), 7.78 (1H, d, J=10.0 Hz), 7.56 (1H, d, J=8.6 Hz), 7.41 (1H, m), 7.05 (1H, d, J=7.1 Hz), 5.91 (2H, s), 3.65 (2H, d, J=13.1 Hz), 3.52 (2H, d, J=11.4 Hz), 3.19 (2H, m), 3.09 (2H, m), 2.96 (2H, d, J=7.4 Hz), 2.84 (3H, d, J=4.5 Hz), 2.30 (1H, m), 1.78 (2H, m), 1.62 (2H, m), 1.52 (2H, m), 1.25 (2H, m).

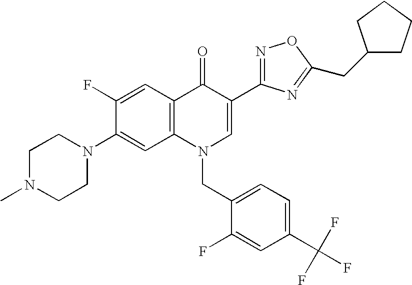

Proceeding as in Example 1, but substituting 1-cyclohexylmethyl-6-fluoro-7-(4-methyl-piperazin-1-yl)-4-oxo-1,4-dihydro-quinoline-3-carbonitrile and 3-methylphenylacetic acid, gave 1-cyclohexylmethyl-6-fluoro-3-[5-(3-methyl-benzyl)-[1,2,4]oxadiazol-3-yl]-7-(4-methyl-piperazin-1-yl)-1H-quinolin-4-one (Compound 19: ++).

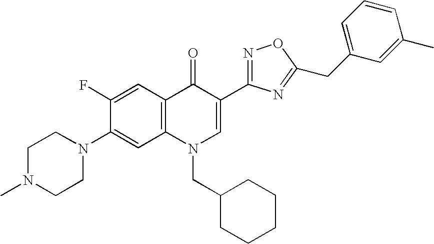

Proceeding as in Example 1, but substituting 1-cyclohexylmethyl-6-fluoro-7-(4-methyl-piperazin-1-yl)-4-oxo-1,4-dihydro-quinoline-3-carbonitrile and 2-methylphenylacetic acid, gave 1-cyclohexylmethyl-6-fluoro-3-[5-(2-methyl-benzyl)-[1,2,4]oxadiazol-3-yl]-7-(4-methyl-piperazin-1-yl)-1H-quinolin-4-one (Compound 20: ++).

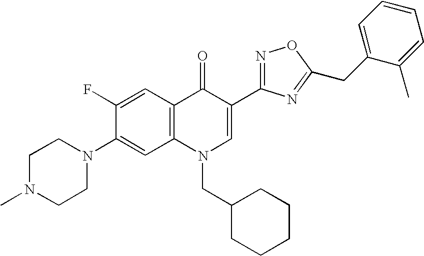

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 3,3-dimethyl-butyric acid, gave 1-(4-chloro-benzyl)-3-[5-(2,2-dimethyl-propyl)-[1,2,4]oxadiazol-3-yl]-6-fluoro-7-(4-methyl-piperazin-1-yl)-1H-quinolin-4-one (Compound 21: +). Mass Spec: 524.2 (M+1). ¹H NMR (400 MHz, CDCl₃, δ): 8.69 (1H, s), 8.14 (1H, d, J=12.35), 7.39 (2H, d, J=7.62), 7.19 (2H, d, J=7.89), 6.37 (1H, s), 5.49 (2H, s), 3.70 (2H, t, J=10.72), 3.54 (2H, d, J=9.42), 3.40 (2H, d, J=13.44), 3.17 (2H, m), 2.90 (2H, d, J=4.35), 2.88 (3H, s), 1.12 (9H, s).

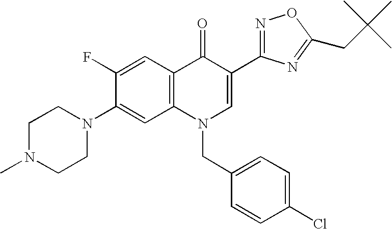

Proceeding as in Example 1, but substituting 1-(4-chloro-benzyl)-6-fluoro-7-(4-methyl-piperazin-1-yl)-4-oxo-1,4-dihydro-quinoline-3-carbonitrile and (2-carboxymethyl-phenyl)-acetic acid, gave (2-{3-[1-(4-chloro-benzyl)-6-fluoro-7-(4-methyl-piperazin-1-yl)-4-oxo-1,4-dihydro-quinolin-3-yl]-[1,2,4]oxadiazol-5-ylmethyl}-phenyl)-acetic acid (Compound 22; +). Mass Spec: 602.3 (M+1). ¹H NMR (400 MHz, DMSO-d₆, δ): 8.88 (1H, s), 7.90 (1H, d, J=13.3), 7.45 (2H, d, J=8.42), 7.33 (5H, m), 7.05 (1H, d, J=7.24), 5.77 (2H, s), 4.44 (2H, s), 3.75 (2H, s), 3.64 (2H, d, J=11.91), 3.53 (2H, d, J=11.1), 3.18 (4H, m), 2.86 (3H, s).

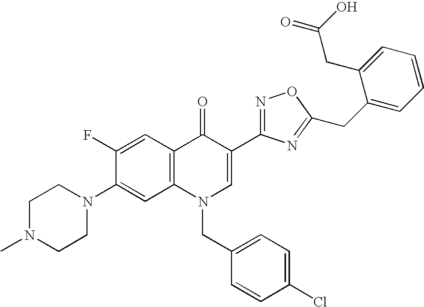

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and 4-fluorobenzylacetic acid, gave 6-fluoro-3-[5-(4-fluoro-benzyl)-[1,2,4]oxadiazol-3-yl]-1-(2-fluoro-4-trifluoromethyl-benzyl)-7-(4-methyl-piperazin-1-yl)-1H-quinolin-4-one (Compound 23; +++). Mass Spec: 614.3 (M+1). ¹H NMR (DMSO-d⁶): 8.88 (1H, s), 7.88 (1H, d, J=13.2 Hz), 7.77 (1H, d, J=10.2 Hz), 7.56 (1H, d, J=7.3 Hz), 7.41 (3H, m), 7.20 (2H, m), 7.04 (1H, d, J=7.1 Hz), 5.90 (2H, s), 4.40 (2H, s), 3.14-3.63 (8H, m), 2.79 (3H, s).

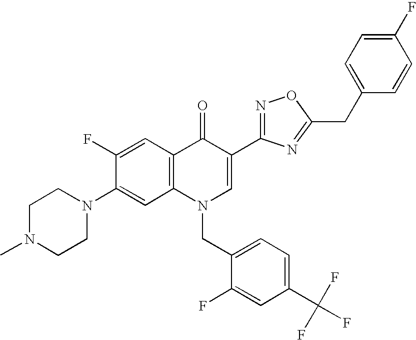

Proceeding as in Example 1, but substituting 1-cyclohexylmethyl-6-hydroxy-4-oxo-1,4-dihydro-quinoline-3-carbonitrile, gave 3-(5-benzyl-[1,2,4]oxadiazol-3-yl)-1-cyclohexylmethyl-6-hydroxy-1H-quinolin-4-one (Compound 24: +). Mass Spec: 416.1 (M+1), 438.3 (M+23), 414.2 (M−1).

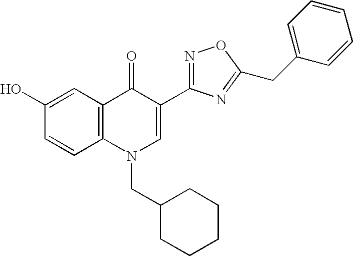

Proceeding as in Example 1, but substituting 1-(4-chloro-benzyl)-6-fluoro-4-oxo-7-piperazin-1-yl-1,4-dihydro-quinoline-3-carbonitrile, gave 3-(5-benzyl-[1,2,4]oxadiazol-3-yl)-1-(4-chloro-benzyl)-6-fluoro-7-piperazin-1-yl-1H-quinolin-4-one (Compound 25: +++). Mass Spec: 530.2 (M+1), 552.3 (M+23), 528.4 (M−1). ¹H NMR: 9.07 (br s, 1.5H), 8.92 (s, 1H), 7.86 (d, 1H, J=13.3 Hz), 7.44 (d, 2H, J=8.6 Hz), 7.40-7.37 (br m, 4H), 7.35-7.30 (br d, 3H), 7.03 (d, 1H, J=7.3 Hz), 6.54 (s, 0.5H), 5.76 (s, 2H), 4.41 (s, 2H), 3.32-3.29 (br m, 4H), 3.27-3.22 (br m, 4H).

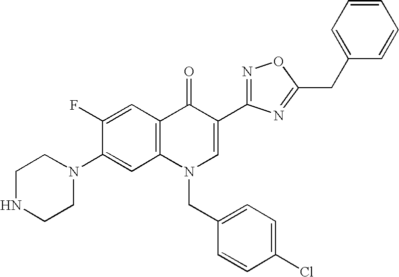

Proceeding as in Example 1, but substituting 1-(4-chloro-benzyl)-6-fluoro-7-(4-hydroxy-piperidin-1-yl)-4-oxo-1,4-dihydro-quinoline-3-carbonitrile, gave 3-(5-benzyl-[1,2,4]oxadiazol-3-yl)-1-(4-chloro-benzyl)-6-fluoro-7-(4-hydroxy-piperidin-1-yl)-1H-quinolin-4-one (Compound 26; +). Mass Spec: 545.3 (M+1), 677.2 (M+23), 543.2 (M−1). ¹H NMR: 8.91 (s, 1H), 7.80 (d, 1H, J=12.7 Hz), 7.45 (d, 2H, J=8.4 Hz), 7.42-7.39 (m, 4H), 7.35-7.31 (m, 3H), 6.93 (d, 1H, J=7.6 Hz), 5.73 (s, 2H), 4.76 (d, 1H, J=4.2 Hz), 4.42 (s, 2H), 3.33 (br s, 2H), 3.71-3.64 (m, 1H), 2.91-2.83 (m, 2H), 1.86-1.78 (m, 2H), 1.55-1.45 (m, 2H).

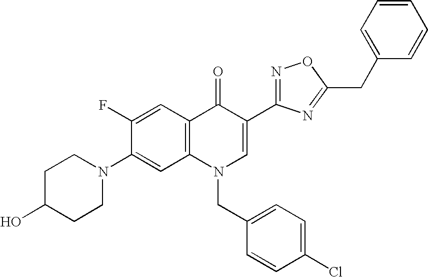

Proceeding as in Example 1, but substituting 7-(4-acetyl-piperazin-1-yl)-1-(4-chloro-benzyl)-6-fluoro-4-oxo-1,4-dihydro-quinoline-3-carbonitrile, gave 7-(4-acetyl-piperazin-1-yl)-3-(5-benzyl-[1,2,4]oxadiazol-3-yl)-1-(4-chloro-benzyl)-6-fluoro-1H-quinolin-4-one (Compound 27: +++). Mass Spec: 572.2 (M+1), 594 (M+23), 570.2 (M−1). ¹H NMR: 8.92 (s, 1H), 7.83 (d, 1H, J=13.7 Hz), 7.48-7.42 (m, 3H), 7.39 (br s, 2H), 7.38 (br s, 2H), 7.33 (d, 2H, J=8.6 Hz), 6.97 (d, 1H, J=7 Hz), 5.72 (s, 2H), 4.41 (s, 2H), 3.60-3.54 (br m, 4H), 3.13-3.08 (br t, 2H), 3.06-3.02 (br t, 2H), 2.04 (s, 3H).

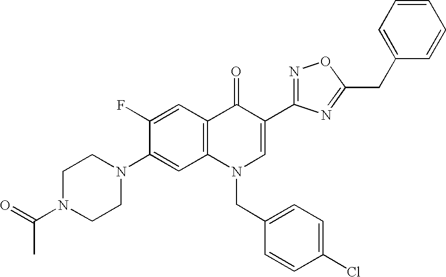

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 4-fluoroacetic acid, gave 1-(4-chloro-benzyl)-6-fluoro-3-[5-(4-fluoro-benzyl)-[1,2,4]oxadiazol-3-yl]-7-(4-methyl-piperazin-1-yl)-1H-quinolin-4-one (Compound 28: +++). Mass Spec: 562.0 (M+1). ¹H NMR (400 MHz, DMSO-d₆, δ): 8.83 (1H, s), 7.79 (1H, d, J=12.7), 7.36 (3H, m), 7.26 (2H, m), 7.14 (2H, m), 6.97 (1H, d, J=7.6), 5.68 (2H, s), 4.34 (2H, s), 3.35 (4H, m), 3.20 (4H, m), 2.75 (3H, s).

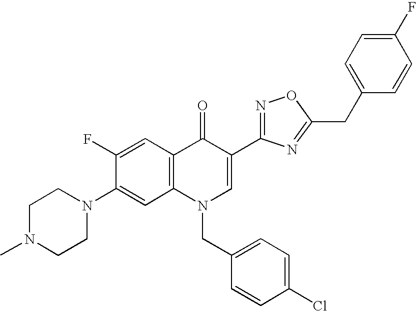

Proceeding as in Example 1, but substituting 1-(4-chloro-benzyl)-6-fluoro-7-(4-methyl-piperazin-1-yl)-4-oxo-1,4-dihydro-quinoline-3-carbonitrile and (2-aminomethylphenyl)acetic acid, gave 3-[5-(2-aminomethyl-benzyl)-[1,2,4]oxadiazol-3-yl]-1-(4-chloro-benzyl)-6-fluoro-7-(4-methyl-piperazin-1-yl)-1H-quinolin-4-one (Compound 29). Mass Spec: 573.2 (M+1). ¹H NMR (400 MHz, DMSO-d₆, δ): 8.93 (1H, s), 8.37 (2H, br-s), 7.84 (1H, d, J=12.7), 7.57 (1H, d, J=6.75), 7.43 (3H, m), 7.32 (2H, m), 7.06 (2H, d, J=7.3), 5.77 (2H, s), 4.61 (2H, s), 4.22 (2H, q, J=5.7), 3.40 (4H, m), 3.28 (4H, m), 2.83 (3H, s).

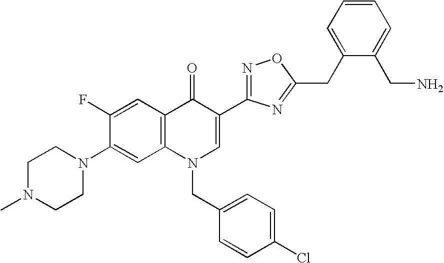

Proceeding as in Example 1, but substituting 1-(4-chloro-benzyl)-6-fluoro-7-(4-methyl-piperazin-1-yl)-4-oxo-1,4-dihydro-quinoline-3-carbonitrile and [2-(tert-butoxycarbonylamino-methyl)-phenyl]-acetic acid, gave tert-butyl (2-{3-[1-(4-chloro-benzyl)-6-fluoro-7-(4-methyl-piperazin-1-yl)-4-oxo-1,4-dihydro-quinolin-3-yl]-[1,2,4]oxadiazol-5-ylmethyl}-benzyl)-carbamate (Compound 30). Mass Spec: 673.3 (M+1). ¹H NMR (400 MHz, DMSO-d₆, δ): 8.90 (1H, s), 7.84 (1H, d, J=13.2), 7.43 (3H, m), 7.29 (5H, m), 7.02 (2H, d, J=6.17), 5.75 (2H, s), 4.46 (2H, s), 4.28 (2H, d, J=5.88), 3.42 (4H, m), 3.20 (4H, m), 2.84 (3H, s), 1.36 (9H, s).

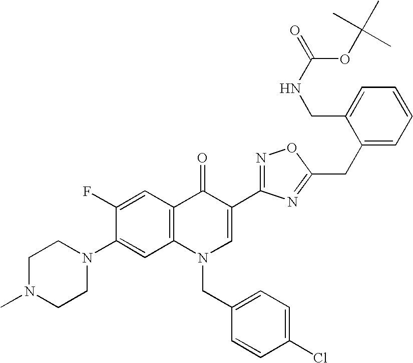

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 2-phenyl-succinic acid, gave 3-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-3-phenyl-propionic acid (Compound 31: +). Mass Spec: 602.3 (M+1). ¹H NMR (400 MHz, DMSO-d₆, δ): 8.89 (1H, s), 7.88 (1H, d, J=12.62), 7.39 (5H, m), 7.05 (2H, d, J=6.73), 5.77 (2H, s), 4.26 (1H, dd, J=9.29, 6.66), 3.69 (2H, d, J=9.44), 3.53 (4H, m), 3.16 (4H, m), 2.84 (3H, s).

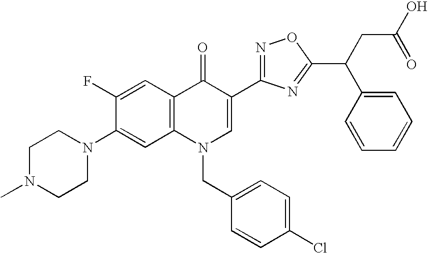

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 2,4-difluorophenylacetic acid, gave 1-(4-chloro-benzyl)-3-[5-(2,4-difluoro-benzyl)-[1,2,4]oxadiazol-3-yl]-6-fluoro-7-(4-methyl-piperazin-1-yl)-1H-quinolin-4-one (Compound 32: +). Mass Spec: 580.0 (M⁺). ¹H NMR (400 MHz, DMSO-d₆, δ): 8.85 (1H, s), 7.86 (1H, d, J=13.02), 7.48 (1H, m), 7.43 (2H, d, J=8.4), 7.31 (2H, d, J=8.22), 7.21 (2H, t, J=7.72), 7.03 (2H, d, J=7.1), 5.75 (2H, s), 4.45 (2H, s), 3.62 (2H, d, J=10.31), 3.49 (2H, d, J=10.03), 3.14 (4H, m), 2.82 (3H, s).

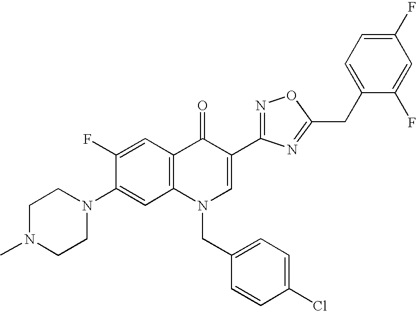

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 2,6-difluorophenylacetic acid, gave 1-(4-chloro-benzyl)-3-[5-(2,6-difluoro-benzyl)-[1,2,4]oxadiazol-3-yl]-6-fluoro-7-(4-methyl-piperazin-1-yl)-1H-quinolin-4-one (Compound 33: ++). Mass Spec: 580.4 (M⁺). ¹H NMR (400 MHz, DMSO-d₆, δ): 8.86 (1H, s), 7.86 (1H, d, J=13.57), 7.57 (1H, m), 7.43 (2H, d, J=8.82), 7.31 (3H, m), 7.15 (1H, dt, J=8.85, 2.1), 7.04 (1H, d, J=7.1), 5.75 (2H, s), 4.44 (2H, s), 3.60 (2H, d, J=11.34), 3.50 (2H, d, J=11.40), 3.14 (4H, m), 2.82 (3H, s).

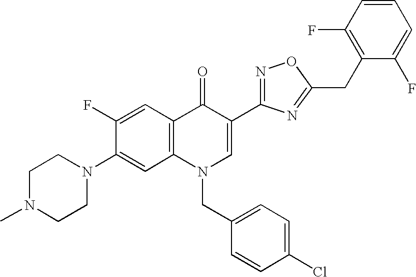

Proceeding as in Example 1, but substituting 1-(4-chloro-2-fluoro-benzyl)-6-fluoro-7-(4-methyl-piperazin-1-yl)-4-oxo-1,4-dihydro-quinoline-3-carbonitrile and 4-fluorophenylacetic acid, gave 1-(4-chloro-2-fluoro-benzyl)-6-fluoro-3-[5-(4-fluoro-benzyl)-[1,2,4]oxadiazol-3-yl]-7-(4-methyl-piperazin-1-yl)-1H-quinolin-4-one (Compound 34: +++). Mass Spec: 580.2 (M+1). ¹H NMR (DMSO-d⁶): 8.86 (1H, s), 7.86 (1H, d, J=13.1 Hz), 7.51 (1H, dd J=10.5 and 1.9 Hz), 7.42 (2H, dd, J=5.5 and 3.0 Hz), 7.29 (2H, m), 7.20 (2H, m), 7.07 (1H, d, J=7.2 Hz), 5.79 (2H, s), 4.40 (2H, s), 3.63 (2H, d, J=8.5 Hz), 3.51 (2H, d, J=7.3 Hz), 3.19 (4H, m), 2.81 (3H, br d, J=2.5 Hz).

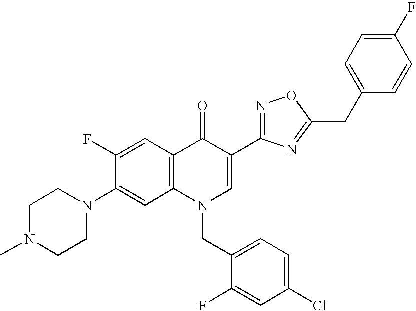

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile, gave 3-(5-benzyl-[1,2,4]oxadiazol-3-yl)-6-fluoro-1-(2-fluoro-4-trifluoromethyl-benzyl)-7-(4-methyl-piperazin-1-yl)-1H-quinolin-4-one (Compound 35: +++). Mass Spec: 561.8 (M+1). ¹H NMR (DMSO-d⁶): 10.37 (1H, br s), 8.88 (1H, s), 7.87 (1H, d, J=13.1 Hz), 7.51 (1H, dd, J=10.5 and 2.0 Hz), 7.26-7.37 (6H, m), 7.06 (1H, d, J=7.2 Hz), 5.79 (2H, s), 4.39 (2H, s), 3.64 (2H, bs), 3.50 (2H, bs), 3.16 (4H, bs), 2.82 (3H, s).

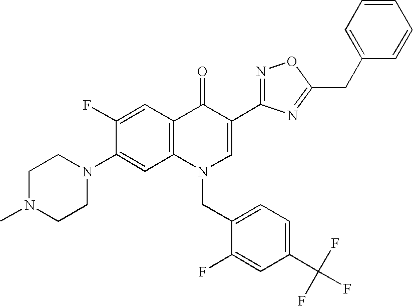

Proceeding as in Example 1, but substituting 6-fluoro-7-(4-methyl-piperazin-1-yl)-4-oxo-1-(4-trifluoromethyl-benzyl)-1,4-dihydro-quinoline-3-carbonitrile, gave 3-(5-benzyl-[1,2,4]oxadiazol-3-yl)-6-fluoro-7-(4-methyl-piperazin-1-yl)-1-(4-trifluoromethyl-benzyl)-1H-quinolin-4-one (Compound 36: +++).

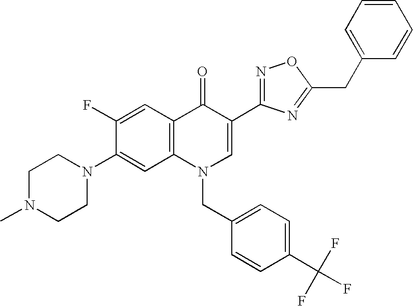

Proceeding as in Example 1, but substituting 6-fluoro-7-(4-methyl-piperazin-1-yl)-4-oxo-1-(4-trifluoromethyl-benzyl)-1,4-dihydro-quinoline-3-carbonitrile and 4-fluorophenylacetic acid, gave 6-fluoro-3-[5-(4-fluoro-benzyl)-[1,2,4]oxadiazol-3-yl]-7-(4-methyl-piperazin-1-yl)-1-(4-trifluoromethyl-benzyl)-1H-quinolin-4-one (Compound 37; +++).

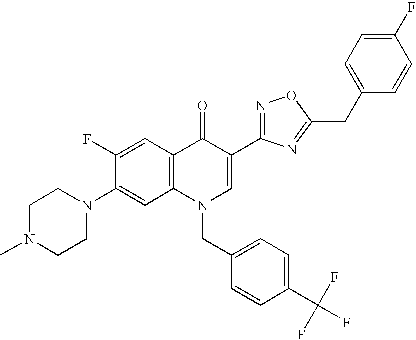

Proceeding as in Example 1, but substituting 7-[1,4]diazepan-1-yl-6-fluoro-1-(2-fluoro-4-trifluoromethyl-benzyl)-4-oxo-1,4-dihydro-quinoline-3-carbonitrile, gave 3-(5-benzyl-[1,2,4]oxadiazol-3-yl)-7-[1,4]diazepan-1-yl-6-fluoro-1-(2-fluoro-4-trifluoromethyl-benzyl)-1H-quinolin-4-one (Compound 38: ++++).

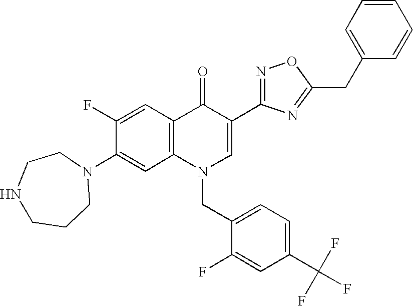

Proceeding as in Example 1, but substituting 1-(2-ethyl-butyl)-6-fluoro-7-(4-methyl-piperazin-1-yl)-4-oxo-1,4-dihydro-quinoline-3-carbonitrile, gave 3-(5-benzyl-[1,2,4]oxadiazol-3-yl)-1-(2-ethyl-butyl)-6-fluoro-7-(4-methyl-piperazin-1-yl)-1H-quinolin-4-one (Compound 39). Mass Spec: 490.2 (M+1), 512.3 (M+23), 488.3 (M−1).

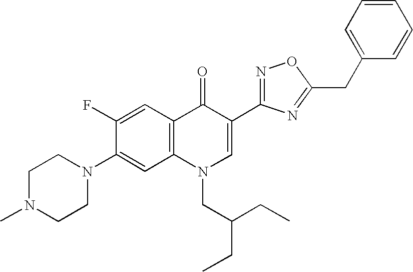

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and hexanoic acid, gave 6-fluoro-1-(2-fluoro-4-trifluoromethyl-benzyl)-7-(4-methyl-piperazin-1-yl)-3-(5-pentyl-[1,2,4]oxadiazol-3-yl)-1H-quinolin-4-one (Compound 40: +++). Mass Spec: 576.5 (M+1). ¹H NMR (DMSO-d⁶): 8.90 (1H, s), 7.89 (1H, d, J=13.2 Hz), 7.77 (1H, d, J=11.5 Hz), 7.57 (1H, d, J=8.0 Hz), 7.42 (1H, m), 7.05 (1H, d, J=7.0 Hz), 5.91 (2H, s), 3.61 (2H, bs), 3.51 (2H, bs), 3.15 (4H, bs), 2.94 (2H, t), 2.80 (3H, s), 1.76 (2H, m), 1.32 (4H, m), 0.87 (3H, t).

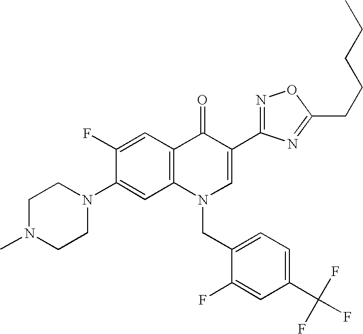

Proceeding as in Example 1, but substituting 1-(2-chloro-6-fluoro-benzyl)-6-fluoro-7-(4-methyl-piperazin-1-yl)-4-oxo-1,4-dihydro-quinoline-3-carbonitrile, gave 3-(5-benzyl-[1,2,4]oxadiazol-3-yl)-1-(2-chloro-6-fluoro-benzyl)-6-fluoro-7-(4-methyl-piperazin-1-yl)-1H-quinolin-4-one (Compound 41: +). Mass Spec: 562.3 (M+1). ¹H NMR (DMSO-d⁶): 8.75 (1H, d, J=2.2 Hz), 7.87 (1H, d, J=13.1 Hz), 7.46-7.55 (3H, m), 7.29-7.39 (5H, m), 7.03 (1H, d, J=7.0 Hz), 5.84 (2H, s), 4.36 (2H, s), 3.19-3.65 (8H, m), 2.82 (3H, s).

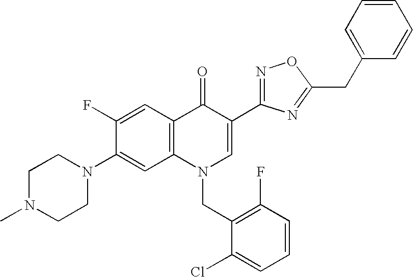

Proceeding as in Example 1, but substituting 1-(2-chloro-6-fluoro-benzyl)-6-fluoro-7-(4-methyl-piperazin-1-yl)-4-oxo-1,4-dihydro-quinoline-3-carbonitrile and 4-fluorophenylacetic acid, gave 1-(2-chloro-6-fluoro-benzyl)-6-fluoro-3-[5-(4-fluoro-benzyl)-[1,2,4]oxadiazol-3-yl]-7-(4-methyl-piperazin-1-yl)-1H-quinolin-4-one (Compound 42: ++). Mass Spec: 580.5 (M+1). ¹H NMR (DMSO-d⁶): 8.72 (1H, d, J=1.9 Hz), 7.87 (1H, d, J=13.1 Hz), 7.45-7.60 (2H, m), 7.29-7.43 (3H, m), 7.20 (2H, m), 7.04 (1H, d, J=6.7 Hz), 5.83 (2H, s), 4.38 (2H, s), 3.64 (2H, bs), 3.51 (2H, bs), 3.20 (4H, bs), 2.82 (3H, s).

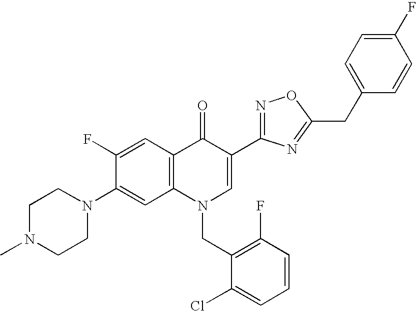

Proceeding as in Example 1, but substituting 3-cyano-1-(2-fluoro-4-trifluoromethyl-benzyl)-4-oxo-1,4-dihydro-quinoline-6-carboxylic acid, gave 3-(5-benzyl-[1,2,4]oxadiazol-3-yl)-1-(2-fluoro-4-trifluoromethyl-benzyl)-4-oxo-1,4-dihydro-quinoline-6-carboxylic acid (Compound 43: +). Mass Spec: 524.4 (M+1), 546 (M+23), 522.1 (M−1). ¹H NMR: 9.02 (s, 1H), 8.88 (d, 1H, J=2.7 Hz), 8.18 (dd, 1H, J=7.0, 2.1 Hz), 7.82 (d, 1H, J=10.5 Hz), 7.74 (d, 1H, J=9.0 Hz), 7.52 (d, 1H, J=8.0 Hz), 7.42-7.39 (br m, 4H), 7.36-7.26 (m, 1H), 5.93 (s, 2H), 4.44 (s, 2H).

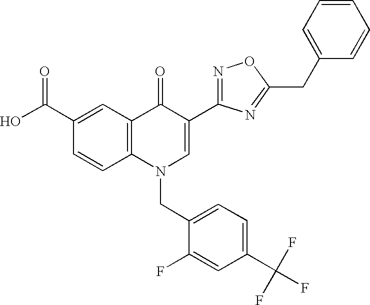

Proceeding as in Example 1, but substituting 1-(2,4-difluoro-benzyl)-6-fluoro-7-(4-methyl-piperazin-1-yl)-4-oxo-1,4-dihydro-quinoline-3-carbonitrile, gave 1-(2,4-difluoro-benzyl)-6-fluoro-3-[5-(4-fluoro-benzyl)-[1,2,4]oxadiazol-3-yl]-7-(4-methyl-piperazin-1-yl)-1H-quinolin-4-one (Compound 44: +++). Mass Spec: 545.8 (M+1). ¹H NMR (DMSO-d⁶): 10.34 (1H, bs), 8.87 (1H, s), 7.86 (1H, d, J=13.2 Hz), 7.28-7.43 (6H, m), 7.05-7.11 (2H, m), 5.77 (2H, s), 4.39 (2H, s), 3.64 (2H, bs), 3.52 (2H, bs), 3.17 (4H, m), 2.82 (3H, s).

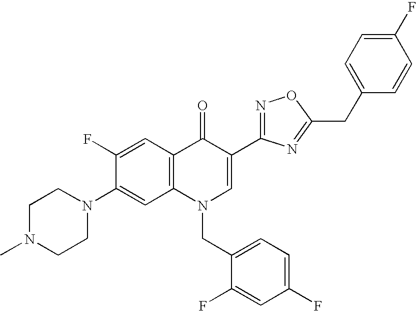

Proceeding as in Example 1, but substituting 6-fluoro-1-(2-fluoro-benzyl)-7-(4-methyl-piperazin-1-yl)-4-oxo-1,4-dihydro-quinoline-3-carbonitrile and 4-fluorophenylacetic acid, gave 3-(5-benzyl-[1,2,4]oxadiazol-3-yl)-6-fluoro-1-(2-fluoro-benzyl)-7-(4-methyl-piperazin-1-yl)-1H-quinolin-4-one (Compound 45: ++). Mass Spec: 528.1 (M+1). ¹H NMR (DMSO-d⁶): 10.36 (1H, bs), 8.89 (1H, s), 7.86 (1H, d, J=13.1 Hz), 7.16-7.44 (8H, m), 7.09 (1H, d, J=7.3 Hz), 5.79 (2H, s), 4.39 (2H, s), 3.15-3.62 (8H, m), 2.81 (3H, s).

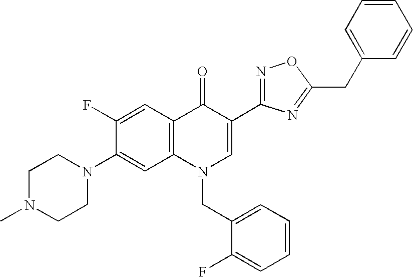

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 3,5-difluorophenylacetic acid, gave 1-(4-chloro-benzyl)-3-[5-(3,5-difluoro-benzyl)-[1,2,4]oxadiazol-3-yl]-6-fluoro-7-(4-methyl-piperazin-1-yl)-1H-quinolin-4-one (Compound 46: ++). Mass Spec: 580.1 (M⁺). ¹H NMR (400 MHz, DMSO-d₆, δ): 8.91 (1H, s), 7.89 (1H, d, J=13.04), 7.44 (2H, d, J=6.16), 7.34 (2H, d, J=8.28), 7.21 (3H, m), 7.05 (1H, d, J=6.32), 5.77 (2H, s), 4.50 (2H, s), 3.64 (2H, d, J=12.03), 3.52 (2H, d, J=11.91), 3.09 (4H, m), 2.86 (3H, s).

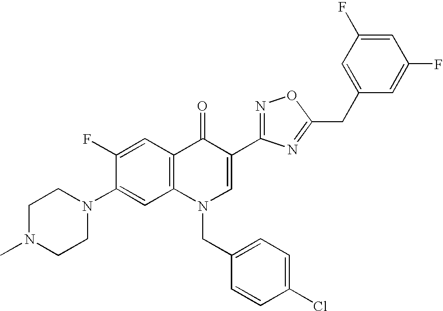

Proceeding as in Example 1, but substituting 6-fluoro-1-(2-fluoro-benzyl)-7-(4-methyl-piperazin-1-yl)-4-oxo-1,4-dihydro-quinoline-3-carbonitrile and 4-fluorophenylacetic acid, gave 6-fluoro-3-[5-(4-fluoro-benzyl)-[1,2,4]oxadiazol-3-yl]-1-(2-fluoro-benzyl)-7-(4-methyl-piperazin-1-yl)-1H-quinolin-4-one (Compound 47: ++). Mass Spec: 545.8 (M+1). ¹H NMR (DMSO-d⁶): 10.71 (1H, bs), 8.87 (1H, s), 7.86 (1H, d, J=13.2 Hz), 7.36-7.50 (3H, m), 7.25-7.33 (2H, m), 7.17-7.24 (3H, m), 7.11 (1H, d, J=7.2 Hz), 5.79 (2H, s), 4.40 (2H, s), 3.62 (2H, d, J=8.7 Hz), 3.51 (2H, d, J=7.1 Hz), 3.17 (4H, m), 2.81 (3H, d, J=4.5 Hz).

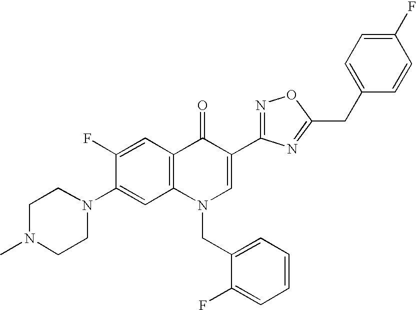

Proceeding as in Example 1, but substituting 1-(2,6-difluoro-benzyl)-6-fluoro-7-(4-methyl-piperazin-1-yl)-4-oxo-1,4-dihydro-quinoline-3-carbonitrile, gave 3-(5-benzyl-[1,2,4]oxadiazol-3-yl)-1-(2,6-difluoro-benzyl)-6-fluoro-7-(4-methyl-piperazin-1-yl)-1H-quinolin-4-one (Compound 48: ++). Mass Spec: 545.8 (M+1). ¹H NMR (DMSO-d⁶): 8.91 (1 H, s), 7.85 (1H, d, J=13.0 Hz), 7.47-7.55 (1H, m), 7.37-7.39 (3H, m), 7.31 (1H, m), 7.18 (2H, m), 7.12 (1H, d, J=7.0 Hz), 5.80 (2H, s), 4.40 (2H, s), 3.65 (2H, d, J=9.7 Hz), 3.55 (2H, d, J=9.0 Hz), 3.22 (4H, m), 2.83 (3H, d, J=4.4 Hz).

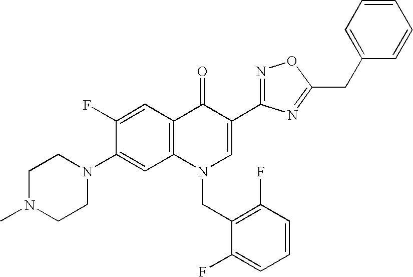

Proceeding as in Example 1, but substituting 1-(2,6-difluoro-benzyl)-6-fluoro-7-(4-methyl-piperazin-1-yl)-4-oxo-1,4-dihydro-quinoline-3-carbonitrile and 4-fluorophenylacetic acid, gave 1-(2,6-difluoro-benzyl)-6-fluoro-3-[5-(4-fluoro-benzyl)-[1,2,4]oxadiazol-3-yl]-7-(4-methyl-piperazin-1-yl)-1H-quinolin-4-one (Compound 49; ++). Mass Spec: 564.0 (M+1). ¹H NMR (DMSO-d⁶): 8.89 (1H, s), 7.85 (1H, d, J=13.1 Hz), 7.51 (1H, m), 7.43 (2H, m), 7.16-7.24 (4H, m), 7.12 (1H, d, J=7.1 Hz), 5.80 (2H, s), 4.40 (2H, s), 3.65 (2H, bs), 3.55 (2H, bs), 3.21 (4H, bs), 2.83 (3H, s).

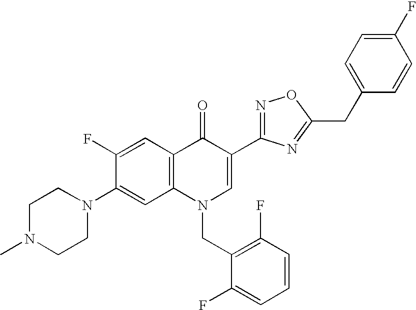

Proceeding as in Example 1, but substituting 1-(4-chlorobenzyl)-6-fluoro-4-oxo-7-(4-(pyridin-2-yl)piperazin-1-yl)-1,4-dihydroquinoline-3-carbonitrile and 4-fluorophenylacetic acid, gave 1-(4-chlorobenzyl)-6-fluoro-3-(5-(4-fluorobenzyl)-1,2,4-oxadiazol-3-yl)-7-(4-(pyridin-2-yl)piperazin-1-yl)quinolin-4(1H)-one (Compound 50: +).

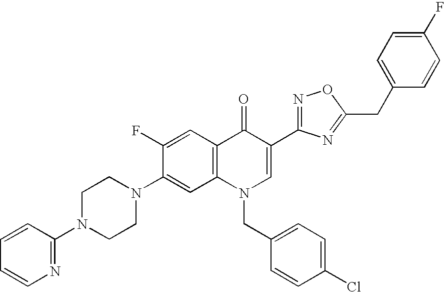

Proceeding as in Example 1, but substituting 7-dimethylamino-6-fluoro-1-(2-fluoro-4-trifluoromethyl-benzyl)-4-oxo-1,4-dihydro-quinoline-3-carbonitrile, gave 3-(5-benzyl-[1,2,4]oxadiazol-3-yl)-7-dimethylamino-6-fluoro-1-(2-fluoro-4-trifluoromethyl-benzyl)-1H-quinolin-4-one (Compound 51: +).

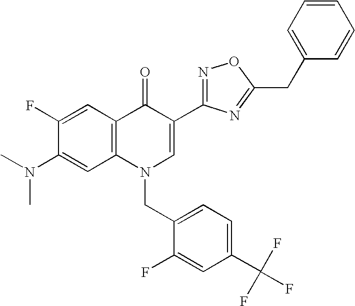

Proceeding as in Example 1, but substituting 1-(4-chloro-benzyl)-6-fluoro-7-(4-methyl-piperazin-1-yl)-4-oxo-1,4-dihydro-quinoline-3-carbonitrile and piperidin-1-ylacetic acid gave 1-(4-chloro-benzyl)-6-fluoro-7-(4-methyl-piperazin-1-yl)-3-(5-piperidin-1-ylmethyl-[1,2,4]oxadiazol-3-yl)-1H-quinolin-4-one (Compound 52: +).

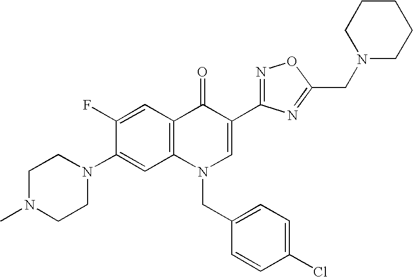

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 3-cyano-2-phenyl-propionic acid gave 3-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-3-phenyl-propionitrile (Compound 53: ++).

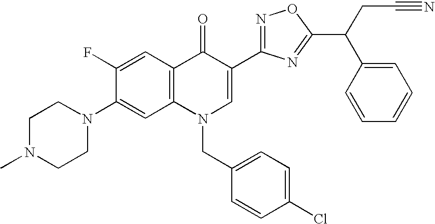

Proceeding as in Example 1, but substituting 1-(4-chloro-benzyl)-6-fluoro-7-(4-methyl-piperazin-1-yl)-4-oxo-1,4-dihydro-quinoline-3-carbonitrile and pyrrolidin-1-yl-acetic acid, gave 1-(4-chloro-benzyl)-6-fluoro-7-(4-methyl-piperazin-1-yl)-3-(5-pyrrolidin-1-ylmethyl-[1,2,4]oxadiazol-3-yl)-1H-quinolin-4-one (Compound 54: +).

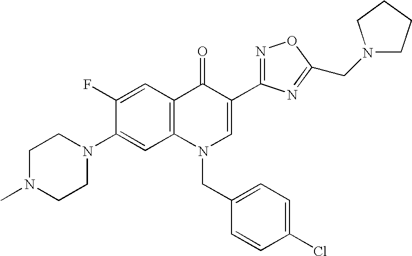

Proceeding as in Example 1, but substituting 6-fluoro-1-(2-fluoro-4-trifluoromethyl-benzyl)-7-methylamino-4-oxo-1,4-dihydro-quinoline-3-carbonitrile, gave 3-(5-Benzyl-[1,2,4]oxadiazol-3-yl)-6-fluoro-1-(2-fluoro-4-trifluoromethyl-benzyl)-7-methylamino-1H-quinolin-4-one (Compound 55: +).

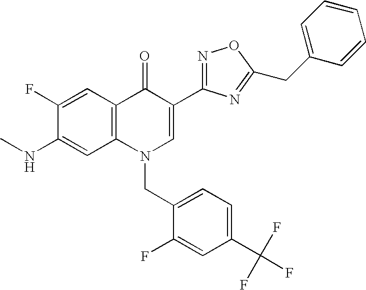

Proceeding as in Example 1, but substituting 6-fluoro-1-(2-fluoro-4-trifluoromethyl-benzyl)-7-imidazol-1-yl-4-oxo-1,4-dihydro-quinoline-3-carbonitrile, gave 3-(5-benzyl-[1,2,4]oxadiazol-3-yl)-6-fluoro-1-(2-fluoro-4-trifluoromethyl-benzyl)-7-imidazol-1-yl-1H-quinolin-4-one (Compound 56: +++).

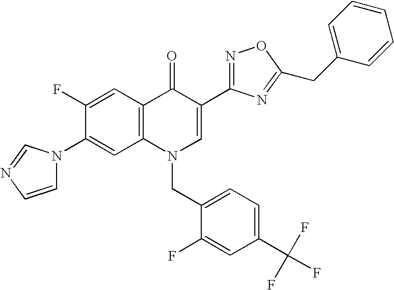

Proceeding as in Example 1, but substituting 6-fluoro-1-(2-fluoro-4-trifluoromethyl-benzyl)-7-(4-methyl-imidazol-1-yl)-4-oxo-1,4-dihydro-quinoline-3-carbonitrile, gave 3-(5-benzyl-[1,2,4]oxadiazol-3-yl)-6-fluoro-1-(2-fluoro-4-trifluoromethyl-benzyl)-7-(4-methyl-imidazol-1-yl)-1H-quinolin-4-one (Compound 57: ++).

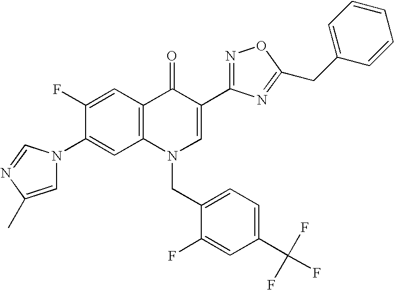

Proceeding as in Example 1, but substituting 6-fluoro-7-(4-methyl-piperazin-1-yl)-1-(2-morpholin-4-yl-ethyl)-4-oxo-1,4-dihydro-quinoline-3-carbonitrile and (4-fluoro-phenyl)-acetic acid, gave 6-fluoro-3-[5-(4-fluoro-benzyl)-[1,2,4]oxadiazol-3-yl]-7-(4-methyl-piperazin-1-yl)-1-(2-morpholin-4-yl-ethyl)-1H-quinolin-4-one (Compound 58: +).

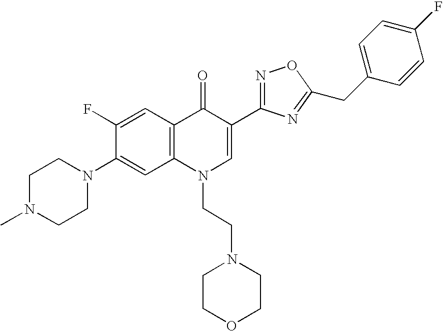

Proceeding as in Example 1, but substituting 6-fluoro-7-(4-methyl-piperazin-1-yl)-1-(2-morpholin-4-yl-ethyl)-4-oxo-1,4-dihydro-quinoline-3-carbonitrile, gave 3-(5-benzyl-[1,2,4]oxadiazol-3-yl)-6-fluoro-7-(4-methyl-piperazin-1-yl)-1-(2-morpholin-4-yl-ethyl)-1H-quinolin-4-one (Compound 59: +).

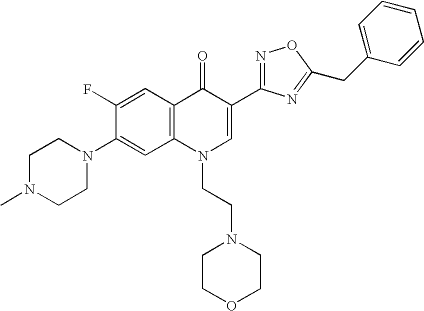

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 4-amino-4-oxo-2-phenylbutanoic acid, gave 3-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-3-phenyl-propionamide (Compound 60: +).

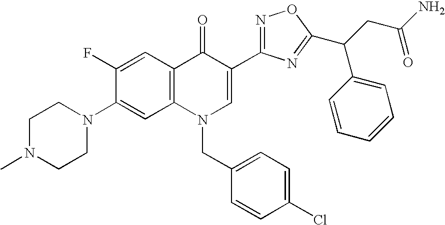

Proceeding as in Example 1, but substituting 1-(4-chloro-benzyl)-6-fluoro-7-(4-methyl-piperazin-1-yl)-4-oxo-1,4-dihydro-quinoline-3-carbonitrile and (pyrid-2-yl)acetic acid, gave 1-(4-chloro-benzyl)-6-fluoro-7-(4-methyl-piperazin-1-yl)-3-(5-pyridin-2-ylmethyl-[1,2,4]oxadiazol-3-yl)-1H-quinolin-4-one (Compound 61: +).

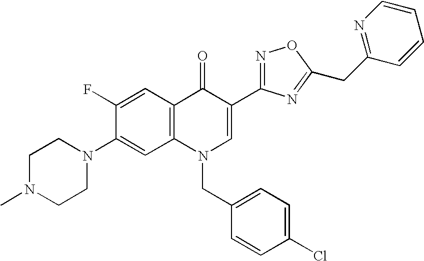

Proceeding as in Example 1, but substituting 1-(4-chloro-benzyl)-6-fluoro-7-(4-methyl-piperazin-1-yl)-4-oxo-1,4-dihydro-quinoline-3-carbonitrile and (pyrid-3-yl)acetic acid, gave 1-(4-chloro-benzyl)-6-fluoro-7-(4-methyl-piperazin-1-yl)-3-(5-pyridin-3-ylmethyl-[1,2,4]oxadiazol-3-yl)-1H-quinolin-4-one (Compound 62: +).

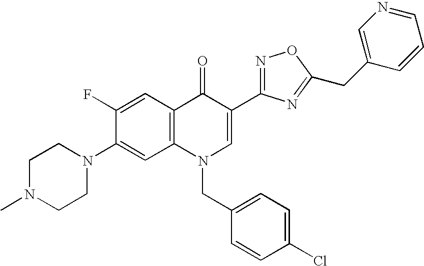

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 5-cyano-2-phenyl-pentanoic acid, gave 5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentanenitrile (Compound 63: +++).

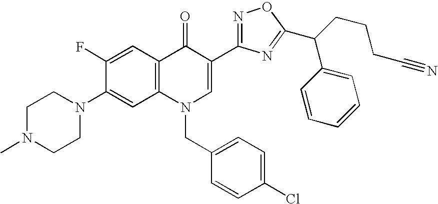

Proceeding as in Example 1, but substituting gave 1-(6-chloro-pyridin-3-ylmethyl)-6-fluoro-7-(4-methyl-piperazin-1-yl)-4-oxo-1,4-dihydro-quinoline-3-carbonitrile and (4-fluoro-phenyl)-acetic acid, gave 1-(6-chloro-pyridin-3-ylmethyl)-6-fluoro-3-[5-(4-fluoro-benzyl)-[1,2,4]oxadiazol-3-yl]-7-(4-methyl-piperazin-1-yl)-1H-quinolin-4-one (Compound 64: ++).

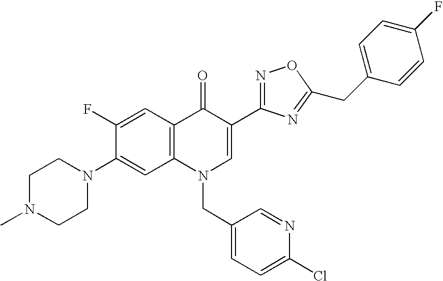

Proceeding as in Example 1, but substituting gave 1-(6-chloro-pyridin-3-ylmethyl)-6-fluoro-7-(4-methyl-piperazin-1-yl)-4-oxo-1,4-dihydro-quinoline-3-carbonitrile and (phenyl)-acetic acid, gave 3-(5-benzyl-[1,2,4]oxadiazol-3-yl)-1-(6-chloro-pyridin-3-ylmethyl)-6-fluoro-7-(4-methyl-piperazin-1-yl)-1H-quinolin-4-one (Compound 65: ++).

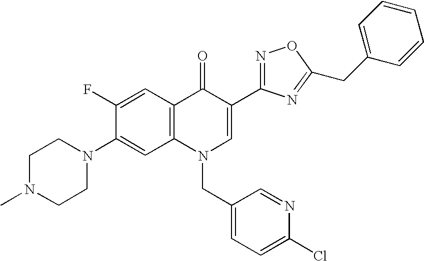

Proceeding as in Example 1, but substituting methyl[3-cyano-6-fluoro-1-(2-fluoro-4-trifluoromethyl-benzyl)-4-oxo-1,4-dihydro-quinolin-7-ylamino]-acetate, gave methyl[3-(5-benzyl-[1,2,4]oxadiazol-3-yl)-6-fluoro-1-(2-fluoro-4-trifluoromethyl-benzyl)-4-oxo-1,4-dihydro-quinolin-7-ylamino]-acetate (Compound 66: +).

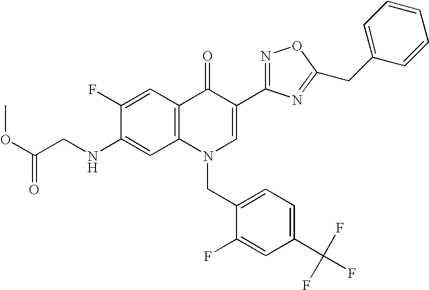

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 4-carbamoyl-2-phenyl-butyric acid, gave 4-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-4-phenyl-butyramide (Compound 67: ++).

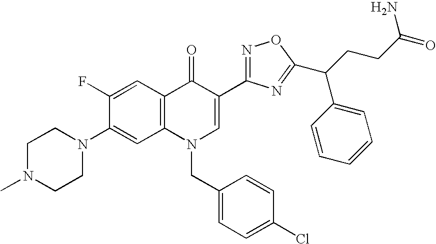

Proceeding as in Example 1, but substituting 7-methoxy-4-oxo-1-(4-trifluoromethyl-benzyl)-1,4-dihydro-quinoline-3-carbonitrile gave 3-(5-benzyl-oxadiazol-3-yl)-7-methoxy-1-(4-trifluoromethyl-benzyl)-1H-quinolin-4-one (Compound 68: +).

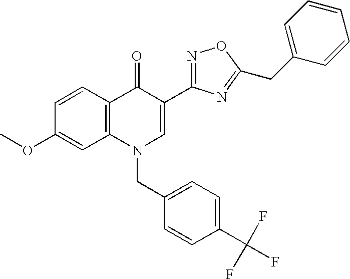

Proceeding as in Example 1, but substituting 2-[3-cyano-6-fluoro-1-(2-fluoro-4-trifluoromethyl-benzyl)-4-oxo-1,4-dihydro-quinolin-7-ylamino]-acetamide gave 2-[3-(5-benzyl-[1,2,4]oxadiazol-3-yl)-6-fluoro-1-(2-fluoro-4-trifluoromethyl-benzyl)-4-oxo-1,4-dihydro-quinolin-7-ylamino]-acetamide (Compound 69: ++).

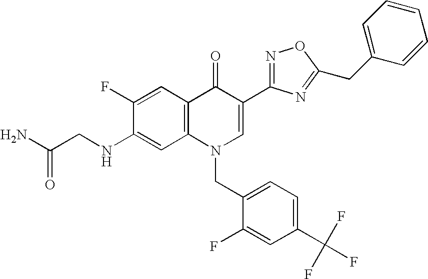

Proceeding as in Example 1, but substituting 6-fluoro-4-oxo-7-piperazin-1-yl-1-(4-trifluoromethyl-benzyl)-1,4-dihydro-quinoline-3-carbonitrile, gave 3-(5-benzyl-[1,2,4]oxadiazol-3-yl)-6-fluoro-7-piperazin-1-yl-1-(4-trifluoromethyl-benzyl)-1H-quinolin-4-one (Compound 70: ++++).

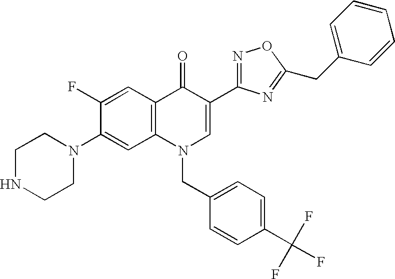

Proceeding as in Example 1, but substituting 7-[1,4]diazepam-1-yl-6-fluoro-4-oxo-1-(4-trifluoromethyl-benzyl)-1,4-dihydro-quinoline-3-carbonitrile gave 3-(5-benzyl-[1,2,4]oxadiazol-3-yl)-7-[1,4]diazepam-1-yl-6-fluoro-1-(4-trifluoromethyl-benzyl)-1H-quinolin-4-one (Compound 71: ++++).

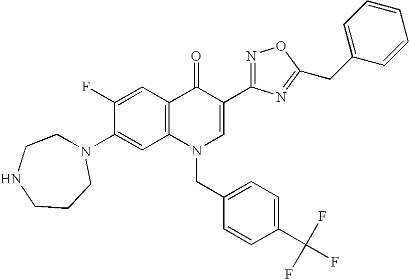

Proceeding as in Example 1, but substituting 6-fluoro-7-(2-morpholin-4-yl-ethylamino)-4-oxo-1-(4-trifluoromethyl-benzyl)-1,4-dihydro-quinoline-3-carbonitrile, gave 3-(5-benzyl-[1,2,4]oxadiazol-3-yl)-6-fluoro-7-(2-morpholin-4-yl-ethylamino)-1-(4-trifluoromethyl-benzyl)-1H-quinolin-4-one (Compound 72: +++).

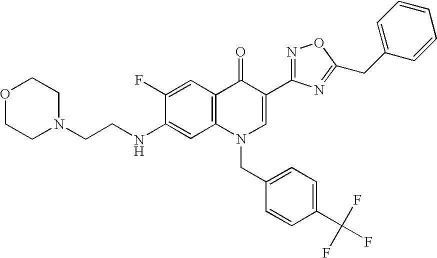

Proceeding as in Example 1, but substituting 6-fluoro-7-[2-(2-hydroxy-ethoxy)-ethylamino]-4-oxo-1-(4-trifluoromethyl-benzyl)-1,4-dihydro-quinoline-3-carbonitrile, gave 3-(5-benzyl-[1,2,4]oxadiazol-3-yl)-6-fluoro-7-[2-(2-hydroxy-ethoxy)-ethylamino]-1-(4-trifluoromethyl-benzyl)-1H-quinolin-4-one (Compound 73: ++).

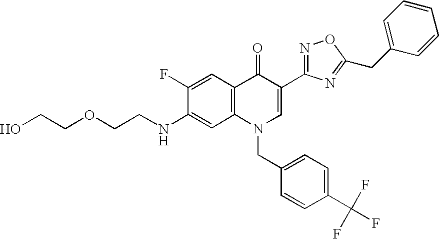

Proceeding as in Example 1, but substituting 7-dimethylamino-6-fluoro-4-oxo-1-(4-trifluoromethyl-benzyl)-1,4-dihydro-quinoline-3-carbonitrile, gave 3-(5-benzyl-[1,2,4]oxadiazol-3-yl)-7-dimethylamino-6-fluoro-1-(4-trifluoromethyl-benzyl)-1H-quinolin-4-one (Compound 74: +++).

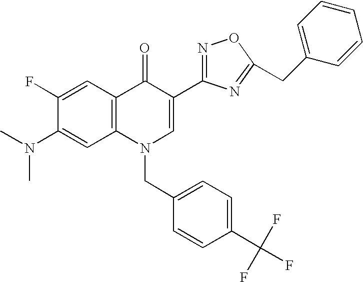

Proceeding as in Example 1, but substituting 7-(2-amino-ethylamino)-6-fluoro-4-oxo-1-(4-trifluoromethyl-benzyl)-1,4-dihydro-quinoline-3-carbonitrile, gave 7-(2-amino-ethylamino)-3-(5-benzyl-[1,2,4]oxadiazol-3-yl)-6-fluoro-1-(4-trifluoromethyl-benzyl)-1H-quinolin-4-one (Compound 75: +).

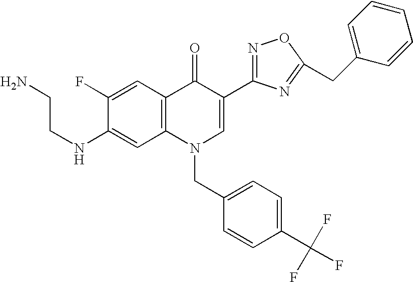

Proceeding as in Example 1, but substituting [3-cyano-1-(2-fluoro-4-trifluoromethyl-benzyl)-4-oxo-1,4-dihydro-quinolin-6-yl]-acetic acid, gave [3-(5-benzyl-[1,2,4]oxadiazol-3-yl)-1-(2-fluoro-4-trifluoromethyl-benzyl)-4-oxo-1,4-dihydro-quinolin-6-yl]-acetic acid (Compound 76). Mass Spec: 538.1, 560.2 (M+1, M+23). +.

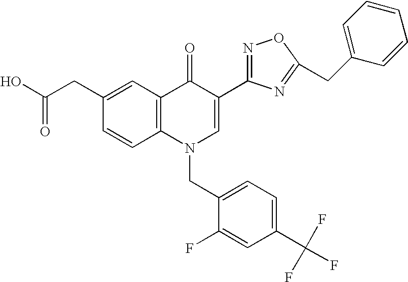

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and 2-methyl2-phenylpropionic acid, gave 6-fluoro-1-(2-fluoro-4-trifluoromethyl-benzyl)-3-[5-(1-methyl-1-phenyl-ethyl)-[1,2,4]oxadiazol-3-yl]-7-(4-methyl-piperazin-1-yl)-1H-quinolin-4-one (Compound 77: ++++).

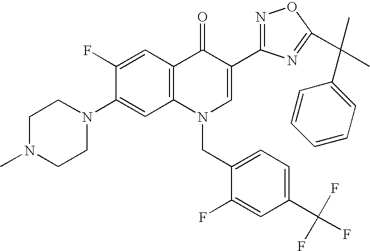

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and 1-phenyl-cyclopropanecarboxylic acid, gave 6-fluoro-1-(2-fluoro-4-trifluoromethyl-benzyl)-7-(4-methyl-piperazin-1-yl)-3-[5-(1-phenyl-cyclopropyl)-[1,2,4]oxadiazol-3-yl]-1H-quinolin-4-one (Compound 78: +++).

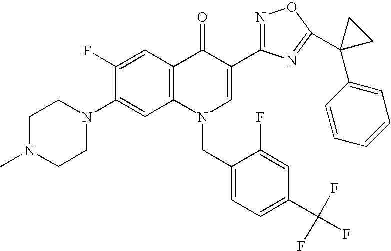

Proceeding as in Example 1, but substituting 6-fluoro-7-(4-methyl-piperazin-1-yl)-4-oxo-1-phenethyl-1,4-dihydro-quinoline-3-carbonitrile, gave 3-(5-benzyl-[1,2,4]oxadiazol-3-yl)-6-fluoro-7-(4-methyl-piperazin-1-yl)-1-phenethyl-1H-quinolin-4-one (Compound 79: +).

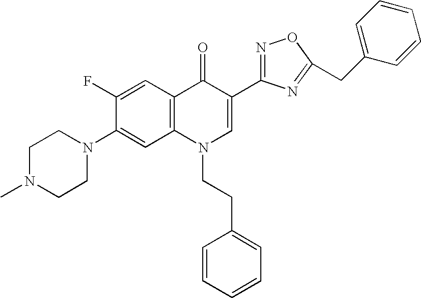

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 5-carbamoyl-2-phenyl-pentanoic acid, gave 5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentanamide (Compound 80: +++).

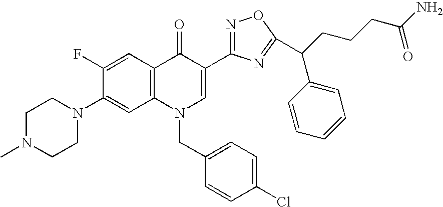

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 2-phenyl-hexanedioic acid, gave 5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentanoic acid (Compound 81: ++).

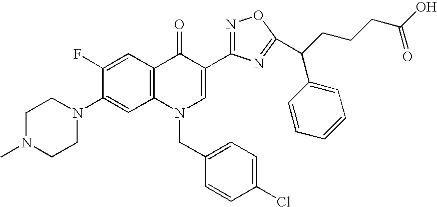

Proceeding as in Example 1, but substituting 1-(4-chloro-benzyl)-6-fluoro-7-(4-methyl-piperazin-1-yl)-4-oxo-1,4-dihydro-quinoline-3-carbonitrile and 6-amino-2-phenyl-hexanoic acid, gave 3-[5-(5-amino-1-phenyl-pentyl)-[1,2,4]oxadiazol-3-yl]-1-(4-chloro-benzyl)-6-fluoro-7-(4-methyl-piperazin-1-yl)-1H-quinolin-4-one (Compound 82: ++).

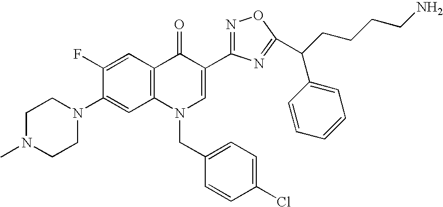

Proceeding as in Example 1, but substituting 7-[bis-(2-hydroxy-ethyl)-amino]-6-fluoro-4-oxo-1-(4-trifluoromethyl-benzyl)-1,4-dihydro-quinoline-3-carbonitrile, gave 3-(5-benzyl-[1,2,4]oxadiazol-3-yl)-7-[bis-(2-hydroxy-ethyl)-amino]-6-fluoro-1-(4-trifluoromethyl-benzyl)-1H-quinolin-4-one (Compound 83: ++).

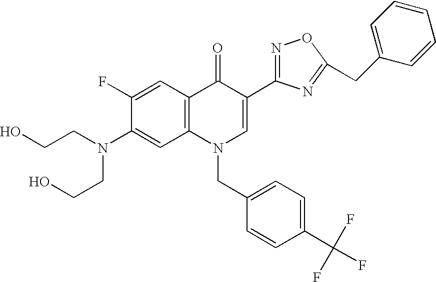

Proceeding as in Example 1, but substituting 6-fluoro-7-imidazol-1-yl-4-oxo-1-(4-trifluoromethyl-benzyl)-1,4-dihydro-quinoline-3-carbonitrile, gave 3-(5-benzyl-[1,2,4]oxadiazol-3-yl)-6-fluoro-7-imidazol-1-yl-1-(4-trifluoromethyl-benzyl)-1H-quinolin-4-one (Compound 84: +++).

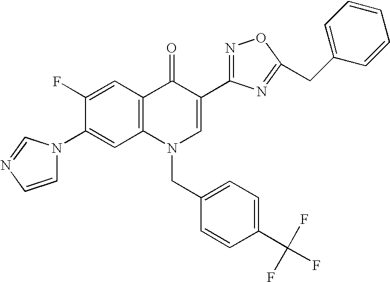

Proceeding as in Example 1, but substituting 3-[1-(4-chloro-benzyl)-6-fluoro-7-(4-methyl-piperazin-1-yl)-4-oxo-1,4-dihydro-quinolin-3-yl]-[1,2,4]oxadiazole-5-carbonitrile and 6-acetylamino-2-phenyl-hexanoic acid, gave N-(5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentyl)-acetamide (Compound 85: +++).

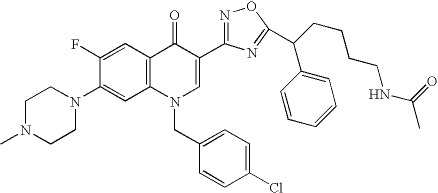

Proceeding as in Example 1, but substituting 3-[1-(4-chloro-benzyl)-6-fluoro-7-(4-methyl-piperazin-1-yl)-4-oxo-1,4-dihydro-quinolin-3-yl]-[1,2,4]oxadiazole-5-carbonitrile and 6-tert-butoxyaminocarbonyl-1-phenyl-hexanoic acid, gave tert-butyl (5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentyl)-carbamate (Compound 86: ++).

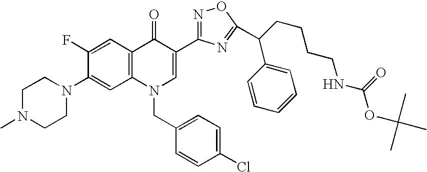

Proceeding as in Example 1, but substituting 6-fluoro-1-(2-fluoro-4-trifluoromethyl-benzyl)-4-oxo-7-(3-oxo-piperazin-1-yl)-1,4-dihydro-quinoline-3-carbonitrile and 4-fluorophenyl-acetic acid gave 6-fluoro-3-[5-(4-fluoro-benzyl)-[1,2,4]oxadiazol-3-yl]-1-(2-fluoro-4-trifluoromethyl-benzyl)-7-(3-oxo-piperazin-1-yl)-1H-quinolin-4-one (Compound 87: ++++).

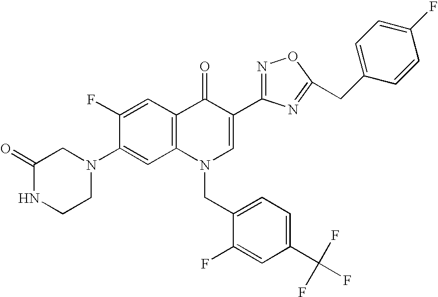

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and 5-cyano-2-phenyl-pentanoic acid, gave 5-{3-[6-fluoro-1-(2-fluoro-4-trifluoromethyl-benzyl)-7-(4-methyl-piperazin-1-yl)-4-oxo-1,4-dihydro-quinolin-3-yl]-[1,2,4]oxadiazol-5-yl}-5-phenyl-pentanenitrile (Compound 88a: ++++).

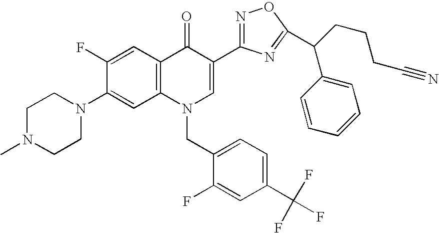

Proceeding as in Example 1, but substituting 6-fluoro-7-(4-methyl-piperazin-1-yl)-4-oxo-1-[1-(4-trifluoromethyl-phenyl)-cyclopropyl]-1,4-dihydro-quinoline-3-carbonitrile and 4-fluorophenyl-acetic acid, gave 6-fluoro-3-[5-(4-fluoro-benzyl)-[1,2,4]oxadiazol-3-yl]-7-(4-methyl-piperazin-1-yl)-1-[1-(4-trifluoromethyl-phenyl)-cyclopropyl]-1H-quinolin-4-one (Compound 88b: +++).

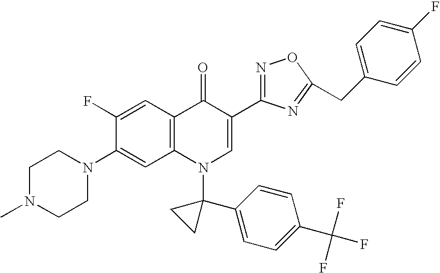

Proceeding as in Example 1, but substituting 6-fluoro-1-(2-fluoro-4-trifluoromethyl-benzyl)-7-(4-methyl-piperazin-1-yl)-4-oxo-1,4-dihydro-quinoline-3-carbonitrile and 5-carbamoyl-2-phenyl-pentanoic acid, gave 5-{3-[6-fluoro-1-(2-fluoro-4-trifluoromethyl-benzyl)-7-(4-methyl-piperazin-1-yl)-4-oxo-1,4-dihydro-quinolin-3-yl]-[1,2,4]oxadiazol-5-yl}-5-phenyl-pentanamide (Compound 89: ++++).

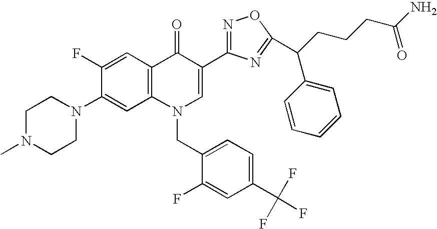

Proceeding as in Example 1, but substituting 6-fluoro-7-(4-methyl-piperazin-1-yl)-4-oxo-1-[1-(4-trifluoromethyl-phenyl)-cyclopropyl]-1,4-dihydro-quinoline-3-carbonitrile and 1-phenyl-cyclopropanecarboxylic acid, gave 6-fluoro-7-(4-methyl-piperazin-1-yl)-3-[5-(1-phenyl-cyclopropyl)-[1,2,4]oxadiazol-3-yl]-1-[1-(4-trifluoromethyl-phenyl)-cyclopropyl]-1H-quinolin-4-one (Compound 90: +).

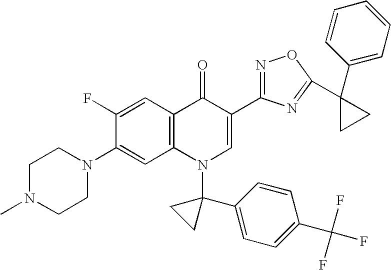

Proceeding as in Example 1, but substituting 6-fluoro-7-(4-methyl-piperazin-1-yl)-4-oxo-1-(3-trifluoromethyl-benzyl)-1,4-dihydro-quinoline-3-carbonitrile and 4-fluorophenyl-acetic acid gave 6-fluoro-3-[5-(4-fluoro-benzyl)-[1,2,4]oxadiazol-3-yl]-7-(4-methyl-piperazin-1-yl)-1-(3-trifluoromethyl-benzyl)-1H-quinolin-4-one (Compound 91: +++).

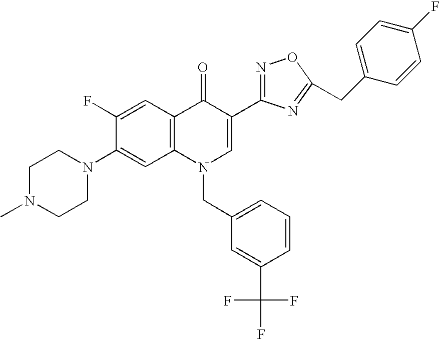

Proceeding as in Example 1, but substituting 1-(6-chloro-pyridin-3-ylmethyl)-6-fluoro-7-(4-methyl-piperazin-1-yl)-4-oxo-1,4-dihydro-quinoline-3-carbonitrile and 2-methyl-2-phenyl-propionic acid, gave 1-(6-chloro-pyridin-3-ylmethyl)-6-fluoro-3-[5-(1-methyl-1-phenyl-ethyl)-[1,2,4]oxadiazol-3-yl]-7-(4-methyl-piperazin-1-yl)-1H-quinolin-4-one (Compound 92: ++++).

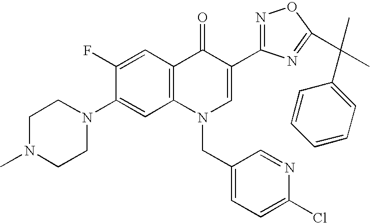

Proceeding as in Example 1, but substituting 7-[1,4]diazepan-1-yl-6-fluoro-4-oxo-1-(4-trifluoromethyl-benzyl)-1,4-dihydro-quinoline-3-carbonitrile and 2-methyl-2-phenyl-propionic acid, gave 7-[1,4]diazepan-1-yl-6-fluoro-3-[5-(1-methyl-1-phenyl-ethyl)-[1,2,4]oxadiazol-3-yl]-1-(4-trifluoromethyl-benzyl)-1H-quinolin-4-one (Compound 93).

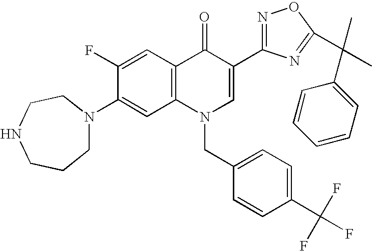

Proceeding as in Example 1, but substituting 1-(2-fluoro-4-trifluoromethyl-benzyl)-4-oxo-1,4-dihydro-quinoline-3-carbonitrile, gave 3-(5-benzyl-[1,2,4]oxadiazol-3-yl)-1-(2-fluoro-4-trifluoromethyl-benzyl)-1H-quinolin-4-one (Compound 94: +).

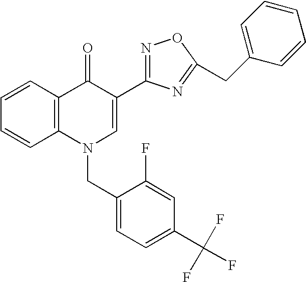

Proceeding as in Example 1, but substituting 1-(2-fluoro-4-trifluoromethyl-benzyl)-4-oxo-1,4-dihydro-quinoline-3-carbonitrile and 5-cyano-2-phenyl-pentanoic acid, gave 5-{3-[1-(2-fluoro-4-trifluoromethyl-benzyl)-4-oxo-1,4-dihydro-quinolin-3-yl]-[1,2,4]oxadiazol-5-yl}-5-phenyl-pentanenitrile (Compound 95: +).

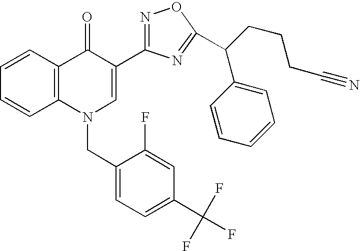

Proceeding as in Example 1, but substituting 6-fluoro-7-(4-methyl-piperazin-1-yl)-4-oxo-1-[1-(4-trifluoromethyl-phenyl)-cyclopropyl]-1,4-dihydro-quinoline-3-carbonitrile and 2-methyl-2-phenyl-propionic acid, gave 6-fluoro-3-[5-(1-methyl-1-phenyl-ethyl)-[1,2,4]oxadiazol-3-yl]-7-(4-methyl-piperazin-1-yl)-1-[1-(4-trifluoromethyl-phenyl)-cyclopropyl]-1H-quinolin-4-one (Compound 96: ++++).

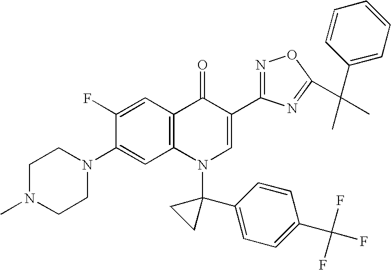

Proceeding as in Example 1, but substituting 2-{[3-cyano-6-fluoro-4-oxo-1-(4-trifluoromethyl-benzyl)-1,4-dihydro-quinolin-7-yl]-methyl-amino}-acetamide, gave 2-{[3-(5-benzyl-[1,2,4]oxadiazol-3-yl)-6-fluoro-4-oxo-1-(4-trifluoromethyl-benzyl)-1,4-dihydro-quinolin-7-yl]-methyl-amino}-acetamide (Compound 97: +).

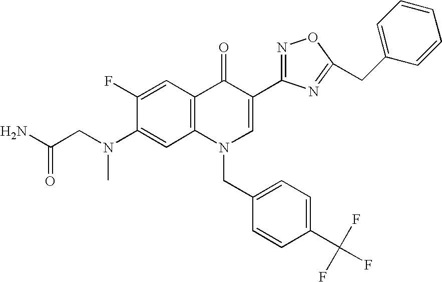

Proceeding as in Example 1, but substituting 6-fluoro-7-(4-methyl-piperazin-1-yl)-4-oxo-1-(3-trifluoromethyl-benzyl)-1,4-dihydro-quinoline-3-carbonitrile and 2-methyl-2-phenyl-propionic acid, gave 6-fluoro-3-[5-(1-methyl-1-phenyl-ethyl)-[1,2,4]oxadiazol-3-yl]-7-(4-methyl-piperazin-1-yl)-1-(3-trifluoromethyl-benzyl)-1H-quinolin-4-one (Compound 98: ++++).

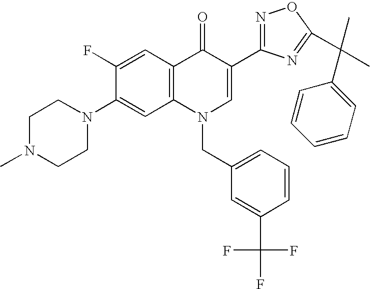

Proceeding as in Example 1, but substituting 1-(2-fluoro-4-trifluoromethyl-benzyl)-4-oxo-1,4-dihydro-quinoline-3-carbonitrile and 5-carbamoyl-2-phenyl-pentanoic acid, gave 5-{3-[1-(2-fluoro-4-trifluoromethyl-benzyl)-4-oxo-1,4-dihydro-quinolin-3-yl]-[1,2,4]oxadiazol-5-yl}-5-phenyl-pentanoic acid amide (Compound 99: +).

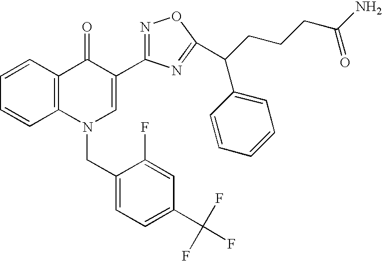

Proceeding as in Example 1, but substituting 7-(1,1-dioxo-thiomorpholin-4-yl)-6-fluoro-4-oxo-1-(4-trifluoromethyl-benzyl)-1,4-dihydro-quinoline-3-carbonitrile and 2-methyl-2-phenyl-propionic acid, gave 7-(1,1-dioxo-thiomorpholin-4-yl)-6-fluoro-3-[5-(1-methyl-1-phenyl-ethyl)-[1,2,4]oxadiazol-3-yl]-1-(4-trifluoromethyl-benzyl)-1H-quinolin-4-one (Compound 100: ++++). TLC system: 10% MeOH/CHCl₃ (Rf Value: 0.5); ¹H NMR (DMSO-d₆; 300 MHz): 8.88 (s, 1H); 7.87-7.72 (dd, 3H); 7.49-7.46 (d, J=9 Hz, 2H); 7.39-7.37 (t, 2H); 7.20-7.14 (t, 2H); 7.04-7.02 (d, J=6 Hz, 1H) 5.85 (s, 2H); 3.5 (m, 4H); 3.2 (m, 4H); IR vₘₐₓ (cm⁻¹): 2982, 2905, 1634, 1563, 1492, 1413, 1324, 1288, 1259, 1168, 1122, 1067. Mass: (M+H)⁺ 640

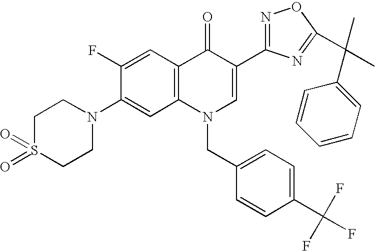

Proceeding as in Example 1, but substituting 6-fluoro-4-oxo-7-thiomorpholin-4-yl-1-(4-trifluoromethyl-benzyl)-1,4-dihydro-quinoline-3-carbonitrile and 2-methyl-2-phenyl-propionic acid, gave 6-fluoro-3-[5-(1-methyl-1-phenyl-ethyl)-[1,2,4]oxadiazol-3-yl]-7-thiomorpholin-4-yl-1-(4-trifluoromethyl-benzyl)-1H-quinolin-4-one (Compound 101: ++++).

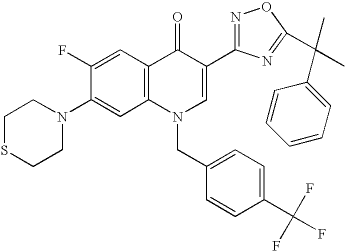

Proceeding as in Example 1, but substituting 1-(4-tert-butyl-benzyl)-7-[1,4]diazepan-1-yl-6-fluoro-4-oxo-1,4-dihydro-quinoline-3-carbonitrile, gave 3-(5-benzyl-[1,2,4]oxadiazol-3-yl)-1-(4-tert-butyl-benzyl)-7-[1,4]diazepan-1-yl-6-fluoro-1H-quinolin-4-one (Compound 102: +++).

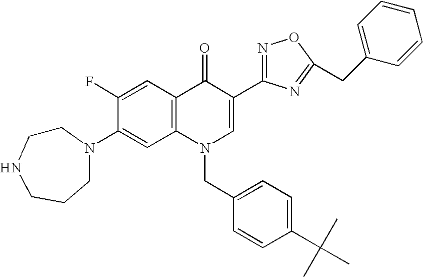

Proceeding as in Example 1, but substituting 6-fluoro-7-(4-methyl[1,4]diazepam-1-yl)-4-oxo-1-(4-trifluoro-methylbenzyl) 1,4-dihydro[1,8]naphthyridine-3-carbonitrile and 2-methyl-2-phenyl-propionic acid, gave 6-fluoro-7-(4-methyl-[1,4]diazepan-1-yl)-3-[5-(1-methyl-1-phenyl-ethyl)-[1,2,4]oxadiazol-3-yl]-1-(4-trifluoromethyl-benzyl)-1H-[1,8]naphthyridin-4-one (Compound 103: +++). LCMS (MH+)=622.2; (M+23)=643.4; actual 620.65. NMR (DMSO-d₆) δ 1.81 (s, 6H), 2.15 (m, 2H), 2.69 (s, 3H), 2.96 (m, 1H), 3.21 (m, 1H), 3.40 (m, 2H), 3.73 (m, 3H), 3.96 (m, 1H), 5.77 (q, J=9 Hz, 2H), 7.35 (m, 5H), 7.42 (d, J=7 Hz, 2H), 7.73 (d, J=7 Hz, 2H), 8.04 (d, J=13 Hz, 1H), 8.91 (s, 1H).

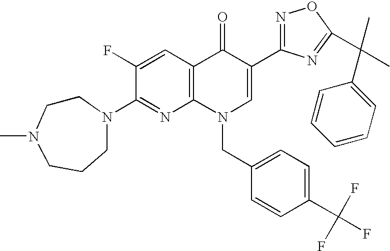

Proceeding as in Example 1, but substituting (Z)-1-(6-fluoro-1-(2-fluoro-4-(trifluoromethyl)benzyl)-3-(N′-hydroxycarbamimidoyl)-4-oxo-1,4-dihydroquinolin-7-yl)-1H-imidazole-4-carboxylic acid gave 1-(3-(5-benzyl-1,2,4-oxadiazol-3-yl)-6-fluoro-1-(2-fluoro-4-(trifluoromethyl)benzyl)-4-oxo-1,4-dihydroquinolin-7-yl)-1H-imidazole-4-carboxylic acid. (Compound 104: +).

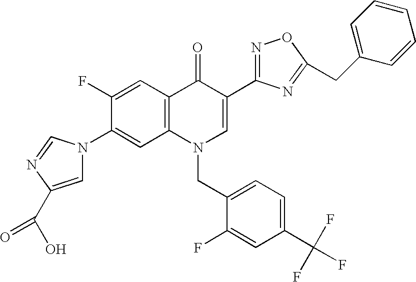

Proceeding as in Example 1, but substituting (Z)-2-(1-(2-fluoro-4-(trifluoromethyl)benzyl)-3-(N′-hydroxycarbamimidoyl)-4-oxo-1,4-dihydroquinolin-6-yl)acetamide gave 2-(3-(5-benzyl-1,2,4-oxadiazol-3-yl)-1-(2-fluoro-4-(trifluoromethyl)benzyl)-4-oxo-1,4-dihydroquinolin-6-yl)acetamide (Compound 105), Mass Spec: 537.3, 559.3 (M+1, M+23). +.

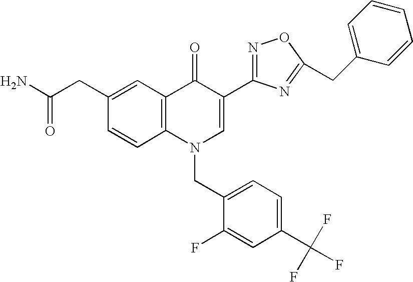

### Example 1b

### 6-fluoro-N-hydroxy-7-morpholino-4-oxo-1-(1-(4-(trifluoromethyl)phenyl)ethyl)-1,4-dihydroquinoline-3-carboximidamide

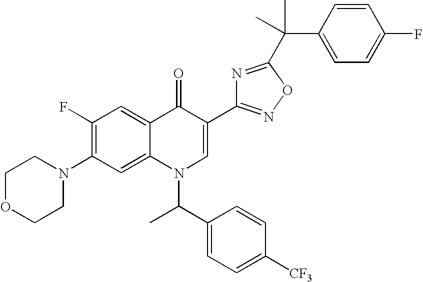

To a stirred solution of 6-fluoro-7-morpholino-4-oxo-1-(1-(4-(trifluoromethyl)phenyl)ethyl)-1,4-dihydroquinoline-3-carbonitrile, compound 1b.1 (1.6 g, 3.6 mmol) in MeOH (50 mL) was treated with 50% hydroxylamine solution (1 mL, 18.3 mmol). The reaction mixture was stirred at reflux for 4 h. Once the reaction was completed then solvent was removed under reduced pressure to give solid, diluted with water, filtered, washed with ethyl ether and dried with high vacuum at 60° C. 1 h to provided 6-fluoro-N-hydroxy-7-morpholino-4-oxo-1-(1-(4-(trifluoromethyl)phenyl)ethyl)-1,4-dihydroquinoline-3-carboximidamide compound 1b.2 (1.7 g, 97%). ¹H-NMR (DMSO-d₆) δ 9.31 (1H, s, —NOH), 8.48 (1H, s, Ar—H), 7.93-7.56 (5H, m, Ar—H), 7.10-7.03 (2H, m, Ar—H, NH), 3.88 (4H, m, OCH₂), 3.31-2.99 (4H, m, NCH₂), 1.96 (3H, d, —CH₃). EIMS (m/z): 479.4 (M+H)⁺.

To a stirred solution of 4-Fluoro-αα-dimethylphenyl acetic acid 1b.3 (150 mg, 0.96 mmol) in DMF (5 mL) was treated with HATU (441 mg, 1.1 mmol) and DIPEA (625 mg, 4.8 mmol). The reaction mixture stirred for 1 h under nitrogen atmosphere at room temperature, then 6-fluoro-N-hydroxy-7-morpholino-4-oxo-1-(1-(4-(trifluoromethyl)phenyl)ethyl)-1,4-dihydroquinoline-3-carboximidamide, compound 1b.2 (460 mg, 0.96 mmol) was added and left the reaction over night. The reaction mixture portioned between water and ethyl acetate. The organic layer was washed with brine solution, dried over MgSO₄, filtered and concentrated. The crude residue was purified by silica gel flash column chromatography eluting with 30% ethyl acetate in n-hexane to provide 6-fluoro-N-(2-(4-fluorophenyl)-2-methylpropanoyloxy)-7-morpholino-4-oxo-1-(1-(4-(trifluoromethyl)phenyl)ethyl)-1,4-dihydroquinoline-3-carboximidamide, compound 1b.4 (289 mg, 46.8%). ¹H-NMR (DMSO-d₆) δ 8.64 (1H, s, Ar—H), 7.83-7.42 (5H, m, Ar—H), 7.22-6.98 (2H, m, Ar—H), 7.2 (2H, t, Ar—H), 7.0 (1H, d, Ar—H), 6.4 (1H, q, —CH), 3.98 (4H, s, OCH₂), 2.7-3.2-2.98 (4H, m, NCH₂), 1.92 (3H, d, CH₃), 1.64 (6H, s, CH₃CCH₃). EIMS (m/z): 643.7 (M+H)⁺.

The uncyclized intermediate 6-fluoro-N-(2-(4-fluorophenyl)-2-methylpropanoyloxy)-7-morpholino-4-oxo-1-(1-(4-(trifluoromethyl)phenyl)ethyl)-1,4-dihydroquinoline-3-carboximidamide, 1b.4 (289 mg) was dissolved in toluene (30 mL) under nitrogen atmosphere and heated at reflux for overnight. Once the reaction was completed then solvent was removed under reduced pressure. The residue was purified by flash column chromatography eluting with 30% ethyl acetate in n-hexane to provide 6-fluoro-3-(5-(2-(4-fluorophenyl)propan-2-yl)-1,2,4-oxadiazol-3-yl)-7-morpholino-1-(1-(4-(trifluoromethyl)phenyl)ethyl)quinolin-4(1H)-one, compound 1b.5 (Compound 109: +++). (170 mg, 60.7%). ¹H-NMR (DMSO-d₆) δ 8.72 (1H, s, N—CH), 7.83 (1H, d, Ar—H), 7.78-7.62 (4H, ABq, Ar—H), 7.39 (2H, m, Ar—H), 7.20 (2H, m, Ar—H), 7.03 (1H, m, —CH), 6.41 (1H, q, —CH), 3.76 (4H, m, OCH₂), 3.07-2.98 (4H, m, NCH₂), 1.99 (3H, d, CH₃), 1.82 (6H, s, CH₃CCH₃). EIMS (m/z): 625.6 (M+H)⁺.

Proceeding as in Example 1b but substituting 7-(dimethylamino)-6-fluoro-1-(2-methyl-2-(4-(trifluoromethyl)phenyl)propyl)-4-oxo-1,4-dihydroquinoline-3-carbonitrile and 2-methyl-2-phenylpropanoic acid gave 7-(dimethylamino)-6-fluoro-1-(2-methyl-2-(4-(trifluoromethyl)phenyl)propyl)-3-(5-(2-phenylpropan-2-yl)-1,2,4-oxadiazol-3-yl)quinolin-4(1H)-one (Compound 110).

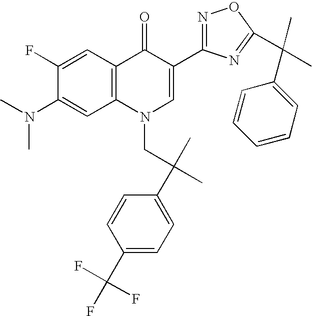

Proceeding as in Example 1b but substituting 6-fluoro-7-morpholino-4-oxo-1-(2-(4-(trifluoromethyl)phenyl)propan-2-yl)-1,4-dihydroquinoline-3-carbonitrile and 2-methyl-2-phenylpropanoic acid gave 6-fluoro-7-morpholino-3-(5-(2-phenylpropan-2-yl)-1,2,4-oxadiazol-3-yl)-1-(2-(4-(trifluoromethyl)phenyl)propan-2-yl)quinolin-4(1H)-one (Compound 111). TLC system: 50% ethyl acetate/pet-ether; (Rf value: 0.2); ¹H NMR (DMSO-d₆; 300 MHz): 9.09 (s, 1H); 7.87-7.82 (m, 3H); 7.79-7.76 (d, J=9 Hz, 2H); 7.62-7.59 (m, 5H); 6.28-6.26 (d, J=6 Hz, 2H); 3.58 (s, 4H); 2.68 (s, 4H); 2.27 (s, 6H); 1.98 (s, 6H); IR v_{Max} (cm⁻¹): 3343, 3064, 2980, 1624, 1484, 1409, 1329, 1258, 1171, 1118, 1067; Mass: (M+H)⁺ 621; Purity by HPLC: 97.9%; Pale brown solid; 250 mg; (49.6%)

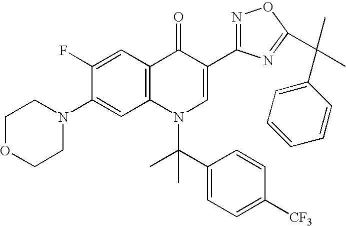

Proceeding as in Example 1b but substituting gave 6-fluoro-7-morpholino-4-oxo-1-(2-(4-(trifluoromethyl)phenyl)propan-2-yl)-1,4-dihydroquinoline-3-carbonitrile and 2-(4-fluorophenyl)-2-methylpropanoic acid gave 6-fluoro-3-(5-(2-(4-fluorophenyl)propan-2-yl)-1,2,4-oxadiazol-3-yl)-7-morpholino-1-(2-(4-(trifluoromethyl)phenyl)propan-2-yl)quinolin-4(1H)-one. (Compound 112). ¹H-NMR (DMSO-d₆) δ 9.08 (1H, s, N—CH), 7.92 (1H, d, Ar—H), 7.88 (2H, d, Ar—H), 7.60 (2H, d, Ar—H), 7.42 (2H, m, Ar—H), 7.22 (2H, m, Ar—H), 6.25 (1H, q, Ar—H), 3.60 (4H, m, OCH₂), 2.64 (4H, m, NCH₂), 2.16 (6H, s, CH₃CCH₃), 1.94 (6H, s, CH₃CCH₃). EIMS (m/z): 639.0 (M+H)+

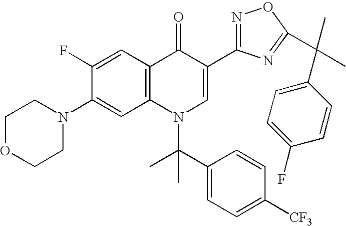

Proceeding as in Example 1b but substituting 5-(4-fluorophenyl)-2,2-dimethyl-1,3-dioxane-5-carboxylic acid and 6-fluoro-7-morpholino-4-oxo-1-(4-(trifluoromethyl)benzyl)-1,4-dihydroquinoline-3-carbonitrile gave 6-fluoro-3-(5-(5-(4-fluorophenyl)-2,2-dimethyl-1,3-dioxan-5-yl)-1,2,4-oxadiazol-3-yl)-7-morpholino-1-(4-(trifluoromethyl)benzyl)quinolin-4(1H)-one. Synthesis of 5-(4-fluorophenyl)-2,2-dimethyl-1,3-dioxane-5-carboxylic acid is described: Para-Formaldehyde (1.785 gm; 59.52 mmoles) was added to a solution of Compound (1 (11.0 gm; 5.952 mmoles) in DMSO (15 mL) at room temperature and the reaction mixture was heated to 120° C. for 5 h. The reaction mixture was diluted with water and extracted into ethyl acetate. The organic layer was separated and washed with water, brine and dried.

The crude compound 2-(4-fluorophenyl)-3-hydroxy-2-(hydroxymethyl)propanoic methylester was purified by silica gel column chromatography eluting with 20% EtOAc in pet ether. TLC system: 40% Ethyl acetate/Pet Ether; (R_{f} value: 0.3); ¹H NMR (DMSO-d₆; 300 MHz): 7.2-7.1 (m, 4H); 4.75 (d, J=6 Hz, 2H); 3.93-3.97 (m, 4H); 3.58 (s, 3H); Mass: (M+H)⁺ 229; Pale yellow solid; 215 mg (16%). PTSA (catalytic amount) was added to a solution of compound 2-(4-fluorophenyl)-3-hydroxy-2-(hydroxymethyl)propanoic methylester (215 mg; 0.942 mmoles) in acetone (5 mL) at room temperature and the reaction mixture was stirred at room temperature for 24 h. Acetone was evaporated from the reaction mixture and water was added to it. Extracted with ethyl acetate and washed with brine, dried over anhydrous Na₂SO₄, filtered and evaporated at reduced pressure to get the crude compound. The crude compound was purified by Preparative TLC using 30% ethyl acetate/pet-ether as eluents to give methyl 5-(4-fluorophenyl)-2,2-dimethyl-1,3-dioxane-5-carboxylate.TLC system: 30% Ethyl acetate/Pet-ether; (R_{f} value: 0.4); ¹H NMR (DMSO-d₆; 300 MHz): 7.26-7.29 (m, 2H); 7.03 (t, J=7.5 Hz, 2H); 4.56 (d, J=12 Hz, 2H); 4.13 (d, J=12 Hz, 2H); 3.74 (s, 3H); 1.4 (s, 6H). Mass: (M+H)⁺ 269; Pale yellow solid; 80 mg (31%). LiOH (25.04 mg; 0.597 mmoles) was added to a solution of compound methyl 5-(4-fluorophenyl)-2,2-dimethyl-1,3-dioxane-5-carboxylate (80 mg; 0.298 mmoles) in ethanol (8 mL) at 0° C. and the reaction mixture was stirred at room temperature for 6 h. Ethanol was evaporated from the reaction mixture. Water was added to it and cooled to 0° C.; pH was adjusted to 6 with acetic acid and extracted with ethyl acetate. The ethyl acetate layer was washed with water, brine, dried over anhydrous Na₂SO₄, filtered and evaporated at reduced pressure to get the crude compound. No further purification has been done, compound as such taken for the next step. 5-(4-fluorophenyl)-2,2-dimethyl-1,3-dioxane-5-carboxylic acid TLC system: 10% MeOH/CHCl₃; (R_{f} value: 0.2); ¹H NMR (DMSO-d₆; 300 MHz): 7.31-7.33 (m, 2H); 7.02-7.08 (m, 2H); 4.62 (d, J=12 Hz, 2H); 4.09 (d, J=12 Hz, 2H); 1.4 (s, 6H); Mass: (M+H)⁺ 255. White solid; 58 mg (76.5%). Treatment of 6-fluoro-3-(5-(5-(4-fluorophenyl)-2,2-dimethyl-1,3-dioxan-5-yl)-1,2,4-oxadiazol-3-yl)-7-morpholino-1-(4-(trifluoromethyl)benzyl)quinolin-4(1H)-one with acid afforded 6-fluoro-3-(5-(2-(4-fluorophenyl)-1,3-dihydroxypropan-2-yl)-1,2,4-oxadiazol-3-yl)-7-morpholino-1-(4-(trifluoromethyl)benzyl)quinolin-4(1H)-one. (Compound 113).

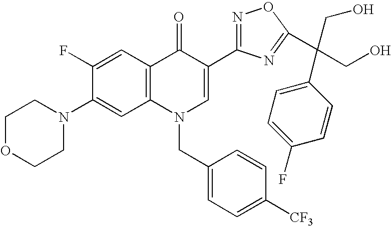

Proceeding as in Example 1b but substituting 7-(dimethylamino)-6-fluoro-4-oxo-1-(1-(4-(trifluoromethyl)phenyl)ethyl)-1,4-dihydroquinoline-3-carbonitrile and 2-methyl-2-phenylpropanoic acid gave 7-(dimethylamino)-6-fluoro-3-(5-(2-phenylpropan-2-yl)-1,2,4-oxadiazol-3-yl)-1-(1-(4-(trifluoromethyl)phenyl)ethyl)quinolin-4(1H)-one. (Compound 114).

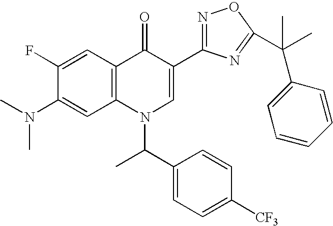

Proceeding as in Example 1b but substituting 6-fluoro-7-morpholino-4-oxo-1-(4-(trifluoromethyl)benzyl)-1,4-dihydroquinoline-3-carbonitrile and 2-oxo-2-phenylacetyl chloride gave 3-(5-benzoyl-1,2,4-oxadiazol-3-yl)-6-fluoro-7-morpholino-1-(4-(trifluoromethyl)benzyl)quinolin-4(1H)-one. (Compound 115).

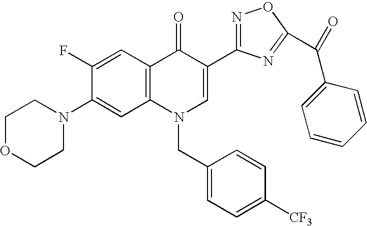

Proceeding as in Example 1b but substituting (R)-6-fluoro-7-morpholino-4-oxo-1-(1-(4-(trifluoromethyl)phenyl)ethyl)-1,4-dihydroquinoline-3-carbonitrile and 2-methyl-2-phenylpropanoic acid gave (R)-6-fluoro-7-morpholino-3-(3-(2-phenylpropan-2-yl)-1,2,4-oxadiazol-5-yl)-1-(1-(4-(trifluoromethyl)phenyl)ethyl)quinolin-4(1H)-one (Compound 116) after separation from its enantiomer by HPLC with a chiral stationary phase, Chiral pack AD-H 250 mm×4.6 mm and a mobile phase of Isopropanol:Hexane (20:80). ¹H-NMR (DMSO-d₆) δ 8.71 (1H, s, N—CH), 7.93 (1H, d, Ar—H), 7.78 (2H, d, Ar—H), 7.62 (2H, d, Ar—H), 7.40-7.24 (5H, m, PhH), 7.04 (1H, bs, Ar—H), 6.40 (1H, q, —CH), 3.76 (4H, m, OCH₂), 3.18-3.0 (4H, m, NCH₂), 1.99 (3H, d, CH₃), 1.80 (6H, s, CH₃CCH₃). EIMS (m/z): 607.1 (M+H).

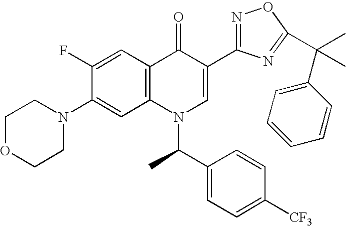

Proceeding as in Example 1b but substituting (S)-6-fluoro-7-morpholino-4-oxo-1-(1-(4-(trifluoromethyl)phenyl)ethyl)-1,4-dihydroquinoline-3-carbonitrile and 2-methyl-2-phenylpropanoic acid gave (s)-6-fluoro-7-morpholino-3-(3-(2-phenylpropan-2-yl)-1,2,4-oxadiazol-5-yl)-1-(1-(4-(trifluoromethyl)phenyl)ethyl)quinolin-4(1H)-one (Compound 117) (after separation from its enantiomer by HPLC with a chiral stationary phase, Chiral pack AD-H 250 mm×4.6 mm and a mobile phase of Isopropanol:Hexane (20:80). ¹H-NMR (DMSO-d₆) δ 8.71 (1H, s, N—CH), 7.93 (1H, d, Ar—H), 7.78 (2H, d, Ar—H), 7.62 (2H, d, Ar—H), 7.40-7.24 (5H, m, PhH), 7.04 (1H, bs, Ar—H), 6.40 (1H, q, —CH), 3.76 (4H, m, OCH₂), 3.18-3.0 (4H, m, NCH₂), 1.99 (3H, d, CH₃), 1.80 (6H, s, CH₃CCH₃). EIMS (m/z): 607.1 (M+H).

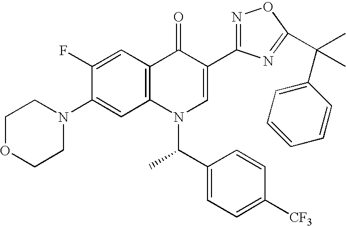

Proceeding as in Example 1b but substituting (R,S)-6-fluoro-7-morpholino-4-oxo-1-(1-(4-(trifluoromethyl)phenyl)ethyl)-1,4-dihydroquinoline-3-carbonitrile and 2-methyl-2-phenylpropanoic acid gave (R,S)-6-fluoro-7-morpholino-3-(3-(2-phenylpropan-2-yl)-1,2,4-oxadiazol-5-yl)-1-(1-(4-(trifluoromethyl)phenyl)ethyl)quinolin-4(1H)-one. (Compound 118) ¹H-NMR (DMSO-d₆) δ 8.71 (1H, s, N—CH), 7.93 (1H, d, Ar—H), 7.78 (2H, d, Ar—H), 7.62 (2H, d, Ar—H), 7.40-7.24 (5H, m, PhH), 7.04 (1H, bs, Ar—H), 6.40 (1H, q, —CH), 3.76 (4H, m, OCH₂), 3.18-3.0 (4H, m, NCH₂), 1.99 (3H, d, CH₃), 1.80 (6H, s, CH₃CCH₃) EIMS (m/z): 607.1 (M+H).

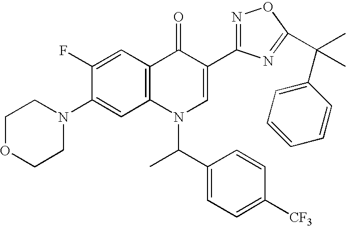

Proceeding as in Example 1b but substituting (S)-6-fluoro-7-morpholino-4-oxo-1-(1-(4-(trifluoromethyl)phenyl)ethyl)-1,4-dihydroquinoline-3-carbonitrile and 2-(4-fluorophenyl)-2-methylpropanoic acid gave (S)-6-fluoro-3-(3-(2-(4-fluorophenyl)propan-2-yl)-1,2,4-oxadiazol-5-yl)-7-morpholino-1-(1-(4-(trifluoromethyl)phenyl)ethyl)quinolin-4(1H)-one (Compound 119) after separation from its enantiomer by HPLC with a chiral stationary phase, Chiral pack AD-H 250 mm×4.6 mm and a mobile phase of isopropanol:hexane (20:80). ¹H-NMR (CD₃OD) δ 8.99 (1H, s, N—CH), 8.02 (1H, d, Ar—H), 7.78 (2H, d, Ar—H), 7.58 (2H, d, Ar—H), 7.40 (2H, m, Ar—H), 7.08 (2H, m, Ar—H), 7.01 (1H, d, Ar—H), 6.28 (1H, q, NCHPh), 3.82 (4H, m, OCH₂), 3.22-3.04 (4H, m, NCH₂), 2.08 (3H, d, CH₃), 1.88 (6H, s, CH₃CCH₃), EIMS (m/z): 625.0 (M+H)+

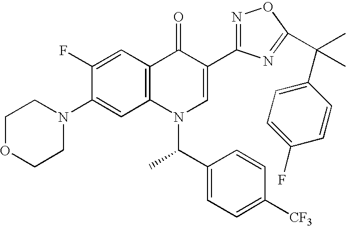

Proceeding as in Example 1b but substituting (R)-6-fluoro-7-morpholino-4-oxo-1-(1-(4-(trifluoromethyl)phenyl)ethyl)-1,4-dihydroquinoline-3-carbonitrile and 2-(4-fluorophenyl)-2-methylpropanoic acid gave (R)-6-fluoro-3-(3-(2-(4-fluorophenyl)propan-2-yl)-1,2,4-oxadiazol-5-yl)-7-morpholino-1-(1-(4-(trifluoromethyl)phenyl)ethyl)quinolin-4(1H)-one (Compound 120) after separation from its enantiomer by HPLC with a chiral stationary phase, Chiral pack AD-H 250 mm×4.6 mm and a mobile phase of Isopropanol:Hexane (20:80). (2H, d, Ar—H), 7.40 (2H, m, Ar—H), 7.08 (2H, m, Ar—H), 7.01 (1H, d, Ar—H), 6.28 (1H, q, NCHPh), 3.82 (4H, m, OCH₂), 3.22-3.04 (4H, m, NCH₂), 2.08 (3H, d, CH₃), 1.88 (6H, s, CH₃CCH₃), EIMS (m/z): 625.0 (M+H)

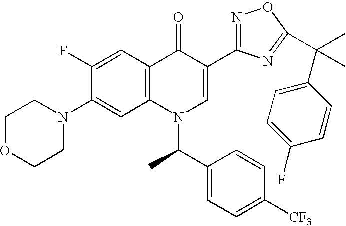

Proceeding as in Example 1b but substituting 1-(cyclopropylmethyl)-6,7-difluoro-4-oxo-1,4-dihydroquinoline-3-carbonitrile and 2-(4-fluorophenyl)-2-methylpropanoic acid gave 1-(cyclopropylmethyl)-6,7-difluoro-3-(3-(2-(4-fluorophenyl)propan-2-yl)-1,2,4-oxadiazol-5-yl)quinolin-4(1H)-one. (Compound 121). TLC system: 5% MeOH/DCM; (Rf value: 0.6); ¹H NMR (DMSO-d₆; 300 MHz): 8.75 (s, 1H); 8.23 (m, 2H); 8.14 (m, 2H); 7.37 (m, 2H); 7.19 (t, 2H); 4.31-4.29 (d, 2H, J=6 Hz); 1.83 (s, 6H); 0.55-0.6 (m, 5H); IR V_{Max} (cm⁻¹): 3447, 1649, 1622, 1568, 1497, 1407, 1321, 1288, 1232, 1176, 1036. Mass: (M+H)⁺ 439; Purity by HPLC: 98%; pale yellow solid; 50 mg; (13.34%).

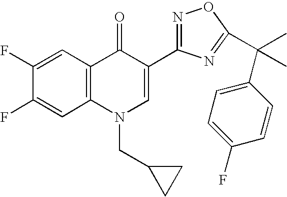

Proceeding as in Example 1b but substituting 6-fluoro-1-(4-fluorobenzyl)-7-morpholino-4-oxo-1,4-dihydroquinoline-3-carbonitrile and 2-(4-fluorophenyl)-2-methylpropanoic acid gave 6-fluoro-1-(4-fluorobenzyl)-3-(3-(2-(4-fluorophenyl)propan-2-yl)-1,2,4-oxadiazol-5-yl)-7-morpholinoquinolin-4(1H)-one. (Compound 122).

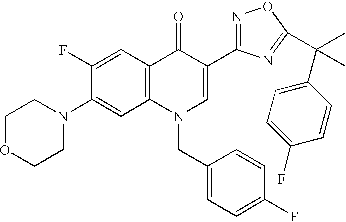

Proceeding as in Example 1b but substituting 1-(cyclopropylmethyl)-7-(dioxo-thiomorpholino)-6-fluoro-4-oxo-1,4-dihydroquinoline-3-carbonitrile and 2-(4-fluorophenyl)-2-methylpropanoic acid gave 1-(cyclopropylmethyl)-6-fluoro-3-(3-(2-(4-fluorophenyl)propan-2-yl)-1,2,4-oxadiazol-5-yl)-7-dioxo-thiomorpholinoquinolin-4(1H)-one. (Compound 123) TLC system: 10% MeOH/CHCl₃; (R_{f} value: 0.5); ¹H NMR (DMSO-d₆; 300 MHz): 8.88 (s, 1H); 7.9 (dd, 3H); 7.4 (q, 2H); 7.3 (t, 3H); 5.83 (s, 1H); 4.3 (d, 2H); 3.8 (s, 4H); 3.4 (s, 4H); 1.87 (s, 6H). IR (KBr, vₘₐₓ): 3450, 2985, 1627, 1595, 1493, 1392, 1317, 1236, 1182, 1124, 1038 cm⁻¹; Mass: (M+1)⁺ 555; Purity by HPLC: 95%; Pale yellow solid; 98 mg (35%).

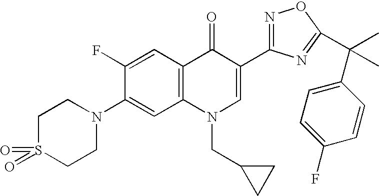

Proceeding as in Example 1b, but substituting 7-(1,1-dioxo-thiomorpholin-4-yl)-6-fluoro-4-oxo-1-(4-trifluoromethyl-benzyl)-1,4-dihydro-quinoline-3-carbonitrile and 2-(4-fluorophenyl)-2-methylpropanoic acid gave 3-(5-(2-(4-fluorophenyl)propan-2-yl)-1,2,4-oxadiazol-3-yl)-7-dioxo-thiomorpholino-1-(4-(trifluoromethyl)benzyl)quinolin-4(1H)-one. (Compound 124) TLC system: 10% MeOH/CHCl₃; (R_{f} value: 0.5). ¹H NMR (DMSO-d₆; 300 MHz): 8.9 (s, 1H); 7.89-7.85 (d J=9, 3H); 7.77-7.74 (d, J=9 Hz 2H); 7.40-7.37 (d, J=9 Hz, 2H); 7.35-7.32 (dd, 2H); 7.20-7.17 (t, 2H); 7.06-7.04 (d, J=6 Hz); 5.87 (s, 2H); 3.59 (m, 4H); 3.22 (m, 4H); 1.83 (s, 6H). IR v_{Max} (cm⁻¹): 3445, 2982, 1630, 1599, 1563, 1492, 1414, 1388, 1325, 1288, 1169, 1124, 1067. White Solid; 19 mg; (7.39%); Mass: (M+H)⁺ 658 ¹H NMR (DMSO-d₆; 300 MHz)

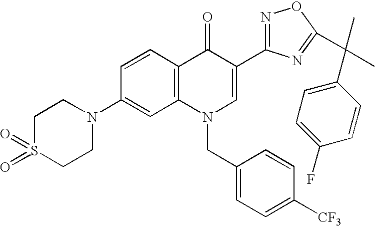

Proceeding as in Example 1b, but substituting 6-fluoro-4-oxo-7-(3-oxopiperazin-1-yl)-1-(4-(trifluoromethyl)benzyl)-1,4-dihydroquinoline-3-carbonitrile and 2-(4-fluorophenyl)-2-methylpropanoic acid gave 6-fluoro-3-(5-(2-(4-fluorophenyl)propan-2-yl)-1,2,4-oxadiazol-3-yl)-7-(3-oxopiperazin-1-yl)-1-(4-(trifluoromethyl)benzyl)quinolin-4(1H)-one. (Compound 125: ++++). TLC system: 5% MeOH/DCM; (R_{f} value: 0.3); ¹H NMR (DMSO-d₆; 300 MHz): 8.87 (s, 1H); 8.06 (s, 1H); 7.88-7.85 (d, J=3 Hz, 1H); 7.84-7.82 (d, J=6 Hz, 2H); 7.57-7.55 (d, J=6 Hz, 2H); 7.43 (m, 4H); 6.87-6.85 (d, J=6 Hz, 1H); 5.83 (s, 2H); 3.78 (s, 2H); 3.4 (s, 2H); 3.31 (s, 2H); 1.8 (s, 6H); IR (KBr, vₘₐₓ): 3322, 2930, 1670, 1628, 1493, 1459, 1414, 1380, 1326, 1260, 1167, 1123, 1067 cm⁻¹; Mass: (M+H)⁺ 624; Purity by HPLC: 97.5%; Pale brown solid; 25 mg; (5.4%).

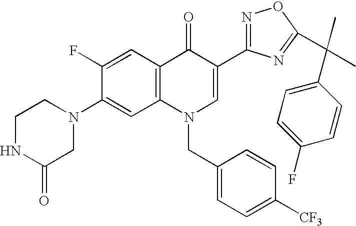

Proceeding as in Example 1b, but substituting 6-fluoro-4-oxo-7-(3-oxopiperazin-1-yl)-1-(4-(trifluoromethyl)benzyl)-1,4-dihydroquinoline-3-carbonitrile and 2-methyl-2-phenyl-propionic acid gave 6-fluoro-7-(3-oxopiperazin-1-yl)-3-(5-(2-phenylpropan-2-yl)-1,2,4-oxadiazol-3-yl)-1-(4-(trifluoromethyl)benzyl)quinolin-4(1H)-one. (Compound 126: ++++). TLC system: 5% MeOH/DCM; (R_{f} value: 0.3); ¹H NMR (DMSO-d₆; 300 MHz): 8.87 (s, 1H); 8.06 (s, 1H); 7.88-7.85 (d, J=3 Hz, 1H); 7.84-7.82 (d, J=6 Hz, 2H); 7.57-7.55 (d, J=6 Hz, 2H); 7.43 (m, 5H); 6.87-6.85 (d, J=6 Hz, 1H); 5.83 (s, 2H); 3.78 (s, 2H); 3.4 (s, 2H); 3.31 (s, 2H); 1.8 (s, 6H); IR (KBr, vₘₐₓ): 3214, 2980, 1675, 1628, 1491, 1418, 1376, 1325, 1259, 1168, 1122, 1066, 1014 cm⁻¹; Mass: (M+H)⁺ 606; Purity by HPLC: 90.3%; White solid; 22 mg; (5%)

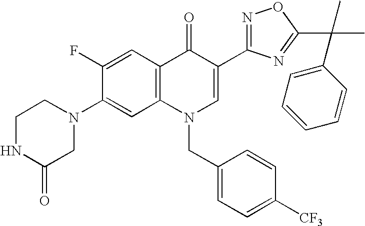

Proceeding as in Example 1b, but substituting 7-(1,1-dioxo-thiomorpholin-4-yl)-6-fluoro-4-oxo-1-(4-trifluoromethyl-benzyl)-1,4-dihydro-quinoline-3-carbonitrile and 2-(4-chlorophenyl)-2-methylpropanoic acid, gave 3-(5-(2-(4-chlorophenyl)propan-2-yl)-1,2,4-oxadiazol-3-yl)-6-fluoro-7-dioxo-thiomorpholino-1-(4-(trifluoromethyl)benzyl)quinolin-4(1H)-one. (Compound 127: ++).

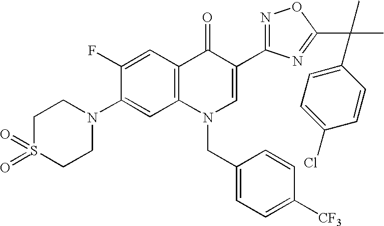

Proceeding as in Example 1b but substituting 6-fluoro-7-morpholino-4-oxo-1-(4-(trifluoromethyl)benzyl)-1,4-dihydroquinoline-3-carbonitrile 2-(4-fluorophenyl)-2-methylpropanoic acid gave 3-(5-(2-(4-fluorophenyl)propan-2-yl)-1,2,4-oxadiazol-3-yl)-7-morpholino-1-(4-(trifluoromethyl)benzyl)quinolin-4(1H)-one. (Compound 128: ++++). TLC system: 10% MeOH/CHCl₃; (Rf Value: 0.6) ¹H NMR (DMSO-d₆; 300 MHz): 8.88 (s, 1H); 8.10-8.07 (d, J=9 Hz); 7.75-7.72 (d, J=9 Hz, 2H); 7.49-7.46 (d, J=9 Hz, 2H); 7.39 (t, 2H); 7.11 (m, 3H); 6.7 (s, 1H); 5.79 (s, 2H); 3.7 (s, 4H); 3.2 (s, 4H); 1.8 (s, 6H). IR vₘₐₓ (cm⁻¹): 3049, 2971, 1625, 1590, 1510, 1476, 1325, 1240, 1166, 1120, 1064. Mass: (M+H)⁺ 593. Purity by HPLC: 97.5%. White Solid; 75 mg; (22.6%).

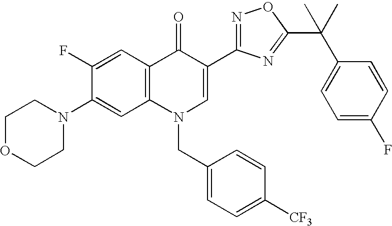

Proceeding as in Example 1b, but substituting 7-(1-oxo-thiomorpholin-4-yl)-6-fluoro-4-oxo-1-(4-trifluoromethyl-benzyl)-1,4-dihydro-quinoline-3-carbonitrile and 2-methylpropanoic acid, gave 6-fluoro-3-(5-(2-phenylpropan-2-yl)-1,2,4-oxadiazol-3-yl)-7-(1-oxo-thiomorpholino-1-(4-(trifluoromethyl)benzyl)quinolin-4(1H)-one. (Compound 129: ++++). TLC system: 10% MeOH/CHCl₃; (R_{f} value: 0.4 and 0.5); ¹H NMR (DMSO-d₆; 300 MHz): 8.92 (s, 1H); 7.83-7.88 (d, J=9 Hz, 2H); 7.74-7.76 (d, J=6 Hz, 1H); 7.49-7.52 (d, J=9 Hz, 2H); 7.27-7.35 (m, 5H); 7.04-7.06 (d, J=6 Hz, 1H); 5.88 (s, 2H); 3.65 (t, 2H); 3.3 (s, 2H); 2.82-2.89 (m, 4H); 1.83 (s, 6H); IR (KBr, vₘₐₓ): 3409, 2984, 1674, 1628, 1493, 1463, 1418, 1380, 1326, 1261, 1167, 1123, 1067 cm⁻¹; Mass: (M+H)⁺ 626; Purity by HPLC: 98.1%; Pale yellow solid; 52 mg; (33%)

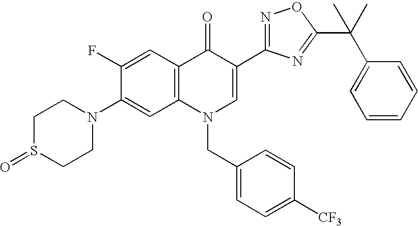

Proceeding as in Example 1b, but substituting 6-fluoro-1-(4-methoxybenzyl)-7-morpholino-4-oxo-1,4-dihydroquinoline-3-carbonitrile and 2-(4-fluorophenyl)-2-methylpropanoic acid gave 6-fluoro-3-(5-(2-(4-fluorophenyl)propan-2-yl)-1,2,4-oxadiazol-3-yl)-1-(4-methoxybenzyl)-7-morpholinoquinolin-4(1H)-one. (Compound 130: +++). ¹H-NMR (DMSO-d₆) δ 8.83 (1H, s, N—CH), 7.82 (1H, d, Ar—H), 7.40 (2H, m, Ar—H), 7.26 (2H, d, Ar—H), 7.21 (2H, m, Ar—H), 7.06 (1H, d, Ar—H), 6.96 (2H, d, Ar—H), 5.62 (2H, s, NCH₂Ph), 3.77-3.70 (7H, m, OCH₂OMe), 3.08 (4H, m, NCH₂), 1.84 (6H, s, CH₃CCH₃), EIMS (m/z): 573.1 (M+H)

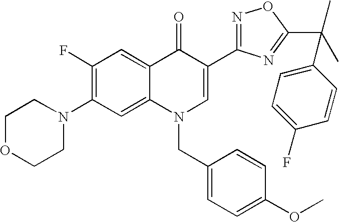

Proceeding as in Example 1b, but substituting 7-((3S,5R)-3,5-dimethylpiperazin-1-yl)-6-fluoro-4-oxo-1-(4-(trifluoromethyl)benzyl)-1,4-dihydroquinoline-3-carbonitrile and 2-methylpropanoic acid gave 7-((3S,5R)-3,5-dimethylpiperazin-1-yl)-6-fluoro-3-(5-(2-phenylpropan-2-yl)-1,2,4-oxadiazol-3-yl)-1-(4-(trifluoromethyl)benzyl)quinolin-4(1H)-one. (Compound 131: ++++). TLC system: 10% MeOH/CHCl₃; (R_{f} value: 0.5); ¹H NMR (DMSO-d₆; 300 MHz): 8.88 (s, 1H); 7.73-7.79 (m, 3H); 7.35-7.73 (d, J=6 Hz, 2H); 7.26-7.30 (m, 5H); 6.72-6.74 (d, J=6 Hz, 1H); 5.82 (s, 2H); 3.19-3.15 (d, J=12 Hz, 2H); 2.76 (m, 2H); 2.18-2.26 (t, 2H); 1.88 (s, 6H); 0.94-0.92 (d, J=6 Hz, 6H); IR (KBr, vₘₐₓ): 2970, 2827, 1633, 1591, 1491, 1455, 1414, 1326, 1263, 1167, 1115, 1066, 1016 cm⁻¹; Mass: (M+H)⁺ 620; Purity by HPLC: 95.7%; Pale brown solid; 70 mg (31.8%).

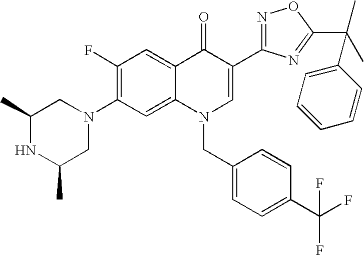

Proceeding as in Example 1b but substituting 6-fluoro-7-thiomorpholino-4-oxo-1-(4-(trifluoromethyl)benzyl)-1,4-dihydroquinoline-3-carbonitrile 2-(4-fluorophenyl)-2-methylpropanoic acid gave 6-fluoro-3-(5-(2-(4-fluorophenyl)propan-2-yl)-1,2,4-oxadiazol-3-yl)-7-thiomorpholino-1-(4-(trifluoromethyl)benzyl)quinolin-4(1H)-one. (Compound 132: ++++). TLC system: 5% MeOH/DCM; (R_{f} value: 0.5); ¹H NMR (DMSO-d₆; 300 MHz): 8.87 (s, 1H); 7.7-7.8 (m, 3H); 7.36-7.48 (m, 4H); 7.14-7.19 (m, 2H); 6.87-6.89 (d, J=6 Hz, 1H); 5.83 (s, 2H); 3.28 (s, 4H); 2.63 (s, 4H); 1.8 (s, 6H). IR (KBr, vₘₐₓ): 2966, 2827, 1635, 1563, 1491, 1455, 1413, 1326, 1253, 1167, 1116, 1067, 1016 cm⁻¹; Mass: (M+H)⁺ 627; Purity by HPLC: 96.1%; White solid; 63 mg; (9.6%)

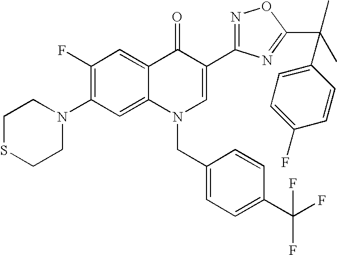

Proceeding as in Example 1b, but substituting 7-((3S,5R)-3,5-dimethylpiperazin-1-yl)-6-fluoro-4-oxo-1-(4-(trifluoromethyl)benzyl)-1,4-dihydroquinoline-3-carbonitrile and 2-(4-fluorophenyl)-2-methylpropanoic acid gave 7-((3S,5R)-3,5-dimethylpiperazin-1-yl)-6-fluoro-3-(5-(2-(4-fluorophenyl)propan-2-yl)-1,2,4-oxadiazol-3-yl)-1-(4-(trifluoromethyl)benzyl)quinolin-4(1H)-one. (Compound 133: ++++). TLC system: 10% MeOH/CHCl₃; (R_{f} value: 0.5); ¹H NMR (DMSO-d₆; 300 MHz): 8.9 (s, 1H); 7.7-7.8 (m, 3H); 7.4-7.5 (m, 4H); 7.2-7.3 (m, 2H); 6.78 (s, 1H); 5.86 (s, 2H); 3.25 (m, 2H); 2.83 (brs, 2H); 2.29 (brs, 2H); 1.8 (s, 6H); 0.99 (s, 6H); IR (KBr, vₘₐₓ): 3462, 2971, 1633, 1595, 1492, 1455, 1414, 1326, 1263, 1169, 1117, 1065, 1015 cm⁻¹; Mass: (M+H)⁺ 638; Purity by HPLC: 95.1%; Pale brown solid; 22 mg; (5.6%)

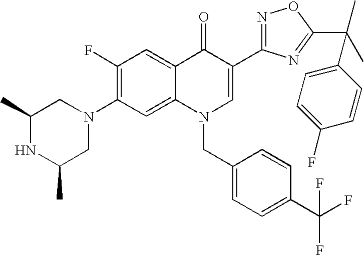

Proceeding as in Example 1b but substituting 6-fluoro-7-thiomorpholino-4-oxo-1-(4-(trifluoromethyl)benzyl)-1,4-dihydroquinoline-3-carbonitrile and 2-methylpropanoic acid gave 6-fluoro-3-(5-(2-phenylpropan-2-yl)-1,2,4-oxadiazol-3-yl)-7-thiomorpholino-1-(4-(trifluoromethyl)benzyl)quinolin-4(1H)-one. (Compound 134: ++++). TLC system: 5% MeOH/DCM; (R_{f} value: 0.5); ¹H NMR (DMSO-d₆; 300 MHz): 8.89 (s, 1H); 7.72-7.79 (m, 3H); 7.47-7.49 (d, J=6 Hz, 2H); 7.26-7.38 (m, 5H); 6.9 (d, J=9 Hz, 1H); 5.84 (s, 2H); 3.29 (m, 4H); 2.65 (m, 4H); 1.81 (s, 6H); IR (KBr, vₘₐₓ): 3445, 2938, 1635, 1563, 1491, 1451, 1413, 1325, 1256, 1166, 1116, 1066, 1017 cm⁻¹; Mass: (M+H)⁺ 609; Purity by HPLC: 96.9%; 53 mg; Off-white solid; (8.7%).

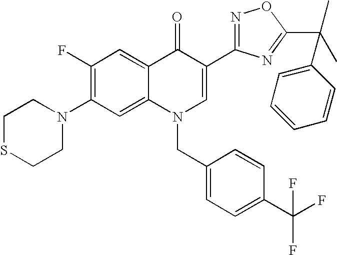

Proceeding as in Example 1b but substituting 1-(4-fluorobenzyl)-4-oxo-1,4-dihydroquinoline-3-carbonitrile and 2-(4-fluorophenyl)-2-methylpropanoic acid gave 3-(5-(2-(4-chlorophenyl)propan-2-yl)-1,2,4-oxadiazol-3-yl)-1-(4-fluorobenzyl)quinolin-4(1H)-one. (Compound 135) TLC system: 10% MeOH/CHCl₃; (R_{f} value: 0.5); ¹H NMR (DMSO-d₆; 300 MHz): 8.94 (s, 1H); 8.298-8.296 (d, J=9 Hz, 1H); 7.69 (m, 2H); 7.65 (m, 5H); 7.56 (t, 2H); 7.45 (t, 2H); 5.72 (s, 2H); 1.808 (s, 6H); IR (KBr, vₘₐₓ): 3443, 3060, 2986, 1636, 1604, 1563, 1490, 1425, 1391, 1321, 1225, 1164, 1102, 1010 cm⁻¹. Mass: (M+H)⁺ 474; Purity by HPLC: 97.7%; Off white solid; 47 mg (17.2%).

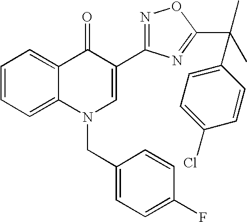

Proceeding as in Example 1b but substituting 4-oxo-1-(4-(trifluoromethyl)benzyl)-1,4-dihydroquinoline-3-carbonitrile and 2-(4-chlorophenyl)-2-methylpropanoic acid gave 3-(5-(2-(4-chlorophenyl)propan-2-yl)-1,2,4-oxadiazol-3-yl)-1-(4-(trifluoromethyl)benzyl)quinolin-4(1H)-one. (Compound 136). TLC system: 10% MeOH/CHCl₃; (R_{f} value: 0.5); ¹H NMR (DMSO-d₆; 300 MHz): 8.94 (s, 1H); 8.29-8.26 (d, J=9 Hz, 1H); 7.69 (m, 2H); 7.65 (m, 5H); 7.56 (t, 2H); 7.45 (t, 2H); 5.89 (s, 2H); 1.89 (s, 6H); IR (KBr, vₘₐₓ): 2982, 2946, 1636, 1563, 1489, 1422, 1393, 1324, 1233, 1166, 1122, 1066, 1013 cm⁻¹. Mass: (M+H)⁺ 524; Purity by HPLC: 96.8%; white solid; 53 mg (26.2%).

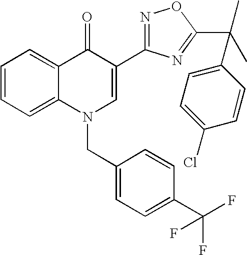

Proceeding as in Example 1b but substituting 7-(2,6-dimethylmorpholino)-6-fluoro-4-oxo-1-(4-(trifluoromethyl)benzyl)-1,4-dihydroquinoline-3-carbonitrile and 2-(4-fluorophenyl)-2-methylpropanoic acid gave 7-(2,6-dimethylmorpholino)-6-fluoro-3-(5-(2-(4-fluorophenyl)propan-2-yl)-1,2,4-oxadiazol-3-yl)-1-(4-(trifluoromethyl)benzyl)quinolin-4(1H)-one. (Compound 137) TLC system: 5% MeOH/DCM; (Rf value: 0.5); ¹H NMR (DMSO-d₆; 300 MHz): 8.8 (s, 1H); 7.77-7.72 (m, 3H); 7.50-7.45 (m, 4H); 7.20-7.17 (t, 2H); 6.80-6.78 (d, J=6 Hz, 1H); 5.83 (s, 2H); 3.59 (t, 2H); 3.28-3.24 (d, J=9 Hz, 2H); 2.37 (t, 2H); 1.80 (s, 6H); 1.07-1.05 (d, J=6 Hz, 6H). IR (KBr, vₘₐₓ): 2977, 2927, 1635, 1599, 1559, 1492, 1452, 1412, 1379, 1326, 1261, 1169, 1119, and 1069. Mass: (M+H)⁺ 639; Purity by HPLC: 96.7%; Off-white solid; 54 mg (16.6%).

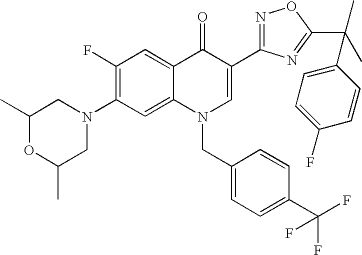

Proceeding as in Example 1b but substituting 7-(2,6-dimethylmorpholino)-6-fluoro-4-oxo-1-(4-(trifluoromethyl)benzyl)-1,4-dihydroquinoline-3-carbonitrile and 2-methylpropanoic acid gave 7-(2,6-dimethylmorpholino)-6-fluoro-3-(5-(2-phenylpropan-2-yl)-1,2,4-oxadiazol-3-yl)-1-(4-(trifluoromethyl)benzyl)quinolin-4(1H)-one. (Compound 138). TLC system: 5% MeOH/DCM; (R_{f} value: 0.5); ¹H NMR (DMSO-d₆; 300 MHz): 8.9 (s, +++0.1H); 7.74-7.80 (m, 3H); 7.51 (d, J=6 Hz, 2H); 7.32-7.38 (m, 5H); 6.83 (d, J=9 Hz, 1H); 5.85 (s, 2H); 3.62 (t, J=6 Hz, 2H); 3.27 (d, J=9 Hz, 2H); 2.40 (t, J=10.5 Hz, 2H); 1.83 (s, 6H); 1.08 (d, J=6 Hz, 6H); IR (KBr, vₘₐₓ (cm⁻¹): 2977, 2928, 1635, 1599, 1563, 1491, 1452, 1412, 1379, 1327, 1262, 1169, 1118 and 1069. Mass: (M+H)⁺ 621; Purity by HPLC: 99.0%; Pale brown solid; (10.6%).

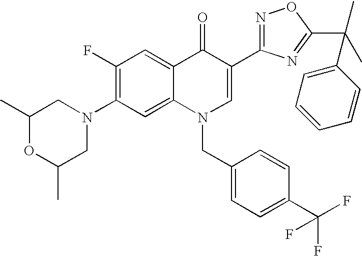

Proceeding as in Example 1b, but substituting 7-(1,1-dioxo-thiomorpholin-4-yl)-6-fluoro-4-oxo-1-(4-trifluoromethyl-benzyl)-1,4-dihydro-quinoline-3-carbonitrile and 2-(4-fluorophenyl)-2-methylpropanoic acid, gave 7-(1,1-dioxo-thiomorpholino)-6-fluoro-3-(5-(2-phenylpropan-2-yl)-1,2,4-oxadiazol-3-yl)-1-(4-(trifluoromethyl)benzyl)quinolin-4(1H)-one. (Compound 139: ++++). TLC system: 10% MeOH/CHCl₃ (Rf Value: 0.5); ¹H NMR (DMSO-d₆; 300 MHz): 8.88 (s, 1H); 8.16-8.14 (d, J=6 Hz, 1H); 7.87-7.85 (d, J=6 Hz, 2H); 7.49-7.46 (m, 4H); 7.39-7.37 (t, 3H); 6.87-6.85 (s, 1H); 5.85 (s, 2H); 3.8 (s, 4H); 2.97 (s, 4H); IR vₘₐₓ (cm⁻¹): 2978, 1625, 1591, 1510, 1475, 1416, 1380, 1324, 1278, 1238, 1168, 1123, 1065 cm⁻¹. Mass: (M+H)⁺ 641; 0.049 g; (34.5%).

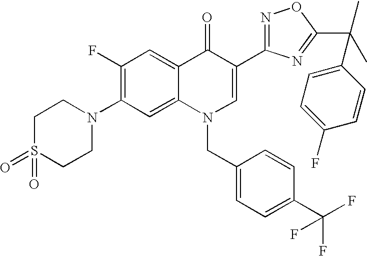

Proceeding as in Example 1b, but substituting 7-(1-oxo-thiomorpholin-4-yl)-6-fluoro-4-oxo-1-(4-trifluoromethyl-benzyl)-1,4-dihydro-quinoline-3-carbonitrile and 2-(4-fluorophenyl)-2-methylpropanoic acid, gave 7-(1-oxo-thiomorpholino)-6-fluoro-3-(5-(2-phenylpropan-2-yl)-1,2,4-oxadiazol-3-yl)-1-(4-(trifluoromethyl)benzyl)quinolin-4(1H)-one. (Compound 140: ++++). TLC system: 10% MeOH/CHCl₃; (R_{f} value: 0.6) 8.88 (s, 1H); 7.74-7.86 (dd, 3H); 7.49-7.45 (m, 4H); 7.38-7.35 (t, 2H); 7.17-7.14 (d, J=6 Hz, 1H); 5.85 (s, 2H); 3.6 (m, 2H); 3.4 (m, 2H); 2.94-2.91 (m, 4H); 1.80 (s, 6H); IR vₘₐₓ (cm⁻¹): Not recorded. Mass: (M+H)⁺ 642; White Solid; 10 mg; (4%).

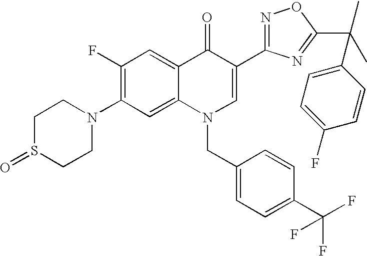

Proceeding as in Example 1b but substituting 1-(4-chlorobenzyl)-4-oxo-1,4-dihydroquinoline-3-carbonitrile and 2-(4-chlorophenyl)-2-methylpropanoic acid gave 1-(4-chlorobenzyl)-3-(5-(2-(4-chlorophenyl)propan-2-yl)-1,2,4-oxadiazol-3-yl)quinolin-4(1H)-one. (Compound 141). TLC system: 10% MeOH/CHCl₃; (R_{f} value: 0.5); ¹H NMR (DMSO-d₆; 300 MHz): 8.94 (s, 1H); 8.24-8.22 (d, J=6 Hz, 1H); 7.73 (m, 2H); 7.5-7.34 (m, 8H); 7.26-7.24 (d, J=6 Hz, 1H); 5.83 (s, 2H); 1.82 (s, 6H); IR (KBr, vₘₐₓ): 2974, 1693, 1642, 1609, 1563, 1509, 1486, 1420, 1363, 1234, 1167, 1098 cm⁻¹. Mass: (M+H)⁺ 490; Purity by HPLC: 95.3%; Pale brown solid; 57 mg (17.3%).

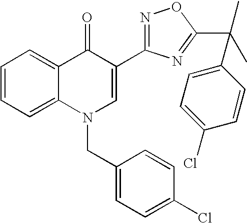

Proceeding as in Example 1b but substituting 6-fluoro-7-morpholino-4-oxo-1-(2-phenylpropan-2-yl)-1,4-dihydroquinoline-3-carbonitrile and 2-(4-fluorophenyl)acetic acid gave 6-fluoro-3-(5-(4-fluorobenzyl)-1,2,4-oxadiazol-3-yl)-7-morpholino-1-(2-phenylpropan-2-yl)quinolin-4(1H)-one. (Compound 142) ¹H-NMR (DMSO-d₆) δ 9.03 (1H, s, N—CH), 7.82 (1H, d, Ar—H), 7.48-7.18 (9H, m, Ar—H), 6.48 (1H, d, Ar—H), 4.43 (2H, s, CH₂Ph), 3.60 (4H, m, OCH₂), 2.64 (4H, m, NCH₂), 2.08 (6H, s, CH₃CCH₃). EIMS (m/z): 543.1 (M+H)

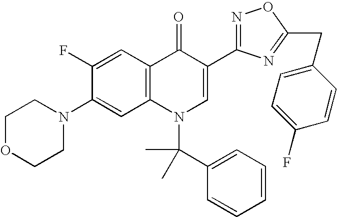

Proceeding as in Example 1b but substituting (S)-6-fluoro-7-morpholino-4-oxo-1-(1-phenylethyl)-1,4-dihydroquinoline-3-carbonitrile and 2-methyl-2-phenylpropanoic acid gave (S)-6-fluoro-7-morpholino-1-(1-phenylethyl)-3-(5-(2-phenylpropan-2-yl)-1,2,4-oxadiazol-3-yl)quinolin-4(1H)-one after separation from its enantiomer by HPLC with a chiral stationary phase, Chiral pack AD-H 250 mm×4.6 mm and a mobile phase of Isopropanol:Hexane (20:80). (Compound 143: +) ¹H-NMR (DMSO-d₆) δ 8.7 (1H, s, N—CH), 7.93 (1H, d, Ar—H), 7.48-7.32 (10H, m, 2×PhH), 7.12 (1H, bs, Ar—H), 6.23 (1H, q, —CH), 3.73 (4H, m, OCH₂), 3.14-3.0 (4H, m, NCH₂), 1.97 (3H, d, CH₃), 1.80 (6H, s, CH₃CCH₃). EIMS (m/z): 539.1 (M+H)+

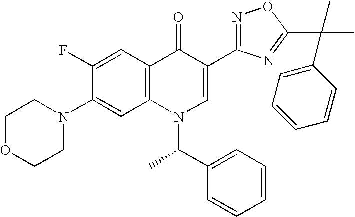

Proceeding as in Example 1b but substituting 6-fluoro-7-morpholino-4-oxo-1-(2-phenylpropan-2-yl)-1,4-dihydroquinoline-3-carbonitrile and 2-methyl-2-phenylpropanoic acid gave 6-fluoro-7-morpholino-1-(2-phenylpropan-2-yl)-3-(5-(2-phenylpropan-2-yl)-1,2,4-oxadiazol-3-yl)quinolin-4(1H)-one. (Compound 144: +) ¹H-NMR (DMSO-d₆) δ 9.08 (1H, s, N—CH), 7.83 (1H, d, Ar—H), 7.42-7.24 (10H, m, 2×PhH), 6.47 (1H, d, Ar—H), 3.60 (4H, m, OCH₂), 2.64 (4H, m, NCH₂), 2.08 (6H, s, CH₃CCH₃), 1.83 (6H, s, CH₃CCH₃) EIMS (m/z): 553.0 (M+H)

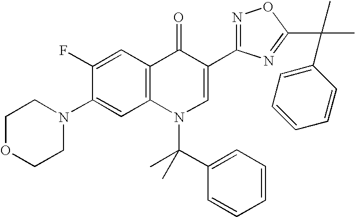

Proceeding as in Example 1b but substituting (R)-6-fluoro-7-morpholino-4-oxo-1-(1-phenylethyl)-1,4-dihydroquinoline-3-carbonitrile and 2-methyl-2-phenylpropanoic acid gave (R)-6-fluoro-7-morpholino-1-(1-phenylethyl)-3-(5-(2-phenylpropan-2-yl)-1,2,4-oxadiazol-3-yl)quinolin-4(1H)-one after separation from its enantiomer by HPLC with a chiral stationary phase, Chiral pack AD-H 250 mm×4.6 mm and a mobile phase of Isopropanol:Hexane (20:80). (Compound 145: +) ¹H-NMR (DMSO-d₆) δ 8.7 (1H, s, N—CH), 7.93 (1H, d, Ar—H), 7.48-7.32 (10H, m, 2×PhH), 7.12 (1H, bs, Ar—H), 6.23 (1H, q, —CH), 3.73 (4H, m, OCH₂), 3.14-3.0 (4H, m, NCH₂), 1.97 (3H, d, CH₃), 1.80 (6H, s, CH₃CCH₃). EIMS (m/z): 539.1 (M+H)

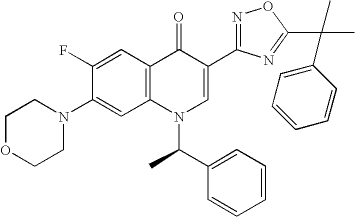

Proceeding as in Example 1b but substituting 6-fluoro-7-morpholino-4-oxo-1-(4-(trifluoromethyl)benzyl)-1,4-dihydroquinoline-3-carbonitrile and 2-methyl-2-phenylpropanoic acid gave 6-fluoro-7-morpholino-3-(5-(2-phenylpropan-2-yl)-1,2,4-oxadiazol-3-yl)-1-(4-(trifluoromethyl)benzyl)quinolin-4(1H)-one. (Compound 146: ++++). TLC system: 10% MeOH/CHCl₃; (R_{f} value: 0.5); ¹H NMR (DMSO-d₆; 300 MHz): 8.88 (s, 1H); 7.7 (dd, 3H); 7.4 (d, J=9 Hz, 2H); 7.25-7.38 (m, 4H); 6.9 (d, J=9 Hz, 1H); 5.83 (s, 1H); 3.68 (s, 4H); 3.0 (s, 4H); 1.8 (s, 6H); IR (KBr, vₘₐₓ): 3443, 2980, 1627, 1491, 1451, 1374, 1326, 1259, 1169, 1118, 1067 cm⁻¹. Mass: (M+H)⁺ 593; Purity by HPLC: 95.8%; Pale brown solid; 70 mg; (27.4%)

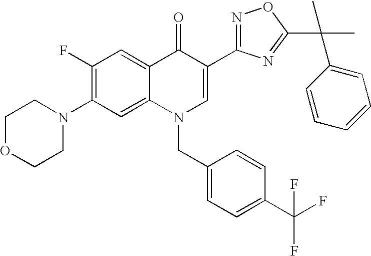

Proceeding as in Example 1b but substituting 6-fluoro-1-(4-fluorobenzyl)-7-morpholino-4-oxo-1,4-dihydroquinoline-3-carbonitrile and 2-methyl-2-phenylpropanoic acid gave 6-fluoro-1-(4-fluorobenzyl)-7-morpholino-3-(5-(2-phenylpropan-2-yl)-1,2,4-oxadiazol-3-yl)quinolin-4(1H)-one. (Compound 147) TLC system: 10% MeOH/CHCl₃; (R_{f} value: 0.5); ¹H NMR (DMSO-d₆; 300 MHz): 8.9 (s, 1H); 7.89-7.85 (d, J=9, 3H); 7.77-7.74 (d, J=9 Hz, 2H); 7.40-7.37 (d, J=9 Hz, 2H); 7.35-7.32 (dd, 2H); 7.20-7.17 (t, 2H); 7.06-7.04 (d, J=6 Hz, 2H); 5.87 (s, 2H); 3.49 (m, 4H); 3.16 (m, 4H); 1.98 (s, 6H). IR vₘₐₓ (cm⁻¹): 2968, 1625, 1566, 1490, 1371, 1314, 1258, 1218, 1168, 1118, 1030; Mass: (M+H)⁺ 543; Pale yellow solid; 30 mg (14%).

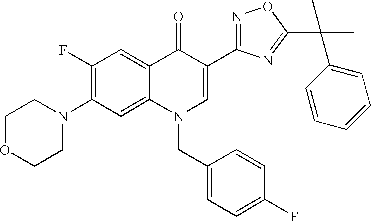

Proceeding as in Example 1b but substituting 1-(4-chlorobenzyl)-4-oxo-1,4-dihydroquinoline-3-carbonitrile and 2-(4-fluorophenyl)-2-methylpropanoic acid gave 1-(4-chlorobenzyl)-3-(5-(2-(4-fluorophenyl)propan-2-yl)-1,2,4-oxadiazol-3-yl)quinolin-4(1H)-one. (Compound 148) TLC system: 10% MeOH/CHCl₃; (R_{f} value: 0.5); ¹H NMR (DMSO-d₆; 300 MHz): 8.921 (s, 1H); 8.29-8.27 (d, J=6 Hz, 1H); 7.70-7.68 (m, 2H); 7.65-7.61 (m, 5H); 7.58-7.47 (m, 4H); 5.72 (s, 2H); 1.81 (s, 6H); IR (KBr, vₘₐₓ): 3443, 3058, 2983, 1634, 1563, 1491, 1395, 1322, 1168, 1096 cm⁻¹. Mass: (M+H)⁺ 474; Purity by HPLC: 99.9%; Off white solid; 36 mg (12.4%).

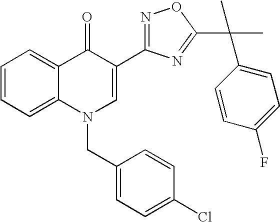

Proceeding as in Example 1b but substituting 4-oxo-1-(4-(trifluoromethyl)benzyl)-1,4-dihydroquinoline-3-carbonitrile and 2-(4-fluorophenyl)-2-methylpropanoic acid gave 3-(5-(2-(4-fluorophenyl)propan-2-yl)-1,2,4-oxadiazol-3-yl)-1-(4-(trifluoromethyl)benzyl)quinolin-4(1H)-one. (Compound 149) TLC system: 10% MeOH/CHCl₃; (Rf value: 0.5); ¹H NMR (DMSO-d₆; 300 MHz): 8.96 (s, 1H); 8.30-8.28 (d, J=6 Hz, 1H); 7.69-7.64 (m, 4H); 7.58-7.55 (d, J=9 Hz, 2H); 7.49 (m, 4H); 7.21 (t, 2H); 5.85 (s, 2H); 1.8 (s, 6H). IR (KBr, vₘₐₓ): 3439, 3066, 1636, 1492, 1425, 1327, 1228, 1068, 1019; Mass: (M+H)⁺ 480; Purity by HPLC: 95.9%; Pale brown solid, 50 mg (15%).

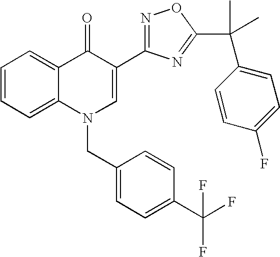

Proceeding as in Example 1b but substituting 6-fluoro-7-(2-morpholinoethylamino)-4-oxo-1-(4-(trifluoromethyl)benzyl)-1,4-dihydroquinoline-3-carbonitrile and 2-(4-fluorophenyl)-2-methylpropanoic acid gave 6-fluoro-3-(5-(2-(4-fluorophenyl)propan-2-yl)-1,2,4-oxadiazol-3-yl)-7-(2-morpholinoethylamino)-1-(4-(trifluoromethyl)benzyl)quinolin-4(1H)-one. (Compound 150). TLC system: 10% MeOH/DCM; (Rf value: 0.5). ¹H NMR (DMSO-d₆; 300 MHz): 8.88 (s, 1H); 7.85 (m, 3H); 7.47-7.50 (m, 4H); 7.28-7.30 (t, 2H); 6.5 (d, J=6 Hz, 2H); 5.89 (s, 2H); 3.5 (m, 8H); 3.1 (m, 4H); 1.89 (s, 6H); IR (KBr, vₘₐₓ cm⁻¹): 3437, 3347, 1632, 1595, 1530, 1499, 1326, 1235, 1167, 1120, 1067 cm⁻¹; Mass: (M+H)⁺ 654; Purity by HPLC: 94.4% Brown solid; 12 mg (6.6%).

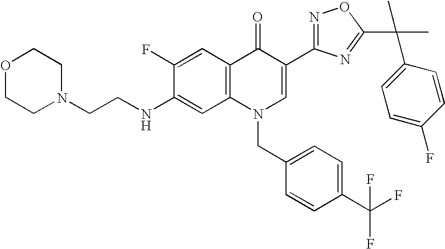

Proceeding as in Example 1b, but substituting 7-(1,1-dioxo-thiomorpholin-4-yl)-6-fluoro-4-oxo-1-(4-fluoro-benzyl)-1,4-dihydro-quinoline-3-carbonitrile and 2-(4-fluorophenyl)-2-methylpropanoic acid gave 7-(1,1-dioxo-thiomorpholine)-6-fluoro-3-(5-(2-(4-fluorophenyl)propan-2-yl)-1,2,4-oxadiazol-3-yl)-1-(4-(fluoro)benzyl)quinolin-4(1H)-one. (Compound 151: ++). TLC system: 10% MeOH/CHCl₃; (R_{f} value: 0.5); ¹H NMR (DMSO-d₆; 300 MHz): 8.9 (s, 1H); 7.89-7.85 (d, J=9, 3H); 7.77-7.74 (d, J=9 Hz, 2H); 7.40-7.37 (d, J=9 Hz, 2H); 7.35-7.32 (dd, 2H); 7.20-7.17 (t, 2H); 7.06-7.04 (d, J=6 Hz, 2H); 5.87 (s, 2H); 3.49 (m, 4H); 3.16 (m, 4H); 1.83 (s, 6H). IR vₘₐₓ (cm⁻¹): 3447, 2984, 1631, 1599, 1495, 1383, 1315, 1284, 1230, 1168, 1124, 1039; Mass: (M+H)⁺ 610; Off-white solid; 20 mg (9.74%).

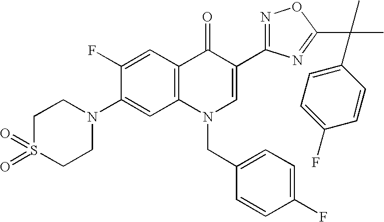

Proceeding as in Example 1b, but substituting 7-(dimethylamino)-6-fluoro-4-oxo-1-(4-(trifluoromethyl)benzyl)-1,4-dihydroquinoline-3-carbonitrile and 2-methyl-2-phenylpropanoic acid, gave 7-(dimethylamino)-6-fluoro-3-(5-(2-phenylpropan-2-yl)-1,2,4-oxadiazol-3-yl)-1-(4-(trifluoromethyl)benzyl)quinolin-4(1H)-one. (Compound 152: ++++). TLC system: 10% MeOH/CHCl₃; (R_{f} value: 0.6); ¹H NMR (DMSO-d₆; 300 MHz): 8.88 (s, 1H); 7.75 (m, 3H); 7.47-7.50 (d, J=9 Hz, 2H); 7.25-7.38 (m, 5H); 6.61-6.64 (d, J=9 Hz, 1H); 5.81 (s, 2H); 2.83 (s, 6H); 1.89 (s, 6H); IR (KBr, vₘₐₓ): 3445, 1630, 1588, 1494, 1414, 1369, 1326, 1261, 1166, 1120, 1065 cm⁻¹; Mass: (M+H)⁺ 551; Purity by HPLC: 96.4%; Off-white solid; 40 mg (25.6%)

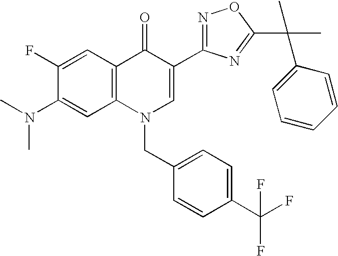

Proceeding as in Example 1b, but substituting 7-(dimethylamino)-6-fluoro-4-oxo-1-(4-(trifluoromethyl)benzyl)-1,4-dihydroquinoline-3-carbonitrile and 2-(4-fluorophenyl)-2-methylpropanoic acid, gave 7-(dimethylamino)-6-fluoro-3-(5-(2-(4-fluorophenyl)propan-2-yl)-1,2,4-oxadiazol-3-yl)-1-(4-(trifluoromethyl)benzyl)quinolin-4(1H)-one. (Compound 153: +++). TLC system: 10% MeOH/CHCl₃; (R_{f} value: 0.6); ¹H NMR (DMSO-d₆; 300 MHz): 8.86 (s, 1H); 7.73-7.78 (t, 3H); 7.37-7.47 (m, 4H); 7.1 (t, 2H); 6.6 (d, J=6 Hz, 1H); 5.81 (s, 2H); 2.91-2.84 (s, 6H); 1.81 (s, 6H). IR (KBr, vₘₐₓ): 3444, 1631, 1591, 1499, 1410, 1367, 1326, 1237, 1168, 1124, 1064 cm⁻¹; Mass: (M+H)⁺ 569; Purity by HPLC: 96.7%; Pale brown solid; 80 mg (29.7%).

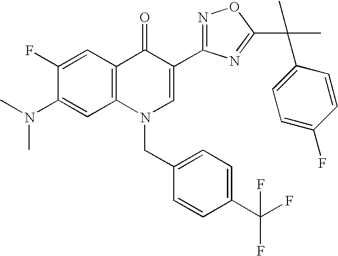

Proceeding as in Example 1b, but substituting 7-(dimethylamino)-6-fluoro-1-(4-fluorobenzyl)-4-oxo-1,4-dihydroquinoline-3-carbonitrile and 2-methyl-2-phenylpropanoic acid, gave 7-(dimethylamino)-6-fluoro-1-(4-fluorobenzyl)-3-(5-(2-phenylpropan-2-yl)-1,2,4-oxadiazol-3-yl)quinolin-4(1H)-one. (Compound 154: +++). TLC system: 10% MeOH/CHCl₃; (R_{f} value: 0.5); ¹H NMR (DMSO-d₆; 300 MHz): 8.88 (s, 1H); 7.75 (m, 3H); 7.47-7.50 (d, J=9 Hz, 2H); 7.25-7.38 (m, 5H); 6.61-6.64 (d, J=9 Hz, 1H); 5.81 (s, 2H); 2.83 (s, 6H); 1.89 (s, 6H); IR (KBr, vₘₐₓ): 3056, 2968, 1631, 1595, 1495, 1403, 1363, 1321, 1257, 1223, 1167, 1104, 1051 cm⁻¹; Mass: (M+H)⁺ 501; Purity by HPLC: 97.9%; Pale brown solid; 42 mg (20.8%).

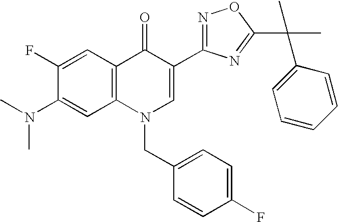

Proceeding as in Example 1b, but substituting 6,7-difluoro-1-(4-fluorobenzyl)-4-oxo-1,4-dihydroquinoline-3-carbonitrile and 2-(4-fluorophenyl)-2-methylpropanoic acid, gave 6,7-difluoro-1-(4-fluorobenzyl)-3-(5-(2-(4-fluorophenyl)propan-2-yl)-1,2,4-oxadiazol-3-yl)quinolin-4(1H)-one. (Compound 155: +). TLC system: 50% ethyl acetate/pet-ether; (Rf value: 0.3); ¹H NMR (DMSO-d₆; 300 MHz): 8.94 (s, 1H); 8.16 (t, 1H); 7.87 (dd, 1H); 7.37-7.33 (m, 4H); 7.23-2.20 (m, 4H); 5.69 (s, 2H); 1.96 (s, 6H); IR v_{Max} (cm⁻¹): 3446, 2987, 1624, 1570, 1503, 1412, 1326, 1290, 1233, 1162, 1100, 1015; Mass: (M+H)⁺ 494; Purity by HPLC: 93%; Pale brown solid; 40 mg; (28.16%)

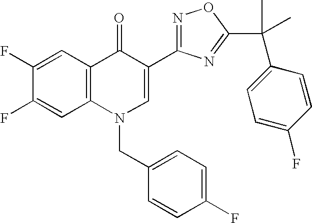

Proceeding as in Example 1b, but substituting 1-(4-fluorobenzyl)-4-oxo-1,4-dihydroquinoline-3-carbonitrile and 2-(4-fluorophenyl)-2-methylpropanoic acid, gave 1-(4-fluorobenzyl)-3-(5-(2-(4-fluorophenyl)propan-2-yl)-1,2,4-oxadiazol-3-yl)quinolin-4(1H)-one. (Compound 156: +).

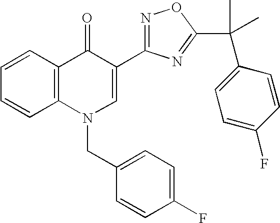

Proceeding as in Example 1b, but substituting 7-(dimethylamino)-6-fluoro-1-(4-fluorobenzyl)-4-oxo-1,4-dihydroquinoline-3-carbonitrile and 2-(4-fluorophenyl)-2-methylpropanoic acid, gave 7-(dimethylamino)-6-fluoro-1-(4-fluorobenzyl)-3-(5-(2-(4-fluorophenyl)propan-2-yl)-1,2,4-oxadiazol-3-yl)quinolin-4(1H)-one. (Compound 157). TLC system: 10% MeOH/CHCl₃; (R_{f} value: 0.5); ¹H NMR (DMSO-d₆; 300 MHz): 8.88 (s, 1H); 7.75-7.73 (d, J=6 Hz, 1H); 7.47-7.50 (m, 8H); 6.76-6.74 (d, J=6 Hz, 1H); 5.75 (s, 2H); 2.83 (s, 6H); 1.89 (s, 6H); IR (KBr, vₘₐₓ): 3070, 2984, 2880, 2804, 1633, 1500, 1407, 1368, 1324, 1230, 1168, 1055 cm⁻¹; Mass: (M+H)⁺ 518; Purity by HPLC: 99.2%; Pale brown solid; 53 mg (24.33%).

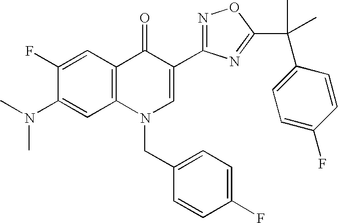

Proceeding as in Example 1b, but substituting 1-(4-trifluorobenzyl)-4-oxo-1,4-dihydroquinoline-3-carbonitrile and 2-(4-fluorophenyl)acetic acid, gave 1-(4-trifluorobenzyl)-3-(5-(4-fluorobenzyl)-1,2,4-oxadiazol-3-yl)quinolin-4(1H)-one. (Compound 158) TLC system: 10% MeOH/CHCl₃; (Rf value: 0.5); ¹H NMR (DMSO-d₆; 300 MHz): 8.96 (s, 1H); 8.30-8.28 (d, J=6 Hz, 1H); 7.69-7.64 (m, 4H); 7.58-7.55 (d, J=9 Hz, 2H); 7.49 (m, 4H); 7.21 (t, 2H); 5.85 (s, 2H); 1.8 (s, 6H). IR (KBr, vₘₐₓ): 3439, 3066, 1636, 1492, 1425, 1327, 1228, 1068, 1019; Mass: (M+H)⁺ 480; Purity by HPLC: 95.9%; Pale brown solid, 50 mg (15%).

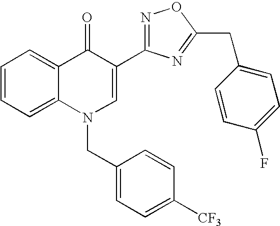

Proceeding as in Example 1b, but substituting 6,7-difluoro-1-(4-fluorobenzyl)-4-oxo-1,4-dihydroquinoline-3-carbonitrile and 2-methyl-2-phenylpropanoic acid, gave 6,7-difluoro-1-(4-fluorobenzyl)-3-(5-(2-phenylpropan-2-yl)-1,2,4-oxadiazol-3-yl)quinolin-4(1H)-one. (Compound 159). TLC system: 50% ethyl acetate/pet-ether; (Rf value: 0.2); ¹H NMR (DMSO-d₆; 300 MHz): 8.94 (s, 1H); 8.16 (t, 1H); 7.87 (dd, 1H); 7.37-7.33 (m, 4H); 7.23-2.20 (m, 4H); 5.69 (s, 2H); 1.96 (s, 6H); IR vₘₐₓ (cm⁻¹): 3446, 2987, 1624, 1570, 1503, 1412, 1326, 1290, 1233, 1162, 1100, 1015; Mass: (M+H)⁺ 476; Purity by HPLC: 97.3%; Pale yellow solid; 20 mg; (19.04%)

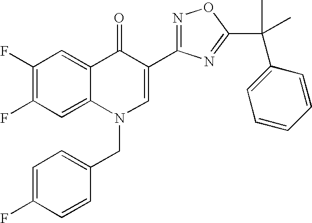

Proceeding as in Example 1b, but substituting 7-chloro-6-fluoro-1-(4-fluorobenzyl)-4-oxo-1,4-dihydroquinoline-3-carbonitrile and 2-methyl-2-phenylpropanoic acid, gave 7-chloro-6-fluoro-3-(5-(2-phenylpropan-2-yl)-1,2,4-oxadiazol-3-yl)-1-(4-(trifluoromethyl)benzyl)quinolin-4(1H)-one. (Compound 160) ¹H-NMR (DMSO-d₆) δ 8.99 (1H, s, N—CH), 8.13 (1H, d, Ar—H), 7.99 (1H, d, Ar—H), 7.77 (2H, d, Ar—H), 7.42 (2H, d, Ar—H), 7.40-7.16 (5H, m, Ar—H), 5.92 (2H, s, NCH₂Ph), 1.82 (6H, s, CH₃CCH₃). EIMS (m/z): 542.0 (M+H)

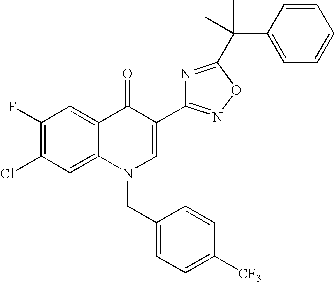

### Example 2

### 6-fluoro-3-(5-(2-(4-fluorophenyl)propan-2-yl)-1,3,4-oxadiazol-2-yl)-7-morpholino-1-(4-(trifluoromethyl)benzyl)quinolin-4(1H)-one

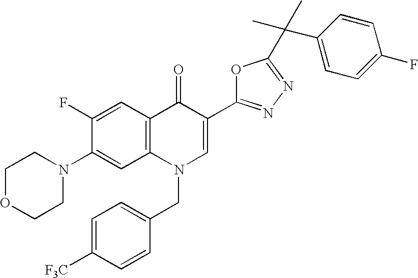

To a stirred solution of uncyclized intermediate 2.12 (92 mg, 0.14 mmol) in dichloromethane (10 mL) was treated with triphenylphosphine (115 mg, 0.43 mmol), triethyl amine (44 mg, 0.43 mmol) and carbon tetrachloride (135 mg, 0.87 mmol) under nitrogen atmosphere at room temperature. The reaction mixture was heated at reflux for over night. On the completion of the reaction, it is portioned between water and dichloromethane. The organic layer was washed with brine solution, dried over MgSO₄, filtered and concentrated. The residue was purified by silica gel flash column chromatography eluting with 1.0% methanol in dichloromethane to provide 1,3,4-oxadiazole 2.13 (Compound 161: +++). (54 mg, 60.6%). ¹H-NMR (DMSO-d₆) δ 9.0 (1H, s, NCH), 7.80 (1H, d, Ar—H), 7.78 (2H, d, Ar—H), 7.56 (2H, d, Ar—H), 7.40 (2H, m, Ar—H), 7.20 (2H, m, Ar—H), 6.92 (1H, d, Ar—H), 5.90 (2H, s, CH₂), 3.76 (4H, m, OCH₂), 3.04 (4H, m, NCH₂). EIMS (m/z): 611.5 (M+H)+.

Proceeding as in Example 2 from analogous starting material gave 1-(4-chloro-benzyl)-3-[5-(4-chloro-benzyl)-[1,3,4]oxadiazol-2-yl]-6,7-dimethoxy-1H-quinolin-4-one (Compound 162: +).

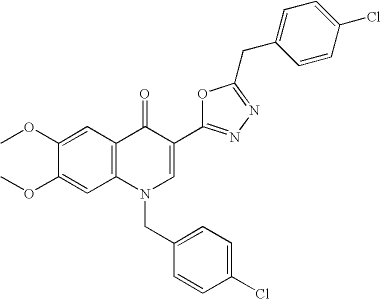

Proceeding as in Example 2 from analogous starting material gave 3-[5-(4-chloro-benzyl)-[1,3,4]oxadiazol-2-yl]-1-cyclohexylmethyl-6,7-dimethoxy-1H-quinolin-4-one (Compound 163: +).

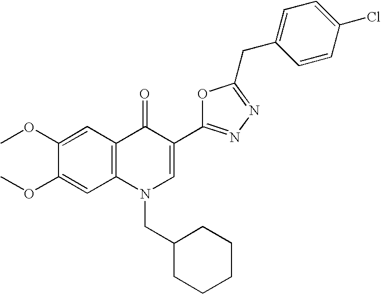

Proceeding as in Example 2 from analogous starting material gave 3-(5-benzyl-[1,3,4]oxadiazol-2-yl)-1-(4-chloro-benzyl)-6-fluoro-7-(4-methyl-piperazin-1-yl)-1H-quinolin-4-one (Compound 164: ++).

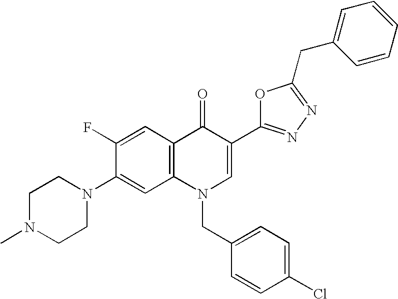

Proceeding as in Example 2 from analogous starting material gave 3-(5-benzyl-[1,3,4]oxadiazol-2-yl)-1-(4-(2-fluoro-4-trifluormethyl)-benzyl)-6-fluoro-7-(4-methyl-piperazin-1-yl)-1H-quinolin-4-one (Compound 165: +++).

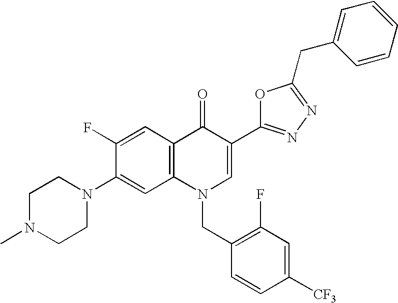

Proceeding as in Example 2 from analogous starting material gave 6-fluoro-3-(5-(2-(4-fluorophenyl)propan-2-yl)-1,3,4-oxadiazol-2-yl)-7-morpholino-1-(4-(trifluoromethyl)benzyl)quinolin-4(1H)-one. (Compound 166). ¹H-NMR (DMSO-d₆) δ 8.99 (1H, s, N—CH), 7.99 (1H, d, Ar—H), 7.76 (2H, d, Ar—H), 7.54 (2H, d, Ar—H), 7.38 (2H, m, Ar—H), 7.20 (2H, m, Ar—H), 6.92 (1H, d, Ar—H), 5.81 (2H, s, NCH₂Ph), 3.68 (4H, m, OCH₂), 3.03 (4H, m, NCH₂), 2.80 (6H, s, CH₃CCH₃), EIMS (m/z): 611.0 (M+H).

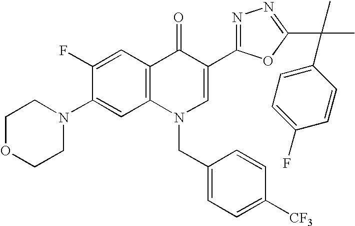

Proceeding as in Example 2 from analogous starting material gave 6-fluoro-7-morpholino-3-(5-(2-phenylpropan-2-yl)-1,3,4-oxadiazol-2-yl)-1-(4-(trifluoromethyl)benzyl)quinolin-4(1H)-one. (Compound 167: +++).

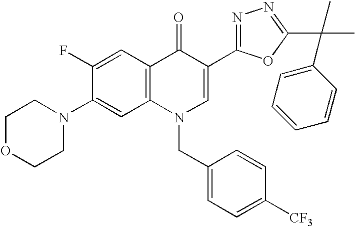

### Example 3

### 6-fluoro-3-(3-(4-fluorobenzyl)-1,2,4-oxadiazol-5-yl)-7-morpholino-1-(4-(trifluoromethyl)benzyl)quinolin-4(1H)-one

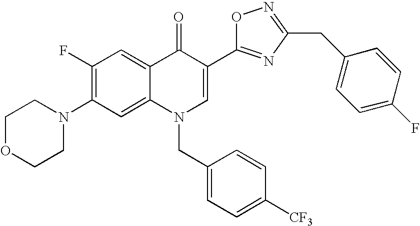

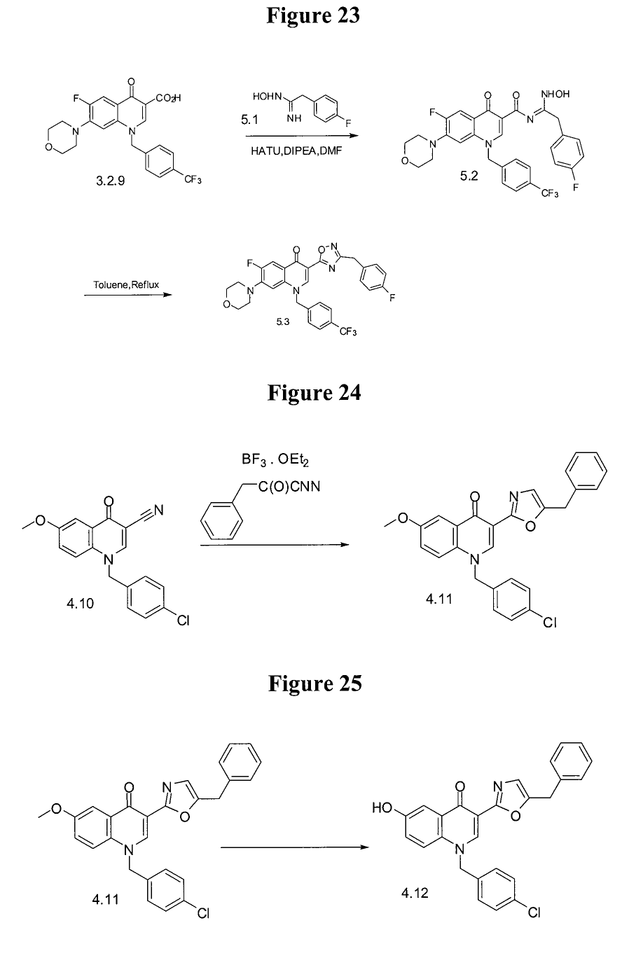

### See FIG. 23

To a stirred solution of acid 3.2.9 (250 mg, 0.55 mmol) in DMF (10 mL) was treated with HATU (253 mg, 0.66 mmol) and DIPEA (358 mg, 2.77 mmol). The reaction mixture stirred for 1 h under nitrogen atmosphere at room temperature, then compound 5.1 (93 mg, 0.55 mmol) was added, and left over night. The reaction mixture was portioned between water and ethyl acetate. The organic layer was washed with brine solution, dried over MgSO₄, filtered and concentrated. The crude residue was purified by silica gel flash column chromatography eluting with 1.0% methanol in dichloromethane to provide compound 5.2 (232 mg, 70%). ¹H-NMR (DMSO-d₆) δ 9.02 (1H, s, NCH), 7.82 (1H, d, Ar—H), 7.78 (2H, d, Ar—H), 7.56 (2H, d, Ar—H), 7.41 (2H, m, Ar—H), 7.19 (2H, m, Ar—H), 6.96 (1H, m, Ar—H, NH), 5.93 (2H, s, CH₂), 3.79, 3.43 (2H, 2×s, CH₂), 3.72 (4H, m, OCH₂), 3.07 (4H, m, NCH₂). EIMS (m/z): 601.3 (M+H)+.

The uncyclized intermediate 5.2 (80 mg) was dissolved in toluene (20 mL) under nitrogen atmosphere at room temperature. The reaction mixture heated to reflux for overnight, once the reaction was completed then solvent was removed under reduced pressure. The crude residue was purified by silica gel flash column chromatography eluting with 2% methanol in dichloromethane to provide compound 5.3, (Compound 168) (36 mg, 46.75%). ¹H-NMR (DMSO-d₆) δ 9.18 (1H, s, CH), 7.83 (1H, d, Ar—H), 7.77 (2H, d, Ar—H), 7.58 (2H, d, Ar—H), 7.41 (2H, m, Ar—H), 7.18 (2H, m, Ar—H), 6.97 (1H, d, Ar—H), 5.86 (2H, s, CH₂), 4.17 (2H, s, CH₂), 3.72 (4H, bs, OCH₂), 3.03 (4H, bs, NCH₂). EIMS (m/z): 583.6 (M+H)+.

Proceeding as in Example 3, but substituting 1-(4-chlorobenzyl)-6,7-dimethoxy-4-oxo-1,4-dihydroquinoline-3-carboxylic acid and N-hydroxy-2-phenylacetimidamide, gave 3-(3-benzyl-[1,2,4]oxadiazol-5-yl)-1-(4-chloro-benzyl)-6,7-dimethoxy-1H-quinolin-4-one. (Compound 169: +).

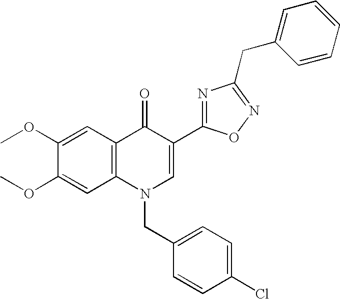

Proceeding as in Example 3, but substituting 1-(cyclohexylmethyl)-6,7-dimethoxy-4-oxo-1,4-dihydroquinoline-3-carboxylic acid and N-hydroxy-2-phenylacetimidamide, gave 3-(3-benzyl-[1,2,4]oxadiazol-5-yl)-1-cyclohexylmethyl-6,7-dimethoxy-1H-quinolin-4-one (Compound 170: +).

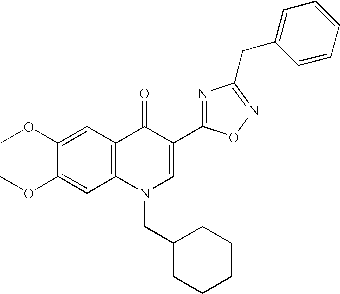

Proceeding as in Example 3, but 6-fluoro-7-morpholino-4-oxo-1-(4-(trifluoromethyl)benzyl)-1,4-dihydroquinoline-3-carboxylic acid and N-hydroxy-2-methyl-2-phenylpropanimidamide gave 6-fluoro-7-morpholino-3-(3-(2-phenylpropan-2-yl)-1,2,4-oxadiazol-5-yl)-1-(4-(trifluoromethyl)benzyl)quinolin-4(1H)-one. (Compound 171: +). ¹H-NMR (DMSO-d₆) δ 9.06 (1H, s, N—CH), 7.84 (1H, d, Ar—H), 7.75 (2H, d, Ar—H), 7.54 (2H, d, Ar—H), 7.38-7.20 (5H, m, Ph-H), 6.88 (1H, d, Ar—H), 5.84 (2H, s, NCH₂Ph), 3.67 (4H, m, OCH₂), 3.03 (4H, m, NCH₂), 1.88 (6H, s, CH₃CCH₃). EIMS (m/z): 593.1 (M+H)

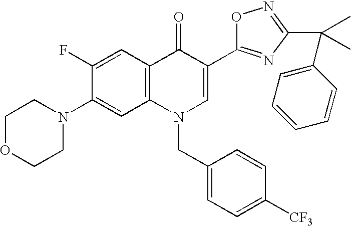

Proceeding as in Example 3, but 6-fluoro-7-morpholino-4-oxo-1-(4-(trifluoromethyl)benzyl)-1,4-dihydroquinoline-3-carboxylic acid and 2-(4-fluorophenyl)-N-hydroxyacetimidamide gave 6-fluoro-3-(3-(4-fluorobenzyl)-1,2,4-oxadiazol-5-yl)-7-morpholino-1-(4-(trifluoromethyl)benzyl)quinolin-4(1H)-one. (Compound 172: +).

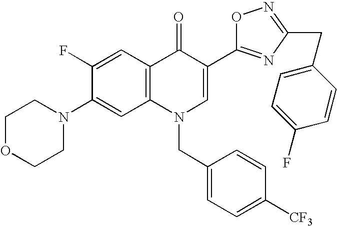

Proceeding as in Example 3, but 6-fluoro-7-morpholino-4-oxo-1-(4-(trifluoromethyl)benzyl)-1,4-dihydroquinoline-3-carboxylic acid and 2-(4-fluorophenyl)-N-hydroxy-2-methylpropanimidamide gave 6-fluoro-3-(3-(2-(4-fluorophenyl)propan-2-yl)-1,2,4-oxadiazol-5-yl)-7-morpholino-1-(4-(trifluoromethyl)benzyl)quinolin-4(1H)-one. (Compound 173: +). ¹H-NMR (DMSO-d₆) δ 9.06 (1H, s, N—CH), 7.84 (1H, d, Ar—H), 7.78 (2H, d, Ar—H), 7.56 (2H, d, Ar—H), 7.39 (2H, m, Ar—H), 7.17 (2H, m, Ar—H), 6.92 (1H, d, Ar—H), 5.84 (2H, s, NCH₂Ph), 3.72 (4H, m, OCH₂), 3.04 (4H, m, NCH₂), 1.78 (6H, s, CH₃CCH₃), EIMS (m/z): 611.1 (M+H)⁺

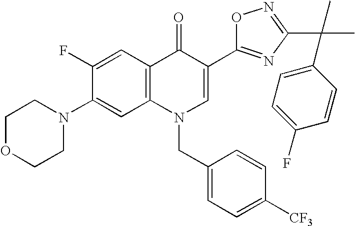

### Example 4

### 3-(5-benzyloxazol-2-yl)-1-(4-chlorobenzyl)-6-methoxyquinolin-4(1H)-one

### See FIG. 24

To a solution of 1-(4-chlorobenzyl)-6-methoxy-4-oxo-1,4-dihydroquinoline-3-carbonitrile (0.62 g; 1.9 mmol), BF₃OEt₂ (0.24 mL; 1.9 mmol) and CH₂Cl₂ (9 mL) was added diazoketone (0.16 g; 0.97 mmol in 0.6 mL of CH₂Cl₂). The mixture stirred at rt and two more equivalents of diazoketon were added. Upon completion of the reaction NaOH (10% aq) was added and the organic layer was portioned and washed with brine. The organic layer was dried, filtered, and evaporated under reduced pressure. The residue was purified by preparative HPLC to afford 3-(5-benzyl-oxazol-2-yl)-1-(4-chloro-benzyl)-6-methoxy-1H-quinolin-4-one (Compound 174), Mass Spec: 456.9, 479 (M+1, M+23). ¹H NMR: 8.91 (1H, s), 7.71 (1H, d, 2.4 Hz), 7.63 (1H, d, J=9.4 Hz), 7.38 (7H, m), 7.28 (3H, m), 7.05 (1H, s), 5.72 (2H, s), 4.14 (2H, s), 3.86 (3H, s);

### Example 4.1

### 3-(5-benzyloxazol-2-yl)-1-(4-chlorobenzyl)-6-hydroxyquinolin-4(1H)-one

### See FIG. 25

To a solution of 3-(5-benzyloxazol-2-yl)-1-(4-chlorobenzyl)-6-methoxyquinolin-4(1H)-one (0.04 g) in dichloromethane was added BBr₃ (0.3 mL). After 4 h at rt was added HBr (0.3 mL) and the mixture stirred overnight. The reaction was evaporated to dryness under reduced pressure and the residue was purified by preparative HPLC to afford 3-(5-benzyl-oxazol-2-yl)-1-(4-chloro-benzyl)-6-hydroxy-1H-quinolin-4-one (30.7 mg) (Compound 175), Mass Spec: 443.2, 465 (M+1, M+23);

### Example 4.2

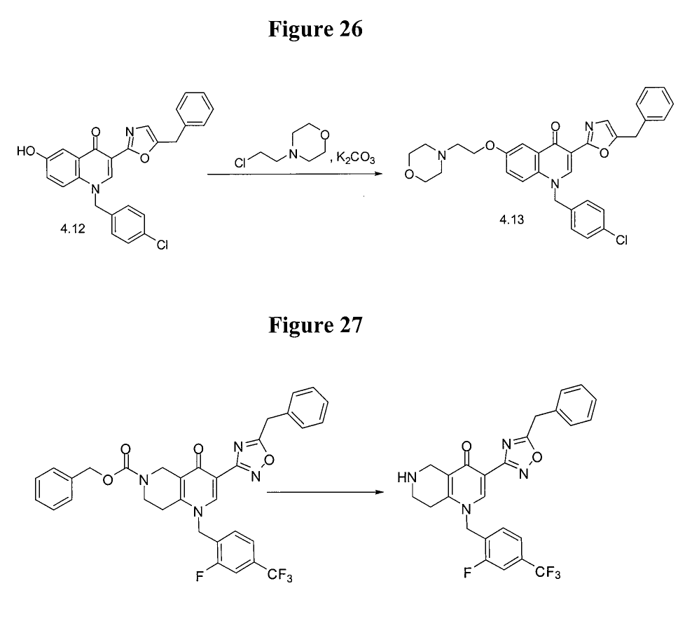

### See FIG. 26

### 3-(5-benzyl-oxazol-2-yl)-1-(4-chloro-benzyl)-6-(3-morpholin-4-yl-propoxy)-1H-quinolin-4-one

To a solution of 3-(5-benzyloxazol-2-yl)-1-(4-chlorobenzyl)-6-hydroxyquinolin-4(1H)-one (0.026 g; 0.058 mmol) in DMF was added K₂CO₃ (0.024 g) and 4-(2-chloroethyl)morpholine (0.011 g; 0.07 mmol) and the mixture was heated at 100° C. for 2 h. The product was evaporated and the residue was purified by preparative HPLC to afford 3-(5-benzyl-oxazol-2-yl)-1-(4-chloro-benzyl)-6-(3-morpholin-4-yl-propoxy)-1H-quinolin-4-one (Compound 176: ++). Mass Spec: 570.2, 592.2 (M+1, M+23);

### Example 5

### 3-(5-Benzyl-[1,2,4]oxadiazol-3-yl)-1-(2-fluoro-4-trifluoromethyl-benzyl)-5,6,7,8-tetrahydro-1H-[1,6]naphthyridin-4-one

### See FIG. 27

A solution of benzyl 3-(5-benzyl-1,2,4-oxadiazol-3-yl)-1-(2-fluoro-4-(trifluoromethyl)benzyl)-4-oxo-4,5,7,8-tetrahydro-1,6-naphthyridine-6(1H)-carboxylate was stirred in HBr/HOAc solution for 30 min. The reaction mixture was evaporated and purified by preparative HPLC to afford 3-(5-Benzyl-[1,2,4]oxadiazol-3-yl)-1-(2-fluoro-4-trifluoromethyl-benzyl)-5,6,7,8-tetrahydro-1H-[1,6]naphthyridin-4-one (Compound 177), Mass Spec: 486.2, 507.1 (M+1, M+23);

Proceeding as Example 5 but substituting benzyl 3-(5-(4-chlorobenzyl)-1,2,4-oxadiazol-3-yl)-1-(2-fluoro-4-(trifluoromethyl)benzyl)-4-oxo-4,5,7,8-tetrahydro-1,6-naphthyridine-6(1H)-carboxylate afforded 3-[5-(4-chloro-benzyl)-[1,2,4]oxadiazol-3-yl]-1-(2-fluoro-4-trifluoromethyl-benzyl)-5,6,7,8-tetrahydro-1H-[1,6]naphthyridin-4-one (Compound 178: ++). Mass Spec: 521.0, 541.3 (M+2, M+23), 517.2 (M−1);

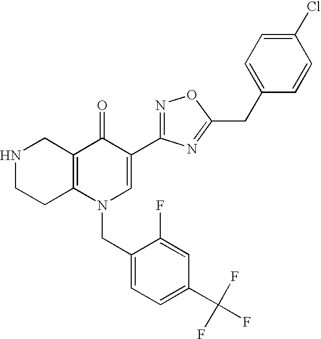

### Example 6

### 6-fluoro-3-(5-(2-(4-fluorophenyl)propan-2-yl)-1,2,4-oxadiazol-3-yl)-7-methoxy-1-(4-(trifluoromethyl)benzyl)quinolin-4(1H)-one

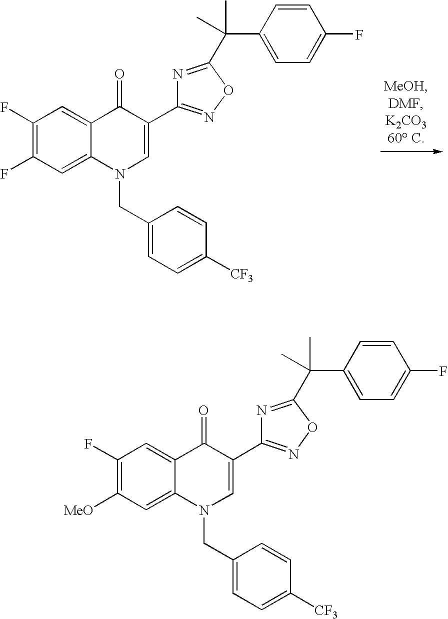

Proceeding as in Reference 3 but substituting methanol and 6,7-difluoro-3-(5-(2-(4-fluorophenyl)propan-2-yl)-1,2,4-oxadiazol-3-yl)-1-(4-(trifluoromethyl)benzyl)quinolin-4(1H)-one gave 6-fluoro-3-(5-(2-(4-fluorophenyl)propan-2-yl)-1,2,4-oxadiazol-3-yl)-7-methoxy-1-(4-(trifluoromethyl)benzyl)quinolin-4(1H)-one. (Compound 179) ¹H-NMR (DMSO-d₆) δ 8.92 (1H, s, N—CH), 7.92 (1H, d, Ar—H), 7.74 (2H, d, Ar—H), 7.52 (2H, d, Ar—H), 7.39 (2H, m, Ar—H), 7.19 (3H, m, Ar—H), 5.88 (2H, s, NCH₂Ph), 3.92 (3H, s, OMe), 1.82 (6H, s, CH₃CCH₃), EIMS (m/z): 556.0 (M+H)⁺.

Proceeding as in Reference 3 but substituting ethanol and 6,7-difluoro-3-(5-(2-(4-fluorophenyl)propan-2-yl)-1,2,4-oxadiazol-3-yl)-1-(4-(trifluoromethyl)benzyl)quinolin-4(1H)-one gave 6-fluoro-3-(5-(2-(4-fluorophenyl)propan-2-yl)-1,2,4-oxadiazol-3-yl)-7-ethoxy-1-(4-(trifluoromethyl)benzyl)quinolin-4(1H)-one. (Compound 180) ¹H-NMR (DMSO-d₆) δ 8.92 (1H, s, N—CH), 7.92 (1H, d, Ar—H), 7.74 (2H, d, Ar—H), 7.48 (2H, d, Ar—H), 7.39 (2H, m, Ar—H), 7.20 (2H, m, Ar—H), 7.12 (1H, d, Ar—H), 5.88 (2H, s, NCH₂Ph), 4.12 (2H, q, OCH₂), 1.82 (6H, s, CH₃CCH₃), 1.22 (3H, t, CH₃). EIMS (m/z): 570.0 (M+H)⁺

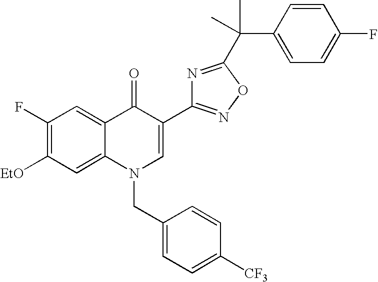

Proceeding as in Reference 3 but substituting 2-methoxyethanol and 6,7-difluoro-3-(5-(2-(4-fluorophenyl)propan-2-yl)-1,2,4-oxadiazol-3-yl)-1-(4-(trifluoromethyl)benzyl)quinolin-4(1H)-one gave 6-fluoro-3-(5-(2-(4-fluorophenyl)propan-2-yl)-1,2,4-oxadiazol-3-yl)-7-(2-methoxyethoxy)-1-(4-(trifluoromethyl)benzyl)quinolin-4(1H)-one. (Compound 181) ¹H-NMR (DMSO-d₆) δ 8.92 (1H, s, N—CH), 7.94 (1H, d, Ar—H), 7.74 (2H, d, Ar—H), 7.48 (2H, d, Ar—H), 7.39 (2H, m, Ar—H), 7.20 (3H, m, Ar—H), 5.88 (2H, s, NCH₂Ph), 4.20 (2H, t, OCH₂), 3.60 (2H, t, OCH₂), 3.12 (3H, s, OCH₃), 1.82 (6H, s, CH₃CCH₃). EIMS (m/z): 600.0 (M+H)⁺.

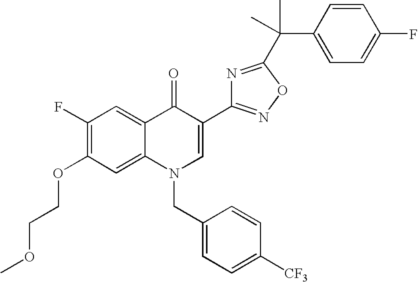

Proceeding as in Reference 3 but substituting (2,2-dimethyl-1,3-dioxolan-4-yl)methanol and 6,7-difluoro-3-(5-(2-(4-fluorophenyl)propan-2-yl)-1,2,4-oxadiazol-3-yl)-1-(4-(trifluoromethyl)benzyl)quinolin-4(1H)-one gave 7-((2,2-dimethyl-1,3-dioxolan-4-yl)methoxy)-6-fluoro-3-(5-(2-(4-fluorophenyl)propan-2-yl)-1,2,4-oxadiazol-3-yl)-1-(4-(trifluoromethyl)benzyl)quinolin-4(1H)-one. (Compound 182). The acetinide was treated with acid and purified by column chromatography to afford 7-(2,3-dihydroxypropoxy)-6-fluoro-3-(5-(2-(4-fluorophenyl)propan-2-yl)-1,2,4-oxadiazol-3-yl)-1-(4-(trifluoromethyl)benzyl)quinolin-4(1H)-one. ¹H-NMR (DMSO-d₆) δ 8.91 (1H, s, N—CH), 7.95 (1H, d, Ar—H), 7.74 (2H, d, Ar—H), 7.48 (2H, d, Ar—H), 7.39 (2H, m, Ar—H), 7.20 (3H, m, Ar—H), 5.88 (2H, s, NCH₂Ph), 5.06 (1H, d, OH), 4.77 (1H, t, OH), 4.04-3.97 (2H, m, OCH₂), 3.78 (1H, m, OCH), 3.43 (2H, m, OCH₂), 1.82 (6H, s, CH₃CCH₃) EIMS (m/z): 616.0 (M+H)⁺.

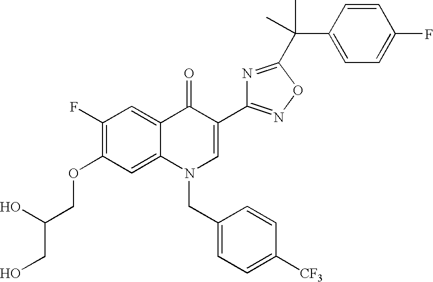

Proceeding as in Reference 3 but substituting 2-morpholinoethanol and 6,7-difluoro-3-(5-(2-(4-fluorophenyl)propan-2-yl)-1,2,4-oxadiazol-3-yl)-1-(4-(trifluoromethyl)benzyl)quinolin-4(1H)-one gave 6-fluoro-3-(5-(2-(4-fluorophenyl)propan-2-yl)-1,2,4-oxadiazol-3-yl)-7-(2-morpholinoethoxy)-1-(4-(trifluoromethyl)benzyl)quinolin-4(1H)-one. (Compound 183) ¹H-NMR (DMSO-d₆) δ 8.92 (1H, s, N—CH), 7.94 (1H, d, Ar—H), 7.74 (2H, d, Ar—H), 7.46 (2H, d, Ar—H), 7.39 (2H, m, Ar—H), 7.18 (3H, m, Ar—H), 5.88 (2H, s, NCH₂Ph), 4.18 (2H, t, OCH₂), 3.52 (4H, m, OCH₂), 2.59 (2H, t, NCH₂), 4.38 (4H, m, NCH₂), 1.82 (6H, s, CH₃CCH₃). EIMS (m/z): 655.1 (M+H)⁺.

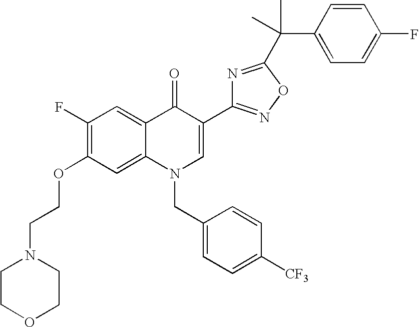

### Biological Example 1

### HCV Replicon Assay

The HCV replicon assay is a cell-culture system that mimics in vivo HCV replication and provides a system to study HCV replication in vitro. It was created by transfecting cloned viral RNA derived from a consensus HCV genomic sequence into human Huh7 hepatoma cells that are semi-permissive for viral RNA production (Lohmann V., Korner F., Koch J.-O., Herian U., Theilmann L. and Bartenschlager R. (1999). Replication of subgenomic Hepatitis C virus RNAs in a hepatoma cell line. Science 285, 110-113 and Blight K. J., Kolykhalov A. A. and Rice C. M. (2000). Efficient initiation of HCV RNA Replication in cell culture. Science 290, 972-1974). These transfected cell lines contain a subgenomic HCV RNA genome that includes (1) the HCV 5′NTR fused to 12 amino acids of the capsid coding region, (2) the neomycin phosphotransferase gene (Neo) as a selectable marker, (3) the internal ribosome entry site (IRES) from encephalomyocarditis virus (EMCV) that directs translation of HCV non-structural proteins (variously NS2 or NS3 to NS5B), and (4) the 3′ NTR. Replicon-containing cells autonomously and persistently replicate HCV RNA that can be measured quantitatively by real-time qPCR. Therefore, the replicon system facilitates quantitative assessment of anti-viral activity by monitoring changes in HCV RNA replication in a cell-based assay.

HCV replicon-containing cells (Huh7/Clone A) were routinely maintained in Clone A growth medium (DMEM medium [Invitrogen], supplemented with 10% Fetal Bovine Serum, 1% Non Essential Amino Acids and 1 g/L G418). Test compounds were dissolved in dimethyl sulfoxide (DMSO) to make 200× stock solutions for all doses prior to treatment.

For the HCV replicon assay, Huh7/Clone A cells were trypsinized from culture flasks, seeded in 1 ml of Clone A growth medium without G418 at 4×10⁴ cells per well in 24-well plates and incubated at 37° C. in a humidified CO₂ (5%) incubator overnight. Following overnight incubation, compound solutions were added into wells in the same volume (5 μl of 200× compound stock per well) to give a final DMSO concentration of 0.5%. Three wells on each plate supplemented with 5 μl of DMSO served as untreated controls. For IC₅₀ determinations, compounds were tested at 7 serial dilutions in triplicates from the starting stock solutions. The plates were incubated at 37° C. for 48 hours. After incubation, cells were harvested, transferred to 96-well plates, and subjected to total RNA extraction using the RNA Isolation Kit (RNeasy 96, Qiagen) according to the protocol described by the manufacture's RNeasy 96 Handbook (Qiagen).

Total RNA eluted in 130 μl of RNase-free dH₂O was quantitated by the RiboGreen Assay according to the supplier's protocol (Molecular Probe). Briefly, 5 μl of RNA samples were aliquoted in duplicate to a 96-well black microplate and a 96-well TaqMan Optical plate. RNA samples in the black microplate were mixed with 95 μl of diluted RiboGreen:reagent (1:250 dilution in TE buffer) and sample fluorescence was measured using a fluorescence microplate reader at standard fluorescein wavelengths (excitation ˜480 nm, emission ˜520 nm). Ribosomal RNA (Molecular Probe) was used as standard.

TaqMan quantitative PCR (RT-qPCR) was used to quantitate the amount of HCV replicon RNA in each sample. The RT-qPCR reactions were performed in 25 μl on an ABI PRISM 7900 HT Sequence Detection System (Applied Biosystems). The reaction mixture contained 5 μl of total RNA (10-100 ng), 1× TaqMan Buffer A (Applied Biosystems), 5.5 mM MgCl₂, 1.2 mM dNTP mix, 0.625 U of AmpliTaq Gold (Applied Biosystems), 5 U of MMLV reverse transcriptase (Promega), 5 U of rRNasin (Promega), 300 nM each of the forward and reverse primers, and 100 nM TaqMan MGB probe. Primers and probe were designed to hybridize to a portion of the neomycin resistance gene (neo) in the replicon and the sequences are as follows: forward primer 5′-GGCTACCTGCCCATTCGA-3′; reverse primer 5′-CCGGCTTCCATCCGAGTAC-3′; MGB probe 5′-CCACCAAGCGAAACA-3′. The RT step was performed at 48° C. for 30 min, followed by 10 min at 95° C. The thermal cycling program consisted of 40 cycles of 15 s at 95° C. and min at 60° C. TaqMan raw data (Ct values) were analyzed using the Sequence Detection System (SDS) software, mathematically converted to HCV RNA genome amount and normalized to total RNA in each sample. The sample without compound treatment served as a control and the HCV replicon RNA level from untreated cells was defined as 100%. Compound inhibitory activity was determined as the ratio of the normalized HCV RNA amount in treated samples relative to the untreated control. Compound IC₅₀s were calculated using a standard 4 parameter curve fit model.

Compounds of the invention that were tested by the above-described assay inhibit HCV replication at IC₅₀ levels or estimated IC₅₀ levels indicated by:

IC₅₀<0.50 uM: ++++

IC₅₀<2.0 uM: +++

IC₅₀<10.0 uM: ++

IC₅₀>10.0 uM: +

Activity levels are provided in the Examples above, immediately following the compound number, in the following format: e.g. (Compound 78: +++) and indicate activity levels in either or both of the HCV replicon assays.

HCV Replicon Assay 2

The HCV replicon assay is a cell-culture system that mimics in vivo HCV replication and provides a system to study HCV replication in vitro. The assay used the cell line ET (luc-ubi-neo/ET) which is a human Huh7 hepatoma cell line containing an HCV genotype 1b RNA replicon with a stable Lucifer ease (Luc) reporter gene and three cell culture-adaptive mutations (Pietschmann et al. 2002. Persistent and transient replication of full-length hepatitis C virus genomes in cell culture. J. Virol. 76:4008-4021). Replicon-containing cells autonomously and persistently replicate HCV RNA that can be measured quantitatively by real-time qPCR. Alternatively, the luciferase activity derived from the replicon-expressed luciferase gene can be used as an indirect measure of HCV RNA replication. The activity of the Luc reporter is directly proportional to the HCV RNA levels and positive control antiviral compounds behave comparably using either the Luc reporter or replicon RNA endpoints. The replicon system facilitates quantitative assessment of anti-viral activity by monitoring changes in HCV RNA replication in a cell-based assay. The replicon used in this assay contains the 5′ end of the HCV genome (with an Internal Ribosome Entry Site IRES and the first few amino acids of the HCV core protein) which drives the production of a Luciferase (Luc), ubiquitin (ubi), and neomycin phosphotransferase fusion protein. The IRES element controls the translation of the HCV structural proteins NS3 through NS5. The 3′ end of the replicon is the authentic 3′ nontranslated region (NTR) of HCV.

Test compounds were dissolved in dimethyl sulfoxide (DMSO) to make 10 mM and 3 mM stock solutions for all doses prior to treatment. For each assay, the antiviral activity of human interferon alpha-2b was tested as a positive control, and DMSO vehicle alone used as a negative control.

For the HCV replicon assay, sub-confluent cultures of the Huh7 ET replicon cell line were plated into 96-well plates dedicated for the analysis of cell numbers (cytotoxicity assay) or antiviral activity (Luc assay). The next day compounds were added to the appropriate wells. For IC₅₀ determinations, compounds were tested at 5 to 8 half-log serial dilutions in triplicates from the starting stock solutions. Cells are assayed for cytoxicity or Luc activity 72 hours after compound addition.

HCV RNA replicon levels were assessed as either HCV RNA replicon-derived Luc activity or as HCV RNA by Taqman RT-PCR. Data was used to derive IC₅₀ (compound concentration at which virus replication was inhibited by 50% from maximal level), IC₉₀ (compound concentration at which virus replication was inhibited by 90%), CC₅₀ (concentration decreasing cell viability by 50%), and TI (therapeutic index, CC₅₀/IC₅₀). The toxic concentration of compound that reduces cell viability was assessed using the calorimetric CytoTox-1 cell proliferation assay (Promega). It is also used as an indication of total cell numbers for the Luciferase assay. Total ribosomal RNA levels are determined by Taqman RT-PCR as an indication of cell numbers in the replicon RNA-based assay. Compound IC₅₀ levels were calculated using a standard 4 parameter curve fit model.

Compounds of the invention were tested by the above-described assays and observed to inhibit HCV replication.

Compounds of the invention that were tested by the above-described assay inhibit HCV replication at IC₅₀ levels or estimated IC₅₀ levels indicated by:

IC₅₀<0.50 uM: ++++

IC₅₀<2.0 uM: +++

IC₅₀<10.0 uM: ++

IC₅₀>10.0 uM: +

### Formulation Example 1

Representative pharmaceutical formulations containing a Compound of Formula I, II, III or IV:

Oral Formulation

| | | |
|---|---|---|
| Compound of Formula I, II, III or IV | 10-100 | mg |
| Citric Acid Monohydrate | 105 | mg |
| Sodium Hydroxide | 18 | mg |
| Flavoring | | |
| Water | q.s. to 100 | mL |

Intravenous Formulation

| Compound of Formula I, II, III or IV | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

Tablet Formulation

| | |
|---|---|
| Compound of Formula I, II, III or IV | 1% |
| Microcrystalline Cellulose | 73% |
| Stearic Acid | 25% |
| Colloidal Silica | 1% |

The foregoing invention has been described in some detail by way of illustration and example, for purposes of clarity and understanding. It will be apparent to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of Formula I, II or III: 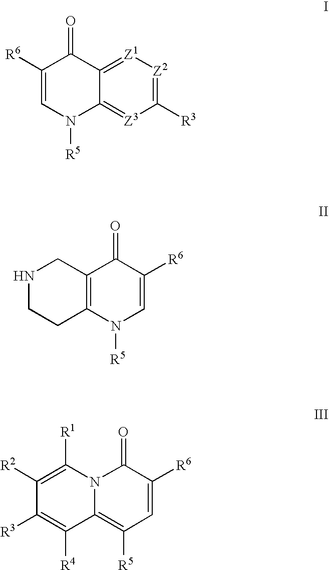
wherein
Z¹, Z² and Z³ are CR¹, N and N, respectively, or
Z¹, Z² and Z³ are N, CR² and N, respectively, or
Z¹, Z² and Z³ are CR¹, N and CR⁴, respectively, or
Z¹, Z² and Z³ are CR¹, CR² and N, respectively, or
Z¹, Z² and Z³ are CR¹, CR² and CR⁴, respectively;
R¹ is halo, —R⁸ or —OR⁸, where R⁸ is hydrogen, alkyl or halo-alkyl, or R¹ together with R² form methylenedioxy or ethylenedioxy;
R² is as defined above or is hydrogen, halo, alkyl, halo-alkyl, —OR⁹, —X²COOR⁹, —NR¹⁰C(O)NR⁹R¹⁰ or —X²C(O)NR⁹R¹⁰, where X² is a bond or alkylene, R⁹ is hydrogen, alkyl, halo-alkyl or heterocycloalkylalkyl and R¹⁰ is hydrogen or alkyl, or R² and R³ together form methylenedioxy, ethylenedioxy, 1,3-diazaprop-1-enylene, 1,3-diazaprop-2-enylene, 1-azaprop-2-enylene or 3-azaprop-1-enylene;
R³ is as defined above or is halo, —X³R¹¹, —X³OR¹², —X³C(O)R¹³, —X³C(O)OR¹², —X³OC(O)R¹³, —X³C(O)OR¹², —X³NR¹⁴R¹², —X³NR¹⁵R¹⁶, —X³OR¹⁵, —X³SR¹⁵, —X³NR¹⁴C(O)R¹³, —X³C(O)NR¹²R¹⁴, —X³S(O)₀₋₂R¹², —X³S(O)₂NR¹²R¹⁴, —X³NR¹⁴C(O)NR¹²R¹⁴ or —X³NR¹⁴C(O)OR¹², wherein X³ is a bond or alkylene, R¹² is hydrogen, alkyl or —X⁴R¹¹, R¹³ is alkyl or —X⁴R¹¹, R¹⁴ is hydrogen or alkyl and R¹⁵ is hydrogen, alkyl or —X⁵R¹⁷ and R¹⁶ is —X⁵R¹⁷, wherein X⁴ is a bond or alkylene, X⁵ is lower alkylene, R¹¹ is aryl, heteroaryl, cycloalkyl or heterocycloalkyl and R¹⁷ is —NR¹⁸R¹⁹, —C(O)OR¹⁸, —C(O)NR¹⁸R¹⁸, —OR¹⁹ or —SR¹⁹, wherein R¹⁸ is hydrogen or alkyl and R¹⁹ is hydrogen, alkyl or —NR²⁰R²¹, —OR²⁰ or —SR²⁰, wherein R²⁰ and R²¹ independently are hydrogen or alkyl, or R³ and R⁴ together form methylenedioxy or ethylenedioxy, wherein within R¹¹ any aromatic or alicyclic ring may be substituted with one R²² and within R³ any aliphatic moiety may be substituted with one to three halo, any alicyclic ring may be substituted with one to three R²³ and any aromatic ring may be substituted with one to three R²⁴;
R⁴ is as defined above or is halo, —R²⁵ or —OR²⁵, where R²⁵ is hydrogen, alkyl or halo-alkyl, or R⁴ together with R⁵ form ethylene, trimethylene or heteroalkylene which may be substituted with one R²⁶;
R⁵ is as defined above or is alkyl or —X⁷R²⁷ where X⁷ is —N—, lower alkylene or cycloprop-1,1-ylene and R²⁷ is cycloalkyl, heterocycloalkyl, heteroaryl or aryl, where within R⁵ any aliphatic moiety may be substituted with one to three halo, any alicyclic ring may be substituted with one to three R²⁸ and any aromatic ring may be substituted with one to three R²⁹;
R⁶ is selected from: 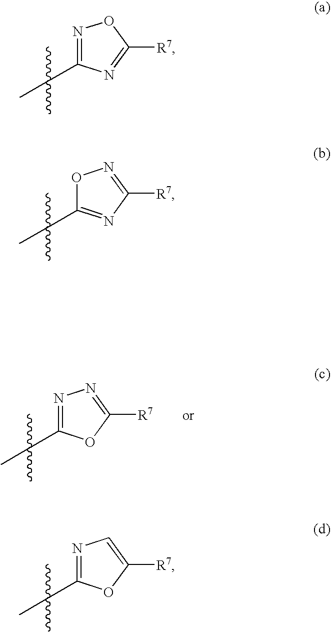
wherein
R⁷ is isopropyl, tert-butyl, pentyl or —X⁸R³⁰, where X⁸ is methylene, —C(CH₃)₂—, —C(CH₂OH)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring and wherein within R⁷ any aliphatic moiety may be substituted with one to three halo;
R²² is aryl, heteroaryl, cycloalkyl or heterocycloalkyl;
R²³ and R²⁸ at each occurrence independently are alkyl, halo-alkyl, hydroxy, alkoxy, acetyl or oxo;
R²⁴ and R²⁹ at each occurrence independently are alkyl, halo-alkyl, hydroxy, alkoxy or acetyl;
R²⁶ is —X⁷R²⁷ where X⁷ and R²⁷ are as defined above;
R³³ at each occurrence independently are methyl or halo; and
R³⁴ is lower alkyl, —CH₂COOR³⁵, —CH₂CONHR³⁵ or —CH₂NH₂ wherein R³⁵ is hydrogen or alkyl; or a pharmaceutically acceptable salt thereof.

2. A compound of claim 1, having formula I.

3. A compound of claim 2, wherein Z¹, Z² and Z³ are CR¹, CR² and CR⁴, respectively.

4. A compound of claim 3, wherein R⁵ is —X⁷R²⁷ wherein X⁷ is lower alkylene and R²⁷ is phenyl, and wherein the phenyl is optionally substituted with one to three R²⁹.

5. A compound of claim 4, wherein R⁶ has subformula (a).

6. A compound of claim 5, wherein R⁷ is —X⁸R³⁰, where X⁸ is —CH₂—, —C(CH₃)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring.

7. A compound of claim 6, wherein R⁷ is —X⁸R³⁰, where X⁸ is —C(CH₃)₂—, or cycloprop-1,1-ylene, and R³⁰ is phenyl, optionally substituted with one to two R³³ at any position on the ring, or with one R³⁴ at the 2- or 3-position on the ring.

8. A compound of claim 7, wherein R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

9. A compound of Formula IV: 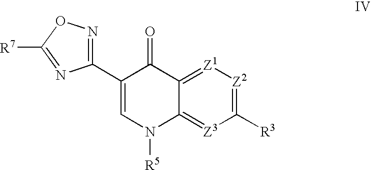
in which:
Z¹, Z² and Z³ are CR¹, N and N, respectively, or
Z¹, Z² and Z³ are N, CR² and N, respectively, or
Z¹, Z² and Z³ are CR¹, N and CR⁴, respectively, or
Z¹, Z² and Z³ are CR¹, CR² and N, respectively, or
Z¹, Z² and Z³ are CR¹, CR² and CR⁴, respectively,
R¹ is halo, —R⁸ or —OR⁸, where R⁸ is hydrogen, alkyl or halo-alkyl, or R¹ together with R² form methylenedioxy or ethylenedioxy;
R² is as defined above or is hydrogen, halo, alkyl, halo-alkyl, —OR⁹, —X²COOR⁹, —NR¹⁰C(O)NR⁹R¹⁰ or —X²C(O)NR⁹R¹⁰, where X² is a bond or alkylene, R⁹ is hydrogen, alkyl, halo-alkyl or heterocycloalkylalkyl and R¹⁰ is hydrogen or alkyl, or R² and R³ together form methylenedioxy, ethylenedioxy, 1,3-diazaprop-1-enylene, 1,3-diazaprop-2-enylene, 1-azaprop-2-enylene or 3-azaprop-1-enylene;
R³ is as defined above or is halo, —X³R¹¹, —X³OR¹², —X³C(O)R¹³, —X³C(O)OR¹², —X³OC(O)R¹³, —X³C(O)OR¹², —X³NR¹⁴R¹², —X³NR¹⁵R¹⁶, —X³OR¹⁵, —X³SR¹⁵, X³NR¹⁴C(O)R¹³, —X³C(O)NR¹²R¹⁴, —X³S(O)₀₋₂R¹², —X³S(O)₂NR¹²R¹⁴, —X³NR¹⁴C(O)NR¹²R¹⁴ or —X³NR¹⁴C(O)OR¹², wherein X³ is a bond or alkylene, R¹² is hydrogen, alkyl or —X⁴R¹¹, R¹³ is alkyl or —X⁴R¹¹, R¹⁴ is hydrogen or alkyl and R¹⁵ is hydrogen, alkyl or —X⁵R¹⁷ and R¹⁶ is —X⁵R¹⁷, wherein X⁴ is a bond or alkylene, X⁵ is lower alkylene, R¹¹ is aryl, heteroaryl, cycloalkyl or heterocycloalkyl and R¹⁷ is —NR¹⁸R¹⁹, —C(O)OR¹⁸, —C(O)NR¹⁸R¹⁸, —OR¹⁹ or —SR¹⁹, wherein R¹⁸ is hydrogen or alkyl and R¹⁹ is hydrogen, alkyl or —NR²⁰R²¹, —OR²⁰ or —SR²⁰ wherein R²⁰ and R²¹ independently are hydrogen or alkyl, or R³ and R⁴ together form methylenedioxy or ethylenedioxy, wherein within R¹¹ any aromatic or alicyclic ring may be substituted with one R²² and within R³ any aliphatic moiety may be substituted with one to three halo, any alicyclic ring may be substituted with one to three R²³ and any aromatic ring may be substituted with one to three R²⁴;
R⁴ is as defined above or is halo, —R²⁵ or —OR²⁵, where R²⁵ is hydrogen, alkyl or halo-alkyl, or R⁴ together with R⁵ form ethylene, trimethylene or heteroalkylene which may be substituted with one R²⁶;
R⁵ is as defined above or is alkyl or —X⁷R²⁷ where X⁷ is —NH—, lower alkylene or cycloprop-1,1-ylene and R²⁷ is cycloalkyl, heterocycloalkyl, heteroaryl or aryl, where within R⁵ any aliphatic moiety may be substituted with one to three halo, any alicyclic ring may be substituted with one to three R²⁸ and any aromatic ring may be substituted with one to three R²⁹;
R⁷ is isopropyl, tert-butyl, pentyl or —X⁸R³⁰, where X⁸ is methylene, —C(CH₃)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring and wherein within R⁷ any aliphatic moiety may be substituted with one to three halo;
R²² is aryl, heteroaryl, cycloalkyl or heterocycloalkyl;
R²³ and R²⁸ at each occurrence independently are alkyl, halo-alkyl, hydroxy, alkoxy, acetyl or oxo;
R²⁴ and R²⁹ at each occurrence independently are alkyl, halo-alkyl, hydroxy, alkoxy or acetyl;
R²⁶ is —X⁷R²⁷ where X⁷ and R²⁷ are as defined above;
R³³ at each occurrence independently are methyl or halo; and
R³⁴ is lower alkyl, —CH₂COOR³⁵, —CH₂CONHR³⁵ or —CH₂NH₂ wherein R³⁵ is hydrogen or alkyl; or a pharmaceutically acceptable salt thereof.

10. A compound of claim 9, wherein:
R¹ hydrogen and R⁴ are hydrogen;
R² is hydrogen, halo, alkyl, halo-alkyl, —OR⁹, —X²COOR⁹, —NR¹⁰C(O)NR⁹R¹⁰ or —C(O)NR⁹R¹⁰, where X² is a bond or alkylene, R⁹ is hydrogen, alkyl, halo-alkyl or heterocycloalkylalkyl and R¹⁰ is hydrogen or alkyl;
R³ is hydrogen, halo, —NHR³⁶ or —N(R³⁷)₂, wherein R³⁶ is alkyl, alkyloxycarbonylalkyl, carbamoylalkyl, heterocylcoalkylalkyl, hydroxyalkoxyalkyl, aminoalkyl, hydroxyalkyl and R³⁷ is hydrogen, alkyl or hydroxyalkyl, or each R³⁷ together with the nitrogen atom to which they are attached form heterocycloalkyl or heteroaryl, wherein heterocycloalkyl may be substituted with one to three groups independently selected from alkyl, hydroxy, acetyl, heteroaryl, carboxy and oxo and heteroaryl may be substituted with one to three groups independently selected from alkyl, hydroxy, acetyl, heteroaryl and carboxy;
R⁵ is alkyl or —X⁶R²⁷ where X⁶ is lower alkylene or cycloprop-1,1-ylene and R²⁷ is cycloalkyl, heterocycloalkyl, heteroaryl or aryl, wherein aryl and heteroaryl may be substituted with one to three halo or halo-alkyl; and
R⁷ is isopropyl, tert-butyl, pentyl or —X⁸R³⁰, where X⁸ is methylene, dimethylmethylene, cycloprop-1,1-ylene or —CHR³¹—, wherein R³¹ is —X⁹R³² wherein X⁹ is alkylene and R³² is carboxy, carbamoyl, cyano, aminoacetylamino or tert-butoxycarbonylamino and R³⁰ is cycloalkyl, heterocycloalkyl, phenyl or heteroaryl, wherein within R³⁰ phenyl may be substituted with one to two groups independently selected from halo and methyl at any position on the ring, cycloalkyl may be substituted with one —CH₂COOR³⁵ at the 1-, 2- or 3-position on the ring and phenyl with one lower alkyl, halo, —CH₂COOR³⁵ or —CH₂NH₂ at the 2- or 3-position on the ring, wherein R³⁵ is hydrogen or alkyl; or a pharmaceutically acceptable salt thereof.

11. A compound of claim 10, wherein:
R¹ is hydrogen;
R² is carboxy, carboxymethyl, fluoro, hydrogen, hydroxy, methoxy, N-(2-morpholin-4-ylethyl)carbamoyl or ureido;
R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl or 4-pyrid-2-ylpiperazin-1-yl;
R⁴ is hydrogen;
R⁵ is 4-chlorobenzyl, 2-chloro-6-fluorobenzyl, 4-chloro-2-fluorobenzyl, 2-chloropyrid-5-ylmethyl, cyclohexylmethyl, 2,4-difluorobenzyl, 2-ethylbutyl, 2-fluorobenzyl, 2-fluoro-4-trifluoromethylbenzyl, 2-morpholin-4-ylethyl, phenethyl, 3-trifluoromethyl benzyl 4-trifluoromethylbenzyl or 1-(4-trifluoromethylphenyl)cyclopropyl; and
R⁷ is 5-acetylamino-1-phenylpentyl, 2-aminomethylbenzyl, 5-amino-1-phenylpentyl, benzyl, 2-(tert-butoxycarbonylaminomethyl)benzyl, 5-tert-butoxycarbonylamino-1-phenylpentyl, tert-butylmethyl, (1-carbamoylmethylcyclohexyl)methyl, 2-carbamoyl-1-phenylethyl, 3-carbamoyl-1-phenylpropyl, 4-carbamoyl-1-phenylbutyl, 2-carboxymethylbenzyl, 4-carboxy-1-phenylbutyl, 2-cyano-1-phenylethyl, 4-cyano-1-phenylbutyl, 5-cyano-1-phenylpentyl, cyclohexylmethyl, cyclopentylmethyl, cyclopropylmethyl, 2,4-difluorobenzyl, 2,6-difluorobenzyl, 3,5-difluorobenzyl, 4-fluorobenzyl, isobutyl, 2-methylbenzyl, 3-methylbenzyl, pentyl, 1-methyl-1-phenylethyl, 1-phenylcyclopropyl, piperidin-1-ylmethyl, pyrid-2-ylmethyl, pyrid-3-ylmethyl, pyrrolidin-1-ylmethyl or thien-3-ylmethyl; or a pharmaceutically acceptable salt thereof.

12. A compound of claim 10, wherein
R¹ and R⁴ are hydrogen;
R² is fluoro;
R³ is —N(R³⁷)₂, wherein each R³⁷ together with the nitrogen atom to which they are attached form heterocycloalkyl wherein heterocycloalkyl may be substituted with alkyl, hydroxy, acetyl, heteroaryl, carboxy or oxo;
R⁵ is 4-chlorobenzyl, 4-chloro-2-fluorobenzyl, 2-chloropyrid-5-ylmethyl, 2-fluoro-4-trifluoromethylbenzyl, 4-trifluoromethylbenzyl or 1-(4-trifluoromethylphenyl)cyclopropyl; and
R⁷ is 5-acetyl-1-phenylpentyl, 5-amino-1-phenylpentyl, benzyl, 4-carbamoyl-1-phenylbutyl, 4-carboxy-1-phenylbutyl, 4-cyano-1-phenylbutyl, cyclopentylmethyl, 4-fluorobenzyl or 1-methyl-1-phenylethyl; or a pharmaceutically acceptable salt thereof.

13. A compound selected from:
3-(5-benzyl-[1,2,4]oxadiazol-3-yl)-6-fluoro-1-(2-fluoro-4-trifluoromethyl-benzyl)-7-(4-methyl-piperazin-1-yl)-1H-quinolin-4-one;
3-(5-benzyl-[1,2,4]oxadiazol-3-yl)-6-fluoro-1-(2-fluoro-4-trifluoromethyl-benzyl)-7-piperazin-1-yl-1H-quinolin-4-one;
5-{3-[6-fluoro-1-(2-fluoro-4-trifluoromethyl-benzyl)-7-(4-methyl-piperazin-1-yl)-4-oxo-1,4-dihydro-quinolin-3-yl]-[1,2,4]oxadiazol-5-yl}-5-phenyl-pentanenitrile;
6-fluoro-3-[5-(4-fluoro-benzyl)-[1,2,4]oxadiazol-3-yl]-7-(4-methyl-piperazin-1-yl)-1-[1-(4-trifluoromethyl-phenyl)-cyclopropyl]-1H-quinolin-4-one;
3-(5-benzyl-[1,2,4]oxadiazol-3-yl)-1-(4-chloro-benzyl)-6-fluoro-7-(4-hydroxy-piperidin-1-yl)-1H-quinolin-4-one;
N-(5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentyl)-acetamide;
3-(5-cyclopentylmethyl-[1,2,4]oxadiazol-3-yl)-6-fluoro-1-(2-fluoro-4-trifluoromethyl-benzyl)-7-(4-methyl-piperazin-1-yl)-1H-quinolin-4-one;
1-(4-chloro-2-fluoro-benzyl)-6-fluoro-3-[5-(4-fluoro-benzyl)-[1,2,4]oxadiazol-3-yl]-7-(4-methyl-piperazin-1-yl)-1H-quinolin-4-one;
3-[5-(5-amino-1-phenyl-pentyl)-[1,2,4]oxadiazol-3-yl]-1-(4-chloro-benzyl)-6-fluoro-7-(4-methyl-piperazin-1-yl)-1H-quinolin-4-one;
6-fluoro-3-[5-(4-fluoro-benzyl)-[1,2,4]oxadiazol-3-yl]-1-(2-fluoro-4-trifluoromethyl-benzyl)-7-(3-oxo-piperazin-1-yl)-1H-quinolin-4-one;
6-fluoro-3-[5-(4-fluoro-benzyl)-[1,2,4]oxadiazol-3-yl]-1-(2-fluoro-4-trifluoromethyl-benzyl)-7-(4-methyl-piperazin-1-yl)-1H-quinolin-4-one;
3-(5-benzyl-[1,2,4]oxadiazol-3-yl)-6-fluoro-7-piperazin-1-yl-1-(4-trifluoromethyl-benzyl)-1H-quinolin-4-one;
3-(5-benzyl-[1,2,4]oxadiazol-3-yl)-7-[1,4]diazepan-1-yl-6-fluoro-1-(4-trifluoromethyl-benzyl)-1H-quinolin-4-one;
1-(6-chloro-pyridin-3-ylmethyl)-6-fluoro-3-[5-(1-methyl-1-phenyl-ethyl)-[1,2,4]oxadiazol-3-yl]-7-(4-methyl-piperazin-1-yl)-1H-quinolin-4-one;
1-(4-chloro-benzyl)-6-fluoro-3-[5-(4-fluoro-benzyl)-[1,2,4]oxadiazol-3-yl]-7-(4-methyl-piperazin-1-yl)-1H-quinolin-4-one;
3-(5-benzyl-[1,2,4]oxadiazol-3-yl)-1-(4-chloro-benzyl)-6-fluoro-7-piperazin-1-yl-1H-quinolin-4-one;
6-fluoro-1-(2-fluoro-4-trifluoromethyl-benzyl)-3-[5-(1-methyl-1-phenyl-ethyl)-[1,2,4]oxadiazol-3-yl]-7-(4-methyl-piperazin-1-yl)-1H-quinolin-4-one;
5-{3-[6-fluoro-1-(2-fluoro-4-trifluoromethyl-benzyl)-7-(4-methyl-piperazin-1-yl)-4-oxo-1,4-dihydro-quinolin-3-yl]-[1,2,4]oxadiazol-5-yl}-5-phenyl-pentanamide;
7-[1,4]diazepan-1-yl-6-fluoro-3-[5-(1-methyl-1-phenyl-ethyl)-[1,2,4]oxadiazol-3-yl]-1-(4-trifluoromethyl-benzyl)-1H-quinolin-4-one;
3-(5-benzyl-[1,2,4]oxadiazol-3-yl)-7-[1,4]diazepan-1-yl-6-fluoro-1-(2-fluoro-4-trifluoromethyl-benzyl)-1H-quinolin-4-one; and
5-{3-[1-(4-chloro-benzyl)-6-fluoro-7-(4-methyl-piperazin-1-yl)-4-oxo-1,4-dihydro-quinolin-3-yl]-[1,2,4]oxadiazol-5-yl}-5-phenyl-pentanoic acid; or a pharmaceutically acceptable salt thereof.

14. A compound of claim 1, having formula I wherein Z¹, Z² and Z³ are CR¹, CR² and CR⁴, respectively; R⁵ is —X⁷R²⁷ wherein X⁷ is lower alkylene and R²⁷ is phenyl, and wherein the phenyl is optionally substituted with one to three R²⁹; R⁶ is subformula (b); and R⁷ is —X⁸R³⁰, where X⁸ is —CH₂—, —C(CH₃)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring.

15. A compound of claim 14, wherein R⁷ is —X⁸R³⁰, where X⁸ is —C(CH₃)₂—, or cycloprop-1,1-ylene, and R³⁰ is phenyl, optionally substituted with one to two R³³ at any position on the ring, or with one R³⁴ at the 2- or 3-position on the ring.

16. A compound of claim 15, wherein R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

17. A compound of claim 1, having formula I wherein Z¹, Z² and Z³ are CR¹, CR² and CR⁴, respectively; R⁵ is —X⁷R²⁷ wherein X⁷ is lower alkylene and R²⁷ is phenyl, and wherein the phenyl is optionally substituted with one to three R²⁹; R⁶ is subformula (c); and R⁷ is —X⁸R³⁰, where X⁸ is —CH₂—, —C(CH₃)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring.

18. A compound of claim 17, wherein R⁷ is —X⁸R³⁰, where X⁸ is —C(CH₃)₂—, or cycloprop-1,1-ylene, and R³⁰ is phenyl, optionally substituted with one to two R³³ at any position on the ring, or with one R³⁴ at the 2- or 3-position on the ring.

19. A compound of claim 18, wherein R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

20. A compound of claim 1, having formula I wherein Z¹, Z² and Z³ are CR¹, CR² and CR⁴, respectively; R⁵ is —X⁷R²⁷ wherein X⁷ is lower alkylene and R²⁷ is phenyl, and wherein the phenyl is optionally substituted with one to three R²⁹; R⁶ is subformula (d); and R⁷ is —X⁸R³⁰, where X⁸ is —CH₂—, —C(CH₃)₂—, cycloprop-1,1-ylene or —CHR³¹—, wherein R³¹ is —X⁹R³² wherein X⁹ is alkylene and R³² is carboxy, carbamoyl, cyano, amino, acetylamino or tert-butoxycarbonylamino and R³⁰ is cyclopentyl, cyclohexyl, heterocycloalkyl containing 5 to 6 ring atoms, phenyl or heteroaryl containing 5 to 6 ring atoms, wherein within R³⁰ phenyl may be substituted with one to two R³³ at any position on the ring, cycloalkyl may be substituted with one R³⁴ at the 1-, 2- or 3-position on the ring and phenyl may be substituted with one R³⁴ at the 2- or 3-position on the ring.

21. A compound of claim 20, wherein R⁷ is —X⁸R³⁰, where X⁸ is —C(CH₃)₂—, or cycloprop-1,1-ylene, and R³⁰ is phenyl, optionally substituted with one to two R³³ at any position on the ring, or with one R³⁴ at the 2- or 3-position on the ring.

22. A compound of claim 21, wherein R³ is 4-acetylpiperazin-1-yl, N-(2-aminoethyl)amino, 1,4-diazepan-1-yl, di(2-hydroxyethyl)amino, dimethylamino, hydrogen, N-[2-(2-hydroxyethoxy)ethyl]amino, 4-hydroxypiperidin-1-yl, imidazol-1-yl, methoxy, 4-methylimidazol-1-yl, 4-methylpiperazin-1-yl, N-methyl-N-methoxycarbonylamino, 1-methylureido, N-(2-morpholin-4-ylethyl)amino, 3-oxopiperazin-1-yl, piperazin-1-yl, 4-pyrid-2-ylpiperazin-1-yl, 1,1-dioxothiomorpholin-4-yl; and 1-oxothiomorpholin-4-yl.

23. A pharmaceutical composition comprising a compound of claim 1 or a pharmaceutically acceptable salt thereof in admixture with one or more suitable excipients.

24. A pharmaceutical composition of claim 23, further comprising a second antiviral agent.

25. A pharmaceutical composition of claim 24, wherein said second antiviral agent is Ribavirin or a protease inhibitor.

26. A method for treating hepatitis C in an animal which method comprises administering to the animal a pharmaceutical composition comprising a therapeutically effective amount of a compound of claim 1 or a pharmaceutically acceptable salt thereof in admixture with one or more suitable excipients.

27. A method of treating HCV infections in a subject, comprising administering to said subject a compound of claim 1 and a second antiviral agent selected from the group consisting of interferon, pegylated or unpegylated congeners of interferon, Ribavirin, a HCV protease inhibitor, an immunophillin antagonist and a toll receptor agonist.

28. A method of claim 27, wherein the protease inhibitor is VX-950.

29. A method of claim 27, wherein the protease inhibitor is Sch-503034.

30. A method of claim 27, wherein the protease inhibitor is ITMN-.

31. A process for preparing a target compound having the formula: 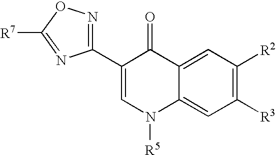
comprising
(a) reacting a compound of Formula 4: 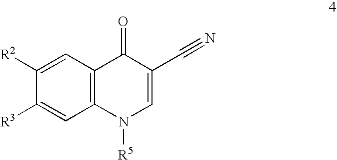
with hydroxyamine to produce a hydroxyamidine of Formula 5: 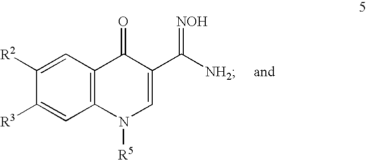
(b) reacting the hydroxyamidine of Formula 5 with an acid of the formula R⁷COOH to provide said target compound;
wherein each of R², R³, R⁵ and R⁷ are as defined in claim 1.
